(19) United States
(12) Patent Application Publication
(10) Pub. No.: US 2011/0318691 A1
(43) Pub. Date: Dec. 29, 2011

(54) RESIST COMPOSITION FOR SEMICONDUCTOR, AND RESIST FILM AND PATTERN FORMING METHOD USING THE SAME

(75) Inventors: Tomotaka TSUCHIMURA, Shizuoka (JP); Hideaki TSUBAKI, Shizuoka (JP); Takeshi KAWABATA, Shizuoka (JP)

(73) Assignee: FUJIFILM CORPORATION, Tokyo (JP)

(21) Appl. No.: 13/170,869

(22) Filed: Jun. 28, 2011

Related U.S. Application Data
(60) Provisional application No. 61/407,723, filed on Oct. 28, 2010.

(30) Foreign Application Priority Data
Jun. 29, 2010 (JP) 2010-148186
Oct. 18, 2010 (JP) 2010-233763

(51) Int. Cl. G03F 7/20; G03F 7/004; C07D 285/16; C07D 241/10; C07D 213/84; C07D 215/00; C07D 213/63; C07D 277/20; C07D 213/20; C07D 213/46

(52) U.S. Cl. 430/285.1; 546/347; 430/270.1; 544/5; 546/314; 546/286; 546/153; 546/290; 548/202; 544/336; 430/325

(57) ABSTRACT

An embodiment of the composition contains any of compounds of general formula (I) below:

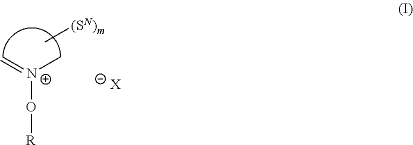

### CROSS-REFERENCE TO RELATED APPLICATIONS

This application claims the benefit of U.S. Provisional Application No. 61/407,723, filed Oct. 28, 2010.

This application is based upon and claims the benefit of priority from prior Japanese Patent Applications No. 2010-148186, filed Jun. 29, 2010; and No. 2010-233763, filed Oct. 18, 2010, the entire contents of both of which are incorporated herein by reference.

### BACKGROUND OF THE INVENTION

1. Field of the Invention

The present invention relates to a resist composition for semiconductor; and a resist film and a pattern forming method using the same. More specifically, the present invention relates to an actinic-ray- or radiation-sensitive resist composition suitable for manufacturing ICs, photomasks, and the like which may be used for semiconductor production.

In the present invention, the terms “actinic rays” and “radiation” mean, for example, a mercury lamp bright line spectrum, far ultraviolet rays represented by an excimer laser, extreme ultraviolet rays, X-rays, electron beams and the like. In the present invention, the term “light” means actinic rays or radiation.

The expression “exposure” used herein, unless otherwise noted, means not only light irradiation using a mercury lamp, far ultraviolet, X-rays, EUV light, etc. but also lithography using particle beams, such as an electron beam and an ion beam.

2. Description of the Related Art

A chemical amplification photosensitive composition is a pattern forming material that is capable of, upon exposure to far ultraviolet or other radiation, generating an acid at the exposed area and, by a reaction catalyzed by the acid, changing the solubility in a developer between the area having been exposed to actinic radiation and the nonexposed area to thereby attain pattern formation on a substrate.

In the use of a KrF excimer laser as an exposure light source, a resin whose fundamental skeleton consists of a poly(hydroxystyrene) exhibiting a low absorption mainly in the region of 248 nm is employed as a major component. Accordingly, there can be attained a high sensitivity, high resolving power and favorable pattern formation. Thus, a system superior to the conventional naphthoquinone diazide/novolak resin system is realized.

On the other hand, in the use of a light source of a further shorter wavelength, for example, an ArF excimer laser (193 nm) as an exposure light source, as the compounds having an aromatic group inherently exhibit a sharp absorption in the region of 193 nm, the above-mentioned chemical amplification system has not been satisfactory.

Therefore, resists for an ArF excimer laser containing a resin with an alicyclic hydrocarbon structure have been developed.

As for photoacid generator which is a main component of a chemical amplification resist, triphenylsulfonium salt is generally known (see, for example, patent reference 1 below).

However, the known acid generators are unsatisfactory in many respects. Thus, there is a demand in the art for the development of a photosensitive composition that is enhanced in the sensitivity, resolution, pattern configuration, roughness characteristic, etc. through the improvement of such acid generators.

In particular, the roughness characteristic becomes serious in accordance with the reduction of pattern dimension. In the field of, for example, the lithography using X-rays, electron beams or EUV, as the formation of a fine pattern of several tens of nanometers is targeted, the demand for excellent roughness characteristic is strong.

Moreover, the electron beam lithography utilized as a nanofabrication technology has also become indispensable as a method of fabricating a photomask blank used in the manufacturing of a photomask for semiconductor production.
[Patent reference 1] U.S. Pat. No. 6,548,221.

### BRIEF SUMMARY OF THE INVENTION

It is an object of the present invention to provide a resist composition for semiconductor; and a resist film and a pattern forming method using the same which excel in sensitivity and roughness characteristic.

Followings are some aspects of the present invention. In the present invention, the term “volume of an acid” means the volume of a region occupied by a van der Waals sphere based on the van der Waals radii of atoms constituting the acid. In particular, the term “volume of an acid” means the volume calculated in the following manner. Namely, first, the most stable conformation of the acid is determined by a molecular force field calculation using an MM3 method. Thereafter, a van der Waals volume is calculated by a molecular orbital calculation using a PM3 method with respect to this most stable conformation. This van der Waals volume is referred to as the “volume of an acid.”

[1] A resist composition for semiconductor comprising any of compounds of general formula (I) below:

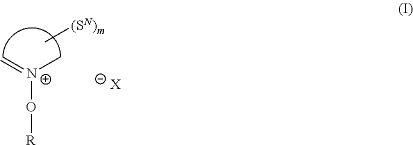

wherein

R represents a monovalent substituent,

the moiety:

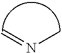

represents a monocyclic or polycyclic heterocycle containing a nitrogen atom,

S^{N} represents a substituent,

m is an integer of 0 or greater, and

X⁻ represents an anion.

[2] The composition according to [1], wherein the anion represented by X⁻ is selected from the group consisting of a sulfonate anion, an imidate anion and a methide anion.

[3] The composition according to [1] or [2], wherein each of the compounds of general formula (I) above is a compound that is configured to decompose when exposed to actinic rays or radiation to thereby generate an acid expressed by general formula HX, the acid having a volume of 130 Å³ or greater.

[4] The composition according to any of [1] to [3], wherein the anion represented by X⁻ is expressed by general formula (SA1) or (SA2) below:

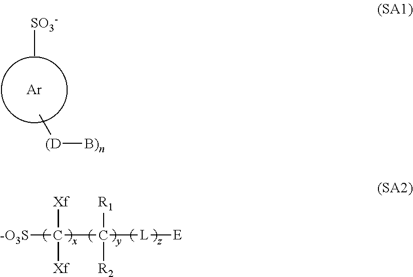

in formula (SA1),

Ar represents an aryl group, in which a substituent other than -(D-B) groups may further be introduced,

n is an integer of 1 or greater,

D represents a single bond or a bivalent connecting group, and

B represents a monovalent substituent, and

in formula (SA2),

each of Xf's independently represents a fluorine atom or an alkyl group having at least one hydrogen atom thereof replaced by a fluorine atom,

each of R₁ and R₂ independently represents a group, selected from the group consisting of a hydrogen atom, a fluorine atom, an alkyl group and an alkyl group having at least one hydrogen atom thereof replaced by a fluorine atom,

L, or each of L's independently, represents a single bond or a bivalent connecting group,

E represents a group with a cyclic structure, and

x is an integer of 1 to 20, y an integer of 0 to 10, and z an integer of 0 to 10.

[5] The composition according to any of [1] to [4], wherein the heterocycle is an aromatic ring.

[6] The composition according to any of [1] to [5], further comprising a resin that is configured to decompose when acted on by an acid to thereby increase its solubility in an alkali developer.

[7] The composition according to [6], wherein the resin contains any of repeating units of general formula (VI) below:

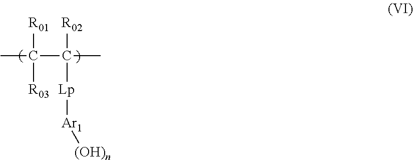

wherein

each of R₀₁, R₀₂ and R₀₃ independently represents a hydrogen atom, an alkyl group, a cycloalkyl group, a halogen atom, a cyano group or an alkoxycarbonyl group, provided that R₀₃ may represent an alkylene group and may be bonded to Lp or Ar₁ to thereby form a ring,

Ar₁ represents an aromatic ring group,

n is an integer of 1 or greater, and

Lp represents a single bond or a bivalent connecting group.

[8] The composition according to any of [1] to [5], further comprising a resin that is soluble in an alkali developer and an acid crosslinking agent that is configured to crosslink with the resin when acted on by an acid.

[9] The composition according to [8], wherein the resin contains any of repeating units of general formula (VI) below:

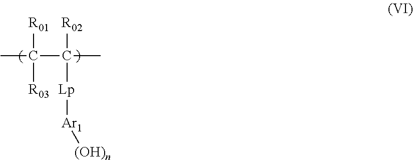

wherein

each of R₀₁, R₀₂ and R₀₃ independently represents a hydrogen atom, an alkyl group, a cycloalkyl group, a halogen atom, a cyano group or an alkoxycarbonyl group, provided that R₀₃ may represent an alkylene group and may be bonded to Lp or Ar₁ to thereby form a ring,

Ar₁ represents an aromatic ring group,

n is an integer of 1 or greater, and

Lp represents a single bond or a bivalent connecting group.

[10] The composition according to any of [1] to [9], further comprising a basic compound.

[11] The composition according to any of [1] to [10], which is to be exposed to electron beams, X-rays or EUV light.

[12] A resist film formed from the composition according to any of [1] to [11].

[13] A method of forming a pattern, comprising: forming the composition according to any of [1] to [11] into a film, exposing the film to light, and developing the exposed film.

[14] The method according to [13], wherein the exposure is performed using electron beams, X-rays or EUV light.

### DETAILED DESCRIPTION OF THE INVENTION

The embodiments of the present invention will be described in detail below.

Note that, with respect to the expression of a group (or an atomic group) used in this specification, the expression without explicitly referring to whether the group is substituted or unsubstituted encompasses not only groups with no substituents but also groups having one or more substituents. For example, the expression “alkyl group” encompasses not only alkyl groups having no substituents (viz. unsubstituted alkyl groups) but also alkyl groups having one or more substituents (viz. substituted alkyl groups).

The resist composition for semiconductor according to the present invention comprises [1] a compound that is configured to generate an acid when exposed to actinic rays or radiation (hereinafter also referred to as a photoacid generator).

This composition may be used as a positive composition or a negative composition.

In the use as a positive composition, the composition according to the present invention typically further contains [2] a resin that is configured to decompose when acted on by an acid to thereby increase its solubility in an alkali developer (hereinafter also referred to as an acid-decomposable resin). This composition may still further contain [4] a compound of 3000 or less molecular weight that when acted on by an acid, is decomposed to thereby increase its solubility in an alkali developer (hereinafter also referred to as a dissolution-inhibiting compound).

In the use as a negative composition, the composition according to the present invention may further contain [3] a resin soluble in an alkali developer (hereinafter also referred to as an “alkali-soluble resin”) and [5] an acid crosslinking agent capable of crosslinking with the above alkali-soluble resin under the action of an acid.

Moreover, the composition according to the present invention may still further contain at least one of [6] a compound that is configured to generate an acid by the action of an acid (hereinafter also referred to as an acid amplifier), [7] a basic compound, [8] a fluorinated and/or siliconized surfactant, [9] a hydrophobic resin, [10] an organic solvent and [11] other additives.

Each of these components will be explained in sequence below.

[1] Photoacid Generator

The composition of the present invention comprises, as a photoacid generator, any of compounds of general formula (I) below:

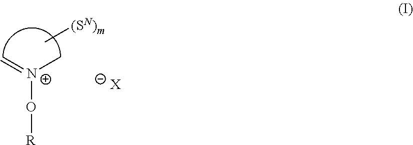

In the formula, R represents a monovalent substituent.

The moiety:

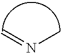

represents a monocyclic or polycyclic heterocycle containing a nitrogen atom. S^{N} represents a substituent, and m is an integer of 0 or greater. X⁻ represents an anion.

The inventors have found that the compounds of general formula (I) above exhibit a strikingly high acid generating efficiency. Further, the inventors have found that strikingly excellent sensitivity and roughness characteristics can be attained by using the composition comprising any of these compounds.

Still further, the inventors have found that these compounds exhibit an especially high acid generating efficiency when exposed to electron beams, X-rays or EUV light. Yet still further, the inventors have found that especially excellent sensitivity and roughness characteristics can be attained when the film formed from the composition comprising any of these compounds is exposed to electron beams, X-rays or EUV light.

In general formula (I), R represents a monovalent substituent. This substituent may be an organic group or an inorganic group. As this substituent, there can be mentioned, for example, an alkyl group, a cycloalkyl group, an aryl group, an alkenyl group, an alkynyl group, a substituted carbonyl group or a substituted sulfonyl group. A further substituent may be introduced in these substituent groups.

The alkyl group represented by R may be in the form of a linear or branched chain. This alkyl group preferably has 1 to 50 carbon atoms, more preferably 1 to 30 carbon atoms and further more preferably 1 to 20 carbon atoms. As such an alkyl group, there can be mentioned, for example, a methyl group, an ethyl group, a propyl group, a butyl group, a hexyl group, an octyl group, a decyl group, a dodecyl group, an octadecyl group, an isopropyl group, an isobutyl group, a sec-butyl group, a t-butyl group, a 1-ethylpentyl group or a 2-ethylhexyl group.

A substituent may be introduced in the alkyl group represented by R. Namely, R may be a substituted alkyl group. As such a substituted alkyl group, there can be mentioned, for example, a trifluoromethyl group, a phenacyl group, a 1-naphthoylmethyl group, a 2-naphthoylmethyl group, a 4-methylsulfanylphenacyl group, a 4-phenylsulfanylphenacyl group, a 4-dimethylaminophenacyl group, a 4-cyanophenacyl group, a 4-methylphenacyl group, a 2-methylphenacyl group, a 3-fluorophenacyl group, a 3-trifluoromethylphenacyl group, a 3-nitrophenacyl group, a chloromethyl group, a bromomethyl group, a 2-chloroethyl group, a methoxymethyl group, a methoxycarbonylmethyl group, an isopropoxymethyl group, a butoxymethyl group, an s-butoxybutyl group, a methoxyethoxyethyl group, an allyloxymethyl group, a phenoxymethyl group, an acetyloxymethyl group, a methylthiomethyl group, a tolylthiomethyl group, a pyridylmethyl group, a tetramethylpiperidinylmethyl group, an N-acetyltetramethylpiperidinylmethyl group, a trimethylsilylmethyl group, a methoxyethyl group, an ethylaminoethyl group, a diethylaminopropyl group, a morpholinopropyl group, a benzoyloxymethyl group, an N-cyclohexylcarbamoyloxyethyl group, an N-phenylcarbamoyloxyethyl group, an acetylaminoethyl group, an N-methylbenzoylaminopropyl group, a 2-oxoethyl group, a 2-oxopropyl group, a carboxypropyl group, a methoxycarbonylethyl group, an allyloxycarbonylbutyl group, a chlorophenoxycarbonylmethyl group, a carbamoylmethyl group, an N-methylcarbamoylethyl group, an N,N-dipropylcarbamoylmethyl group, an N-(methoxyphenyl)carbamoylethyl group, an N-methyl-N-(sulfophenyl)carbamoylmethyl group, a sulfobutyl group, a sulfonatobutyl group, a sulfamoylbutyl group, an N-ethylsulfamoylmethyl group, an N,N-dipropylsulfamoylpropyl group, an N-tolylsulfamoylpropyl group, an N-methyl-N-(phosphonophenyl)sulfamoyloctyl group, a phosphonobutyl group, a phosphonatohexyl group, a diethylphosphonobutyl group, a diphenylphosphonopropyl group, a methylphosphonobutyl group, a methylphosphonatobutyl group, a tolylphosphonohexyl group, a tolylphosphonatohexyl group, a phosphonooxypropyl group, a phosphonatooxybutyl group, a benzyl group, a phenethyl group, an α-methylbenzyl group, a 1-methyl-1-phenylethyl group or a p-methylbenzyl group.

As substituents that can be introduced in the alkyl groups represented by R, there can be mentioned, for example, not only the substituents set forth above in the description of substituted alkyl groups but also monovalent substituents composed of nonmetallic atoms whose examples are given below. As preferred examples containing these substituents, there can be mentioned a halogen atom (—F, —Br, —CI or —I), a hydroxyl group, an alkoxy group, an aryloxy group, a mercapto group, an alkylthio group, an arylthio group, an amino group, an acyloxy group, a carbamoyloxy group, an alkylsulfoxy group, an arylsulfoxy group, an acylthio group, an acylamino group, a ureido group, an alkoxycarbonylamino group, an aryloxycarbonylamino group, an N-alkyl-N-alkoxycarbonylamino group, an N-alkyl-N-aryloxycarbonylamino group, an N-aryl-N-alkoxycarbonylamino group, an N-aryl-N-aryloxycarbonylamino group, a formyl, group, an acyl group, a carboxyl group, a carbamoyl group, an alkylsulfinyl group, an arylsulfinyl group, an alkylsulfonyl group, an arylsulfonyl group, a sulfo group (—SO₃H) or its conjugated base group (referred to as a sulfonato group), an alkoxysulfonyl group, an aryloxysulfonyl group, a sulfinamoyl group, a phosphono group (—PO₃H₂) or its conjugated base group (referred to as a phosphonato group), a phosphonooxy group (—OPO₃H₂) or its conjugated base group (referred to as a phosphonatooxy group), a cyano group, a nitro group, an aryl group, an alkenyl group, an alkynyl group, a heterocyclic group and a silyl group.

As specific examples of the aryl groups that can be contained in the substituents that can be introduced in the alkyl groups represented by R, there can be mentioned a phenyl group, a biphenyl group, a naphthyl group, a tolyl group, a xylyl group, a mesityl group and a cumenyl group.

The cycloalkyl group represented by R may be monocyclic or polycyclic. This cycloalkyl group preferably has 3 to 50 carbon atoms, more preferably 4 to 30 carbon atoms and further more preferably 5 to 20 carbon atoms. As the cycloalkyl group, there can be mentioned, for example, a cyclopentyl group, a cyclohexyl group, an adamantyl group or a norbornyl group.

A further substituent may be introduced in the cycloalkyl group represented by R. As the further substituent, there can be mentioned, for example, any of those mentioned above as the substituents that can be introduced in the alkyl groups.

The aryl group represented by R may be monocyclic or polycyclic. This aryl group may be a heteroaryl group. The aryl group represented by R preferably has 6 to 50 carbon atoms, more preferably 6 to 30 carbon atoms and further more preferably 6 to 20 carbon atoms. As the aryl group, there can be mentioned, for example, a phenyl group, a biphenyl group, a 1-naphthyl group, a 2-naphthyl group, a 9-anthryl group, a 9-phenanthryl group, a 1-pyrenyl group, a 5-naphthacenyl group, a 1-indenyl group, a 2-azulenyl group, a 9-fluorenyl group, a terphenyl group, a quaterphenyl group, an o-, m- or p-tolyl group, a xylyl group, an o-, m- or p-cumenyl group, a mesityl group, a pentalenyl group, a binaphthalenyl group, a ternaphthalenyl group, a quaternaphthalenyl group, a heptalenyl group, a biphenylenyl group, an indacenyl group, a fluoranthenyl group, an acenaphthylenyl group, an aceanthrylenyl group, a phenalenyl group, a fluorenyl group, an anthryl group, a bianthracenyl group, a teranthracenyl group, a quateranthracenyl group, an anthraquinolyl group, a phenanthryl group, a triphenylenyl group, a pyrenyl group, a chrysenyl group, a naphthacenyl group, a pleiadenyl group, a picenyl group, a perylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group or an ovalenyl group.

A further substituent may be introduced in the aryl group represented by R. As the further substituent, there can be mentioned, for example, any of those mentioned above as the substituents that can be introduced in the alkyl groups.

The alkenyl group represented by R may be in the form of a linear or branched chain. This alkenyl group preferably has 2 to 50 carbon atoms, more preferably 2 to 30 carbon atoms and further more preferably 3 to 20 carbon atoms. A further substituent may be introduced in the alkenyl group.

As such an alkenyl group, there can be mentioned, for example, a vinyl group, an allyl group or a styryl group. As the further substituent that can be introduced in the alkenyl group, there can be mentioned, for example, any of those mentioned above as the substituents can be introduced in the alkyl groups.

The alkynyl group represented by R may be in the form of a linear or branched chain. This alkynyl group preferably has 2 to 50 carbon atoms, more preferably 2 to 30 carbon atoms and further more preferably 3 to 20 carbon atoms. A further substituent may be introduced in the alkynyl group.

As such an alkynyl group, there can be mentioned, for example, an ethynyl group, a propynyl group or a propargyl group. As the further substituent that can be introduced in the alkynyl group, there can be mentioned, for example, any of those mentioned above as the substituents can be introduced in the alkyl groups.

The substituted carbonyl group represented by R is any of the groups of general formula: —CO—R⁰¹³. R⁰¹³ represents a group composed of a monovalent nonmetallic atomic group.

As the substituted carbonyl group, there can be mentioned, for example, a formyl group, an acyl group, a carboxyl group, an alkoxycarbonyl group, an aryloxycarbonyl group or a carbamoyl group. As the alkyl group and aryl group contained in these groups, there can be mentioned, for example, those set forth above as the groups represented by R.

The substituted sulfonyl group represented by R is any of the groups of general formula: —SO₂—R⁰¹¹. R⁰¹¹ represents a group composed of a monovalent nonmetallic atomic group.

As the substituted sulfonyl group, there can be mentioned, for example, an alkylsulfonyl group, an arylsulfonyl group or a sulfamoyl group. The sulfamoyl group may be substituted or unsubstituted. As the alkyl group and aryl group contained in these groups, there can be mentioned, for example, those set forth above as the groups represented by R.

The heterocycle containing a nitrogen atom appearing in general formula (AZ) may be an aromatic ring or a nonaromatic ring. This heterocycle may further contain a heteroatom, such as a nitrogen atom, an oxygen atom or a sulfur atom, other than the nitrogen atom of the formula. Moreover, this heterocycle may be monocyclic or polycyclic as mentioned above.

As such a heterocycle, there can be mentioned, for example, an imidazole ring, a pyridine ring, a pyrazine ring, a pyrimidine ring, a pyridazine ring, a 2H-pyrrole ring, a 3H-indole ring, a 1H-indazole ring, a purine ring, an isoquinoline ring, a 4H-quinolizine ring, a quinoline ring, a phthalazine ring, a naphthyridine ring, a quinoxaline ring, a quinazoline ring, a cinnoline ring, a pteridine ring, a phenanthridine ring, an acridine ring, a phenanthroline ring, a phenazine ring, a perimidine ring, a triazine ring, a benzisoquinoline ring, a thiazole ring, a thiadiazine ring, an azepine ring, an azocine ring, an isothiazole ring, an isooxazole ring or a benzothiazole ring. Among these rings, a pyridine ring and a quinoline ring are especially preferred.

As mentioned above, S^{N} represents a substituent. As the substituent, there can be mentioned, for example, any of those set forth above in connection with R. A further substituent may be introduced in this substituent.

As mentioned above, m is an integer of 0 or greater. The upper limit of m is equal to the number of atoms that can be substituted with a substituent among the atoms constituting the heterocycle.

X⁻ represents an anion. The anion represented by X⁻ will be described in detail hereinafter.

The compounds of general formula (I) above are preferably expressed by general formula (II) or (III) below. Namely, the heterocycle containing a nitrogen atom appearing in general formula (I) preferably contains a 6-membered ring or a 5-membered ring.

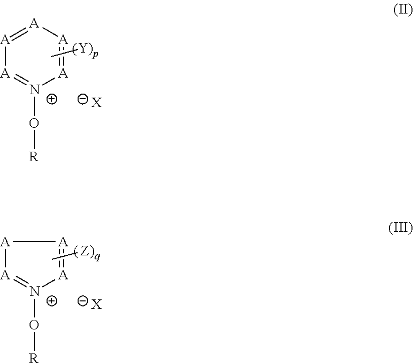

In the formula,

each of A's independently represents a carbon atom, a nitrogen atom, an oxygen atom or a sulfur atom.

Y, or each of Y's independently, represents a substituent. At least two of Y's may be bonded to each other to thereby form a ring, and p is an integer of 0 to 5.

Z, or each of Z's independently, represents a substituent. At least two of Z's may be bonded to each other to thereby form a ring, and q is an integer of 0 to 4.

R and X⁻ are as defined above in connection with general formula (I).

In both of general formulae (II) and (III), the number of A's each representing a nitrogen atom, an oxygen atom or a sulfur atom among all the A's is preferably in the range of 0 to 2, more preferably 0 or 1.

As particular examples of Y's and Z's, there can be mentioned those set forth above in connection with SN of general formula (I). At least two of Y's, or Z's, may be bonded to each other to thereby form a ring. Namely, each of the compounds of general formula (II) or (III) may have a condensed-ring structure.

The ring formed by the mutual bonding of Y's, or Z's may be an aromatic ring or a nonaromatic ring. This ring may be a heterocycle containing a heteroatom. The ring formed by the mutual bonding of Y's, or Z's is preferably a 5- to 7-membered ring, more preferably a 5- or 6-membered ring and most preferably a 6-membered ring.

Further, a substituent may be introduced in the ring formed by the mutual bonding of Y's or Z's. As the substituent, there can be mentioned, for example, any of those set forth above in connection with S^{N} of general formula (I).

When at least one of A's is a nitrogen atom, an oxygen atom or a sulfur atom, more preferably, the compounds of general formula (II) are expressed by general formula (II-1) or (II-2) below.

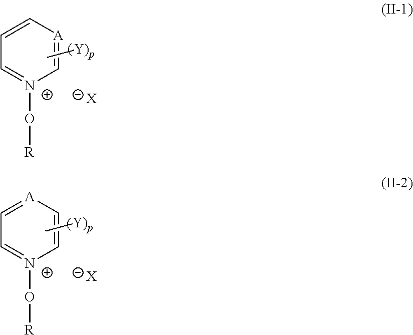

In general formulae (II-1) and (II-2), A represents a nitrogen atom, an oxygen atom or a sulfur atom. Y, p, R and X⁻ are as defined above in connection with general formula (II).

When at least one of A's is a nitrogen atom, an oxygen atom or a sulfur atom, more preferably, the compounds of general formula (III) are expressed by general formula (III-1) below.

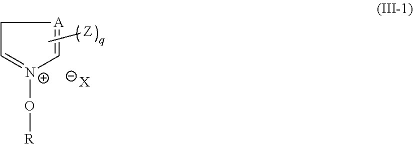

In general formula (III-1), A represents a nitrogen atom, an oxygen atom or a sulfur atom. Z, q, R and X− are as defined above in connection with general formula (III).

Specific examples of the cation moieties of the compounds of general formula (I) are shown below.

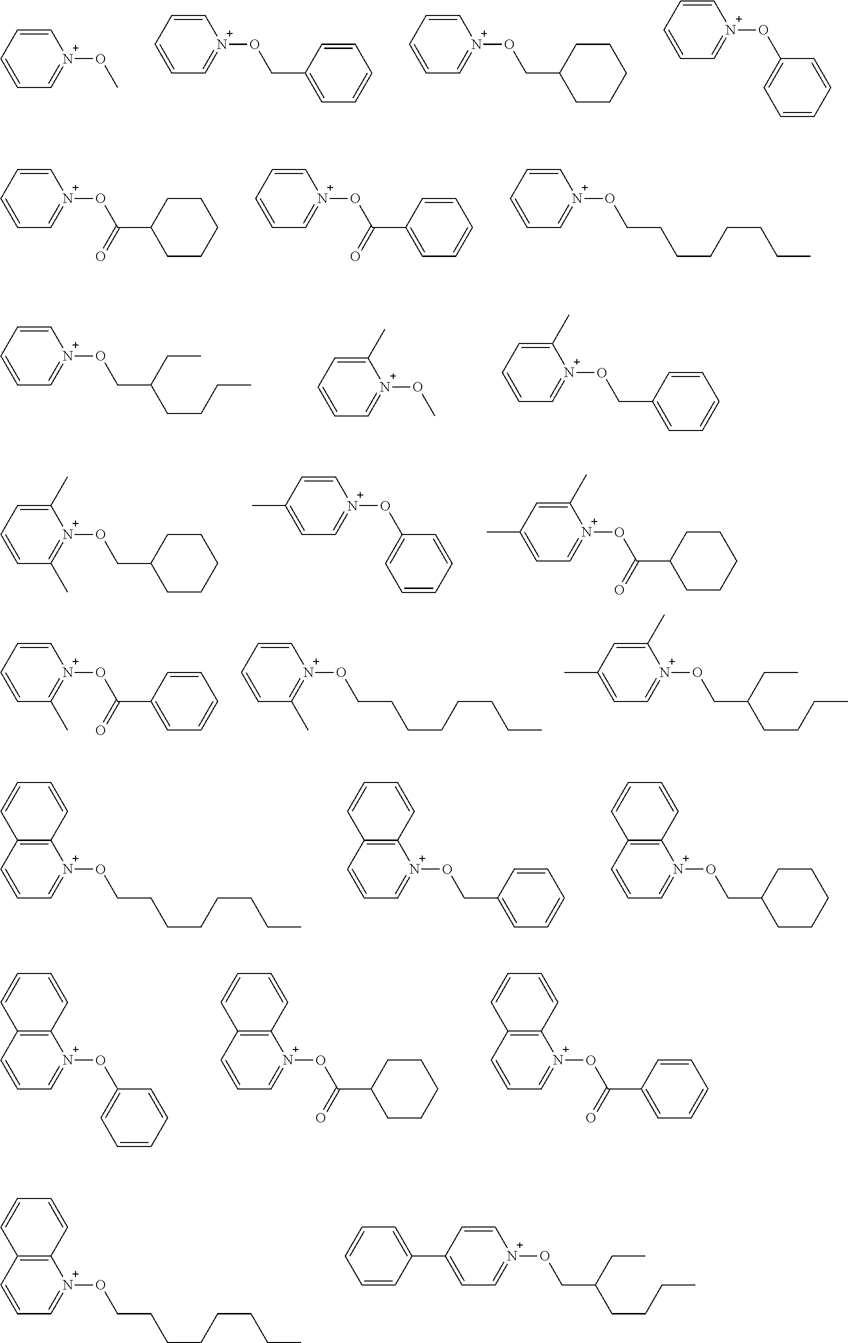

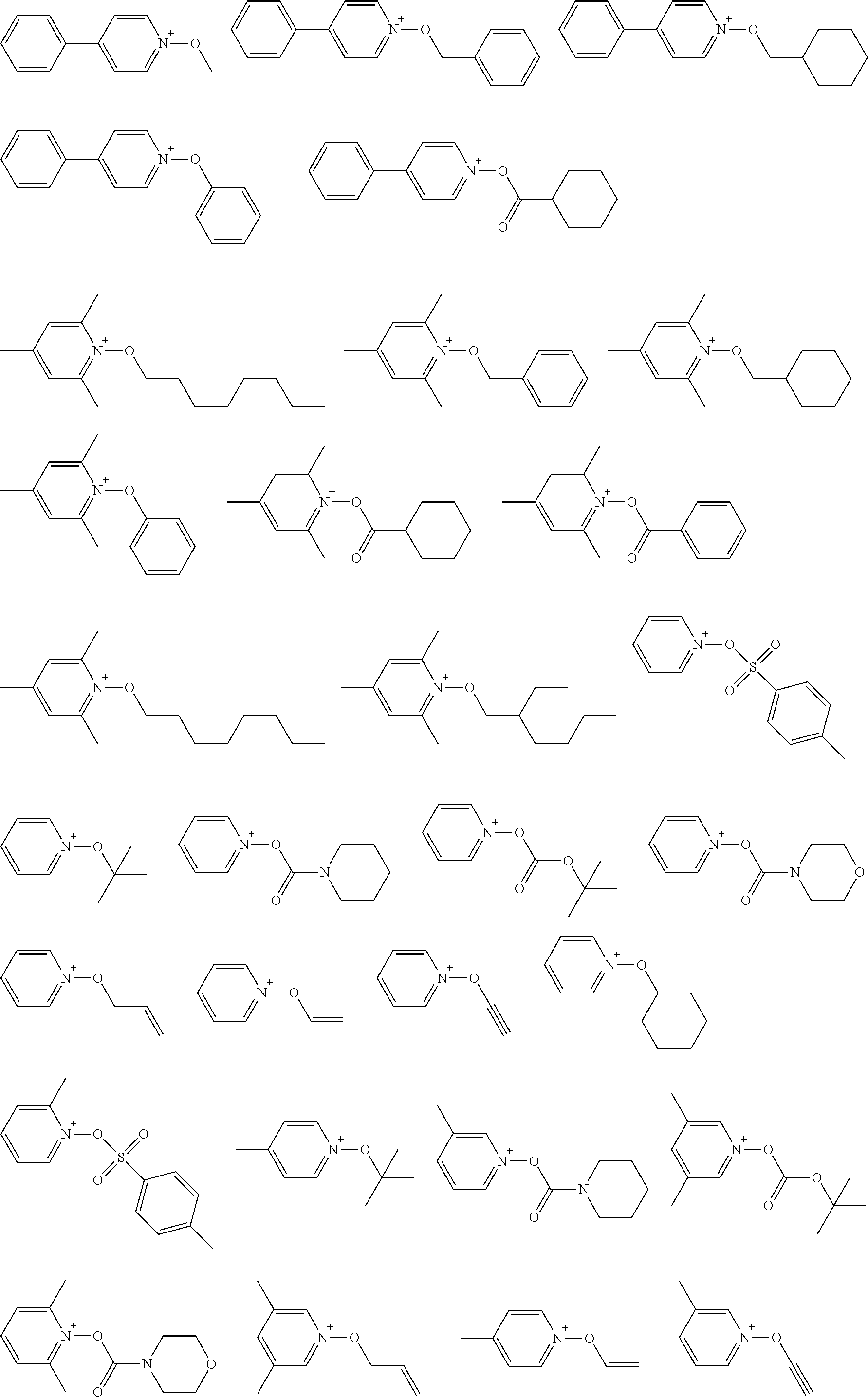

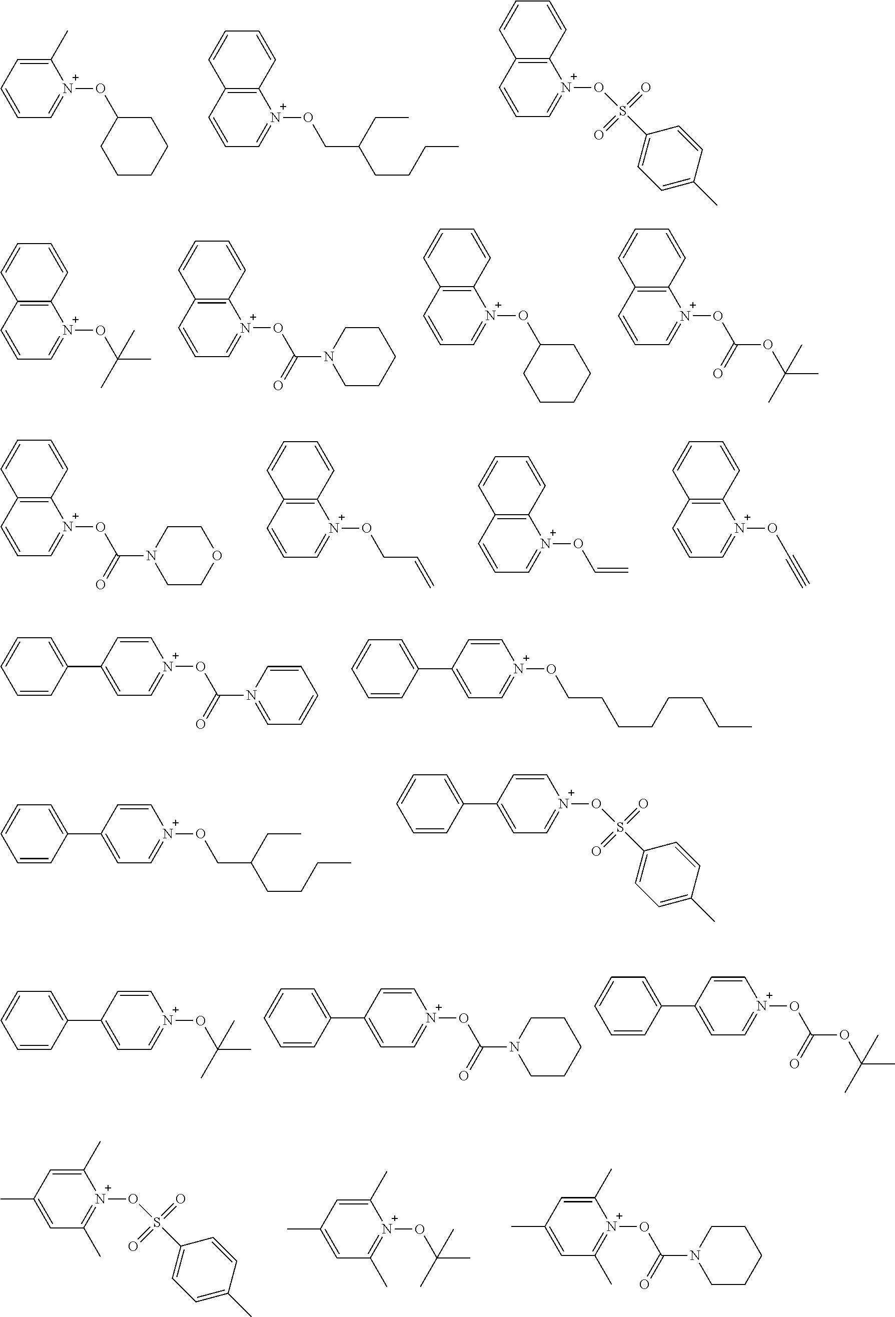

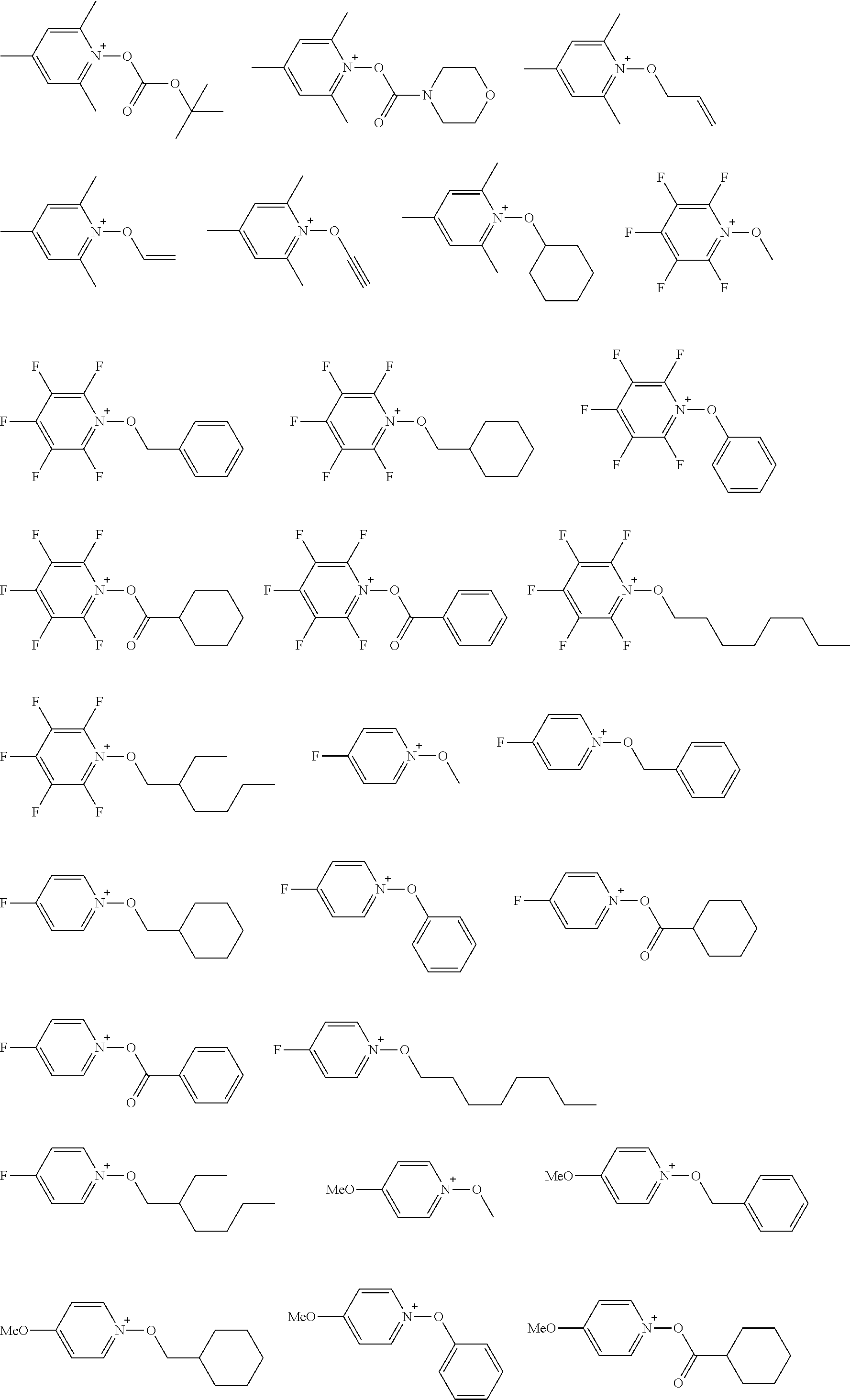

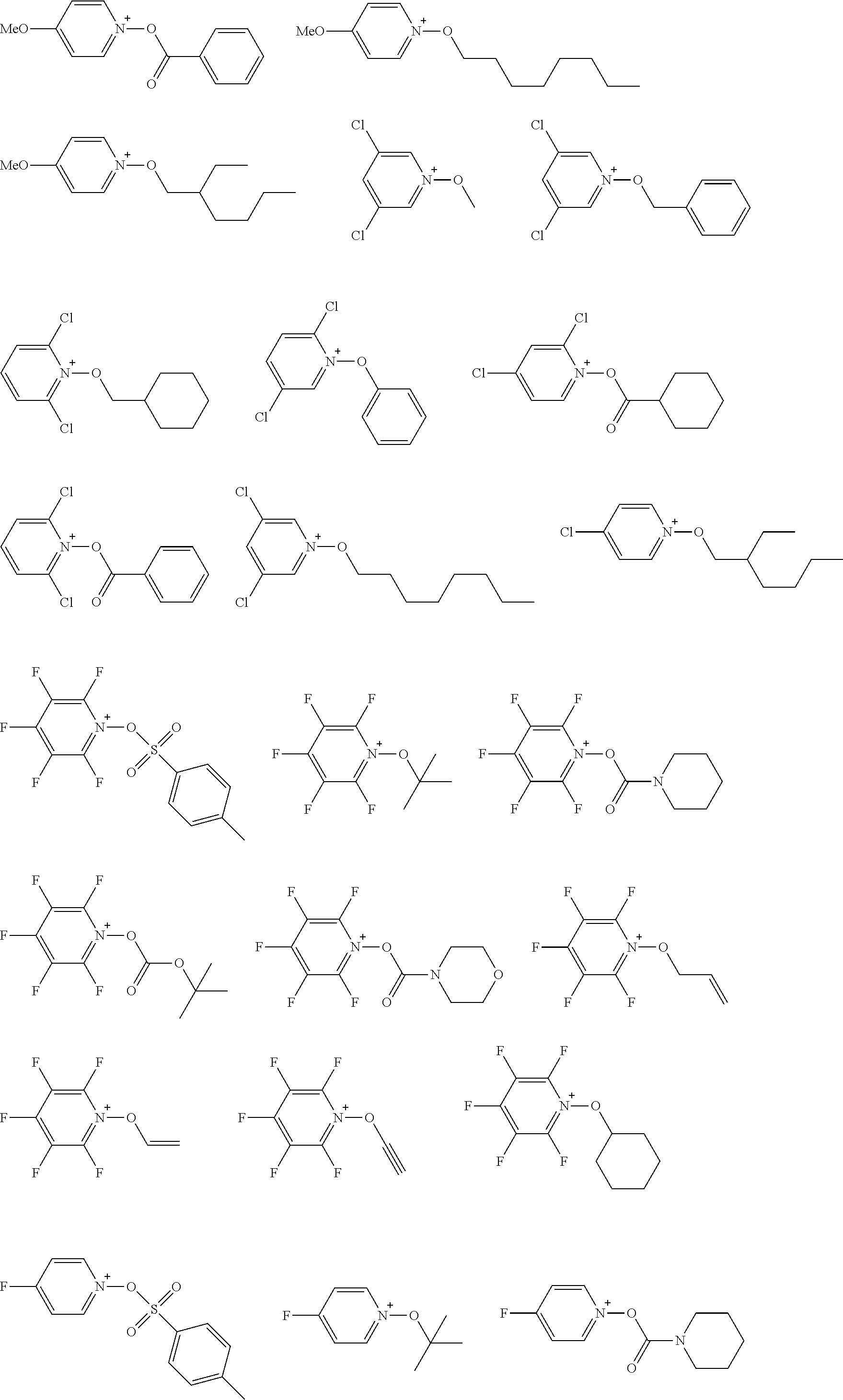

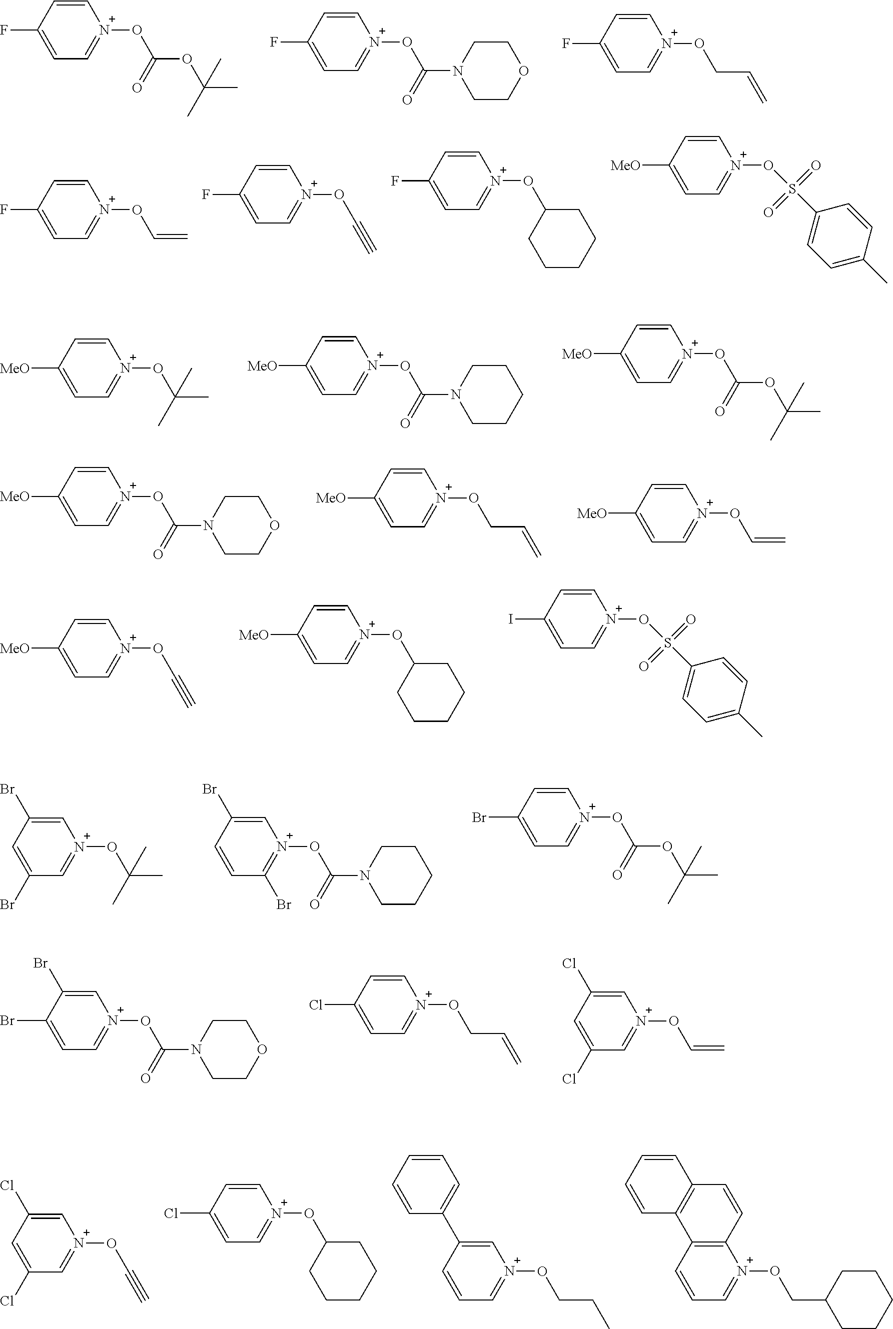

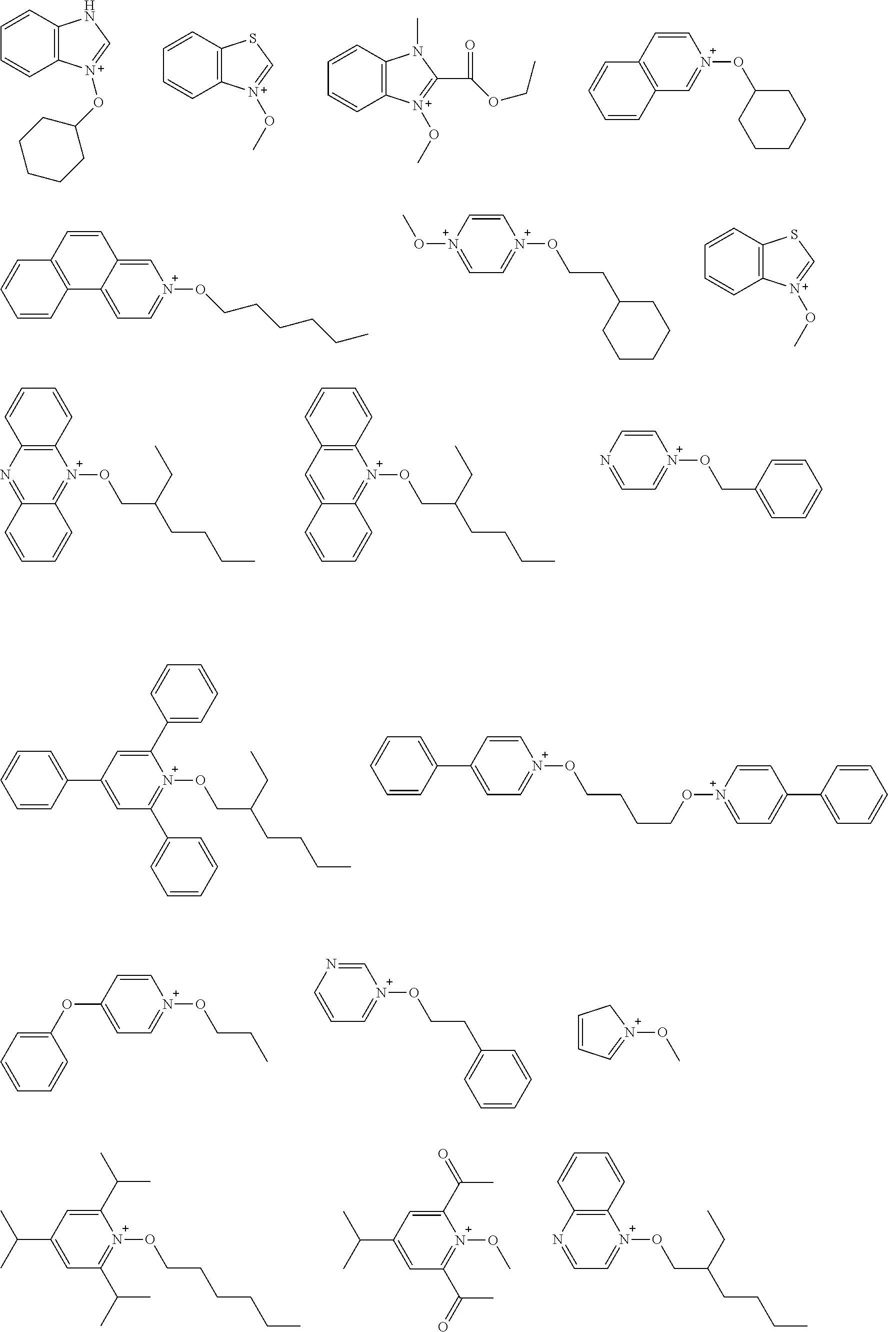

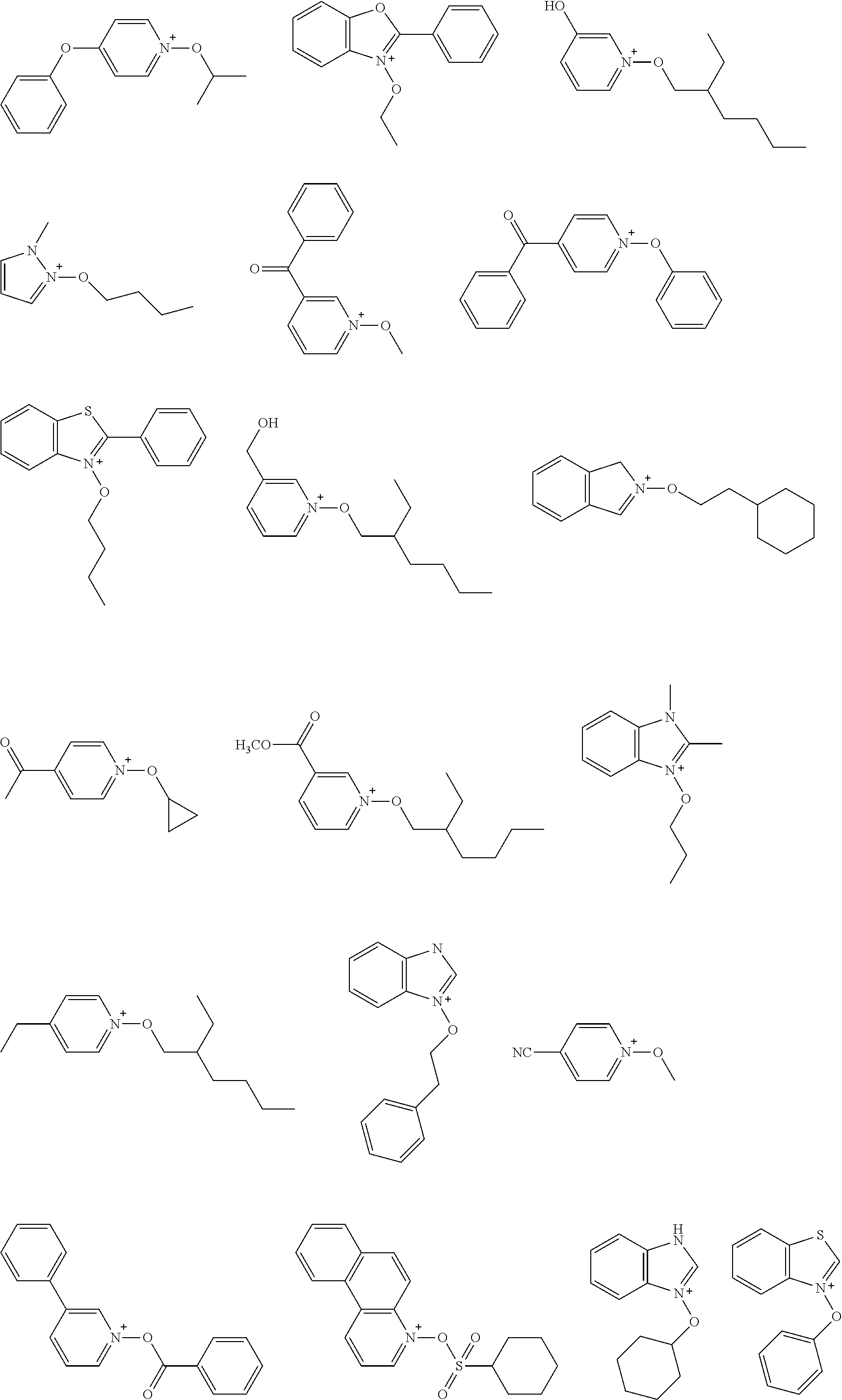

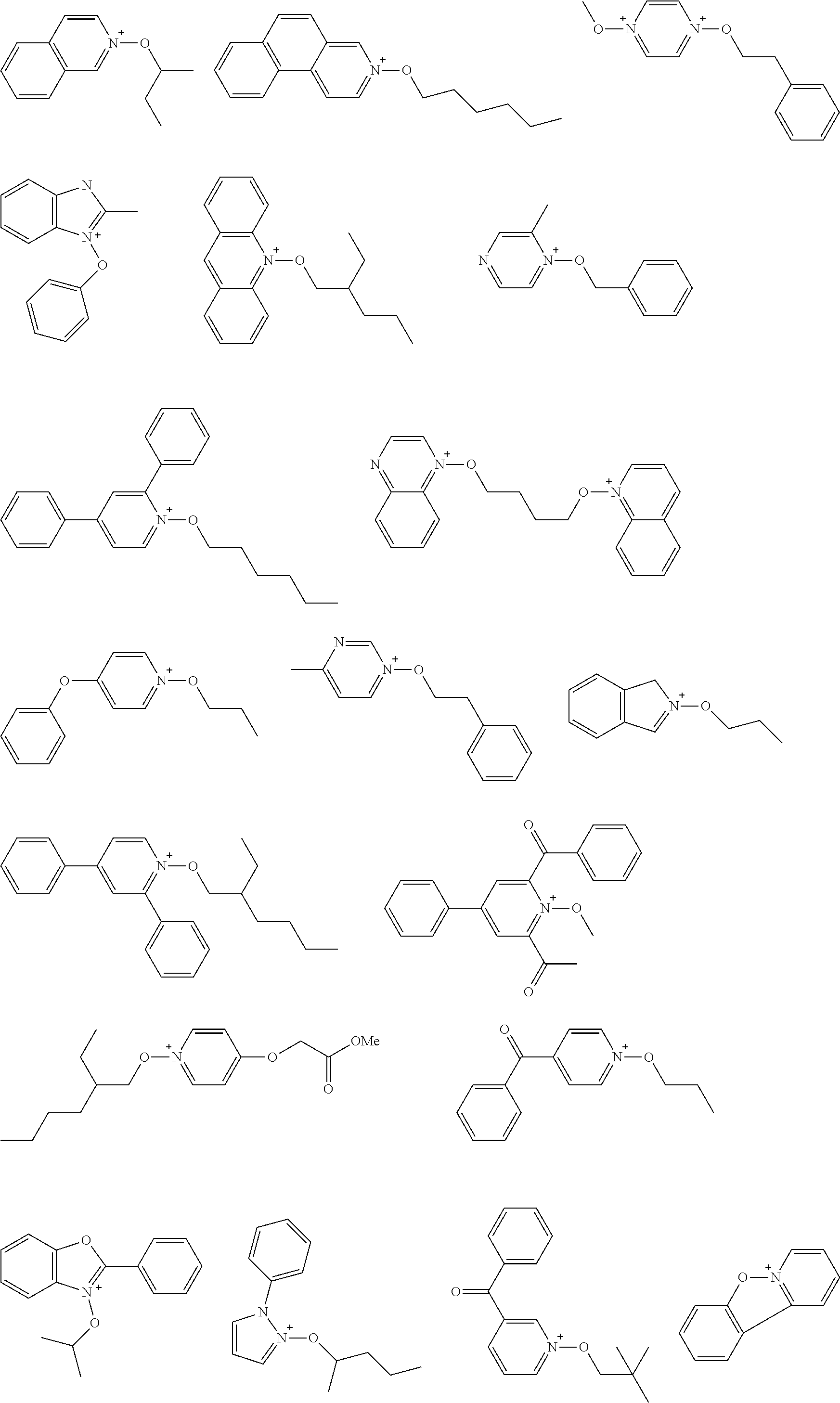

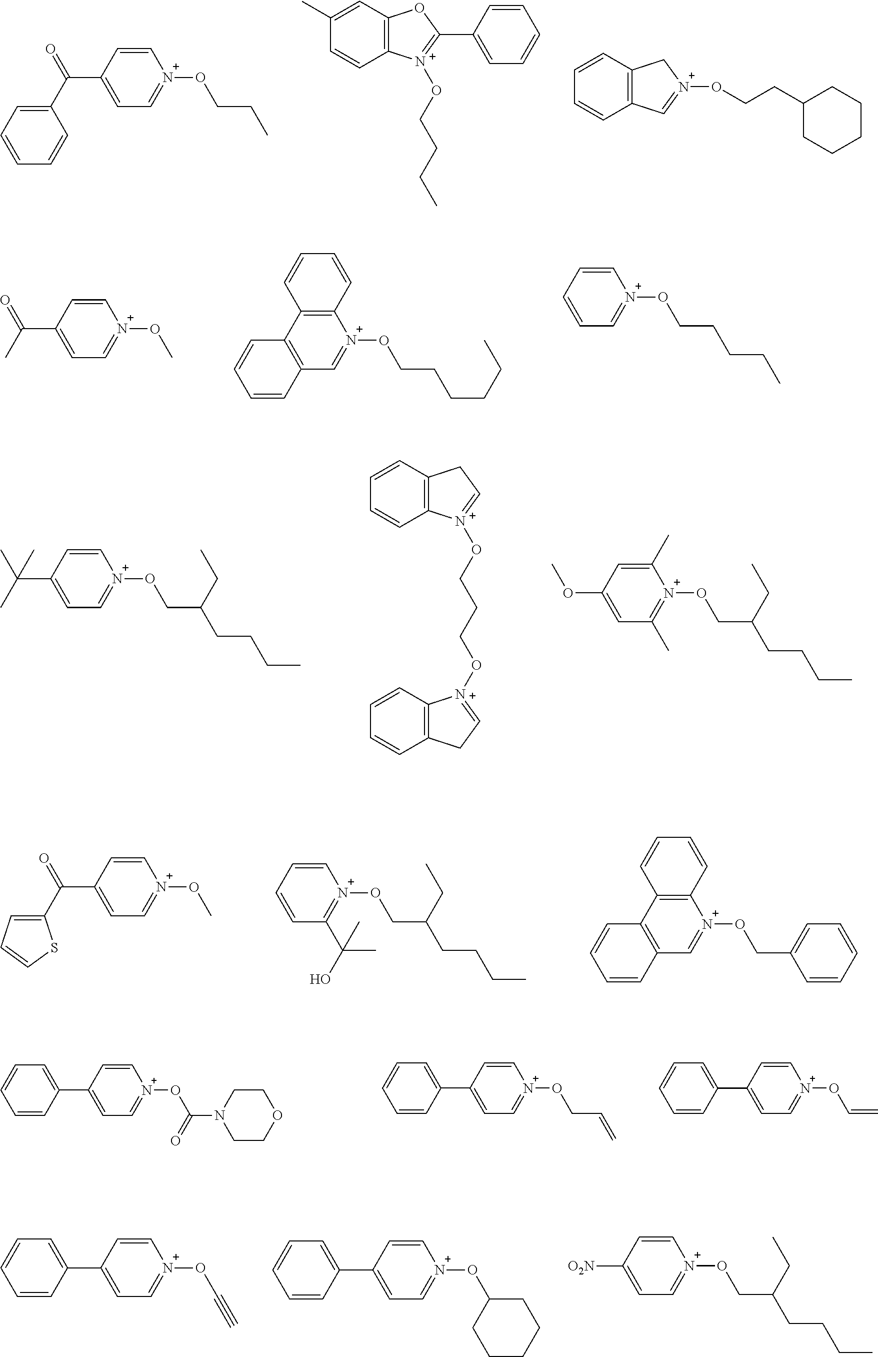

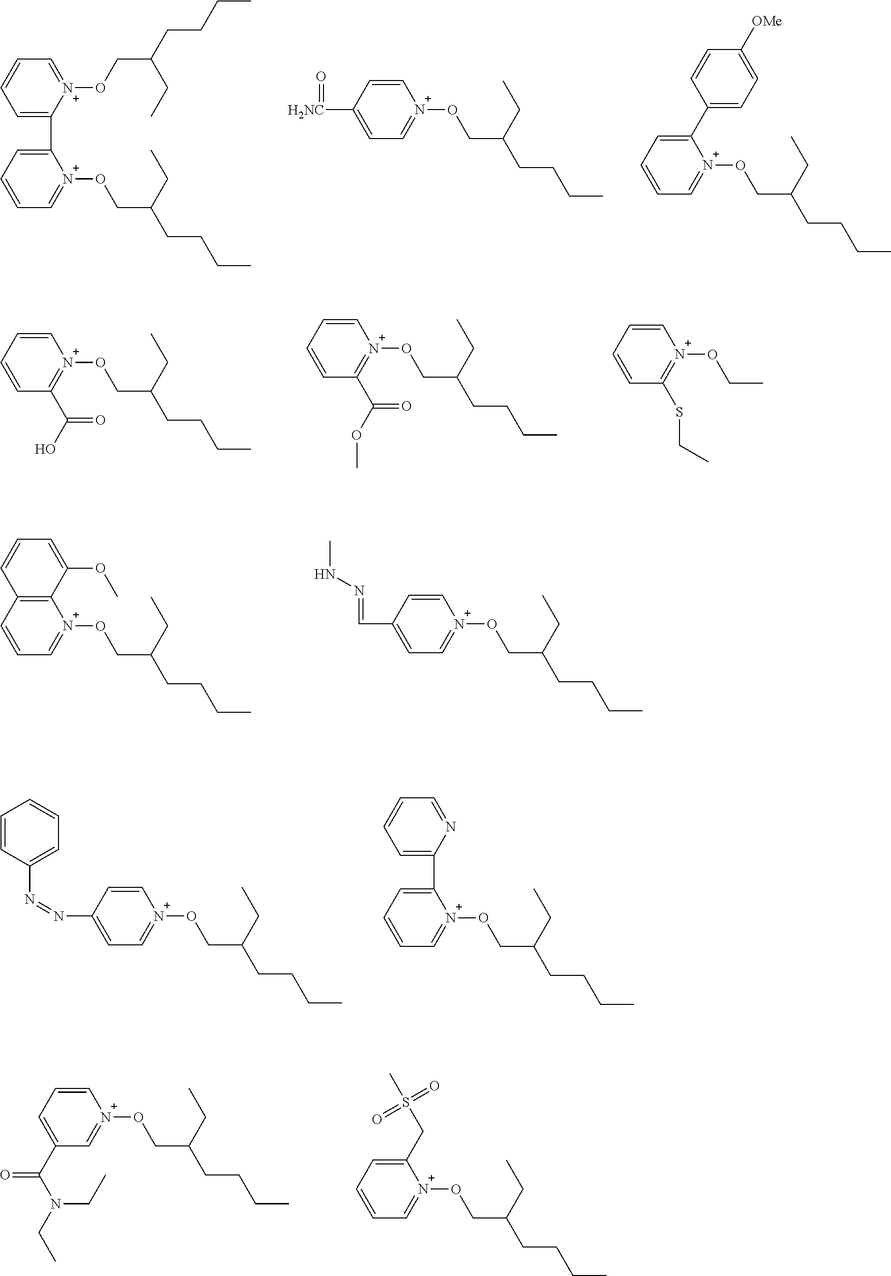

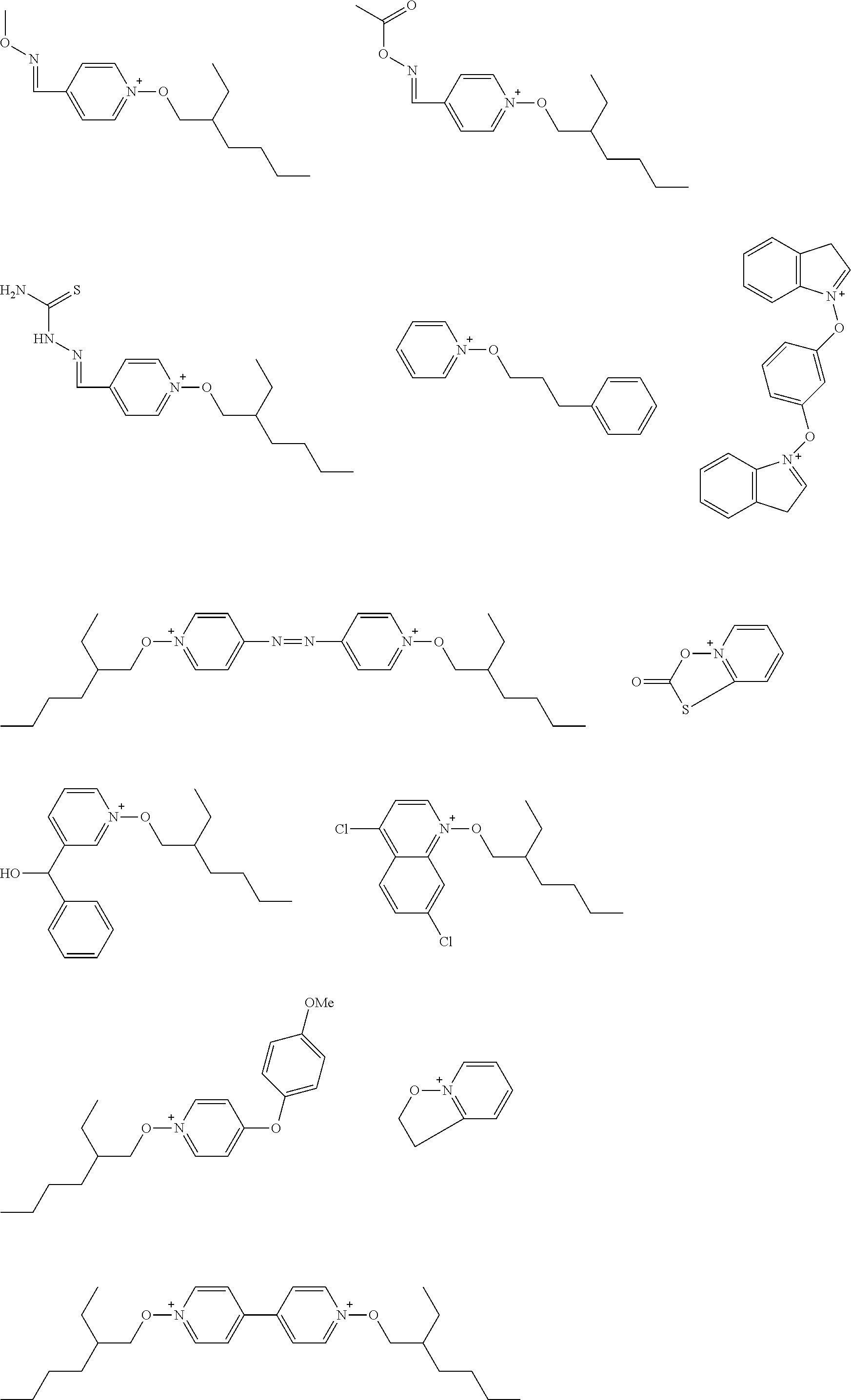

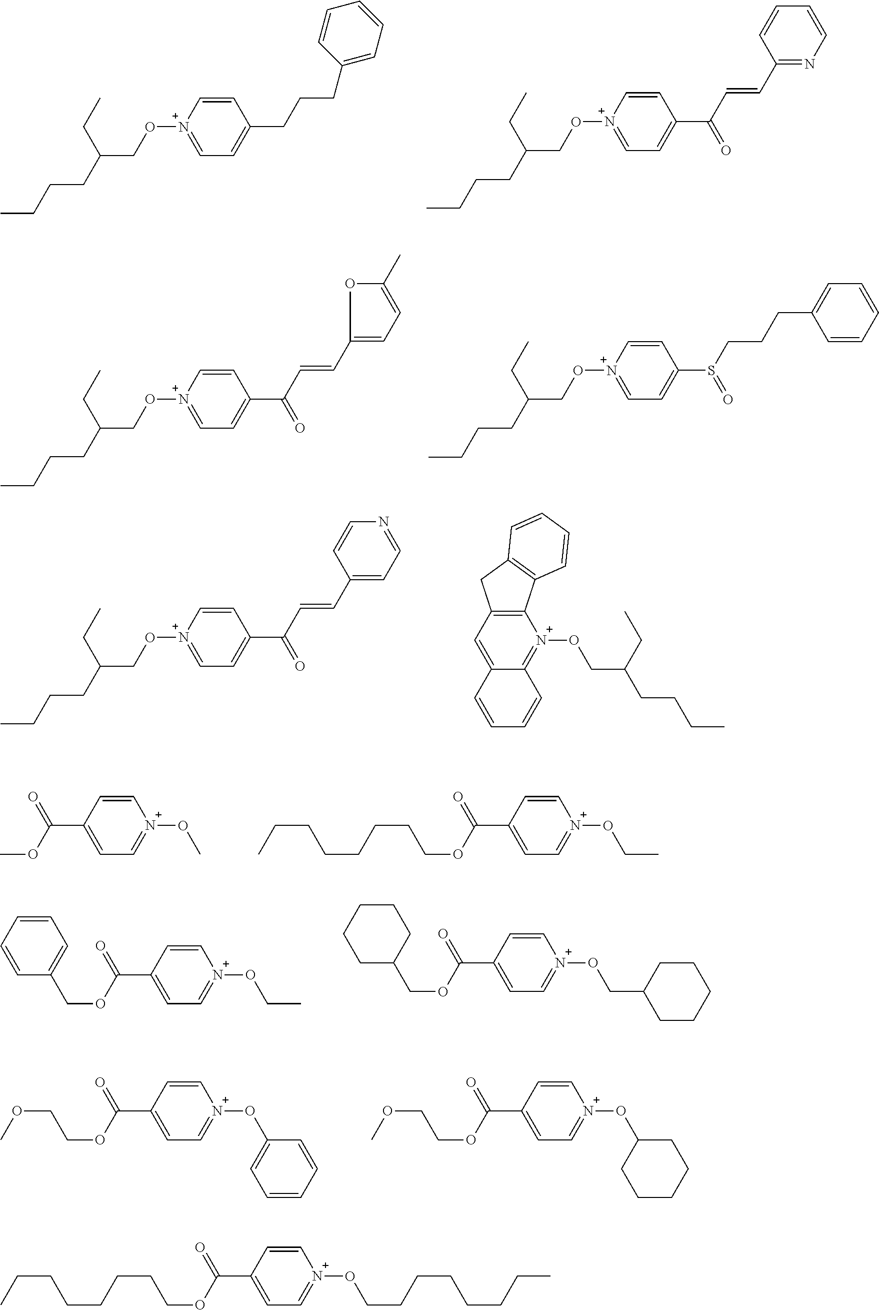

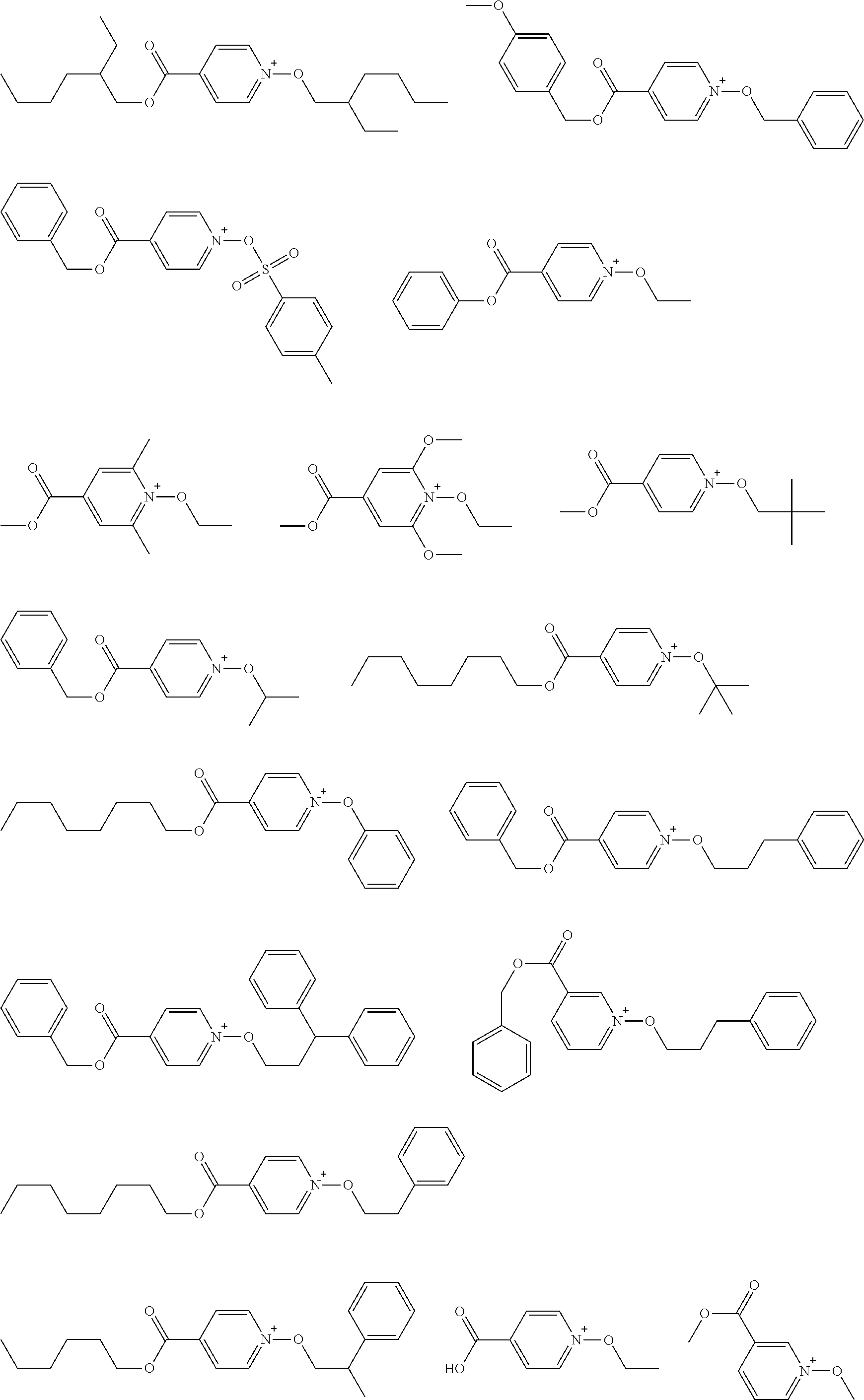

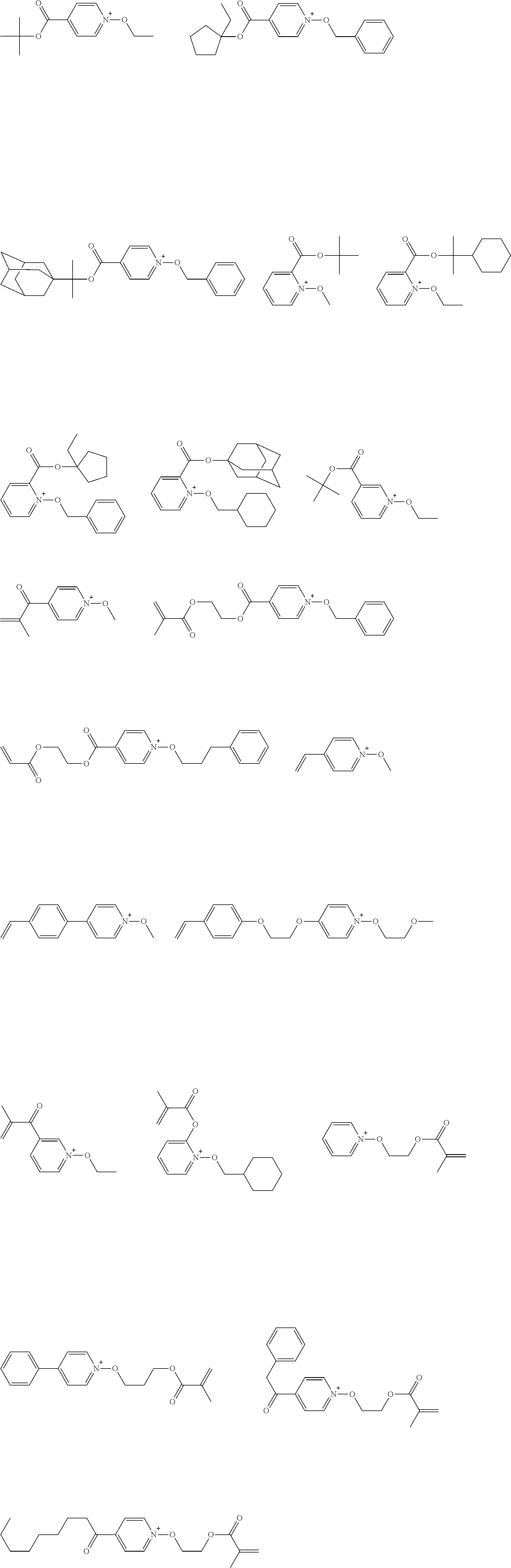

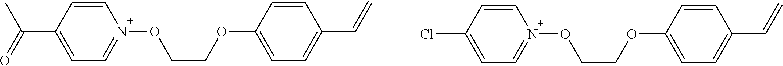

The cation moieties of the compounds of general formula (I) can be synthesized by, for example, the methods described in J. AM. CHEM. SOC. 2004, 126, 14071-14078 or J. AM. CHEM. SOC. 2002, 124, 15225-15238.

Now, the anion represented by X⁻ will be described. Typically, X⁻ represents a normucleophilic anion. The normucleophilic anion means an anion whose capability of inducing a nucleophilic reaction is extremely low. Any decomposition over time attributed to an intramolecular nucleophilic reaction can be suppressed by the use of this anion. Therefore, when this anion is used, the stability over time of the relevant composition and the film formed therefrom can be enhanced.

X− preferably is an organic anion containing one or more carbon atoms. As preferred organic anions, for example, a sulfonate anion, an imido anion, and a methide anion can be exemplified. More specifically, as the preferred organic anions, any of those represented by the following AN1 to AN3 can be exemplified.

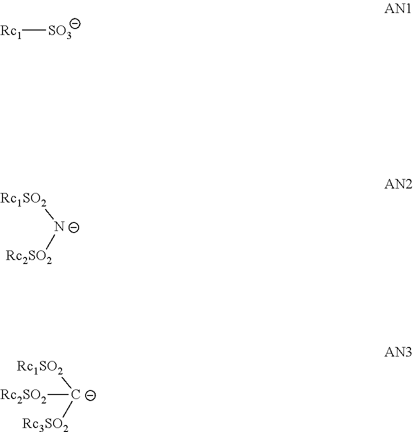

In the formulae AN1 to AN3, Rc₁ to Rc₃ each independently represents an organic group. As the organic group, those having 1 to 30 carbon atoms can be exemplified. Preferably, an alkyl group, an aryl group, or a group in which these groups are connected through a single bond or a connecting group. As the connecting group, for example, —O—, —CO₂—, —S—, —SO₃— and —SO₂N(Rd₁)- can be exemplified. Here, Rd₁ represents a hydrogen atom or an alkyl group, and may form a ring together with a binding alkyl or aryl group.

An organic group represented by Rc₁ to Rc₃ may be an alkyl group whose 1-position is substituted with a fluorine atom or a fluoroalkyl group; or a phenyl group substituted with a fluorine atom or a fluoroalkyl group. Presence of a fluorine atom or a fluoroalkyl group can make an acidity of the acid generated by irradiating light become higher. This can enhance the sensitivity of the composition. Rc₁ to Rc₃ may bond to other alkyl groups, aryl groups, and the like, to thereby form a ring.

As preferred X⁻, a sulfonate anion represented by any of the following general formula (SA1) and (SA2) can be exemplified.

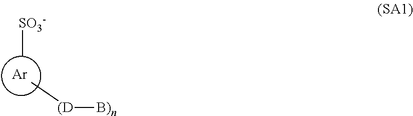

In the formula (SA1),

Ar represents an aryl group, and may further contain one or more substituents other than groups represented by -(D-B).

n is an integer of 1 or greater. n is preferably 1 to 4, more preferably 2 or 3, and most preferably 3.

D represents a single bond or a bivalent connecting group. The bivalent connecting group is, for example, an ether group, a thioether group, a carbonyl group, a sulfoxide group, a sulfone group, a sulfonic ester group, or an ester group.

B represents a hydrocarbon atom.

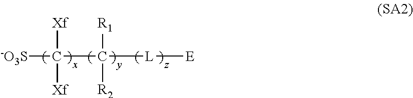

In the formula (SA2),

each of Xfs independently represents a fluorine atom or an alkyl group substituted with at least one fluorine atom.

Each of R₁ and R₂ independently represents a member selected from among a hydrogen atom, a fluorine atom, an alkyl group and an alkyl group substituted with at least one fluorine atom. When two or more R₁s or R₂s are contained, the two or more may be identical to or different from each other.

L represents a single bond or a bivalent connecting group. When two or more Ls are contained, they may be identical to or different from each other.

E represents a group with a cyclic structure.

In the formula, x is an integer of 1 to 20, y an integer of 0 to 10 and z an integer of 0 to 10.

First, a sulfonate anion represented by the formula (SA1) will be described.

In the formula (SA1), Ar is preferably an aromatic ring having 6 to 30 carbon atoms. As the aromatic ring, there can be mentioned, for example, a benzene ring, a naphthalene ring, a pentalene ring, an indene ring, an azulene ring, a heptalene ring, an indecene ring, a perylene ring, a pentacene ring, an acenaphthalene ring, a phenanthrene ring, an anthracene ring, a naphthacene ring, a chrysene ring, a triphenylene ring, a fluorene ring, a biphenyl ring, a pyrrole ring, a furan ring, a thiophene ring, an imidazole ring, an oxazole ring, a thiazole ring, a pyridine ring, a pyrazine ring, a pyrimidine ring, a pyridazine ring, an iodolizine ring, an indole ring, a benzofuran ring, a benzothiophene ring, an isobenzofuran ring, a quinolizine ring, a quinoline ring, a phthalazine ring, a naphthyridine ring, a quinoxaline ring, a quinoxazoline ring, an isoquinoline ring, a carbazole ring, a phenanthridine ring, an acridine ring, a phenanthroline ring, a thianthrene ring, a chromene ring, a xanthene ring, a phenoxathiin ring, a phenothiazine ring, a phenazine ring or the like. Of these, a benzene ring, a naphthalene ring and an anthracene ring are preferred from the viewpoint of the simultaneous attainment of roughness improvement and sensitivity enhancement. A benzene ring is more preferred.

The aromatic ring may have one or more substituents other than the -(D-B) groups. As the substituent, there can be mentioned, for example, those previously explained for the group represented by R. Of these, a linear or branched alkyl group is preferred from the viewpoint of roughness improvement.

In the formula (SA1), D preferably is a single bond, an ether bond, or an ester bond. A single bond is especially preferred.

In the formula (SA1), B preferably is an alkyl group, an alkenyl group, an alkynyl group, an aryl group, or a cycloalkyl group. B preferably is an alkyl group or a cycloalkyl group. an alkyl group, an alkenyl group, an alkynyl group, an aryl group, or a cycloalkyl group represented by B may have one or more substituents.

An alkyl group represented by B preferably is a branched alkyl group. As the branched alkyl group, an isopropyl group, a tert-butyl group, a tert-pentyl group, a neopentyl group, a sec-butyl group, an isobutyl group, an isohexyl group, a 3,3-dimethylpentyl group, and a 2-ethylhexyl group can be exemplified.

A cycloalkyl group represented by B may either be monocyclic or polycyclic. As the monocyclic cycloalkyl group, for example, a cyclopropyl group, a cyclobutyl group, a cyclopentyl group, a cyclohexyl group, a cycloheptyl group, and a cyclooctyl group can be exemplified. As the polycyclic cycloalkyl group, for example, an adamantyl group, a norbornyl group, a bornyl group, a campheryl group, a decahydronaphthyl group, a tricyclodecanyl group, a tetracyclodecanyl group, a camphoroyl group, a dicyclohexyl group, and a pinenyl group can be exemplified.

When an alkyl group, an alkenyl group, an alkynyl group, an aryl group, or a cycloalkyl group represented by B have one or more substituents, those previously explained for the group represented by R can be exemplified as the substituents. A linear alkyl group and a branched alkyl group are preferred for simultaneously achieving roughness improvement and high sensitivity.

Now a sulfonate anion represented by the formula (SA2) will be described in details.

In the formula (SA2), Xf is a fluorine atom or an alkyl group at least one hydrogen atom of which is substituted by a fluorine atom. As the alkyl group, those having 1 to 10 carbon atoms are preferred, and those having 1 to 4 carbon atoms are more preferred. The alkyl group substituted with one or more fluorine atoms preferably is a perfluoroalkyl group.

Xf is preferably a fluorine atom or a perfluoroalkyl group having 1 to 4 carbon atoms. In particular, a fluorine atom, CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉ and CH₂CH₂C₄F₉ can be exemplified. Of these, a fluorine atom and CF₃ are preferred, and a fluorine atom are particularly preferred.

In the formula (SA2), each of R₁ and R₂ is a group selected from a hydrogen atom, a fluorine atom, an alkyl group, and an alkyl group at least one hydrogen atom of which is substituted by a fluorine atom. As the alkyl group which may be substituted with one or more fluorine atoms, those having 1 to 4 carbon atoms are preferred. Further, as the alkyl group substituted with one or more fluorine atoms, a perfluoroalkyl group having 1 to 4 carbon atoms are particularly preferred. In particular, CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉ and CH₂CH₂C₄F₉ can be exemplified. Of these, CF₃ is preferred.

In the formula (SA2), x is preferably 1 to 8, more preferably 1 to 4. y is preferably 0 to 4, more preferably 0. z is preferably 0 to 8, more preferably 0 to 4.

In the formula (SA2), L represents a single bond or a bivalent connecting group. As the a bivalent connecting group, —COO—, —COO—, —CO—, —O—, —S—, —SO—, —SO₂—, an alkylene group, a cycloalkylene group; and an alkenylene group can be exemplified. Of these, —COO—, —COO—, —CO—, —O—, —S—, —SO—, or —SO₂— is more preferred. —COO—, —COO—, or —SO₂— is particularly preferred.

In the formula (SA2), E represents a group with a cyclic structure. As the group, an alicyclic group, an aryl group, and a group with any of heterocyclic structures can be exemplified.

The alicyclic group represented by E may either be monocyclic or polycyclic. As the alicyclic group of a single ring, a monocyclic cycloalkyl group such as a cyclopentyl group, a cyclohexyl group, or a cyclooctyl group is preferred. As the alicyclic group of multiple rings, a polycyclic cycloalkyl group such as a norbornyl group, a tricyclodecanyl group, a tetracyclodecanyl group, a tetracyclododecanyl group or an adamantyl group is preferred. In particular, employing a bulky alicyclic group which has a ring having 6 or more carbon atoms as the group E can reduce in-film diffusion in the PEB (post-exposure bake) stage, thereby enhancing a resolving power and EL (Exposure Latitude).

The aryl group represented by E is, for example, a phenyl group, a naphthyl group, a phenanthryl group, or an anthryl group.

The heterocyclic group represented by E may have aromaticity or no aromaticity. The heteroatom contained therein is preferably a nitrogen atom or an oxygen atom. As particular examples of the heterocycles, there can be mentioned a furan ring, a thiophene ring, a benzofuran ring, a benzothiophene ring, a dibenzofuran ring, a dibenzothiophene ring, a pyridine ring, a piperidine ring, a morpholine ring and the like. Of these, a furan ring, a thiophene ring, a pyridine ring, a piperidine ring and a morpholine ring are preferred.

One or more substituents may be introduced in the group of E. As the substituent, there can be mentioned, for example, an alkyl group (may be linear, branched or cyclic, preferably having 1 to 12 carbon atoms), an aryl group (preferably having 6 to 30 carbon atoms), a hydroxyl group, an alkoxy group, an ester group, an amido group, a urethane group, a ureido group, a thioether group, a sulfonamido group or a sulfonic ester group.

The compounds of general formula (I) are decomposed when exposed to actinic rays or radiation to thereby generate acids of general formula: HX. The inventors have found that when use is made of X⁻ of general formula (I) such that each of the volumes of the acids of general formula: HX is 130 Å³ or greater, scum defects can further be suppressed.

Each of the volumes of the acids of general formula: HX is preferably 130 Å³ or greater, more preferably 200 Å³ or greater and further more preferably 240 Å³ or greater. The volume is preferably 2000 Å³ or less, more preferably 1500 Å³ or less. An excessive increase of the volume invites the possibility of lowering of the sensitivity and/or coating solvent solubility.

The volume of each of the acids was calculated using software “WinMOPAC”, made by Fujitsu Limited, in the following manner. Namely, first, the chemical structure of each of the acids was input. Subsequently, while regarding this structure as an initial structure, the most stable conformation of the acid was determined by a molecular force field calculation using an MM3 method. Thereafter, a molecular orbital calculation using a PM3 method was carried out with respect to the most stable conformation. Thus, the “accessible volume” of each of the acids was determined.

Specific examples of the anions represented by X⁻ are shown in Table 1 below. Table 1 also indicates the calculated values of the volumes of the corresponding generated acids.

TABLE 1

| | Volume of |
|---|---|
| | generated acid |
| X⁻ | (Å³) |
| 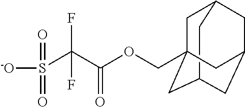 | 271 |
| 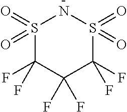 | 136 |
| 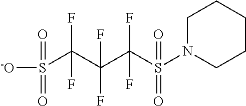 | 244 |
| 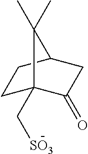 | 216 |
| 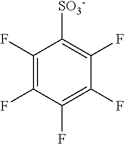 | 127 |
| 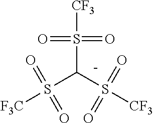 | 189 |
| 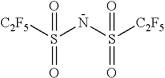 | 173 |
| C₄F₉SO₃⁻ | 113 |
| 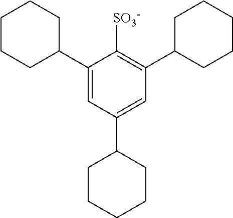 | 437 |
| 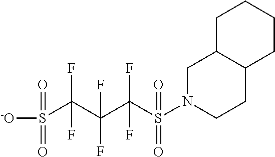 | 311 |
| 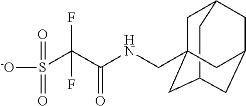 | 277 |
| 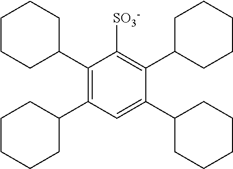 | 529 |
| 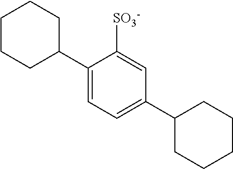 | 338 |
| 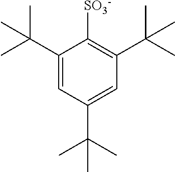 | 357 |
| 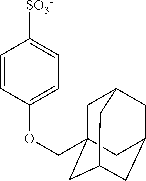 | 310 |
| 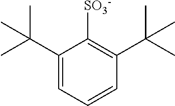 | 280 |
| 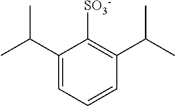 | 244 |
| 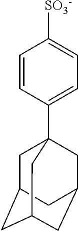 | 281 |
| 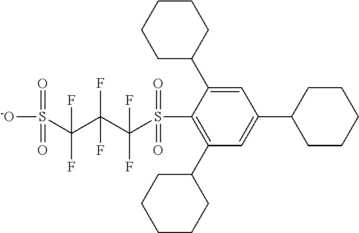 | 525 |
| 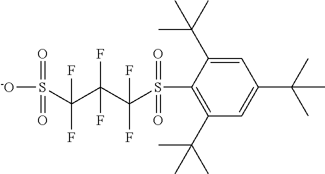 | 395 |
| 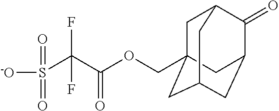 | 266 |
| 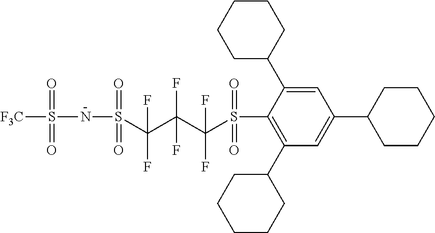 | 582 |
| 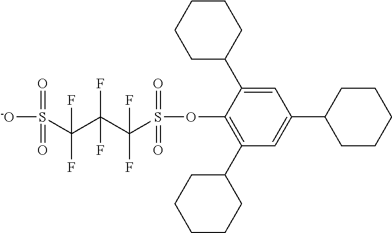 | 535 |
| 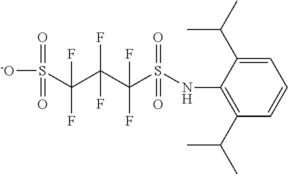 | 339 |
| 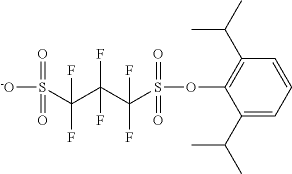 | 347 |
| 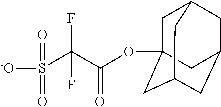 | 271 |
| 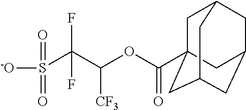 | 291 |
| 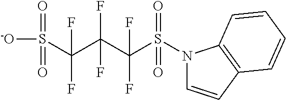 | 250 |
| 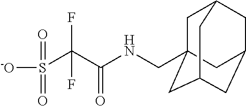 | 277 |
| 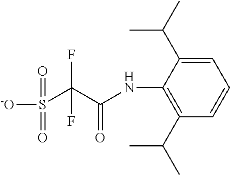 | 297 |
| 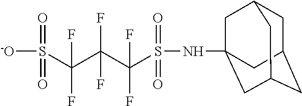 | 315 |
| 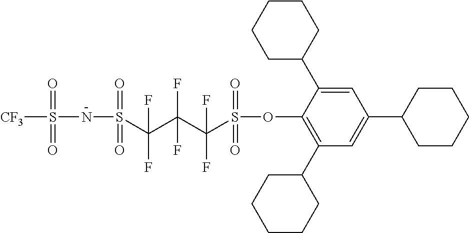 | 591 |
| 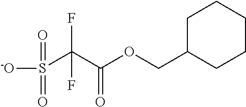 | 221 |
| 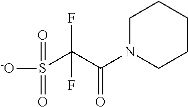 | 187 |
| 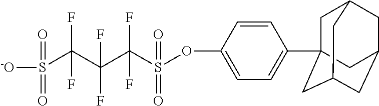 | 380 |
| 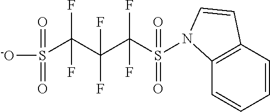 | 250 |
| 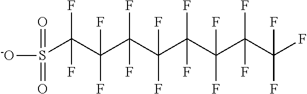 | 220 |
| 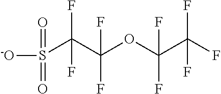 | 141 |
| 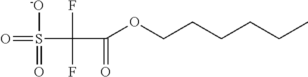 | 216 |
| 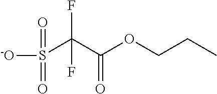 | 154 |
| 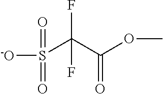 | 113 |
| 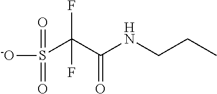 | 161 |
| 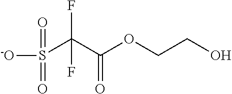 | 139 |
| 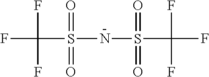 | 130 |
| 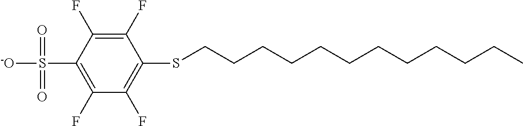 | 393 |
| 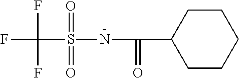 | 202 |
| 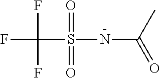 | 113 |
| 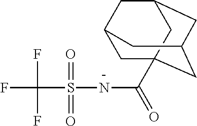 | 251 |
| 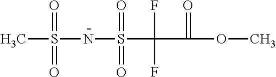 | 171 |
| 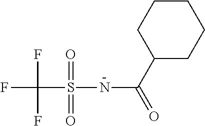 | 202 |
| 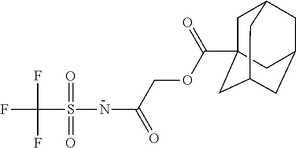 | 295 |
| 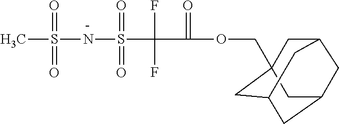 | 326 |
| 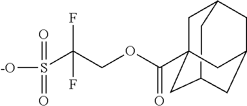 | 270 |
| 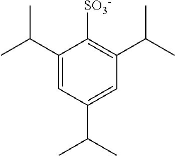 | 303 |

Specific examples of the compounds represented by general formula (I) will be shown below.

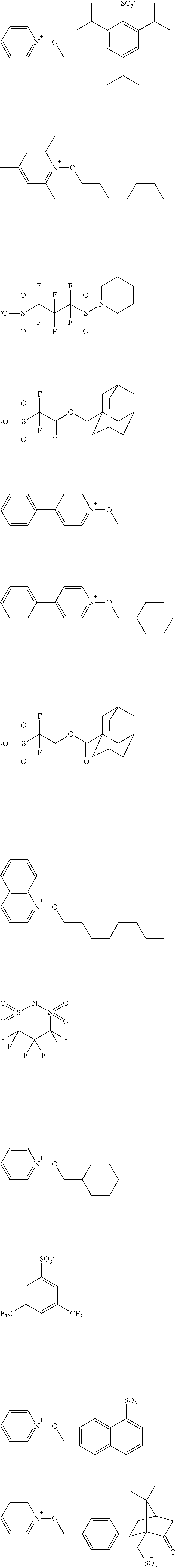

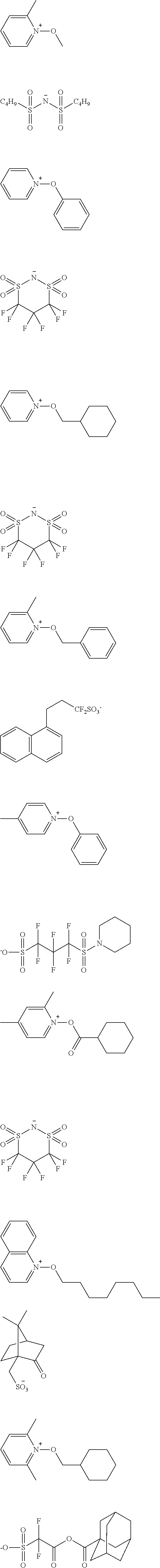

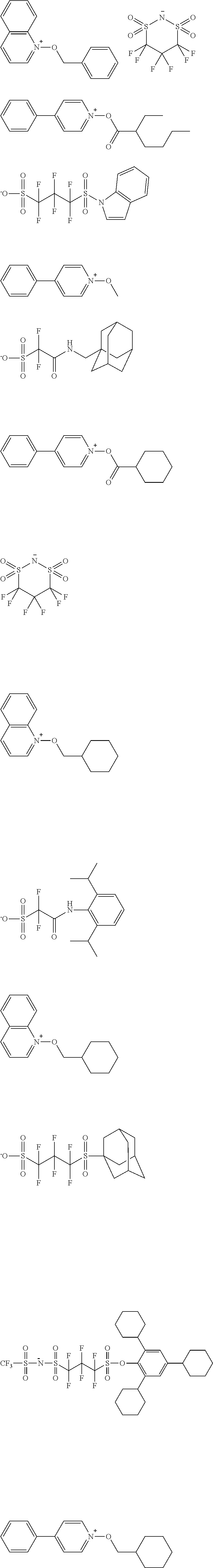

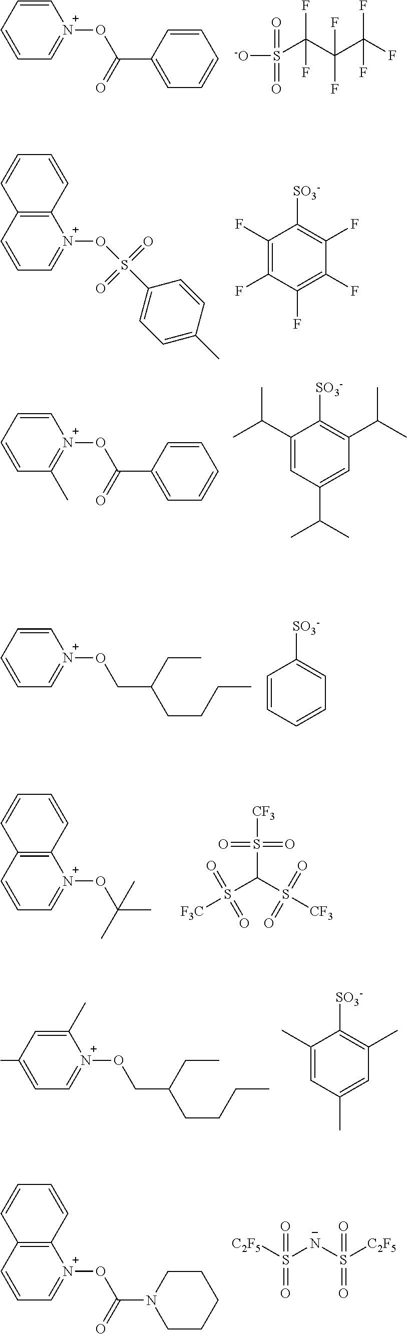

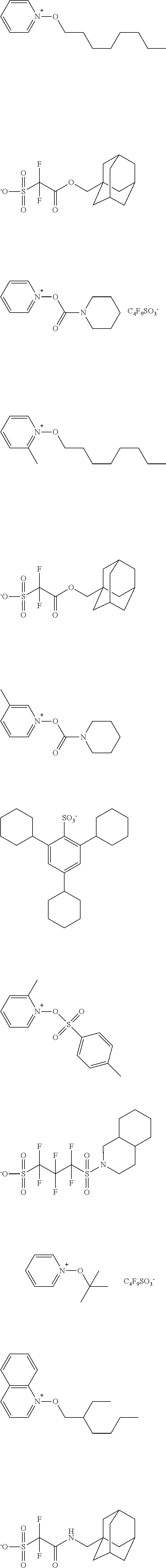

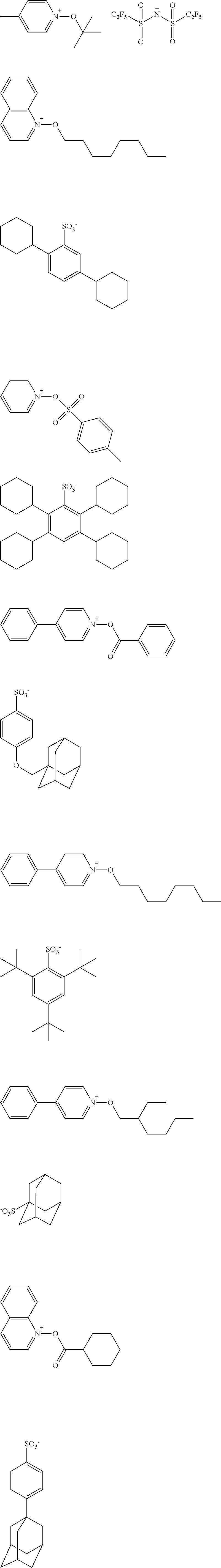

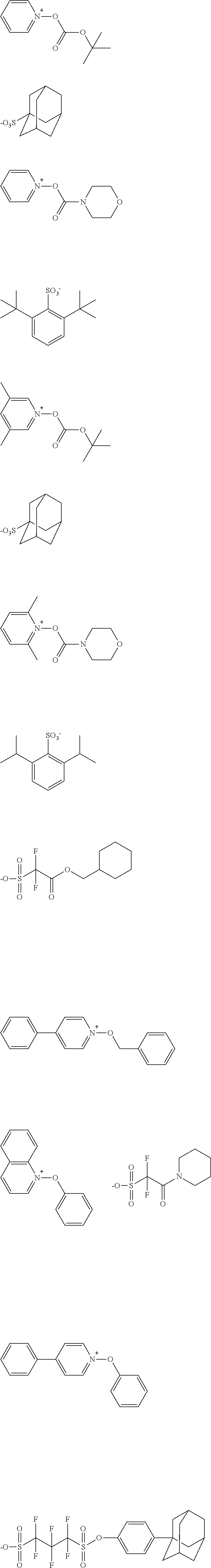

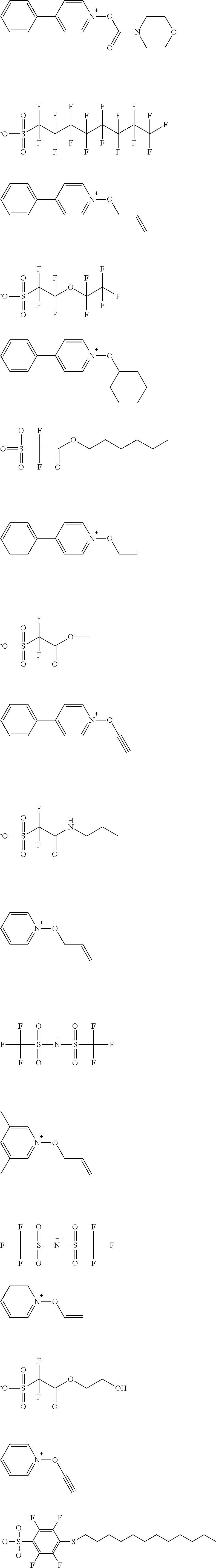

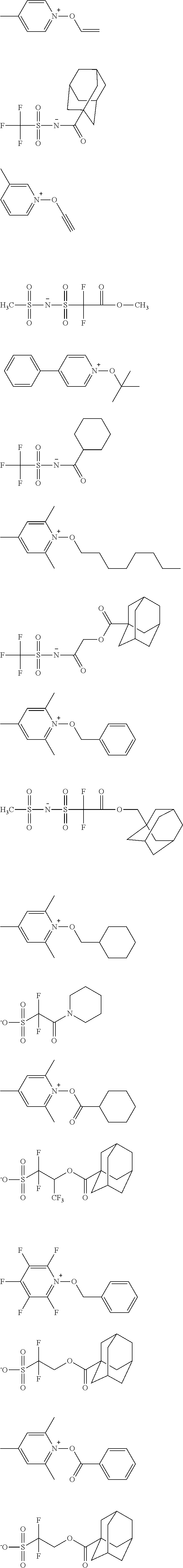

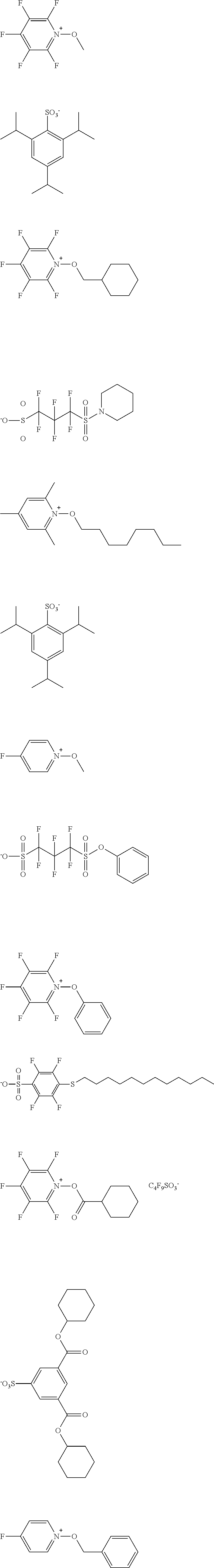

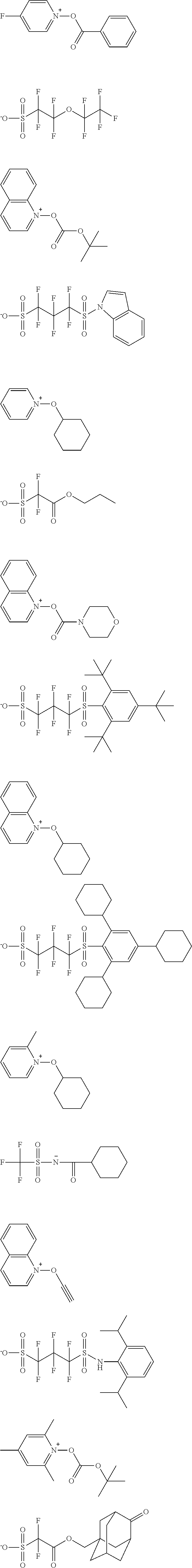

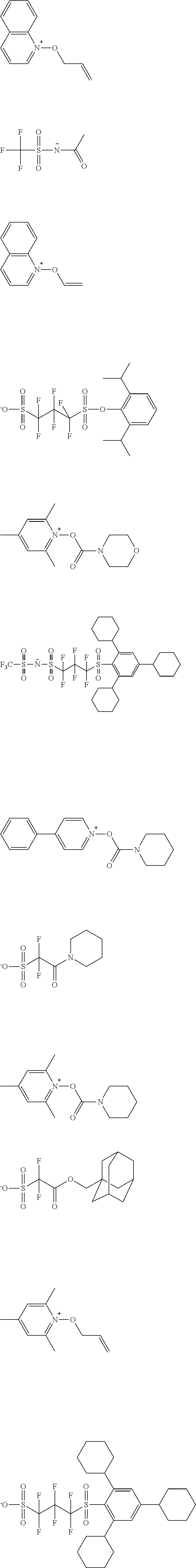

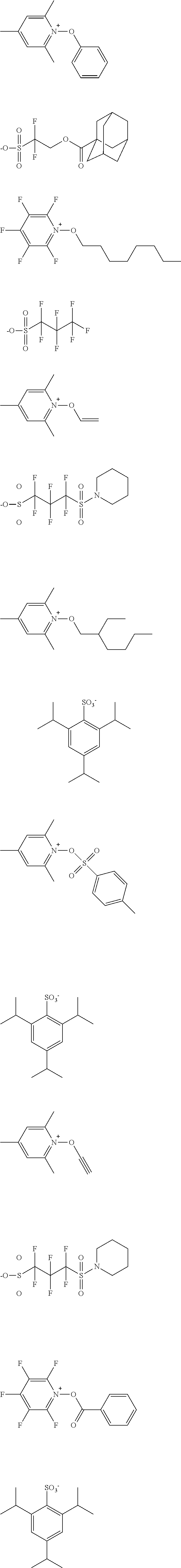

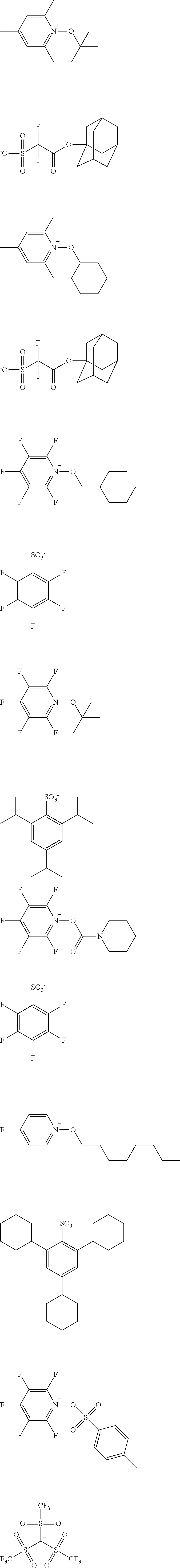

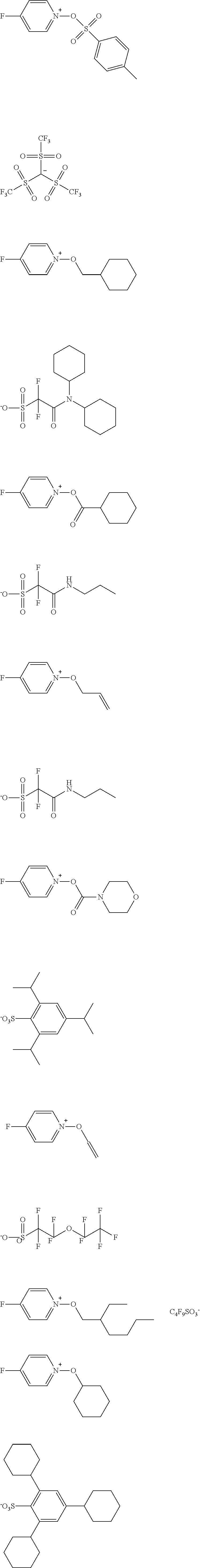

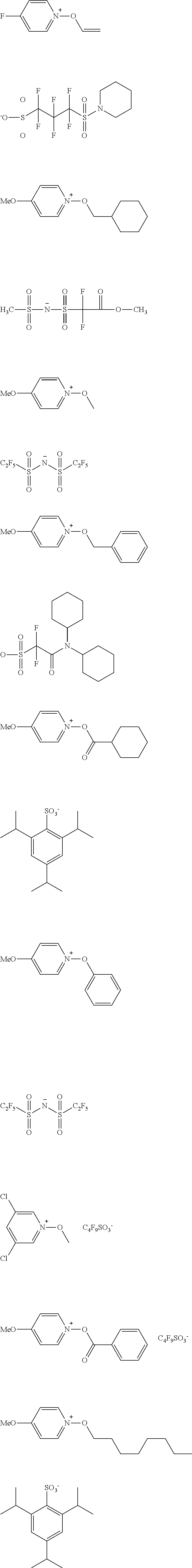

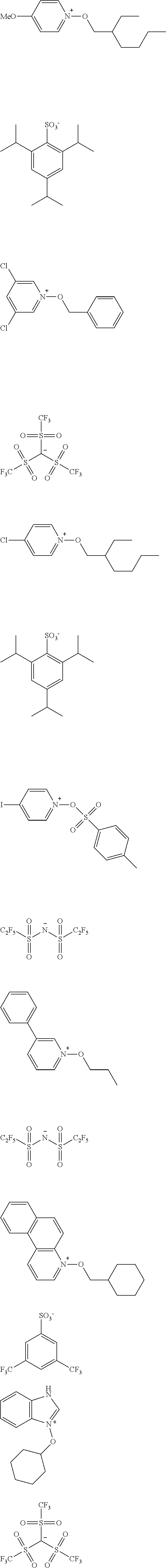

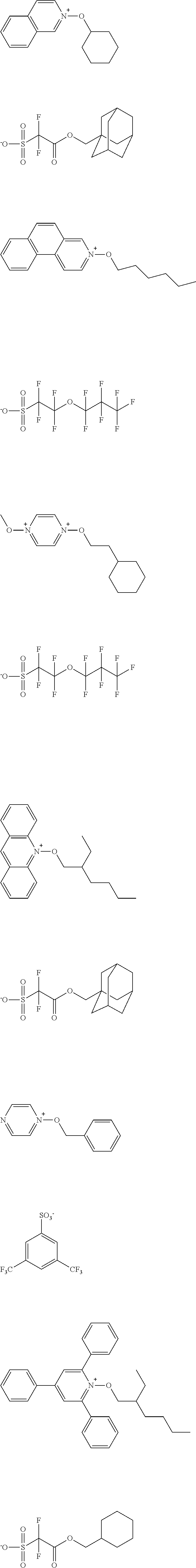

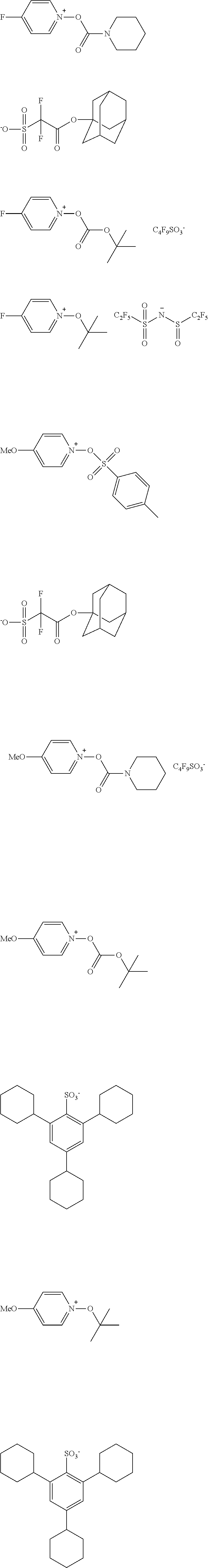

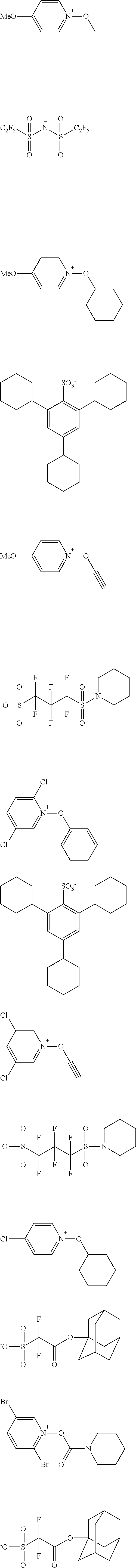

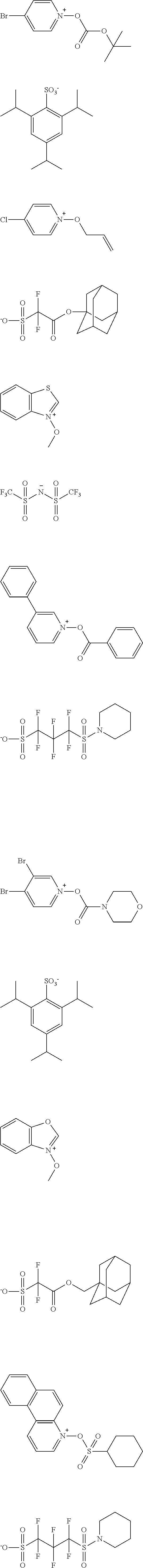

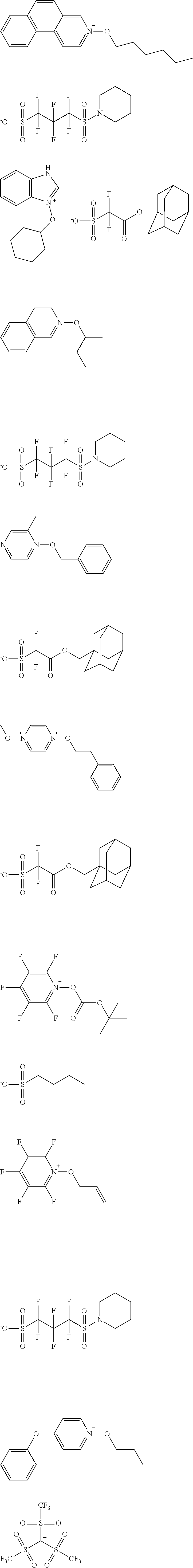

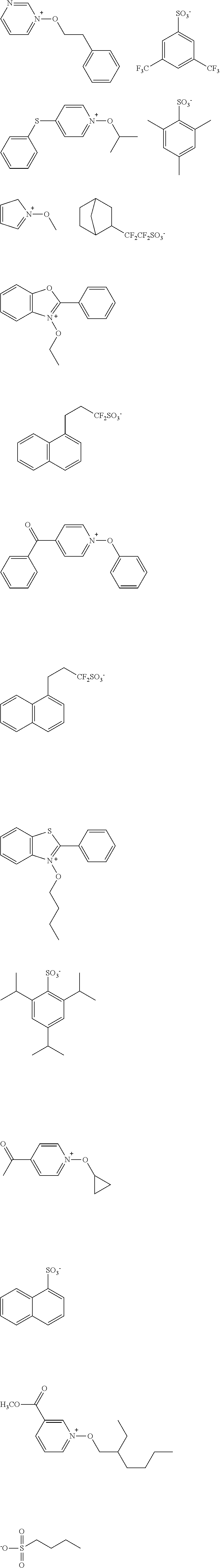

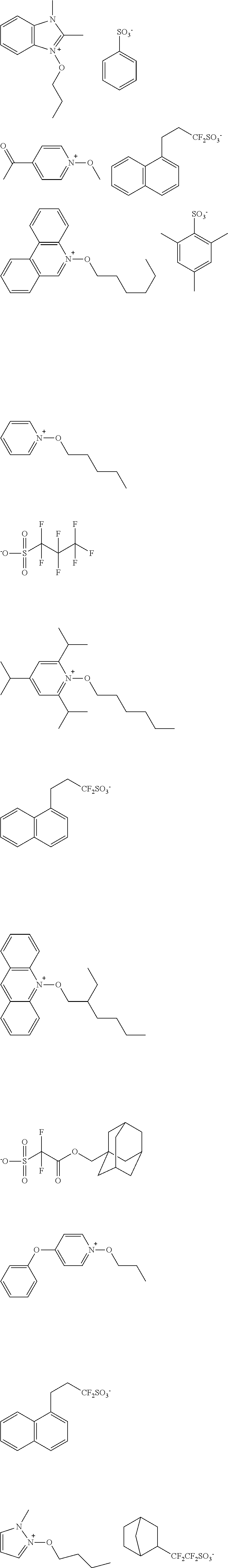

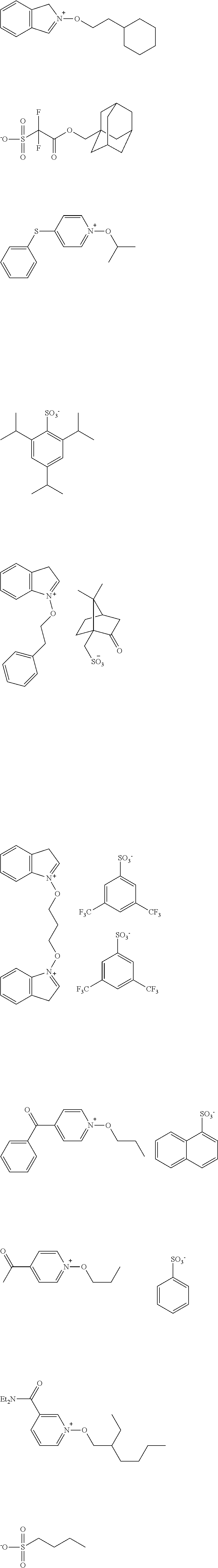

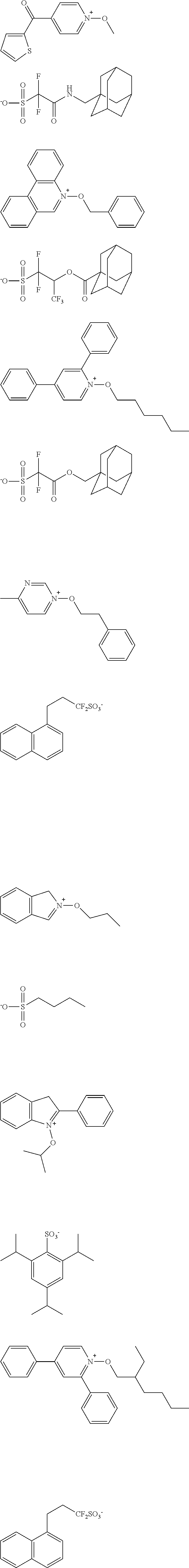

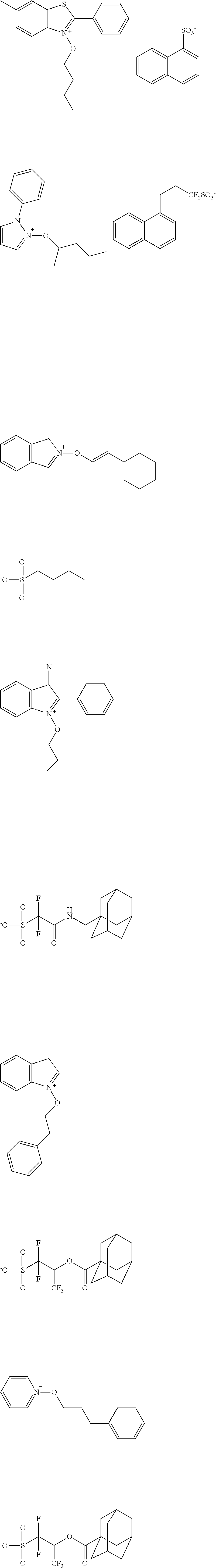

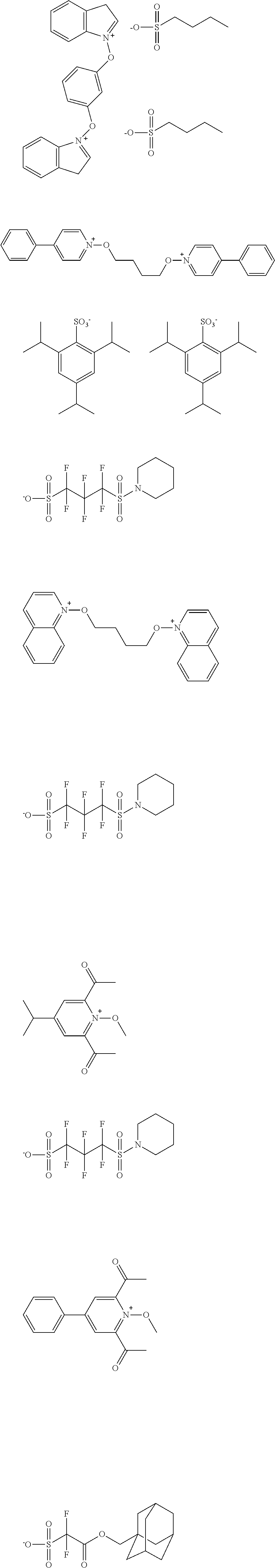

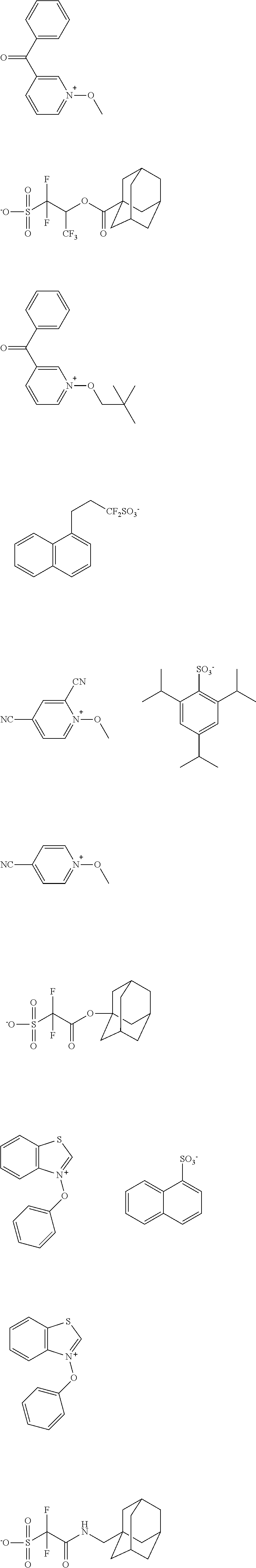

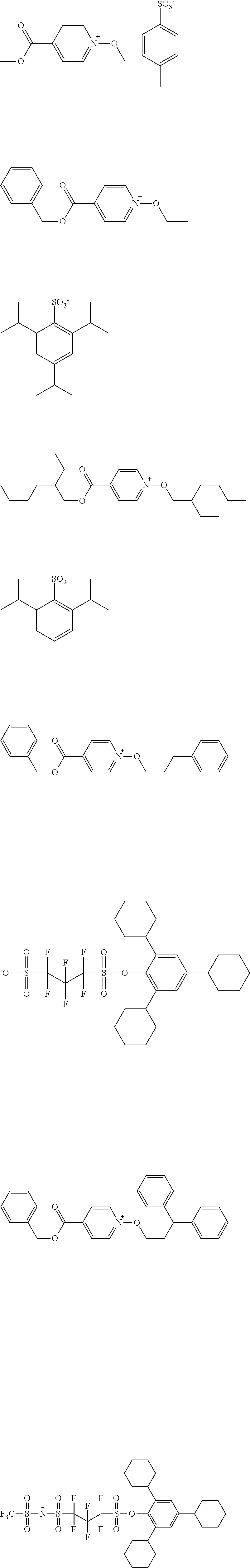

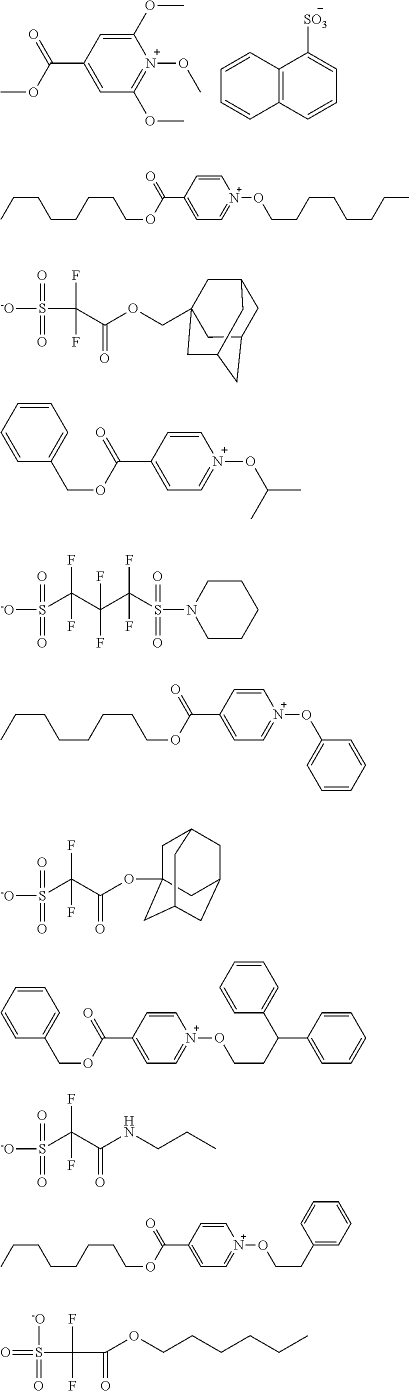

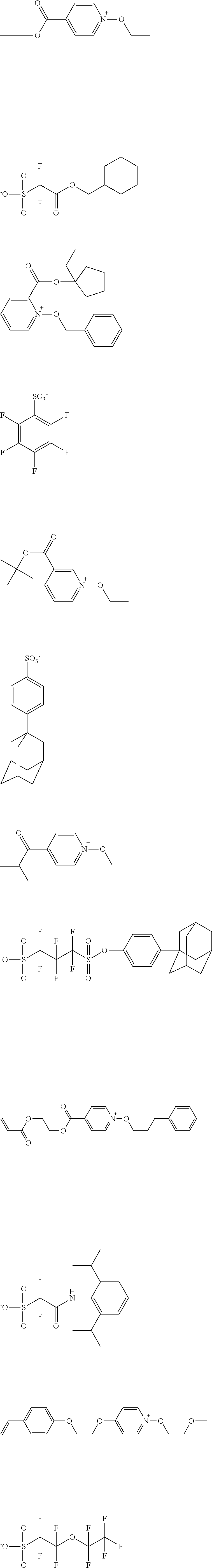

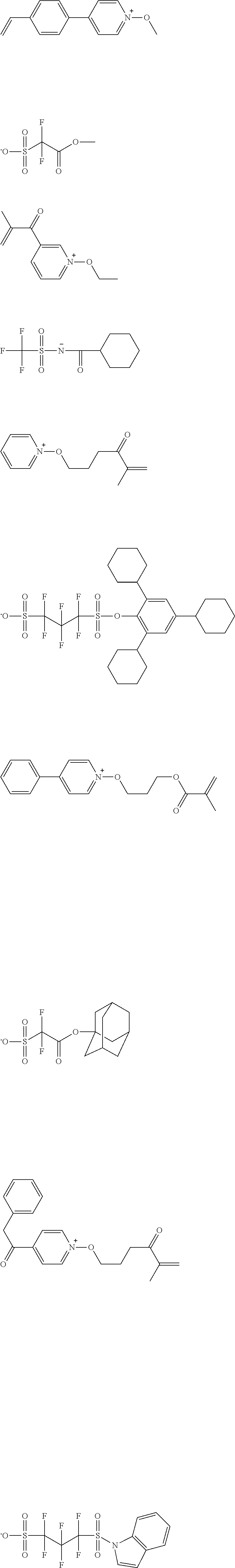

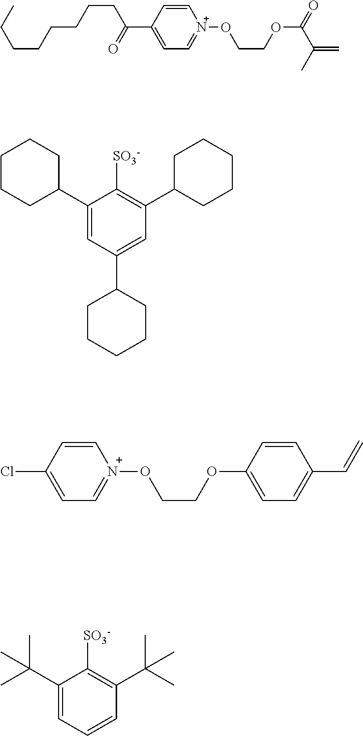

The compounds of general formula (I) are synthesized in, for example, the following manner. First, a hydroxide, bromide or chloride corresponding to each of the above cation moieties is provided. Subsequently, a lithium salt, sodium salt or potassium salt corresponding to each of the above anion moieties is provided. These are reacted with each other by a salt exchange method or a method using an ion exchange resin. Thus, the compounds of general formula (I) are obtained. The salt exchange method and method using an ion exchange resin are described in, for example, Jpn. PCT National Publication No. H11-501909 and JP-A-2003-246786.

The composition of the present invention may further comprise as a photoacid generator a compound other than the compounds of general formula (I).

As acid generators that can be used in combination, there can be mentioned, for example, those represented by the following general formulae (ZX), (ZXI), and (ZXII).

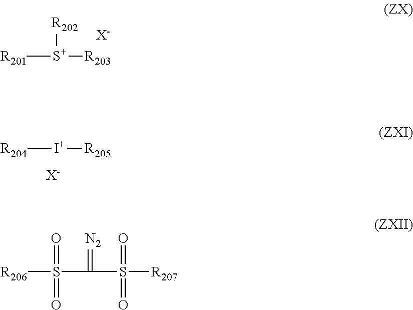

In the above general formula (ZX),

each of R₂₀₁, R₂₀₂ and R₂₀₃ independently represents an organic group.

The number of carbon atoms in the organic group represented by R₂₀₁, R₂₀₂ and R₂₀₃ is generally in the range of 1 to 30, preferably 1 to 20.

Two of R₂₀₁ to R₂₀₃ may be bonded to each other through single bond or connecting group to form a ring. As the connecting group, for example, an ether bond, a thioether bond, an ester bond, an amido bond, a carbonyl group, a methylene group, and an ethylene group can be exemplified. As the group formed by bonding of two of R₂₀₁ to R₂₀₃, for example, an alkylene group such as a butylene group or a pentylene group can be exemplified.

As preferred examples of the photoacid generator which can be used in combination, compounds represented by the following general formula (ZIV), (ZV) or (ZVI) can be exemplified.

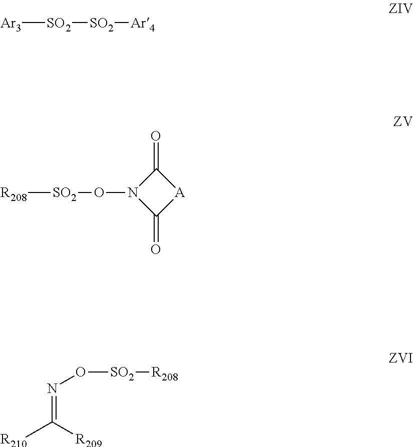

In the general formulae (ZIV) to (ZVI),

each of Ar₃ and Ar₄ independently represents a substituted or an unsubstituted aryl group.

Each of R₂₀₈ independently represents an alkyl group, a cycloalkyl group or an aryl group. These groups may either be substituted or unsubstituted.

It is preferable for these groups to be substituted with one, or more fluorine atoms. This leads to higher acidity of an acid generated by the photoacid generator.

Each of R₂₀₉ and R₂₁₀ independently represents an alkyl group, a cycloalkyl group, an aryl group, or an electron-withdrawing group. These groups may either be substituted or unsubstituted.

As preferred R₂₀₉, substituted or unsubstituted aryl groups can be exemplified.

As preferred R₂₁₀, electron-withdrawing groups can be exemplified. As such, a cyano group and a fluoroalkyl group is preferable.

A represents an alkylene group, an alkenylene group, or an arylene group. These groups may contain one or more substituents.

Specific examples of the photoacid generators which can be used in combination will be shown below.

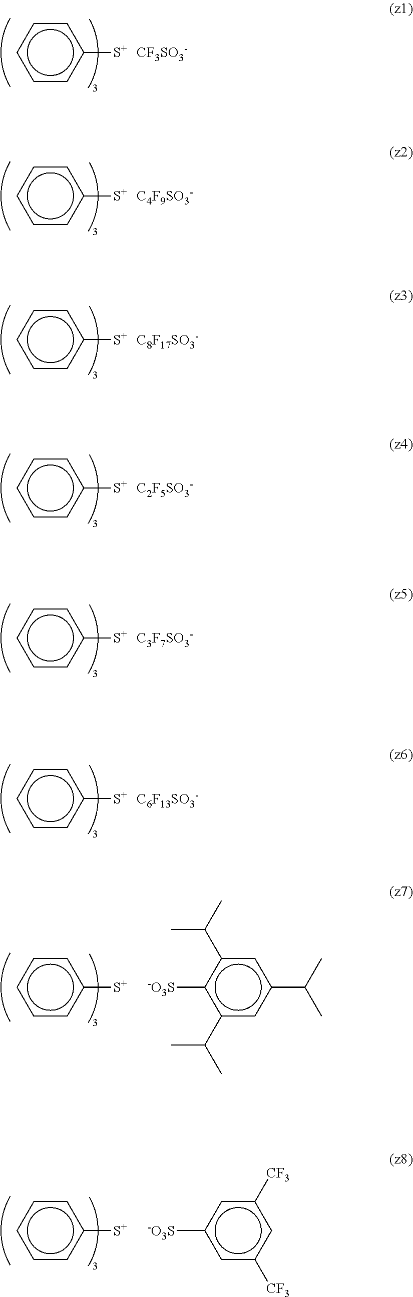

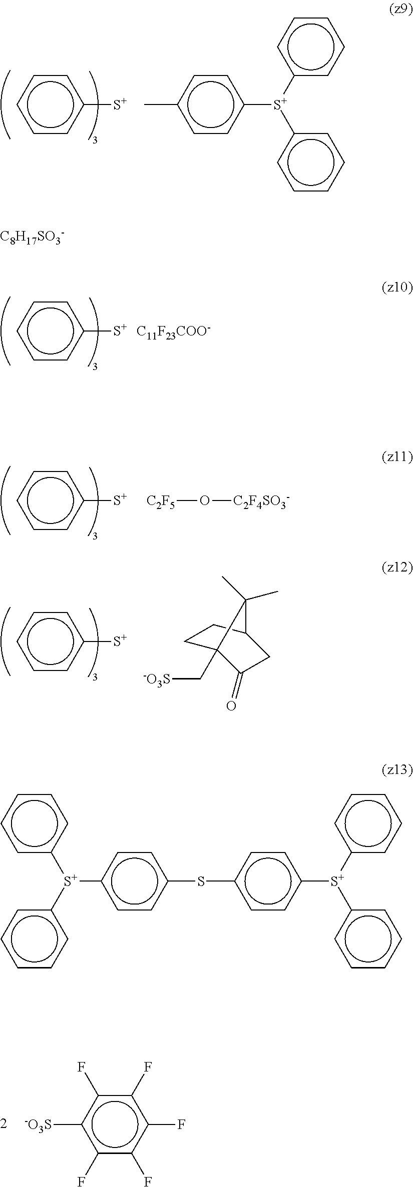

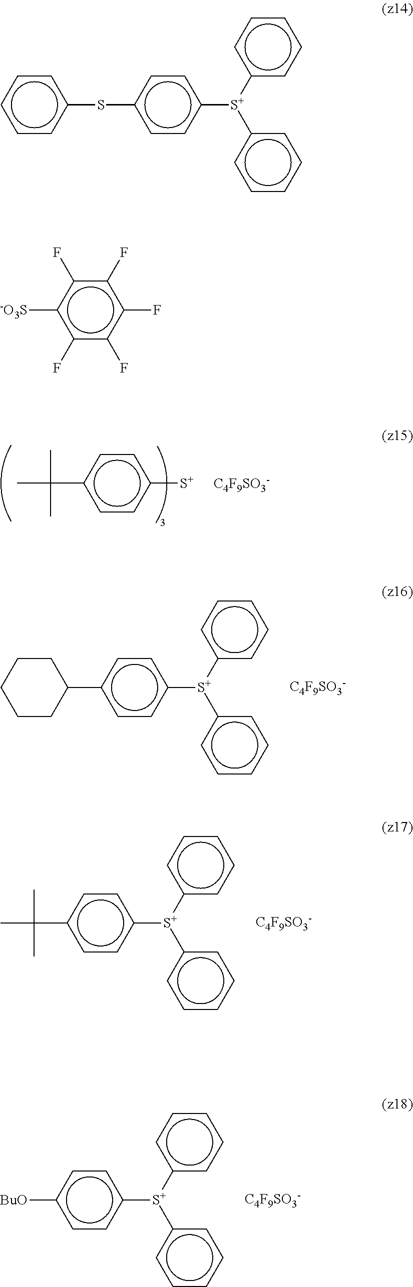

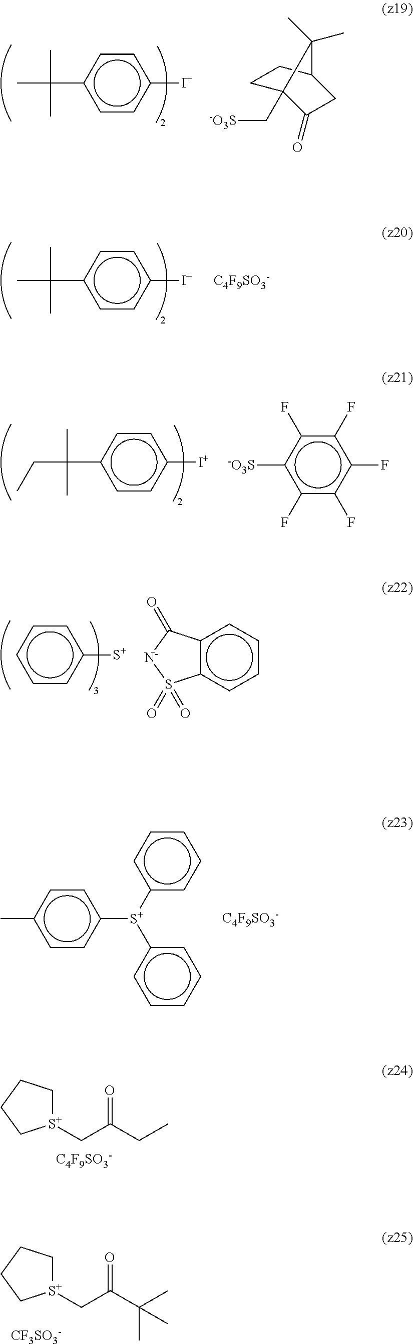

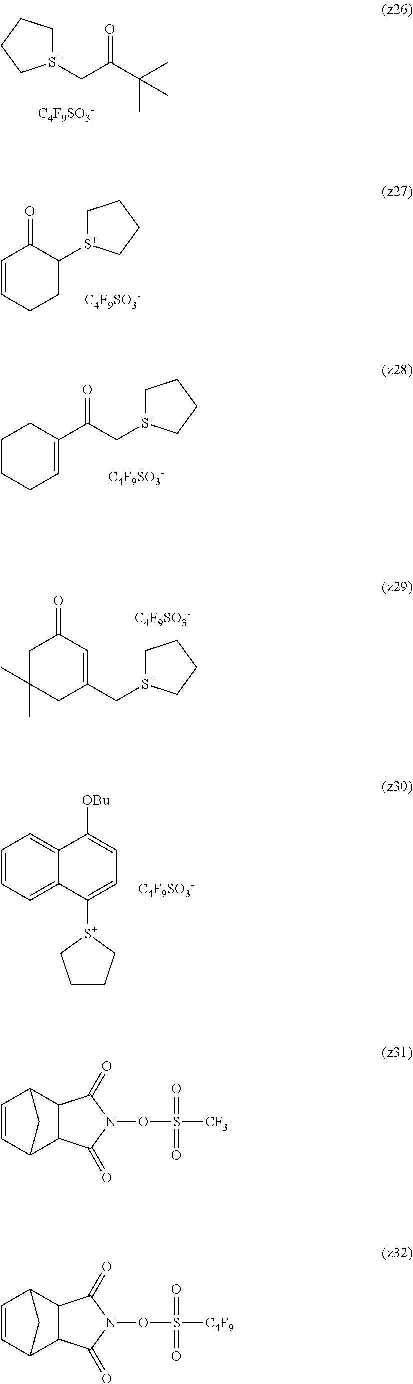

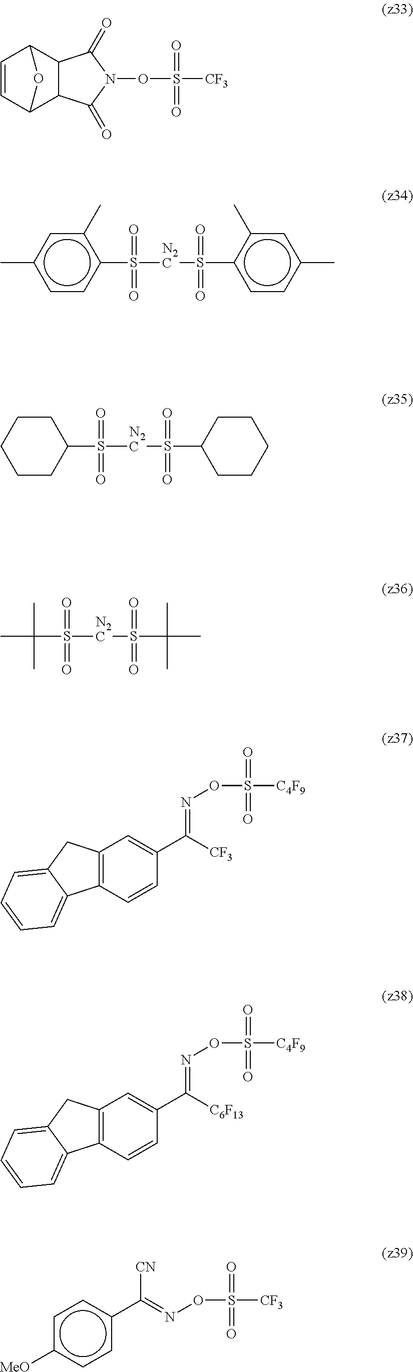

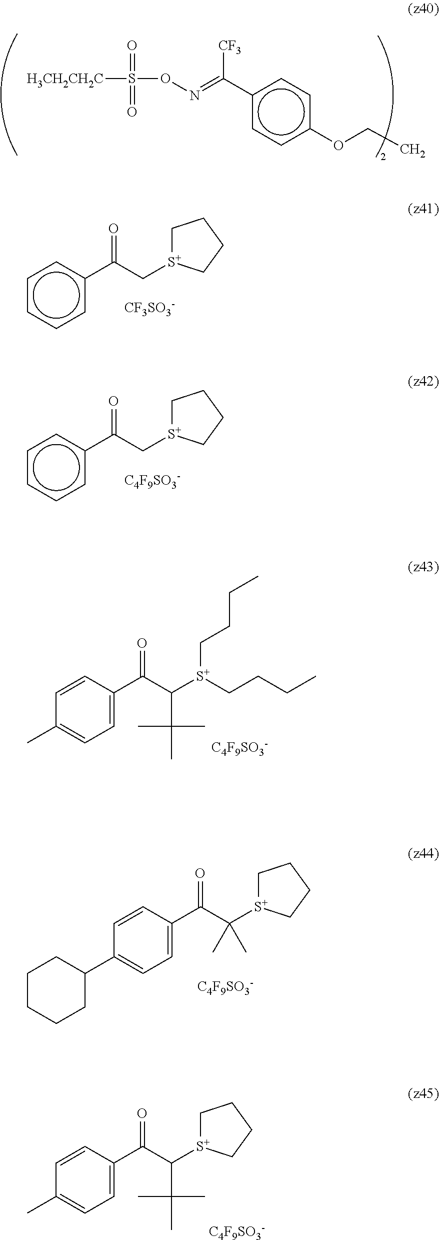

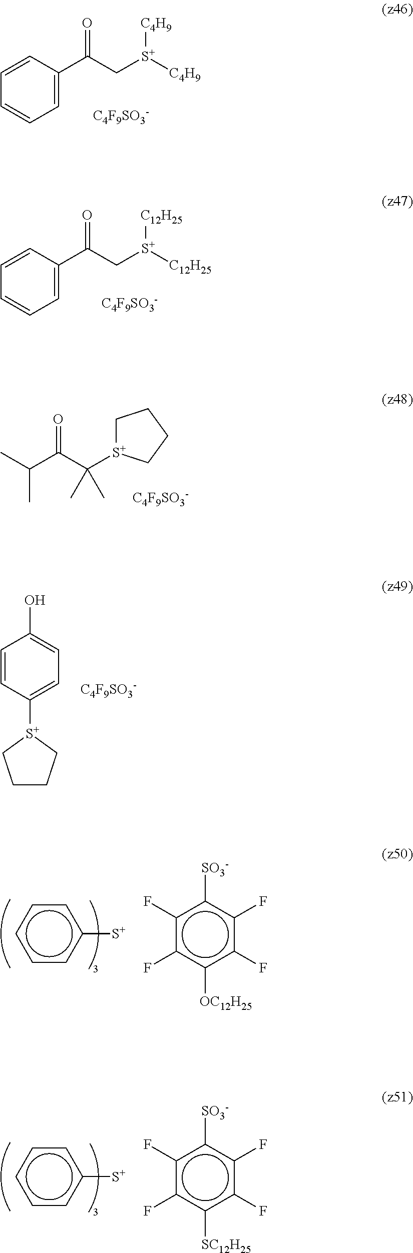

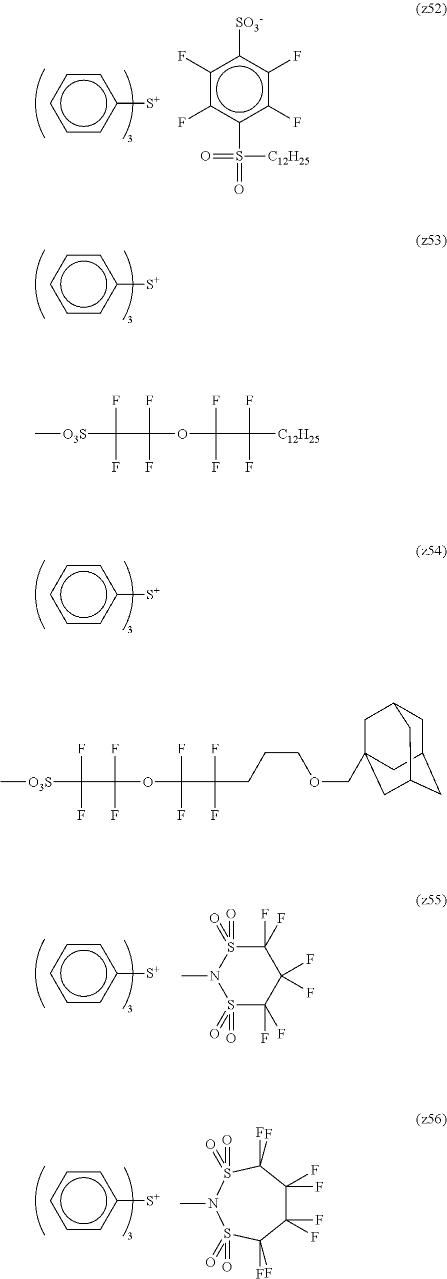

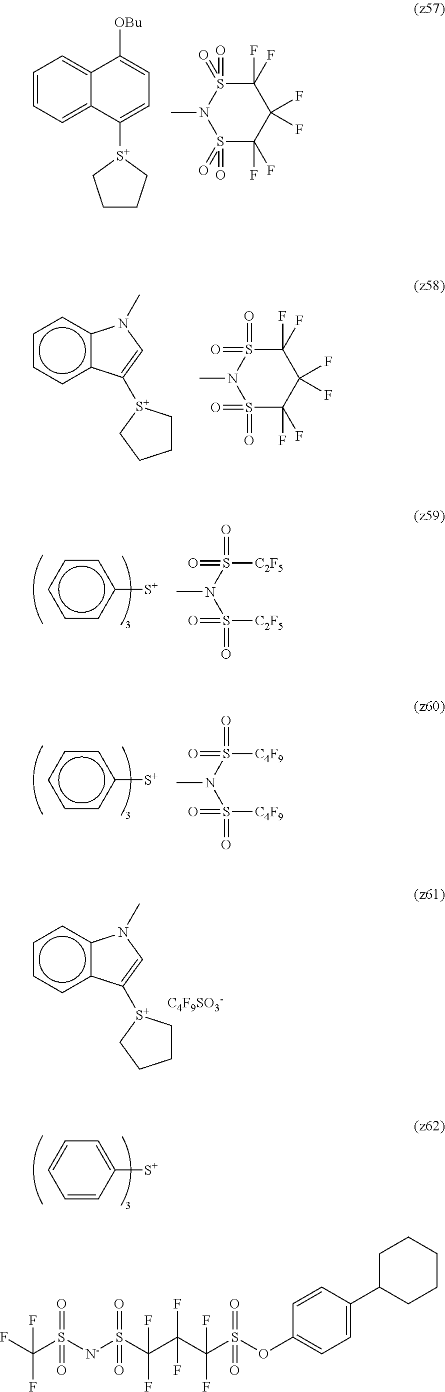

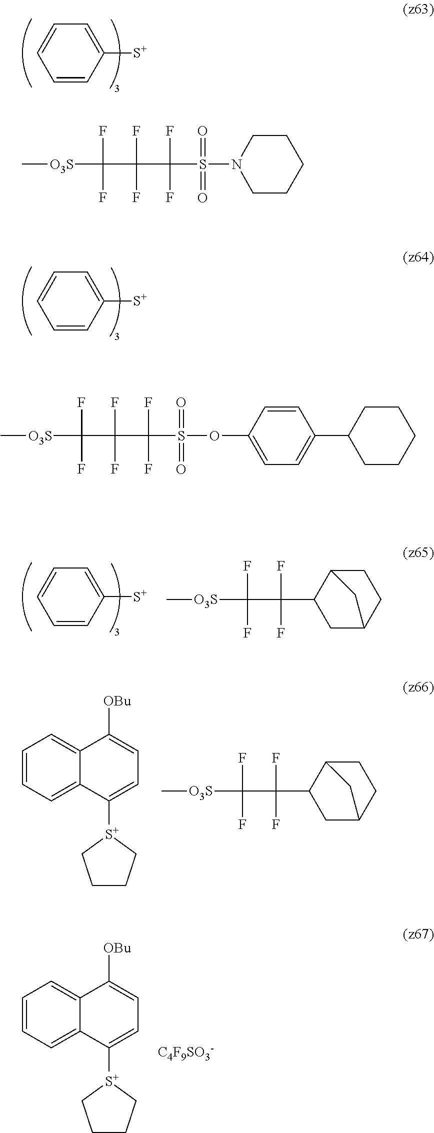

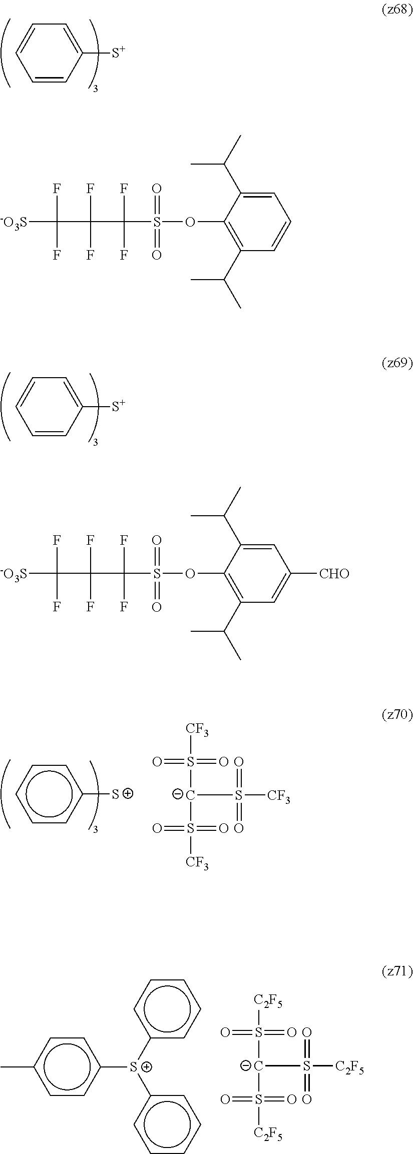

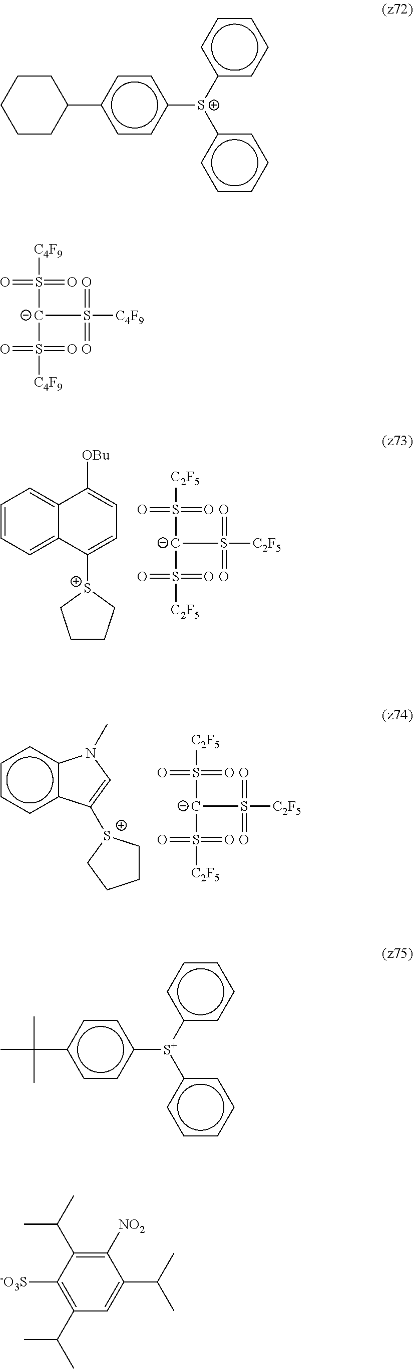

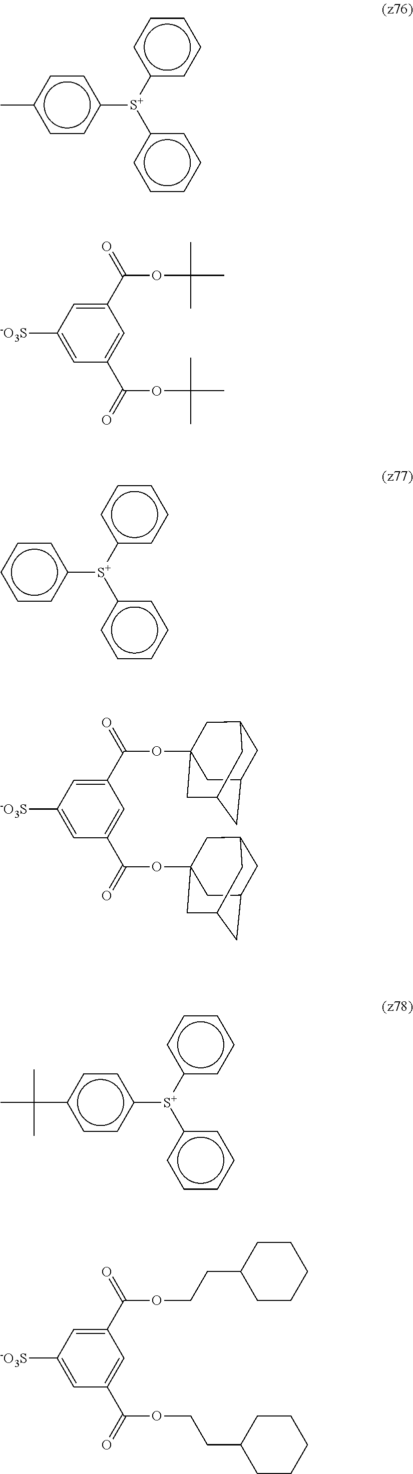

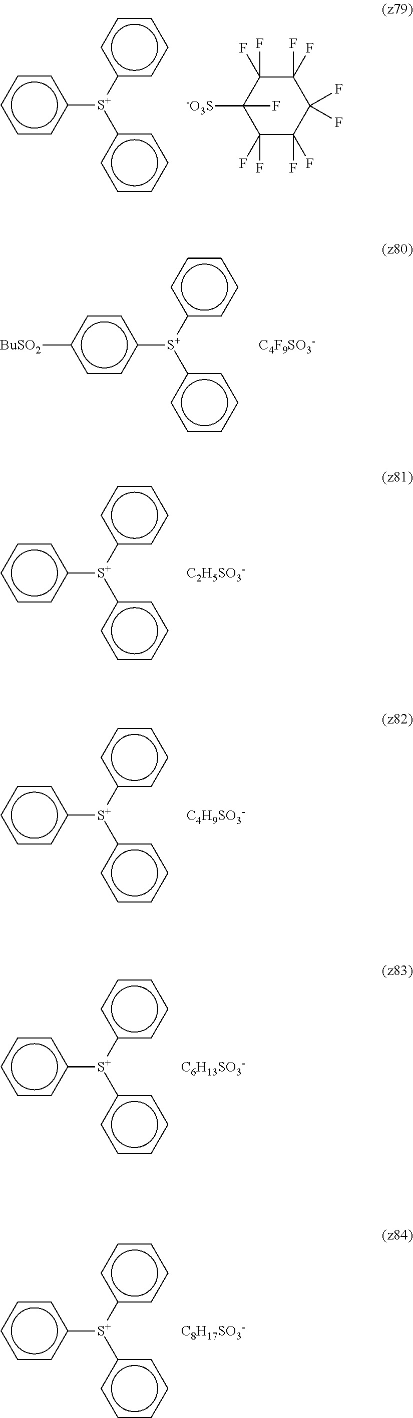

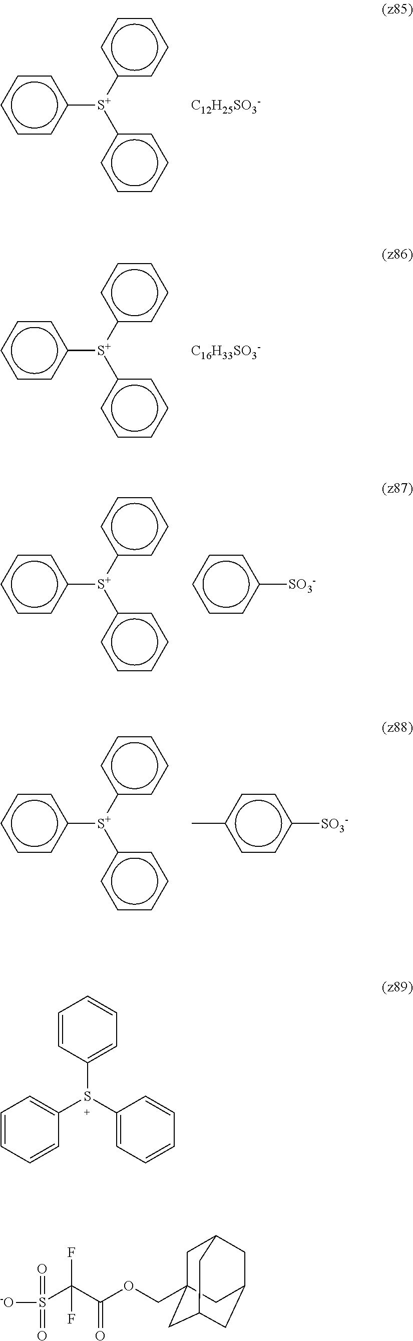

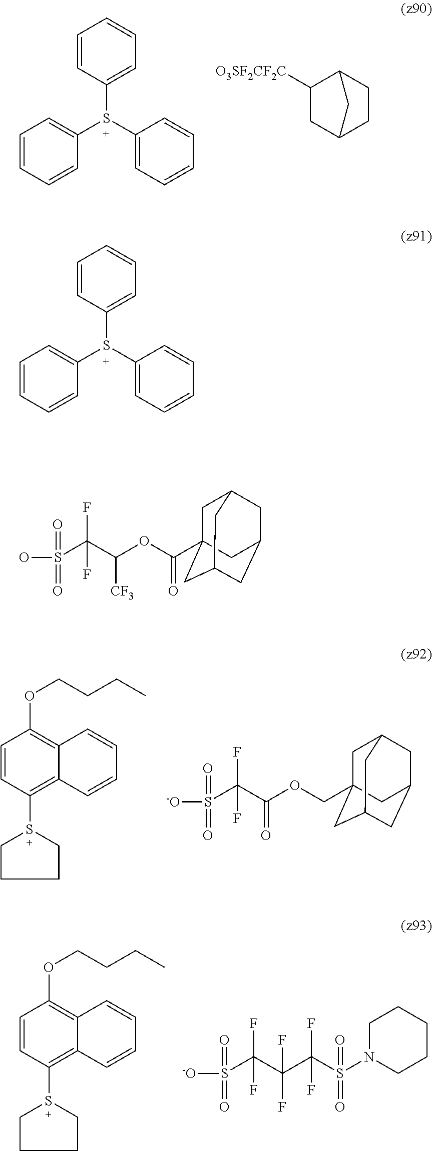

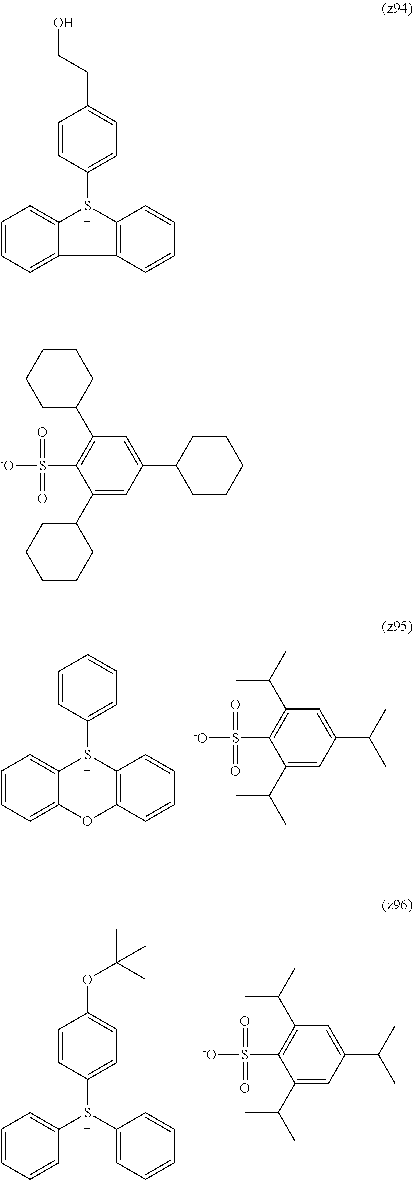

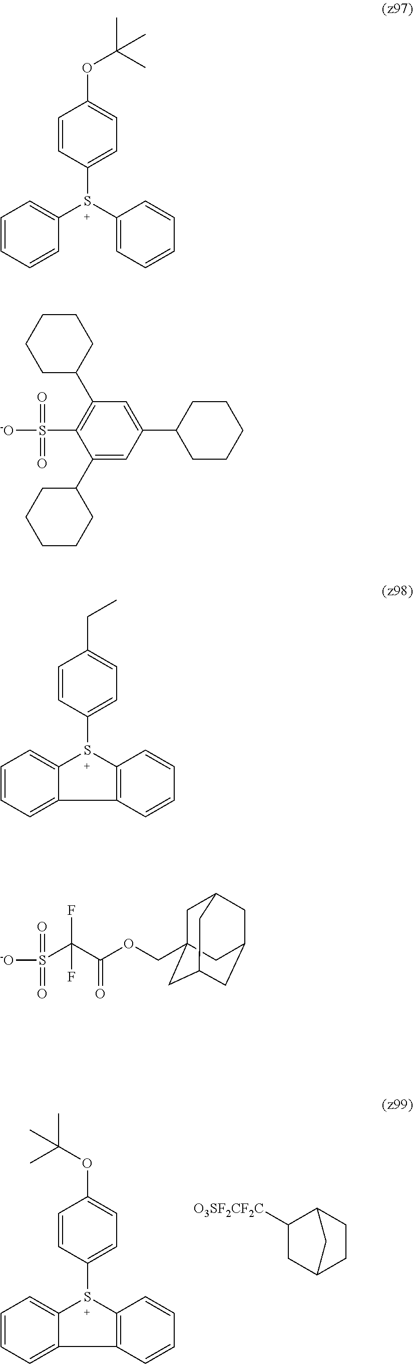

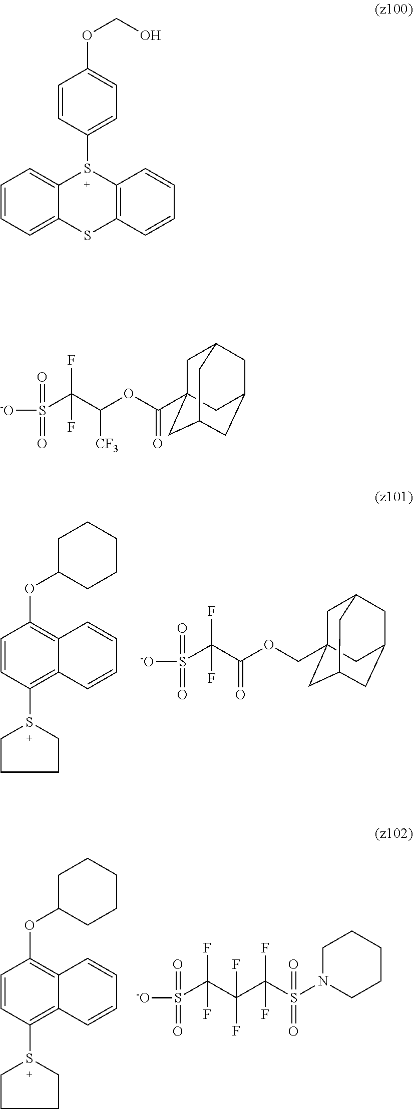

The compound represented by general formula (I) can be used individually or in combination.

The content of the compound represented by general formula (I) based on the total solids of the composition is preferably in the range of 0.1 to 50 mass %, more preferably 0.5 to 45 mass % and further more preferably 1 to 40 mass %.

When any of the compounds of general formula (I) is used in combination with another photoacid generator, the ratio of the former to the latter is preferably in the range of 50:50 to 99:1, more preferably 70:30 to 90:10.

The total content of the photoacid generator based on the total solids of the composition is preferably in the range of 0.1 to 50 mass %, more preferably 0.5 to 45 mass % and further more preferably 1 to 40 mass %.

[2] Acid-Decomposable Resin

The acid-decomposable resin typically contains an acid-decomposable group, that is, a group configured to decompose by the action of an acid, to thereby generate an alkali-soluble group. The resin may contain the acid-decomposable group in its principal chain, in its side chain, or in both thereof. Among them, a resin having an acid-decomposable group in its side chain is preferred.

The acid-decomposable group is preferably a group resulting from substitution of the hydrogen atom of an alkali-soluble group, such as a —COOH group or an —OH group, with an acid-eliminable group. The acid-decomposable group is preferably an acetal group or a tertiary ester group.

The matrix resin for bonding of the acid-decomposable group as a side chain is an alkali-soluble resin having, in its side chain, an —OH or —COOH group. For example, there can be mentioned the alkali-soluble resins to be described hereinafter.

The alkali dissolution rate of the alkali-soluble resin as measured in a 0.261 N tetramethylammonium hydroxide (TMAH) (23° C.) is preferably 17 nm/sec or greater. The alkali dissolution rate is especially preferably 33 nm/sec or greater.

The alkali-soluble resins especially preferred from this viewpoint include alkali-soluble resins having hydroxystyrene structural units, such as o-, m- or p-poly(hydroxystyrene) and copolymers thereof, hydrogenated poly(hydroxystyrene), halogenated or alkylated poly(hydroxystyrene), poly(hydroxystyrene) having its part O-alkylated or O-acylated, styrene-hydroxystyrene copolymer, α-methylstyrene-hydroxystyrene copolymer and hydrogenated novolak resin and include alkali-soluble resins having carboxylated repeating units, such as those of (meth)acrylic acid and norbornene carboxylic acid.

As repeating units having an acid-decomposable group preferred in the present invention, there can be mentioned, for example, repeating units derived from t-butoxycarbonyloxystyrene, a 1-alkoxyethoxystyrene and a (meth)acrylic acid tertiary alkyl ester. Repeating units derived from a 2-alkyl-2-adamantyl(meth)acrylate and a dialkyl(1-adamantyl)methyl(meth)acrylate are more preferred.

The resin for use in the present invention can be obtained by reaction of a precursor of acid-decomposable group with an alkali-soluble resin or by copolymerization of an alkali-soluble resin monomer having an acid-decomposable group bonded thereto with various monomers, as disclosed in, for example, EP 254853 and JP-A's 2-25850, 3-223860 and 4-251259.

When the composition according to the present invention is exposed to KrF excimer laser beams, electron beams, X-rays or high-energy light rays of 50 nm or less wavelength (EUV, etc.), it is preferred for the resin to have hydroxystyrene repeating units. More preferably, the resin is a copolymer of hydroxystyrene/hydroxystyrene protected by an acid-decomposable group or a copolymer of hydroxystyrene/(meth)acrylic acid tertiary alkyl ester.

In particular, the resin is preferably, for example, the one having any of the repeating units of general formula (A) below.

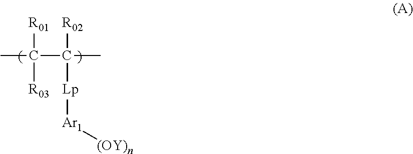

In the formula, each of R₀₁, R₀₂ and R₀₃ independently represents a hydrogen atom, an alkyl group, a cycloalkyl group, a halogen atom, a cyano group or an alkoxycarbonyl group. Ar₁ represents, for example, an aromatic ring group. R03 may be an alkylene group and bonded to Lp or Ar₁ to form a ring in cooperation with —C—C—. Alternatively, R₀₃ and Ar₁ may be simultaneously alkylene groups and bonded to each other so as to form a ring in cooperation with —C—C—. The ring may be, for example, a 5-membered or 6-membered ring.

Each of n Y's independently represents a hydrogen atom or a group that is eliminated by the action of an acid, provided that at least one of the Y's is a group that is eliminated by the action of an acid.

In the formula, n is an integer of 1 to 4, preferably 1 or 2 and more preferably 1.

Lp represents a single bond or a bivalent connecting group.

As preferred alkyl groups represented by R₀₁ to R₀₃ in the general formula, there can be mentioned alkyl groups having up to 20 carbon atoms, such as a methyl group, an ethyl group, a propyl group, an isopropyl group, an n-butyl group, a sec-butyl group, a hexyl group, a 2-ethylhexyl group, an octyl group and a dodecyl group. Alkyl groups having up to 8 carbon atoms are more preferred. These alkyl group may contain one or more substituents.

The alkyl groups contained in the alkoxycarbonyl groups are preferably the same as the above-mentioned alkyl groups represented by R₀₁ to R₀₃.

The cycloalkyl groups may be monocyclic or polycyclic. As preferred examples thereof, there can be mentioned monocyclic alkyl groups having 3 to 8 carbon atoms, such as a cyclopropyl group, a cyclopentyl group and a cyclohexyl group. These cycloalkyl groups may contain one or more substituents.

As the halogen atom, there can be mentioned a fluorine atom, a chlorine atom, a bromine atom or an iodine atom. A fluorine atom is preferred.

As preferred alkylene groups represented by R₀₃, there can be mentioned those having 1 to 8 carbon atoms, such as a methylene group, an ethylene group, a propylene group, a butylene group, a hexylene group and an octylene group.

The aromatic ring group represented by Ar₁ is preferably an aromatic ring group having 6 to 14 carbon atoms. In particular, there can be mentioned a benzene ring, a toluene ring, a naphthalene ring or the like. These aromatic ring groups may contain one or more substituents.

As a bivalent group represented by Lp, for example, an alkylene group, a cycloalkylene group, an arylene group, —COO—, —COO—, —CO—, —O—, —S—, —S(═O)—, —S(═O)₂—, —OS(═O)₂—, —NH—, or the combination of at least two thereof can be exemplified.

The alkylene group represented by Lp may either be substituted or unsubstituted. The alkylene group preferably has 1 to 20 carbon atoms and more preferably has 1 to 10 carbon atoms. As such, there can be mentioned, for example, a methylene group, an ethylene group, and a propylene group.

The cycloalkylene group represented by Lp may either be monocyclic or polycyclic. The cycloalkylene group preferably has 3 to 20 carbon atoms and more preferably has 3 to 10 carbon atoms. As such, there can be mentioned, for example, a 1,4-cyclohexylene group.

The arylene group represented by Lp preferably has 6 to 20 carbon atoms and more preferably has 6 to 10 carbon atoms. As such, there can be mentioned, for example, a phenylene group and a naphthylene group.

The alkylene group, the cycloalkylene group, and the arylene group described above may further contain one or more substitutents. As such, those explained with respect to the group represented by R in general formula (I) can be exemplified.

As the group Y that is eliminated by the action of an acid, there can be mentioned, for example, —C(R₃₆)(R₃₇)(R₃₈), —C(═O)—O—C(R₃₆)(R₃₇)(R₃₈), —C(R₀₁)(R₀₂)(OR₃₉), —C(R₀₁)(R₀₂)—C(═O)—O—C(R₃₆)(R₃₇)(R₃₈), —CH(R₃₆)(Ar) or the like.

In the formulae, each of R₃₆ to R₃₉ independently represents an alkyl group, a cycloalkyl group, an aryl group, an aralkyl group or an alkenyl group. R₃₆ and R₃₇ may be bonded with each other to thereby form a ring structure.

Each of R₀₁ and R₀₂ independently represents a hydrogen atom, an alkyl group, a cycloalkyl group, an aryl group, an aralkyl group or an alkenyl group.

Ar represents an aryl group.

The alkyl groups represented by R₃₆ to R₃₉ and R₀₁ and R₀₂ each preferably have 1 to 8 carbon atoms. For example, there can be mentioned a methyl group, an ethyl group, a propyl group, an n-butyl group, a sec-butyl group, a hexyl group, an octyl group and the like.

The cycloalkyl groups represented by R₃₆ to R₃₉ and R₀₁ and R₀₂ may be monocyclic or polycyclic. The monocyclic alkyl groups are preferably cycloalkyl groups having 3 to 8 carbon atoms. As such, there can be mentioned, for example, a cyclopropyl group, a cyclobutyl group, a cyclopentyl group, a cyclohexyl group, a cyclooctyl group and the like. The polycyclic alkyl groups are preferably cycloalkyl groups having 6 to 20 carbon atoms. As such, there can be mentioned, for example, an adamantyl group, a norbornyl group, an isobornyl group, a camphonyl group, a dicyclopentyl group, an α-pinel group, a tricyclodecanyl group, a tetracyclododecyl group, an androstanyl group and the like. With respect to these, the carbon atoms of each of the cycloalkyl groups may be partially substituted with a heteroatom, such as an oxygen atom.

The aryl groups represented by R₃₆ to R₃₉, R₀₁ and R₀₂ and Ar each preferably have 6 to 10 carbon atoms. For example, there can be mentioned a phenyl group, a naphthyl group, an anthryl group and the like.

The aralkyl groups represented by R₃₆ to R₃₉, R₀₁ and R₀₂ each preferably have 7 to 12 carbon atoms. For example, there can be mentioned a benzyl group, a phenethyl group, a naphthylmethyl group and the like.

The alkenyl groups represented by R₃₆ to R₃₉, R₀₁ and R₀₂ each preferably have 2 to 8 carbon atoms. For example, there can be mentioned a vinyl group, an allyl group, a butenyl group, a cyclohexenyl group and the like.

The ring formed by mutual bonding of R₃₆ and R₃₇ may be monocyclic or polycyclic. The monocyclic structure is preferably a cycloalkane structure having 3 to 8 carbon atoms. As such, there can be mentioned, for example, a cyclopropane structure, a cyclobutane structure, a cyclopentane structure, a cyclohexane structure, a cycloheptane structure, a cyclooctane structure or the like. The polycyclic structure is preferably a cycloalkane structure having 6 to 20 carbon atoms. As such, there can be mentioned, for example, an adamantane structure, a norbornane structure, a dicyclopentane structure, a tricyclodecane structure, a tetracyclododecane structure or the like. With respect to these, the carbon atoms of each of the cycloalkane structure may be partially substituted with a heteroatom, such as an oxygen atom.

Each of the groups represented by R₃₆ to R₃₉, R₀₁, R₀₂, R₀₃, Ar and Ar₁ may have one or more substituents. As the substituent, there can be mentioned, for example, an alkyl group, a cycloalkyl group, an aryl group, an amino group, an amido group, a ureido group, a urethane group, a hydroxyl group, a carboxyl group, a halogen atom, an alkoxy group, a thioether group, an acyl group, an acyloxy group, an alkoxycarbonyl group, a cyano group, a nitro group or the like. Preferably, the number of carbon atoms of each of the substituents is up to 8.

The group Y that is eliminated by the action of an acid more preferably has any of the structures of general formula (B) below.

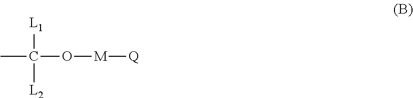

In the formula, each of L₁ and L₂ independently represents a hydrogen atom, an alkyl group, a cycloalkyl group, an aryl group or an aralkyl group.

M represents a single bond or a bivalent connecting group.

Q represents an alkyl group, a cycloalkyl group, an alicyclic group, an aromatic ring group, an amino group, an ammonium group, a mercapto group, a cyano group or an aldehyde group. Note that the alicyclic group or the aromatic ring group may contain one or more hetero-atoms.

At least two of Q, M and L₁ may be bonded to each other to thereby form a 5-membered or 6-membered ring.

The alkyl groups represented by L₁ and L₂ are, for example, alkyl groups having 1 to 8 carbon atoms. As preferred examples thereof, there can be mentioned a methyl group, an ethyl group, a propyl group, an n-butyl group, a sec-butyl group, a hexyl group and an octyl group.

The cycloalkyl groups represented by L₁ and L₂ are, for example, cycloalkyl groups having 3 to 15 carbon atoms. As preferred examples thereof, there can be mentioned a cyclopentyl group, a cyclohexyl group, a norbornyl group and an adamantyl group.

The aryl groups represented by L₁ and L₂ are, for example, aryl groups having 6 to 15 carbon atoms. As preferred examples thereof, there can be mentioned a phenyl group, a tolyl group, a naphthyl group, an anthryl group and the like.

The aralkyl groups represented by L₁ and L₂ are, for example, those having 6 to 20 carbon atoms. There can be mentioned a benzyl group, a phenethyl group and the like.

The bivalent connecting group represented by M is, for example, an alkylene group (e.g., a methylene group, an ethylene group, a propylene group, a butylene group, a hexylene group, an octylene group, etc.), a cycloalkylene group (e.g., a cyclopentylene group, a cyclohexylene group, etc.), an alkenylene group (e.g., an ethylene group, a propenylene group, a butenylene group, etc.), an arylene group (e.g., a phenylene group, a tolylene group, a naphthylene group, etc.), —S—, —O—, —CO—, —SO₂—, —N(R₀)— or a bivalent connecting group resulting from combination of these groups. R₀ represents a hydrogen atom or an alkyl group. The alkyl group is, for example, an alkyl group having 1 to 8 carbon atoms such as a methyl group, an ethyl group, a propyl group, an n-butyl group, a sec-butyl group, a hexyl group, an octyl group or the like.

The alkyl group and cycloalkyl group represented by Q are the same as those mentioned above as L₁ and L₂.

As the alicyclic group and aromatic ring group contained in the alicyclic group optionally containing a heteroatom and aromatic ring group optionally containing a heteroatom represented by Q, there can be mentioned, for example, the cycloalkyl group and aryl group mentioned above as L₁ and L₂. Preferably, each of the alicyclic group and aromatic ring group has 3 to 15 carbon atoms.

As the alicyclic group containing a heteroatom and aromatic ring group containing a heteroatom, there can be mentioned, for example, groups having a heterocyclic structure, such as thiirane, cyclothiorane, thiophene, furan, pyrrole, benzothiophene, benzofuran, benzopyrrole, triazine, imidazole, benzimidazole, triazole, thiadiazole, thiazole and pyrrolidone. However, the alicyclic groups and aromatic ring groups are not limited to these as long as the ring is formed by carbon and a heteroatom or by heteroatoms.

As the ring that may be formed by mutual bonding of at least two of Q, M and L₁, there can be mentioned the 5-membered or 6-membered ring resulting from mutual bonding of at least two of Q, M and L₁ so as to form, for example, a propylene group or a butylene group and subsequent formation of a ring containing an oxygen atom.

In the general formula (B), each of the groups represented by L₁, L₂, M and Q may have one or more substituents. As the substituent, there can be mentioned, for example, an alkyl group, a cycloalkyl group, an aryl group, an amino group, an amido group, an ureido group, an urethane group, a hydroxy group, a carboxy group, a halogen atom, an alkoxy group, a thioether group, an acyl group, an acyloxy group, an alkoxycarbonyl group, a cyano group, and a nitro group. Preferably, the number of carbon atoms of each of the substituents is up to 8.

The groups of the formula -(M-Q) are preferably groups having 1 to 30 carbon atoms, more preferably groups having 5 to 20 carbon atoms. From the viewpoint of outgas suppression, it is especially preferred for the number of carbon atoms to be 6 or greater.

As other preferable resin, those containing repeating units represented by the following general formula (X) can be exemplified.

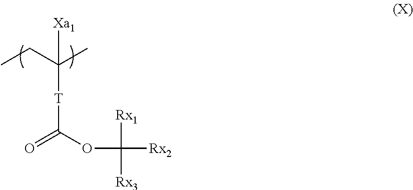

In general formula (X),

Xa₁ represents a hydrogen atom, a methyl group, a trifluoromethyl group or a hydroxymethyl group,

T represents a single bond or a bivalent connecting group, and

each of Rx₁ to Rx₃ independently represents an alkyl group (linear or branched) or a cycloalkyl group (monocyclic or polycyclic), wherein at least two of Rx₁ to Rx₃ may be bonded to each other to form a monocyclic or polycyclic alkyl group.

As the bivalent connecting group represented by T, an alkylene group, a group of the formula —COO-Rt-, and a group of the formula —O-Rt- can be exemplified. In the formulae, Rt represents an alkylene group or a cycloalkylene group.

T is preferably a single bond or a group of the formula —COO-Rt-. Rt is preferably an alkylene group having 1 to 5 carbon atoms, more preferably a —CH₂— group or —(CH₂)₃— group.

The alkyl group represented by each of Rx₁ to Rx₃ is preferably one having 1 to 4 carbon atoms, such as a methyl group, an ethyl group, an n-propyl group, an isopropyl group, an n-butyl group, an isobutyl group or a t-butyl group.

The cycloalkyl group represented by each of Rx₁ to Rx₃ is preferably a monocyclic alkyl group such as a cyclopentyl group or a cyclohexyl group, or a polycyclic alkyl group such as a norbornyl group, a tetracyclodecanyl group, a tetracyclododecanyl group or an adamantyl group.

The cycloalkyl group formed by bonding of at least two of Rx₁ to Rx₃ is preferably a monocyclic alkyl group such as a cyclopentyl group or a cyclohexyl group, or a polycyclic alkyl group such as a norbornyl group, a tetracyclodecanyl group, a tetracyclododecanyl group or an adamantyl group.

Particularly preferred is an embodiment in which Rx₁ is a methyl group or an ethyl group, and Rx₂ and Rx₃ are bonded to each other to form the above-mentioned cycloalkyl group.

Specific examples of the repeating units represented by the general formula (X) will be shown below, which however in no way limit the scope of the present invention.

In the formulae, Rx represents H, CH₃, CF₃, or CH₂OH. Each of Rxa and Rxb independently represents an alkyl group having 1 to 4 carbon atoms.

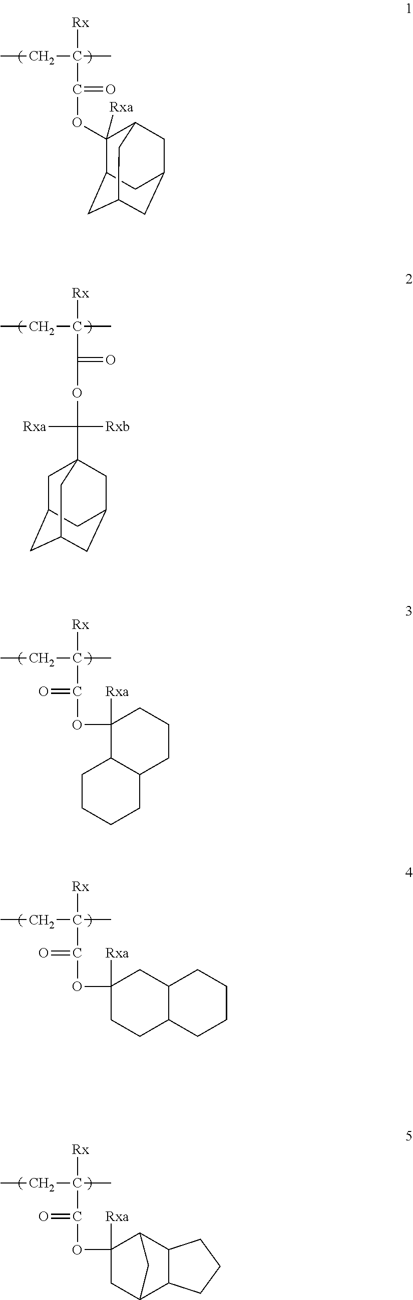

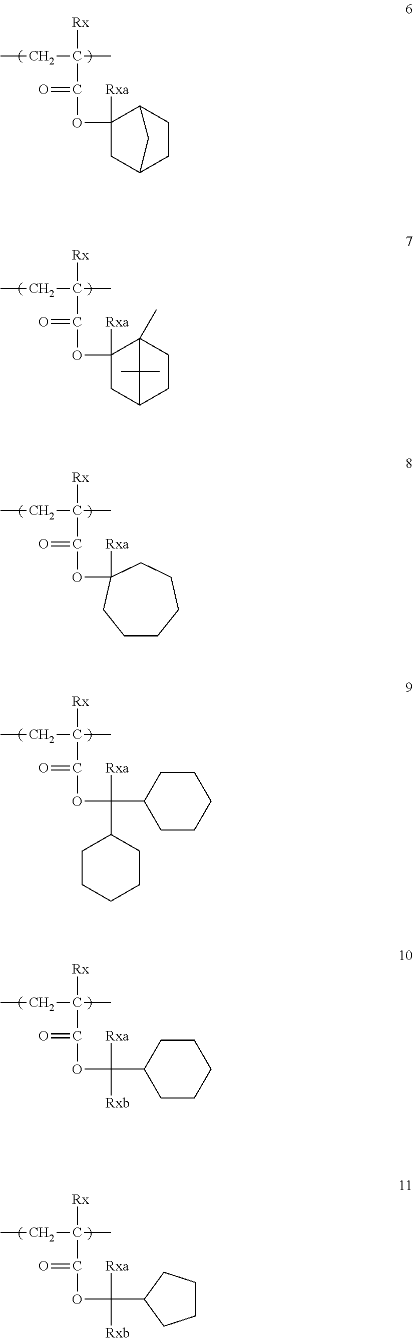

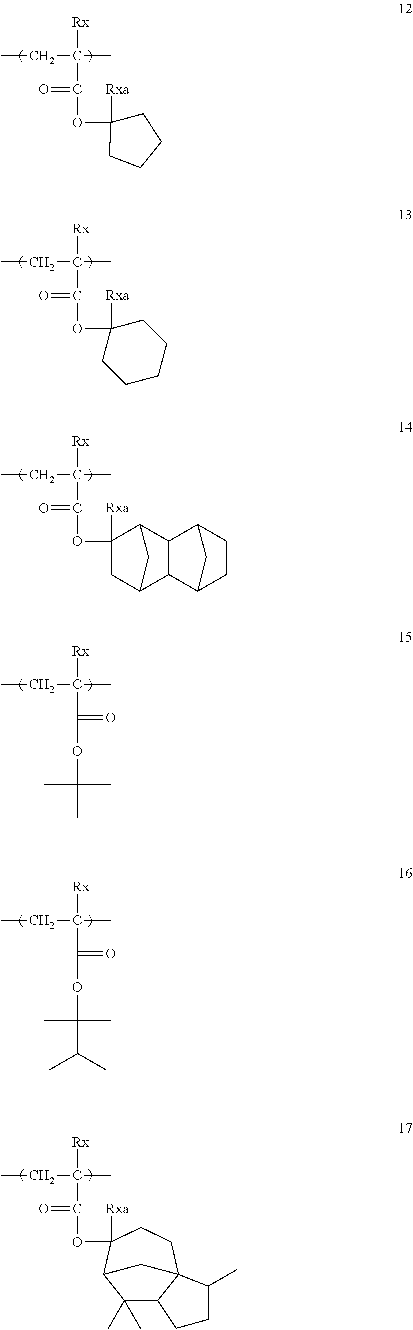

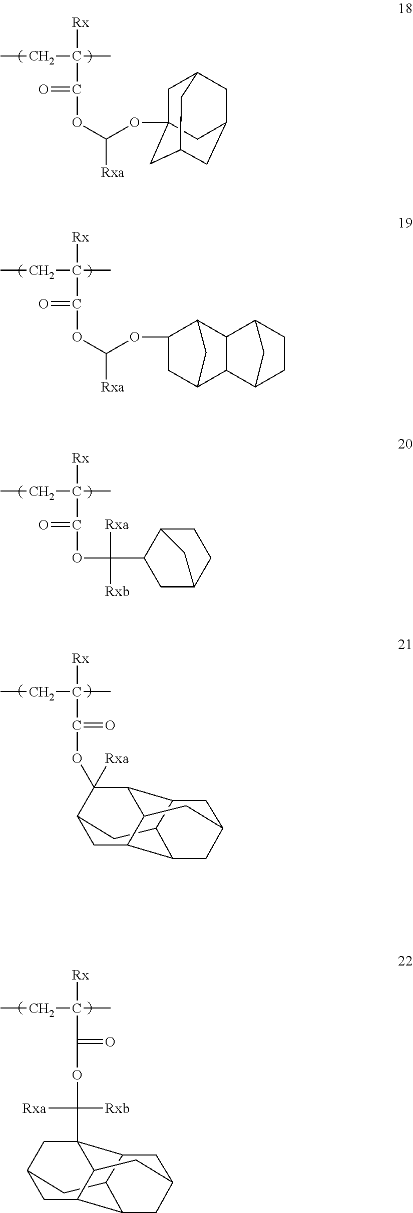

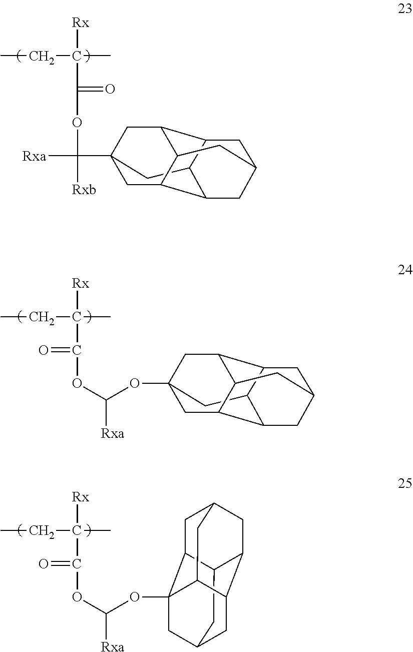

The content of repeating units represented by the general formula (X) based on all the repeating units of the resin is preferably in the range of 3 to 90 mol %, more preferably 5 to 80 mol % and still more preferably 7 to 70 mol %.

When the composition of the present invention is exposed to a KrF excimer laser light, electron beams, X-rays or high-energy light rays of wavelength 50 nm or shorter (for example, EUV), it is preferred for this resin to contain any of the repeating units of general formula (VI). As the repeating units of general formula (VI) contain an alkali-soluble group, the alkali solubility of the resin can be supplemented thereby. Also, the repeating units can enhance the pattern strength, and further can impart the function of Tg control for the film formed from the composition of the present invention.

Furthermore, the inventors have found that the sensitivity of the composition of the present invention can be enhanced by using the acid-decomposable resin containing any of the repeating units of general formula (VI) in combination with any of the aforementioned compounds of general formula (I). The reason therefor is not necessarily apparent. However, the inventors presume that the reason would be that a chain reaction described in J. Org. Chem. 2005, 70, 6809-6819 is likely to occur, so that the amount of generated acid is increased.

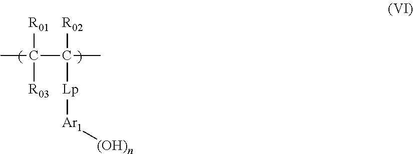

In the formula, each of R₀₁ and R₀₂ independently represents a hydrogen atom, an alkyl group, a cycloalkyl group, a halogen atom, a cyano group or an alkoxycarbonyl group. R₀₃ either represents a hydrogen atom, an alkyl group, a cycloalkyl group, a halogen atom, a cyano group or an alkoxycarbonyl group, or is bonded to the following Lp or Ar₁ to thereby form a ring structure.

Ar₁ represents an aromatic ring group.

In the formula, n is an integer of 1 or greater.

Lp represents a single bond or a bivalent connecting group.

In the formula, n is an integer of 1 to 4, preferably 1 or 2 and more preferably 1.

The alkyl groups represented by R₀₁ to R₀₃ are, for example, alkyl groups having up to 20 carbon atoms, such as a methyl group, an ethyl group, a propyl group, an isopropyl group, an n-butyl group, a sec-butyl group, a hexyl group, a 2-ethylhexyl group, an octyl group and a dodecyl group. Alkyl groups having up to 8 carbon atoms are preferred. One or more substituents may be introduced in these alkyl groups.

As the alkyl groups contained in the alkoxycarbonyl groups represented by R₀₁ to R₀₃, there can be mentioned, for example, those set forth above as examples of R₀₁ to R₀₃.

The cycloalkyl groups represented by R₀₁ to R₀₃ may be monocyclic or polycyclic. As preferred examples thereof, there can be mentioned monocycloalkyl groups each having 3 to 8 carbon atoms, such as a cyclopropyl group, a cyclopentyl group and a cyclohexyl group. Substituents may be introduced in these cycloalkyl groups.

As the halogen atom, there can be mentioned, for example, a fluorine atom, a chlorine atom, a bromine atom or an iodine atom. A fluorine atom is most preferred.

As the alkylene group represented by R₀₃, those having 1 to 8 carbon atoms such as a methylene group, an ethylene group, a propylene group, a butylene group, a hexylene group, and an octylene group can be exemplified.

The aromatic group represented by Ar₁ is preferably the one having 6 to 14 carbon atoms. As the aromatic ring contained in the aromatic group, a benzene ring, a toluene ring and a naphthalene ring can be exemplified. These aromatic groups may further have one or more substituents.

As examples of Lp, those explained with respect to general formula (A) can be exemplified.

Specific examples of the repeating units represented by general formula (VI) will be shown below.

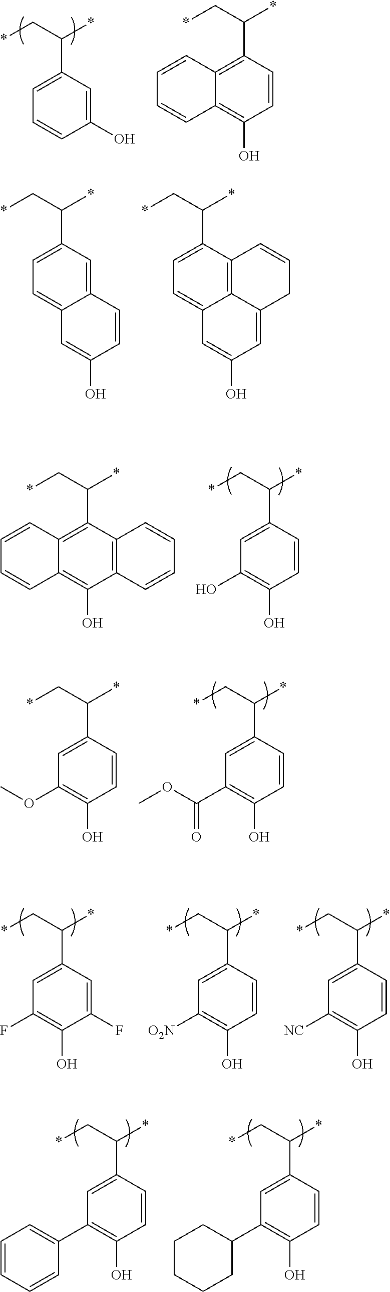

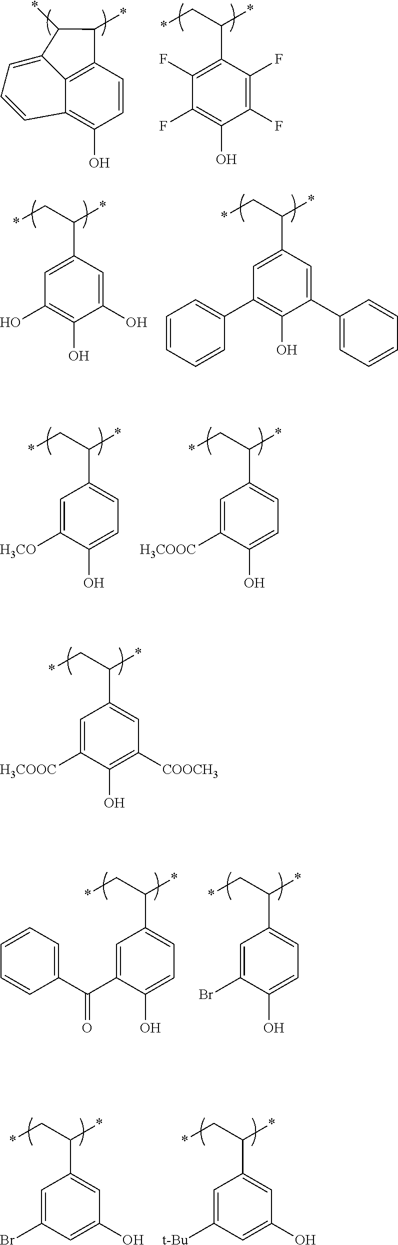

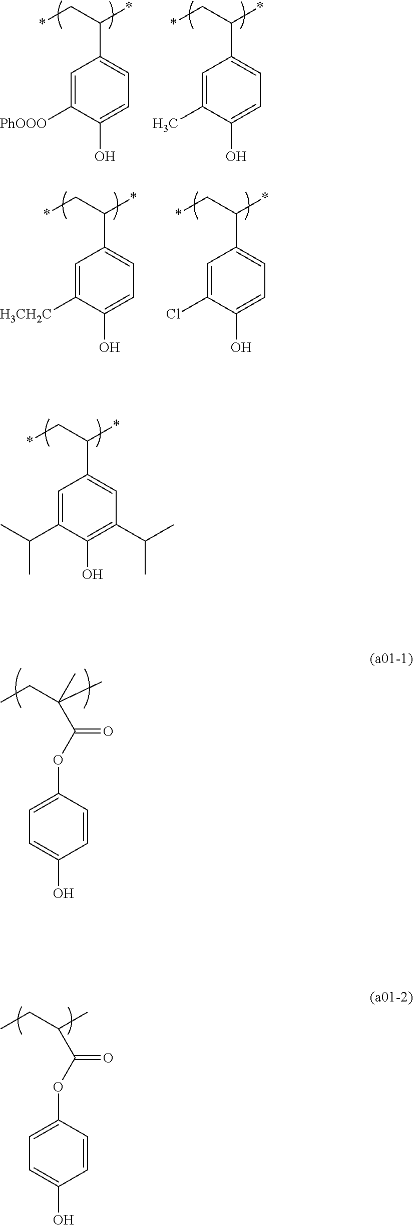

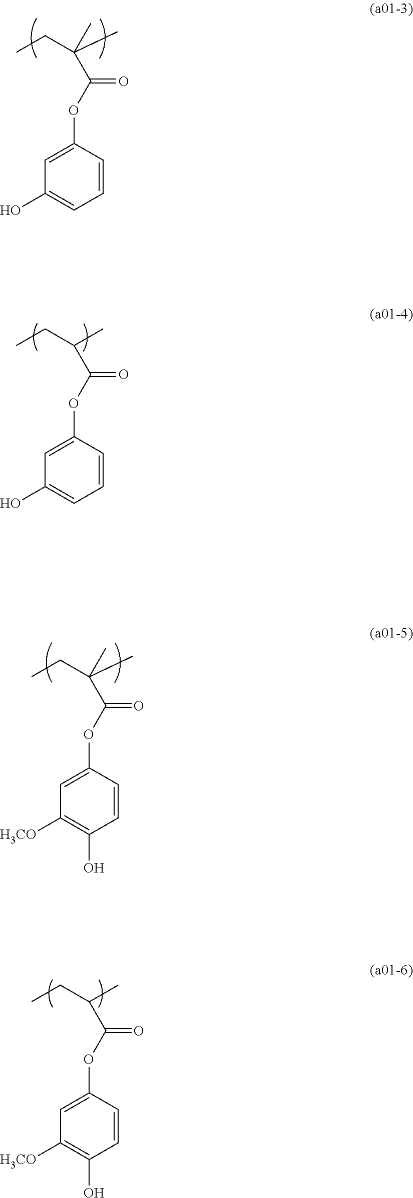

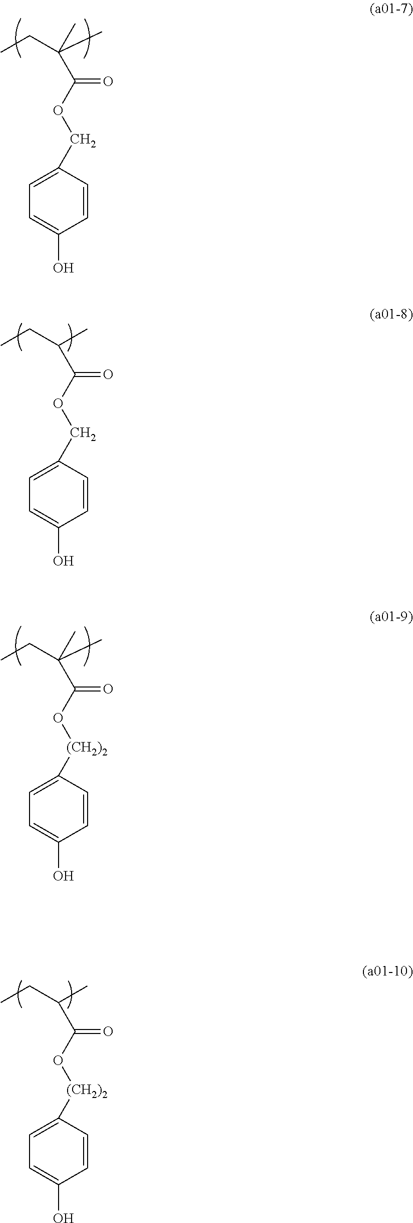

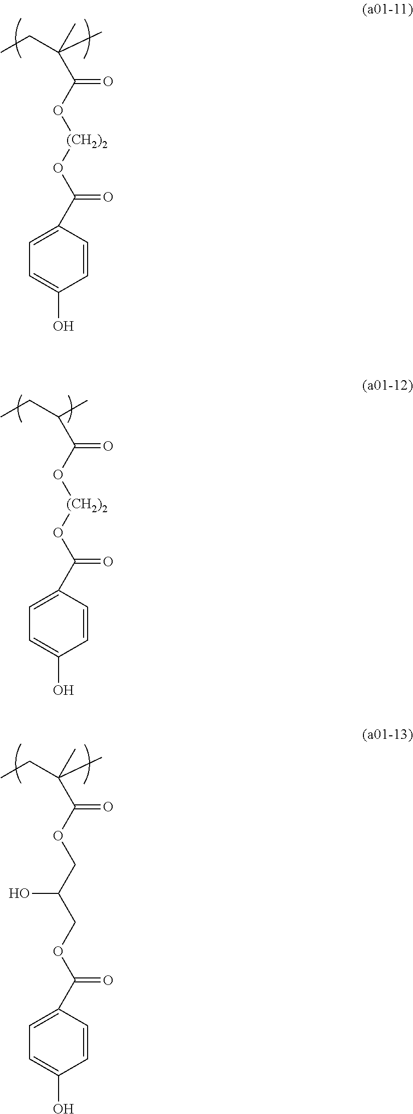

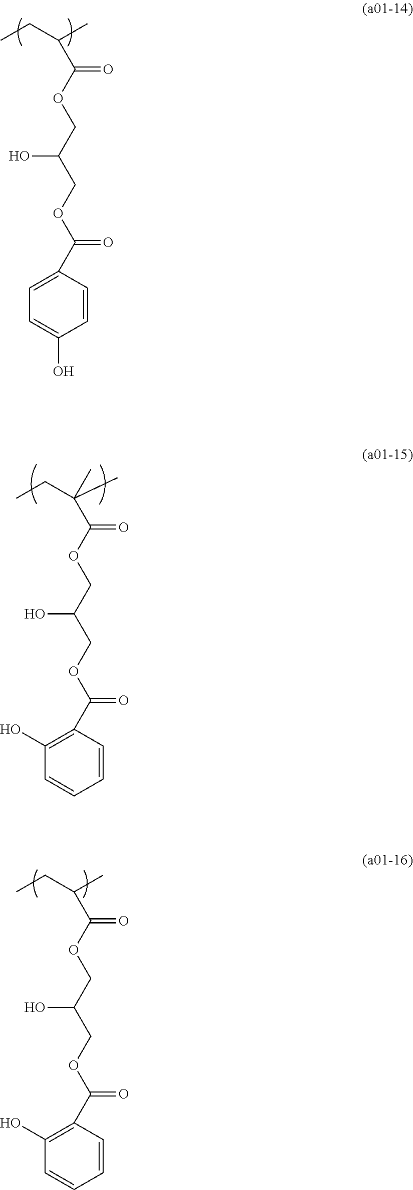

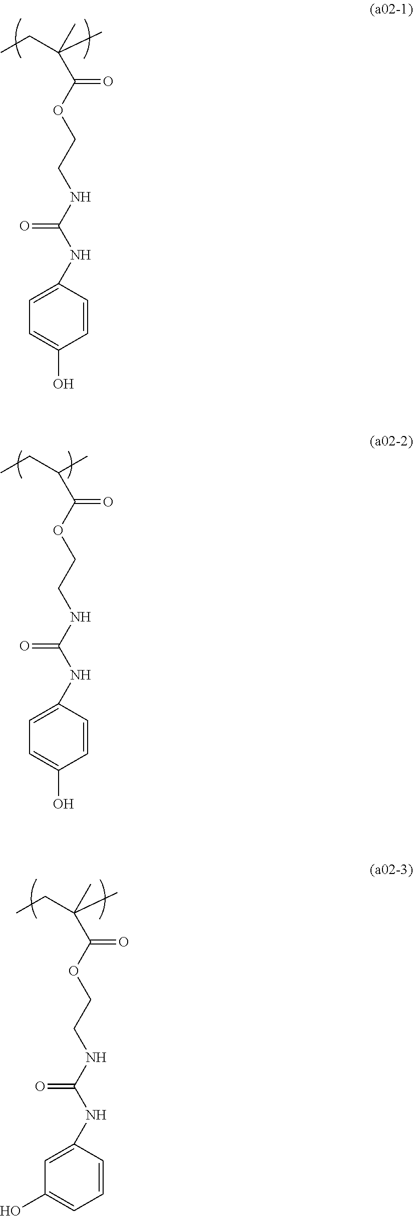

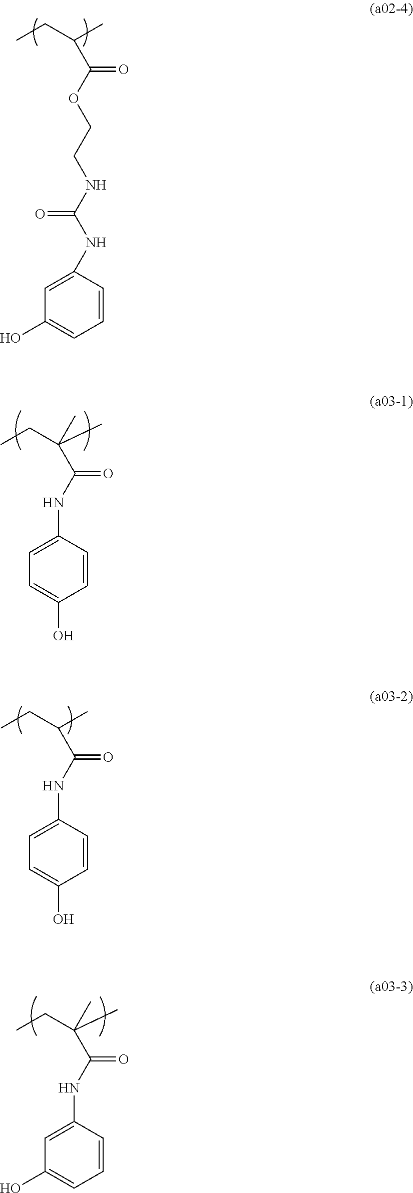

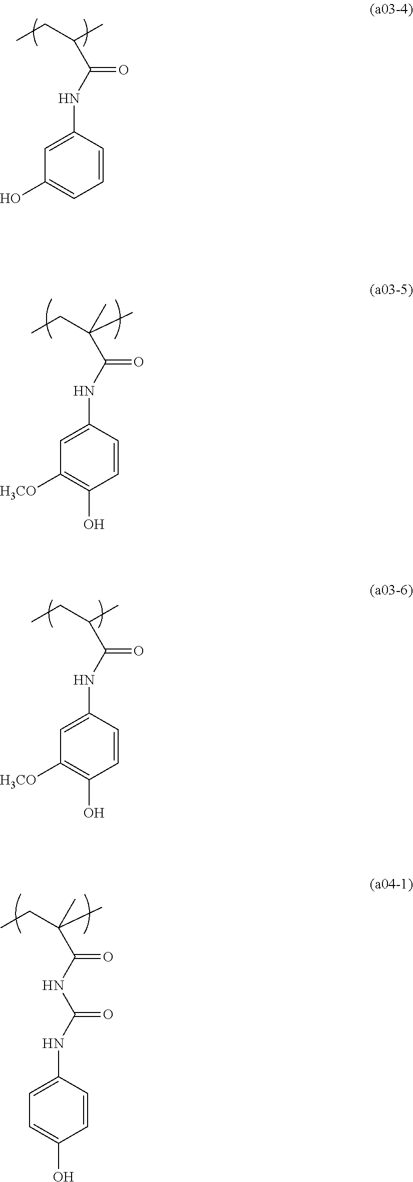

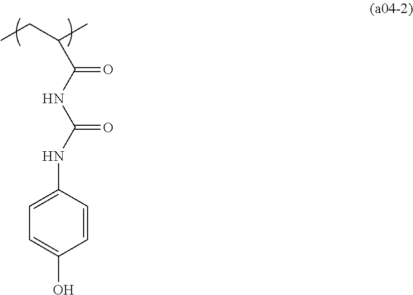

The content of repeating unit represented by general formula (VI) in an acid-decomposable resin, based on all the repeating units thereof, is preferably in the range of 20 to 90 mol % and more preferably 30 to 80 mol %.

Specific examples of the resin explained above will be shown below, which however in no way limit the scope of the present invention.

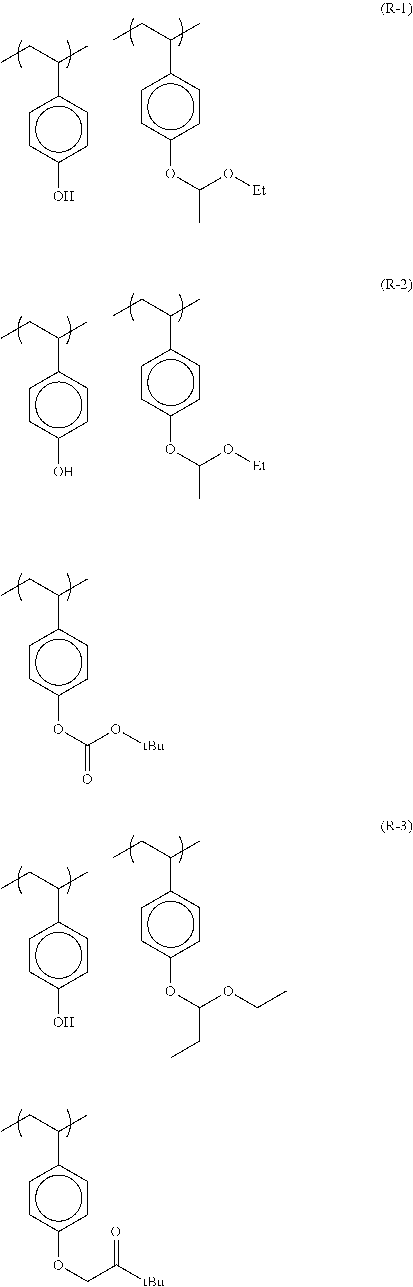

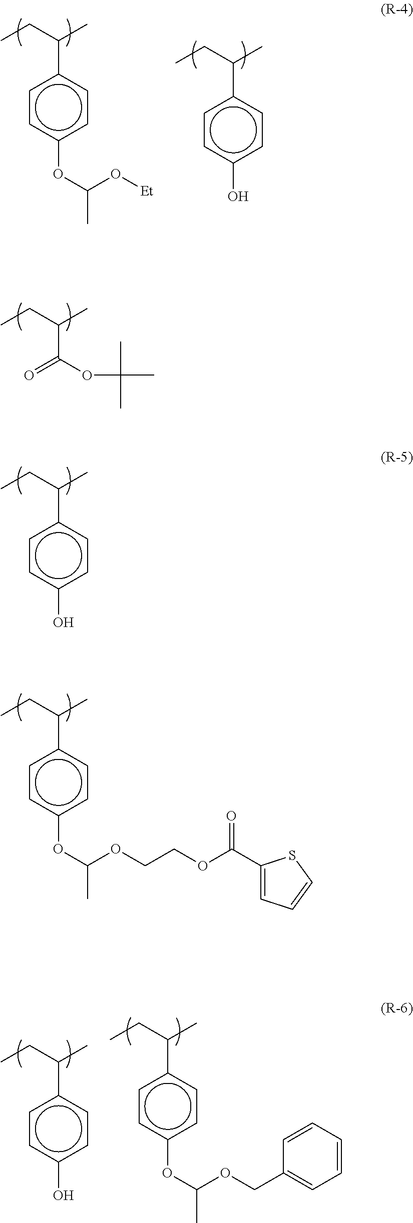

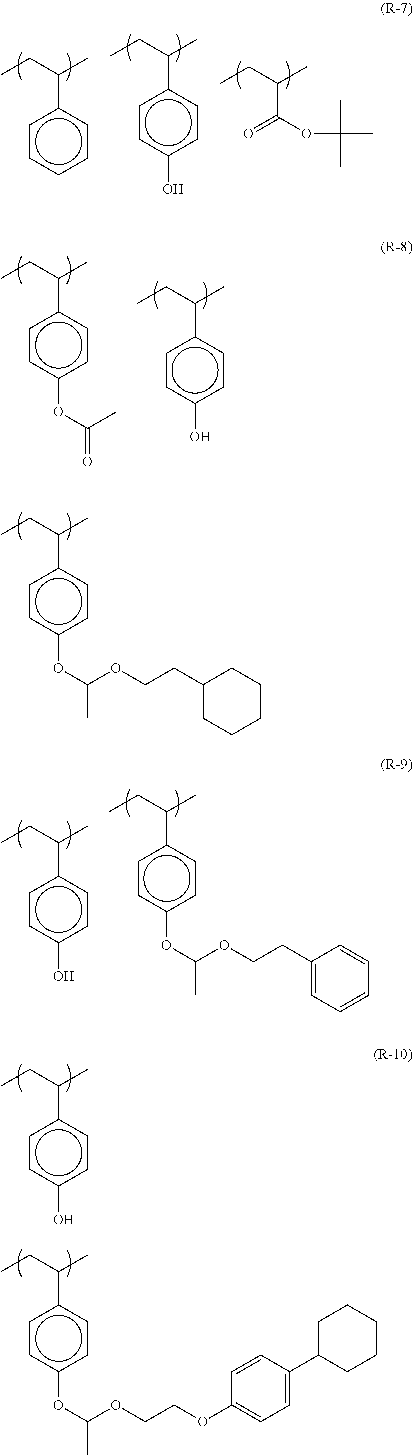

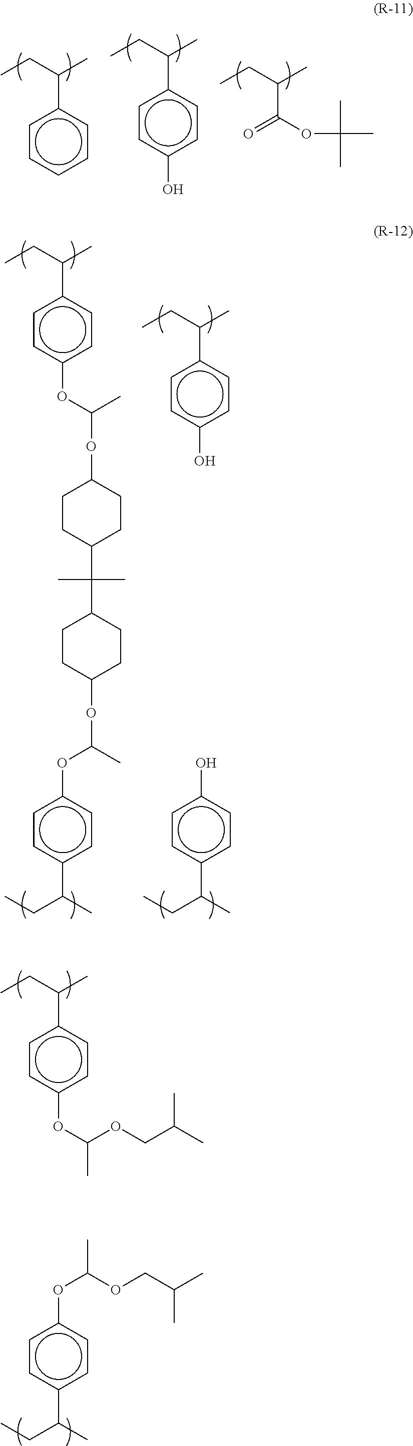

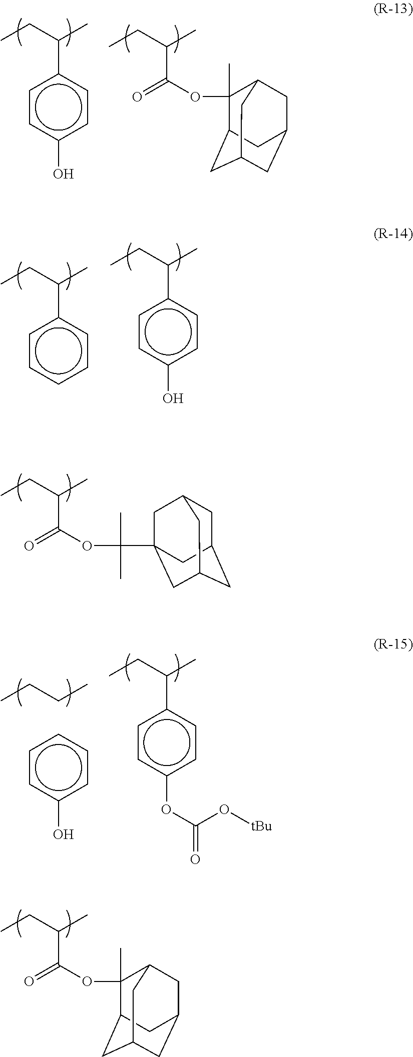

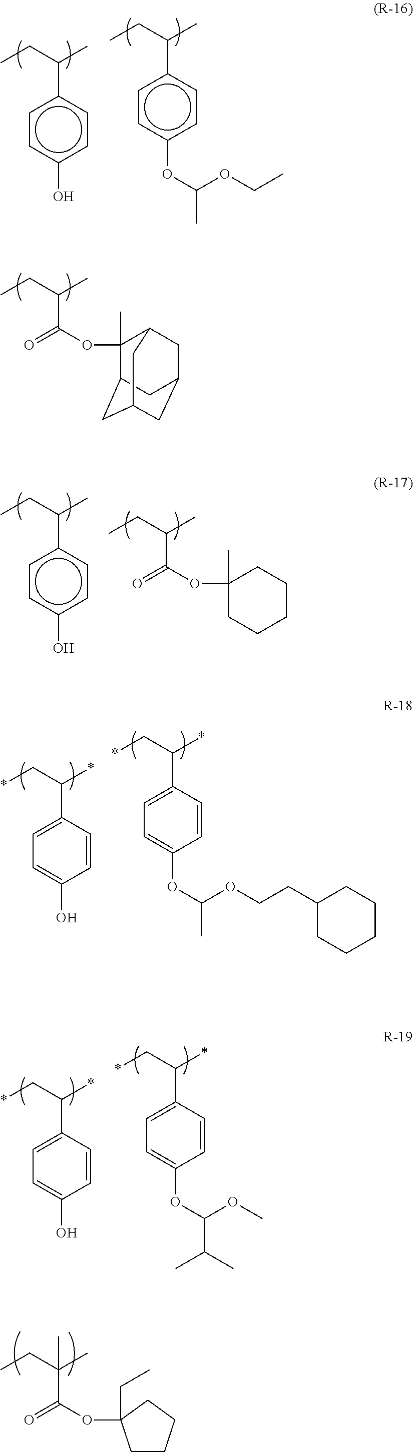

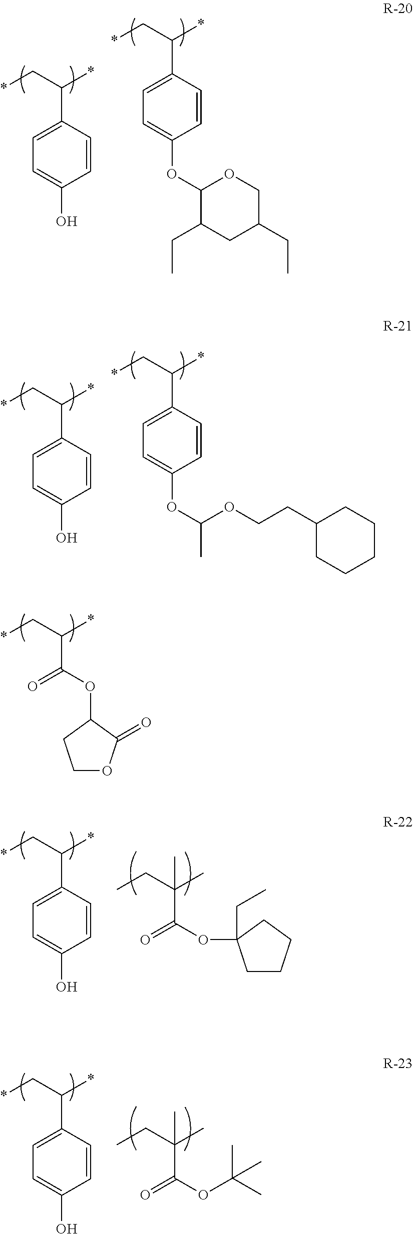

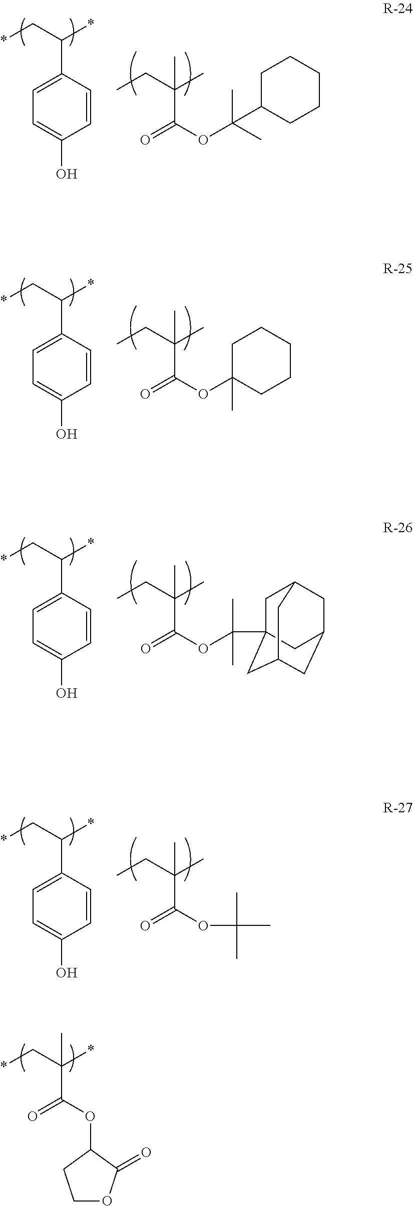

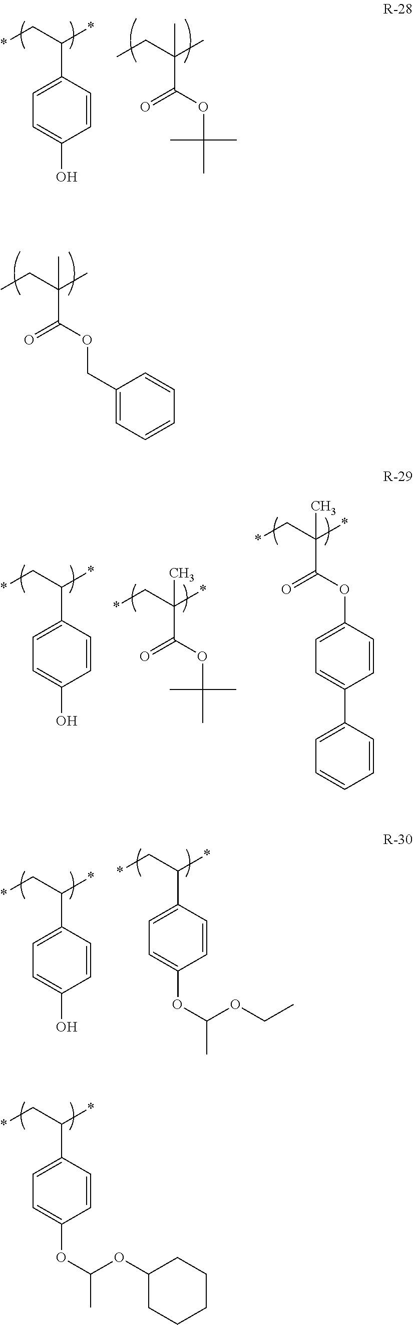

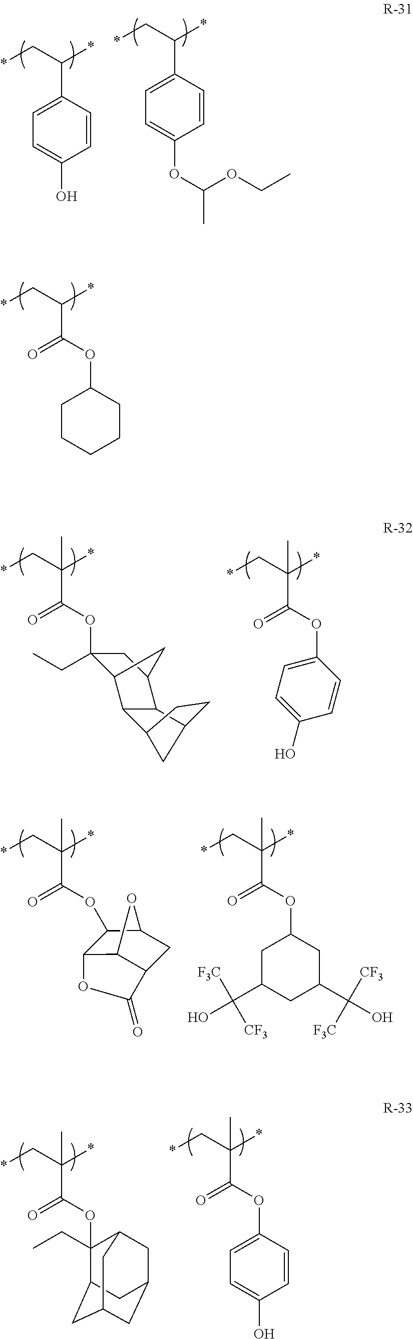

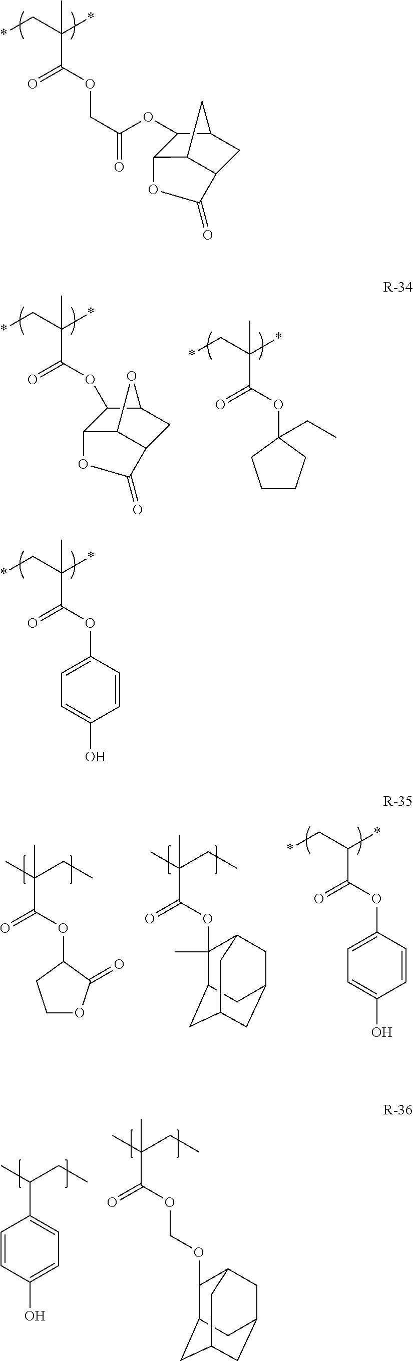

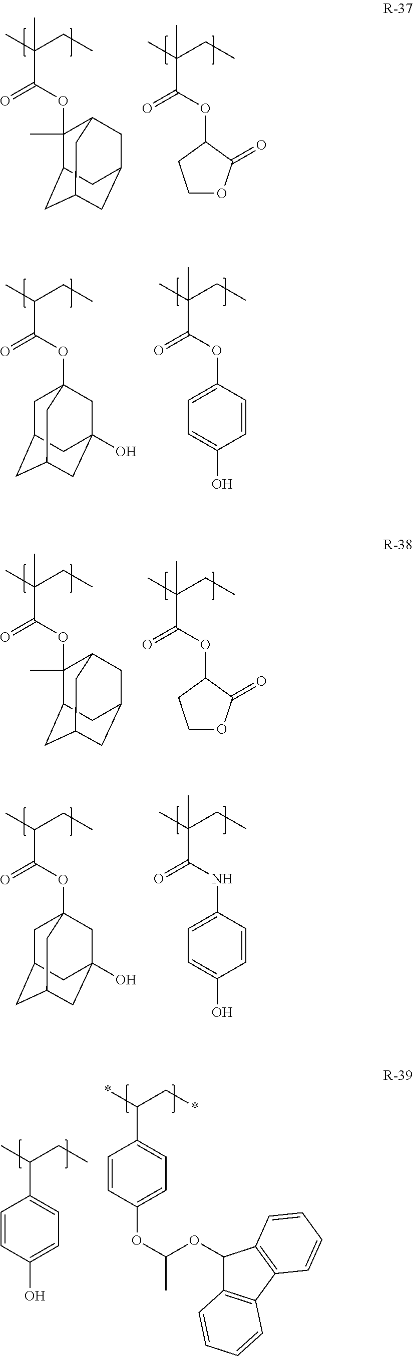

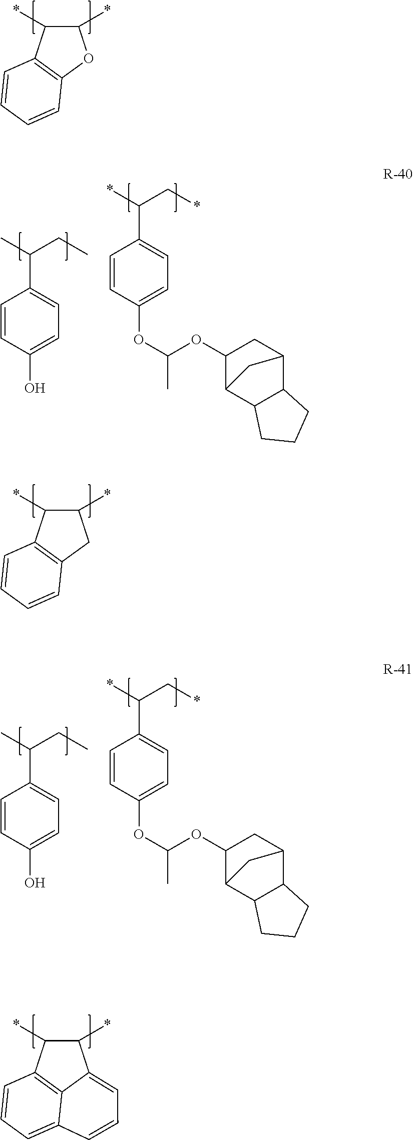

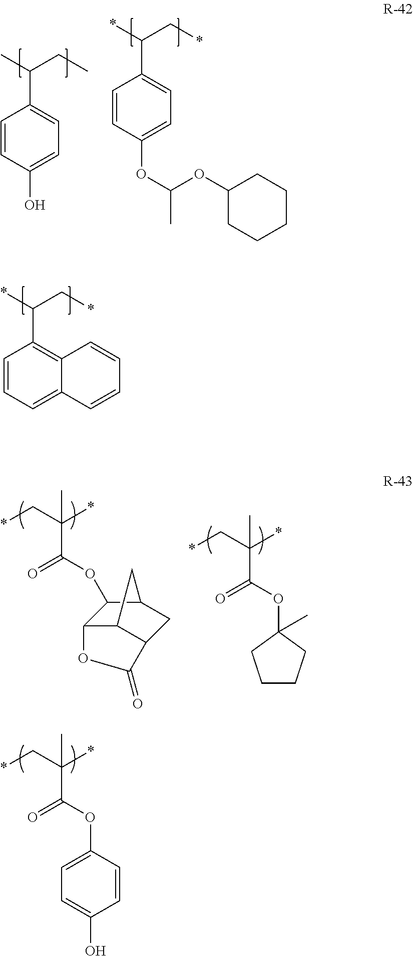

In the specific examples above, tBu represents a t-butyl group.

The content of acid-decomposable group is expressed by the formula B/(B+S) wherein B refers to the number of acid-decomposable groups contained in the resin and S refers to the number of alkali-soluble groups not protected by any acid-eliminable group. The content is preferably in the range of 0.01 to 0.7, more preferably 0.05 to 0.50 and further preferably 0.05 to 0.40.

When the composition according to the present invention is exposed to ArF excimer laser beams, it is preferred for the resin to contain an alicyclic hydrocarbon structure of a single ring or multiple rings. Such resins will be referred to as “alicyclic hydrocarbon based acid-decomposable resin” hereinbelow.

Preferably, the alicyclic hydrocarbon based acid-decomposable resin contains at least one member selected from the group consisting of the repeating units having partial structures containing the alicyclic hydrocarbons of general formulae (pI) to (pV) below and the repeating units of general formula (II-AB) below.

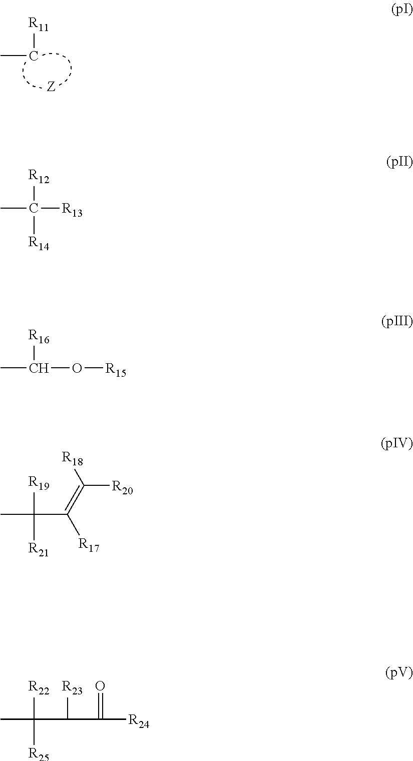

In the general formulae (pI) to (pV),

R₁₁ represents a methyl group, an ethyl group, an n-propyl group, an isopropyl group, an n-butyl group, an isobutyl group or a sec-butyl group, and Z represents an atomic group required for formation of a cycloalkyl group in cooperation with a carbon atom.

Each of R₁₂ to R₁₆ independently represents a cycloalkyl group or a linear or branched alkyl group having 1 to 4 carbon atoms, provided that at least one of R₁₂ to R₁₄ and either R₁₅ or R₁₆ represents a cycloalkyl group.

Each of R₁₇ to R₂₁ independently represents a hydrogen atom or a cycloalkyl group or a linear or branched alkyl group having 1 to 4 carbon atoms, provided that at least one of R₁₇ to R₂₁ represents a cycloalkyl group. Either R₁₉ or R₂₁ represents a cycloalkyl group or a linear or branched alkyl group having 1 to 4 carbon atoms.

Each of R₂₂ to R₂₅ independently represents a hydrogen atom or a cycloalkyl group or a linear or branched alkyl group having 1 to 4 carbon atoms, provided that at least one of R₂₂ to R₂₅ represents a cycloalkyl group. R₂₃ and R₂₄ may be bonded to each other to thereby form a ring.

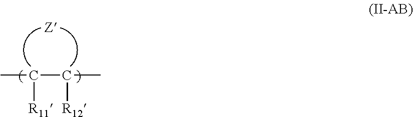

In the general formula (II-AB),

each of R₁₁′ and R₁₂′ independently represents a hydrogen atom, a cyano group, a halogen atom or an alkyl group.

Z′ represents an atomic group for formation of an alicyclic structure wherein two bonded carbon atoms (C—C) are contained.

Further preferably, the general formula (II-AB) is either general formula (II-AB1) or general formula (II-AB2) below.

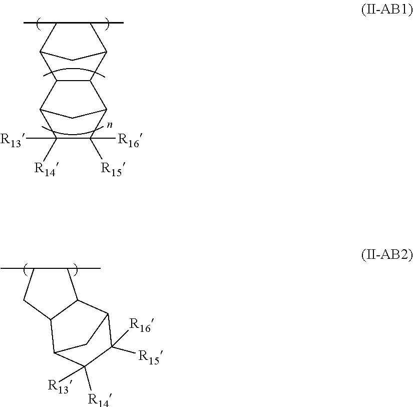

In the general formulae (II-AB1) and (II-AB2),

each of R₁₃′ to R₁₆′ independently represents a hydrogen atom, a halogen atom, a cyano group, a hydroxyl group, —COOH, —COOR₅, a group that is decomposed by the action of an acid, —C(═O)—X-A′—R₁₇′, an alkyl group or a cycloalkyl group. In the above formula, R₅ represents an alkyl group, a cycloalkyl group or a group with a lactone structure. X represents an oxygen atom, a sulfur atom, —NH—, —NHSO₂— or —NHSO₂NH—. A′ represents a single bond or a bivalent connecting group. R₁₇′ represents —COOH, —COOR₅, —CN, a hydroxyl group, an alkoxy group, —CO—NH—R₆, —CO—NH—SO₂—R₆ or a group with a lactone structure. R₆ represents an alkyl group or a cycloalkyl group. At least two of R₁₃′ to R₁₆′ may be bonded to each other to thereby form a ring.

n represents 0 or 1.

In the general formulae (pI) to (pV), each of the alkyl groups represented by R₁₂ to R₂₅ is a linear or branched alkyl group having 1 to 4 carbon atoms. As such, there can be mentioned, for example, a methyl group, an ethyl group, a propyl group, an n-butyl group, a sec-butyl group, a t-butyl group or the like.

The cycloalkyl groups represented by R₁₁ to R₂₅ and the cycloalkyl group formed by Z and a carbon atom may be monocyclic or polycyclic. In particular, there can be mentioned groups of a monocyclo, bicyclo, tricyclo or tetracyclo structure or the like having 5′ or more carbon atoms. The number of carbon atoms thereof is preferably in the range of 6 to 30, especially preferably 7 to 25.

As preferred cycloalkyl groups, there can be mentioned an adamantyl group, a noradamantyl group, a decalin residue, a tricyclodecanyl group, a tetracyclododecanyl group, a norbornyl group, a cedrol group, a cyclopentyl group, a cyclohexyl group, a cycloheptyl group, a cyclooctyl group, a cyclodecanyl group and a cyclododecanyl group. As more preferred cycloalkyl groups, there can be mentioned an adamantyl group, a norbornyl group, a cyclohexyl group, a cyclopentyl group, a tetracyclododecanyl group and a tricyclodecanyl group.

The structures of the general formulae (pI) to (pV) employed in the above resin can be used for the protection of the alkali-soluble groups. As the alkali-soluble groups, there can be mentioned various groups generally known in this technical field.

In particular, there can be mentioned, for example, structures resulting from replacement of a hydrogen atom of a carboxylic acid group, sulfonic acid group, phenol group or thiol group with any of the structures of the general formulae (pI) to (pV). Structures resulting from replacement of a hydrogen atom of a carboxylic acid group or sulfonic acid group with any of the structures of the general formulae (pI) to (pV) are preferred.

As preferred repeating units having any of the alkali-soluble groups protected by the structures of the general formulae (pI) to (pV), there can be mentioned those of general formula (pA) below.

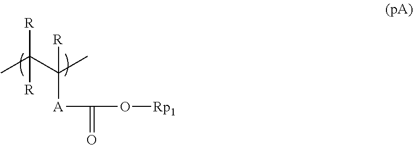

In the general formula (pA), R represents a hydrogen atom, a halogen atom or a linear or branched alkyl group having 1 to 4 carbon atoms. Two or more R's may be identical to or different from each other.

A represents any one or a combination of two or more groups selected from the group consisting of a single bond, an alkylene group, an ether group, a thioether group, a carbonyl group, an ester group, an amido group, a sulfonamido group, a urethane group and a urea group. A single bond is preferred.

Rp₁ represents any of the groups of the above general formulae (pI) to (pV).

The repeating units of the general formula (pA) are most preferably those derived from
a 2-alkyl-2-adamantyl(meth)acrylate and
a dialkyl(1-adamantyl)methyl(meth)acrylate.

Specific examples of the repeating units of the general formula (pA) will be shown below.

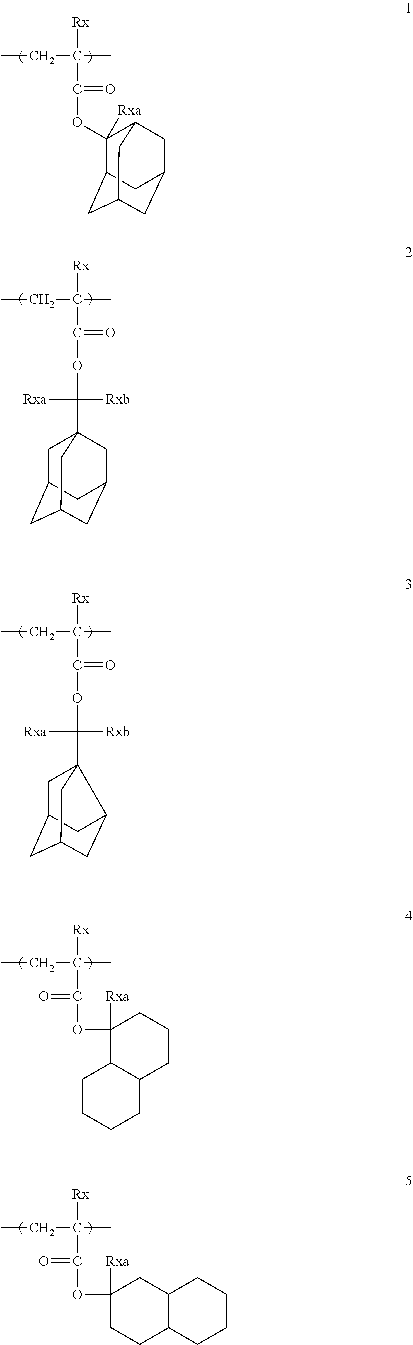

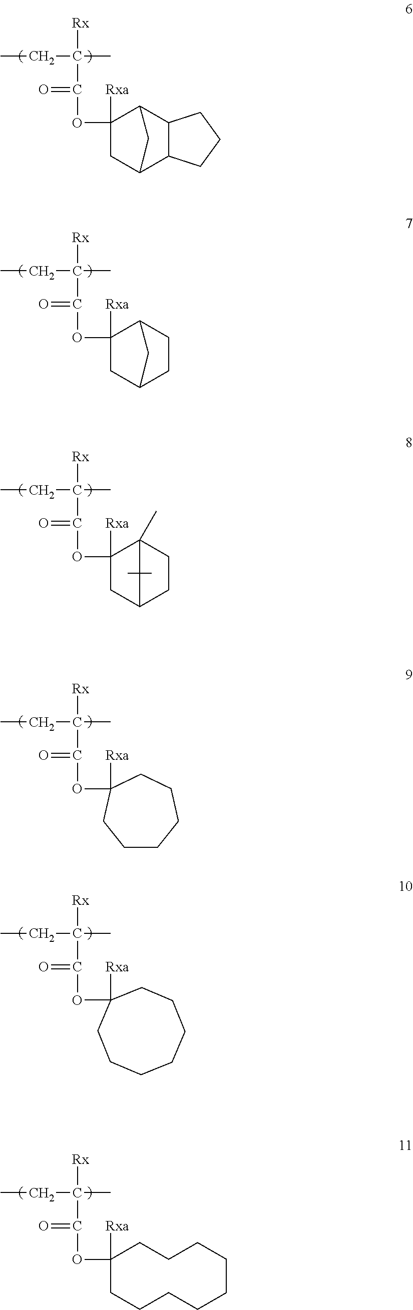

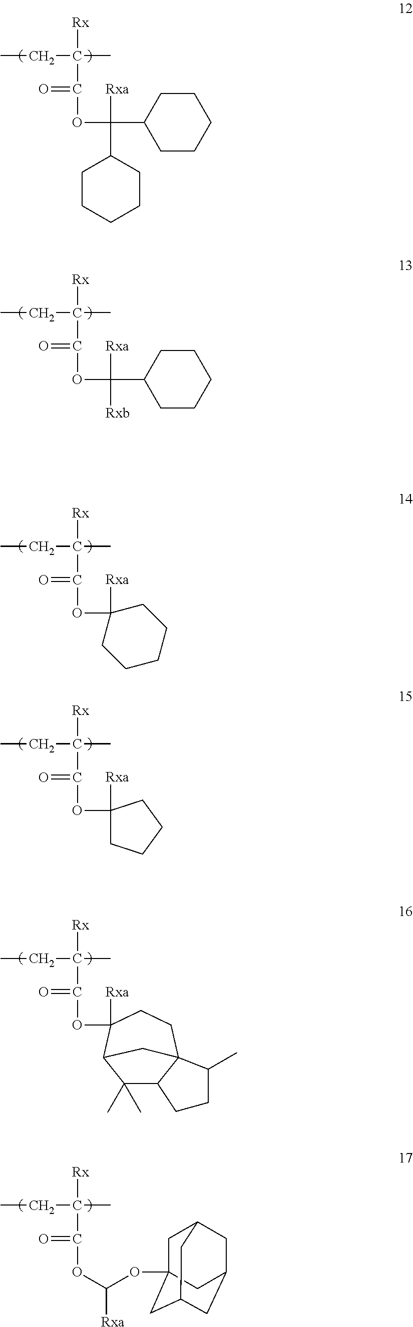

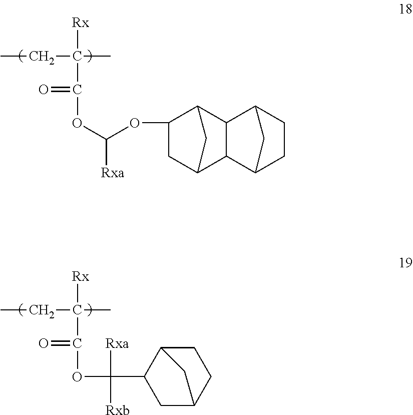

In the above structural formulae, Rx represents H, CH₃, CF₃ or CH₂OH. Each of Rxa and Rxb independently represents an alkyl group having 1 to 4 carbon atoms.

In the general formula (II-AB), the halogen atoms represented by R₁₁′ and R₁₂′ include a chlorine atom, a bromine atom, a fluorine atom, an iodine atom, etc.

The alkyl groups represented by R₁₁′ and R₁₂′ are preferably linear or branched alkyl groups each having 1 to 10 carbon atoms. For example, there can be mentioned a methyl group, an ethyl group, an n-propyl group, an isopropyl group, a linear or branched butyl, pentyl, hexyl or heptyl group, and the like.

The atomic group for formation of the alicyclic structure represented by Z′ is an atomic group capable of providing the resin with a repeating unit of optionally substituted alicyclic hydrocarbon. The atomic group is especially preferably one capable of providing a bridged alicyclic structure for formation of a bridged alicyclic hydrocarbon repeating unit.

The provided alicyclic hydrocarbon skeleton can be the same as that of the cycloalkyl groups represented by R₁₂ to R₂₅ in the general formulae (pI) to (pV).

The alicyclic hydrocarbon skeleton may have one or more substituents. As the substituent, there can be mentioned any of the atoms or groups represented by R₁₃′ to R₁₆′ in the general formulae (II-AB1) and (II-AB2).

In the alicyclic hydrocarbon based acid-decomposable resin, the group that is decomposed by the action of an acid can be contained in at least one repeating unit selected from among the repeating units having partial structures containing the alicyclic hydrocarbons of the general formulae (pI) to (pV), the repeating units of general formula (II-AB) and the repeating units of copolymer components to be described below.

Any of the various substituents that can be introduced in R₁₃′ to R₁₆′ in the general formulae (II-AB1) and (II-AB2) can be a substituent for the atomic groups for formation of the alicyclic structures of the general formula (II-AB) or the atomic groups Z for formation of the bridged alicyclic structures.

Specific examples of the repeating units of the above general formulae (II-AB1) and (II-AB2) will be shown below, which however in no way limit the scope of the present invention.

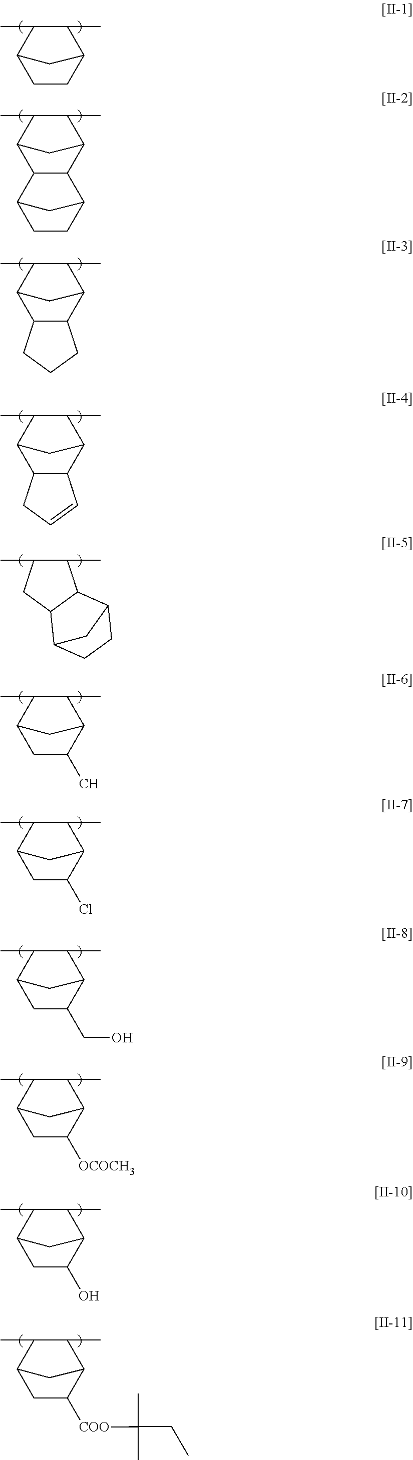

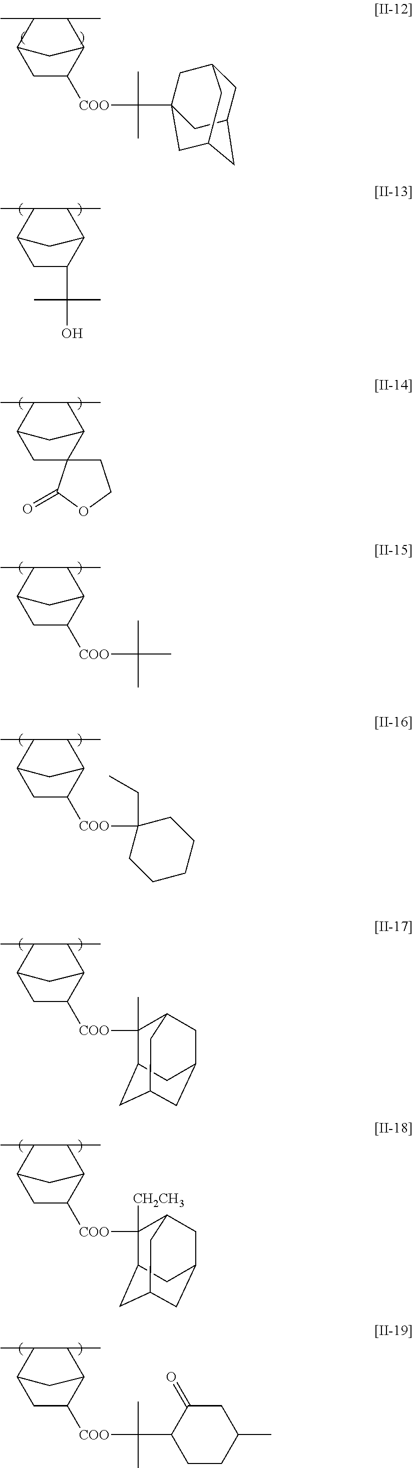

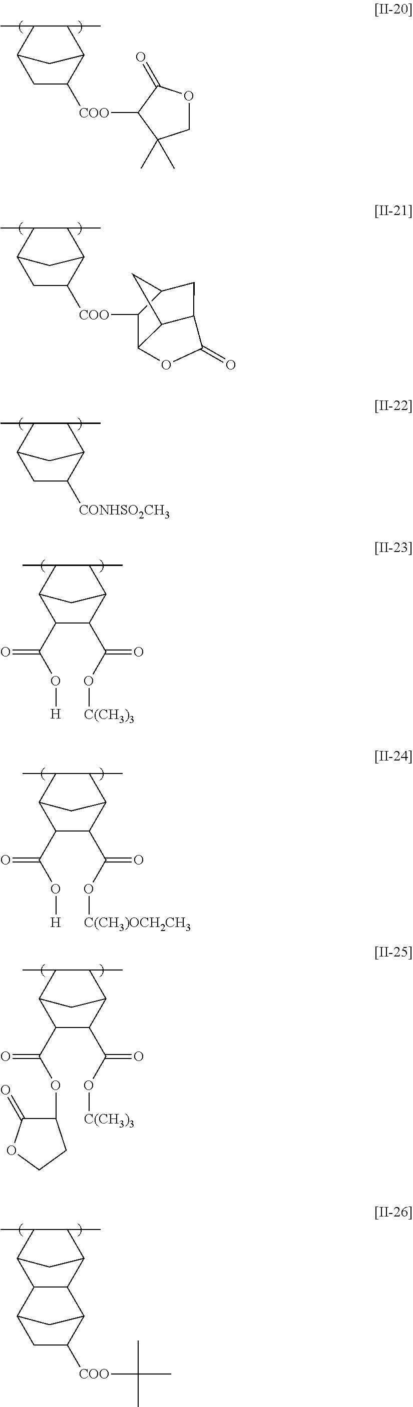

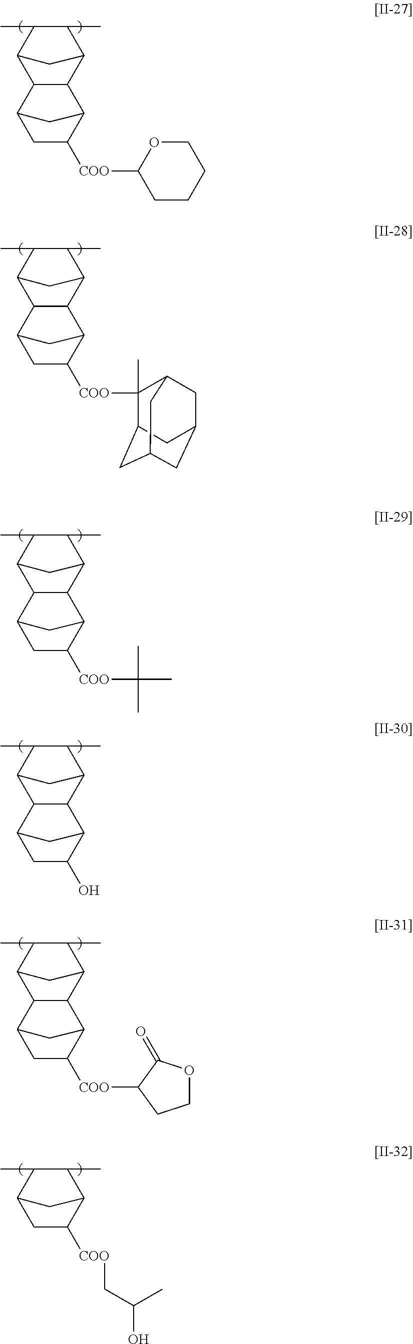

It is preferred for the alicyclic hydrocarbon based acid-decomposable resin to have a repeating unit having a lactone group. Any lactone groups can be employed as long as a lactone structure is possessed therein. However, groups with a 5 to 7-membered ring lactone structure are preferred, and those resulting from condensation of lactone structures of a 5 to 7-membered ring with other cyclic structures effected in a fashion to form a bicyclo structure or spiro structure are especially preferred.

More preferably, the alicyclic hydrocarbon based acid-decomposable resin has a repeating unit having a lactone structure represented by any of general formulae (LC1-1) to (LC1-17) below. The groups with lactone structures may be directly bonded to the principal chain of the resin. Preferred lactone structures are those of the formulae (LC1-1), (LC1-4), (LC1-5), (LC1-6), (LC1-13), (LC1-14), and (LC1-17). The use of these specified lactone structures would realize improvement in the line edge roughness and development defect.

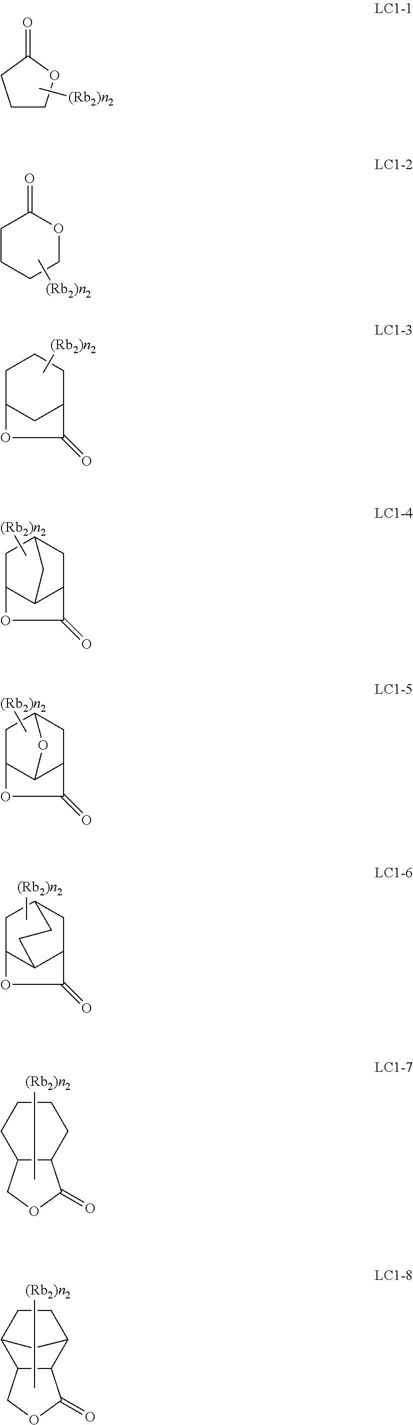

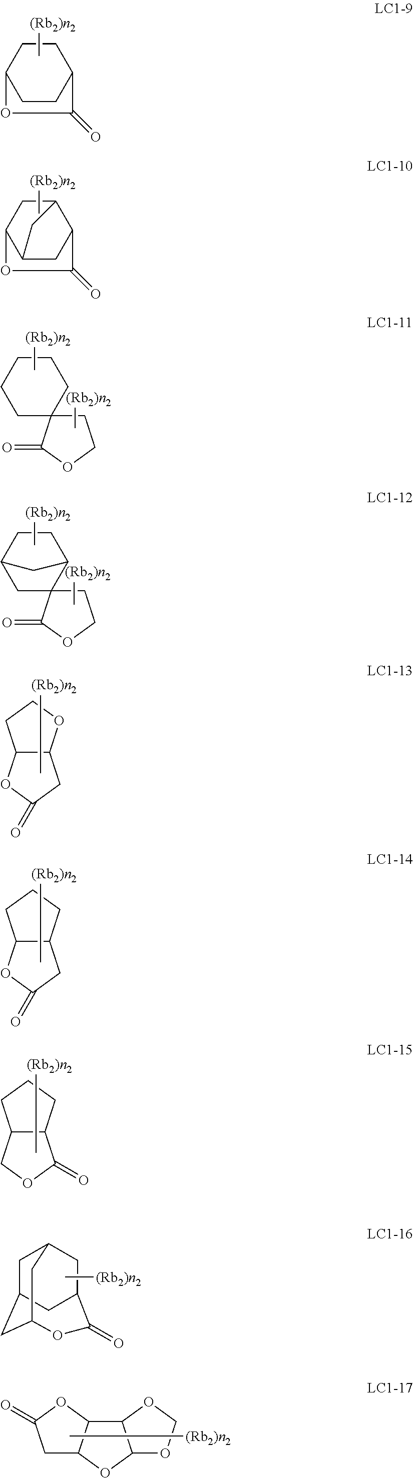

The presence of a substituent (Rb₂) on the portion of the lactone structure is optional. As preferred substituents (Rb₂), there can be mentioned an alkyl group having 1 to 8 carbon atoms, a cycloalkyl group having 3 to 7 carbon atoms, an alkoxy group having 1 to 8 carbon atoms, an alkoxycarbonyl group having 1 to 8 carbon atoms, a carboxyl group, a halogen atom, a hydroxyl group, a cyano group, an acid-decomposable group and the like.

In the formulae, n₂ is an integer of 0 to 4. When n₂ is an integer of 2 or greater, the plurality of present substituents (Rb₂) may be identical to or different from each other. Further, the plurality of present substituents (Rb₂) may be bonded to each other to thereby form a ring.

As the repeating units having the groups with lactone structures of any of the general formulae (LC1-1) to (LC1-17), there can be mentioned the repeating units of the general formulae (II-AB1) and (II-AB2) wherein at least one of R13′ to R16′ has any of the groups of the general formulae (LC1-1) to (LC1-17) as well as the repeating units of general formula (AI) below. Examples of the former include a structure in which the R₅ of —COOR₅ represents any of the groups of the general formulae (LC1-1) to (LC1-17).

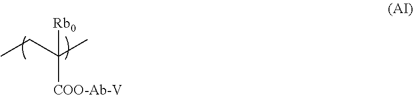

In the general formula (AI), Rb₀ represents a hydrogen atom, a halogen atom or an alkyl group having 1 to 4 carbon atoms.

As the alkyl group represented by Rb₀, there can be mentioned, for example, a methyl group, an ethyl group, a propyl group, an n-butyl group, a sec-butyl group, a t-butyl group or the like. The alkyl group represented by Rb₀ may have one or more substituents. As preferred substituents that may be introduced in the alkyl group represented by Rb₀, there can be mentioned, for example, a hydroxyl group and a halogen atom.

As the halogen atom represented by Rb₀, there can be mentioned a fluorine atom, a chlorine atom, a bromine atom or an iodine atom.

The Rb₀ is preferably a hydrogen atom or a methyl group.

Ab represents an alkylene group, a bivalent connecting group with an alicyclic hydrocarbon structure of a single ring or multiple rings, a single bond, an ether group, an ester group, a carbonyl group, a carboxyl group or a bivalent connecting group resulting from combination of these. A single bond and a connecting group of the formula -Ab₁-CO₂— are preferred.

Ab₁ is a linear or branched alkylene group or a cycloalkylene group of a single ring or multiple rings, being preferably a methylene group, an ethylene group, a cyclohexylene group, an adamantylene group or a norbornylene group.

V represents any of the groups of the general formulae (LC1-1) to (LC1-17).

The repeating unit having a lactone structure is generally present in the form of optical isomers. Any of the optical isomers may be used. It is both appropriate to use a single type of optical isomer alone and to use a plurality of optical isomers in the form of a mixture. When a single type of optical isomer is mainly used, the optical purity thereof is preferably 90% ee or higher, more preferably 95% ee or higher.

Especially preferred repeating units containing lactone group, the followings can be exemplified. Selecting the best lactone group can improve a pattern profile and iso-dense dependence. In the formulae below, each of Rx and R independently represents H, CH₃, CH₂OH, or CF₃.

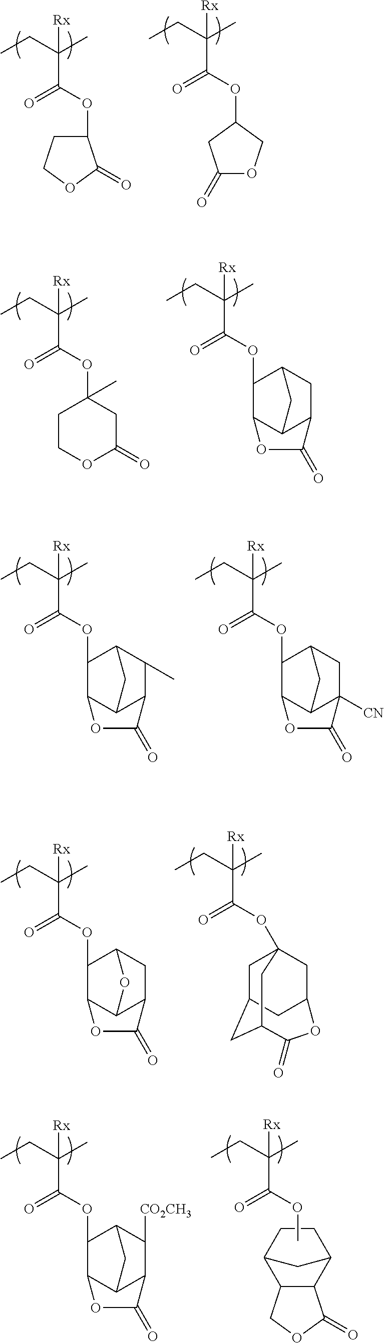

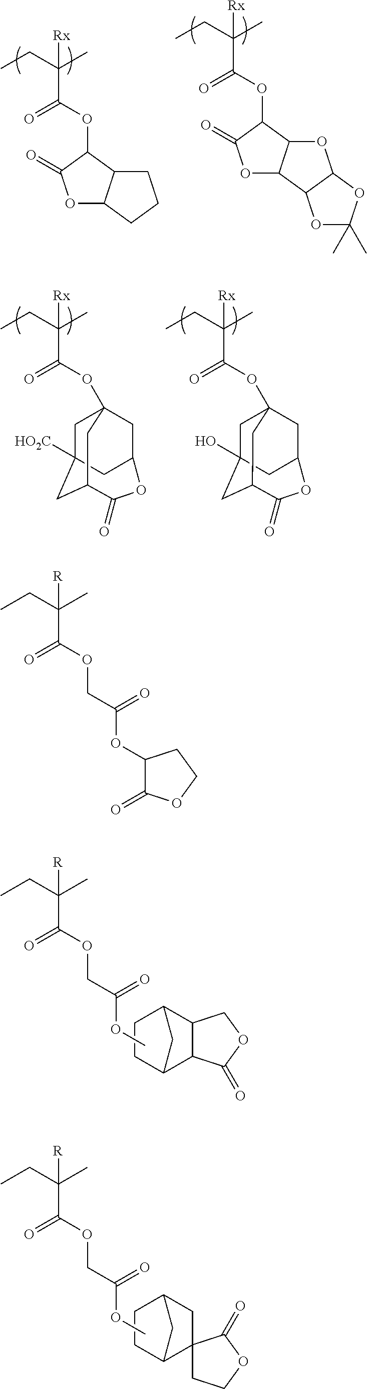

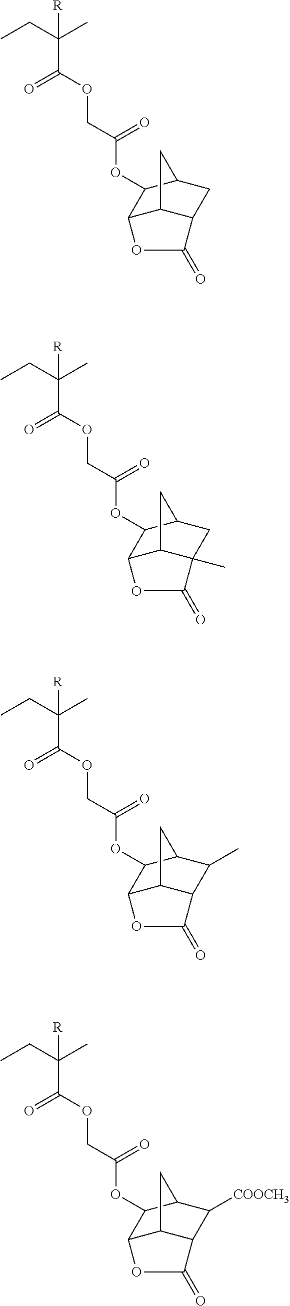

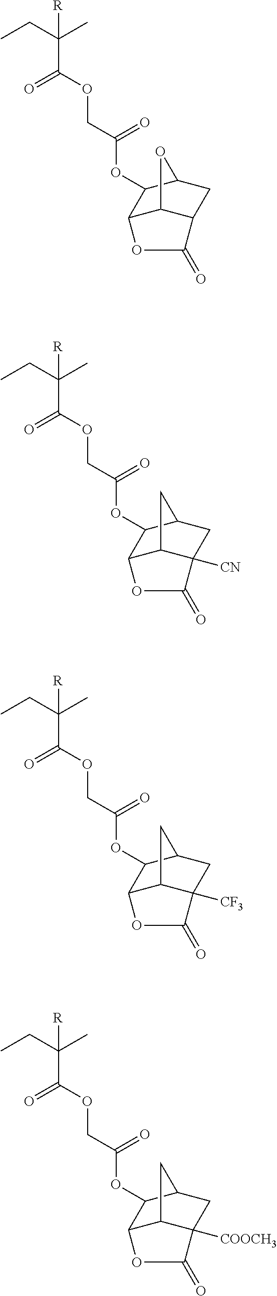

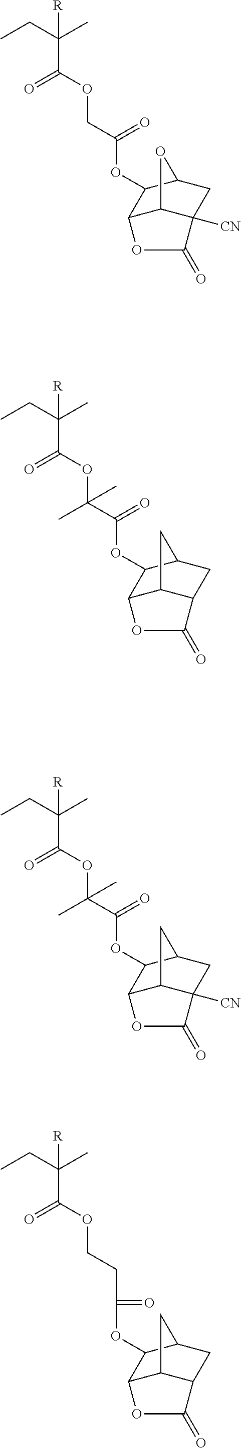

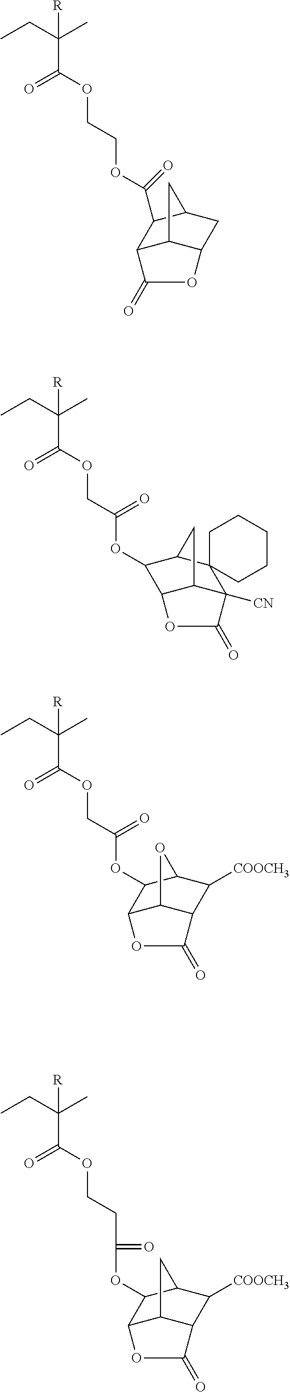

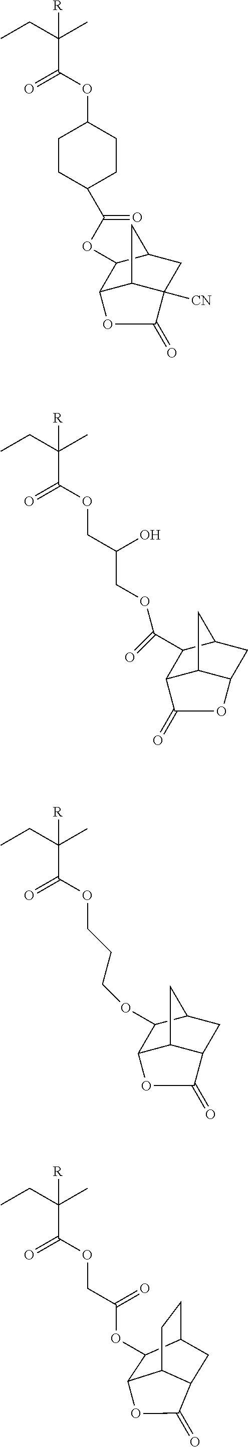

It is preferred for the alicyclic hydrocarbon based acid-decomposable resin to have a repeating unit having an alicyclic hydrocarbon structure substituted with a polar group. The containment of this repeating unit would realize enhancements of adhesion to substrate and developer affinity. The polar group is preferably a hydroxyl group or a cyano group. The hydroxyl group as the polar group constitutes an alcoholic hydroxyl group.

As the alicyclic hydrocarbon structure substituted with a polar group, there can be mentioned, for example, any of the structures of general formulae (VIIa) and (VIIb) below.

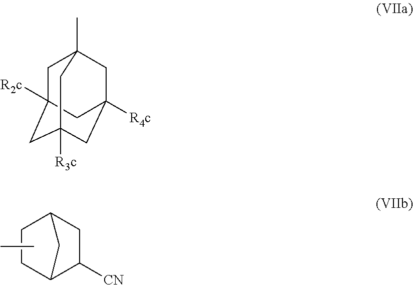

In the general formula (VIIa), each of R₂c to R₄c independently represents a hydrogen atom, a hydroxyl group or a cyano group, provided that at least one of the R₂c to R₄c represents a hydroxyl group or a cyano group. Preferably, one or two of the R₂c to R₄c are hydroxyl groups and the remainder is a hydrogen atom. More preferably, two of the R₂c to R₄c are hydroxyl groups and the remainder is a hydrogen atom.

The groups of the general formula (VIIa) preferably have a dihydroxy form or monohydroxy form, more preferably a dihydroxy form.

As the repeating units having the groups of the general formula (VIIa) or (VIIb), there can be mentioned the repeating units of the general formulae (II-AB1) and (II-AB2) wherein at least one of R13′ to R16′ has any of the groups of the general formula (VIIa) or (VIIb) as well as the repeating units of general formula (AIIa) or (AIIb) below. Examples of the former include a structure in which the R₅ of —COOR₅ represents any of the groups of the general formula (VIIa) or (VIIb).

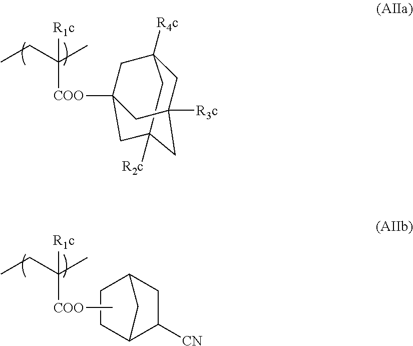

In the general formulae (AIIa) and (AIIb), R₁c represents a hydrogen atom, a methyl group, a trifluoromethyl group or a hydroxymethyl group.

R₂c to R₄c have the same meaning as those of the general formula (VIIa).

Specific examples of the repeating units represented by the general formula (AIIa) or (AIIb) will be shown below, which however in no way limit the scope of the present invention.

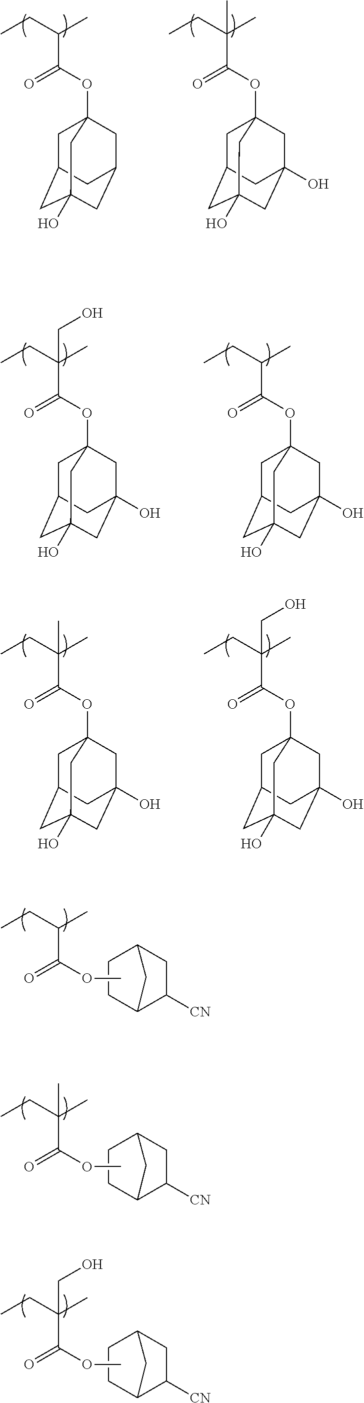

The alicyclic hydrocarbon based acid-decomposable resin according to the present invention may have any of the repeating units of general formula (VIII) below.

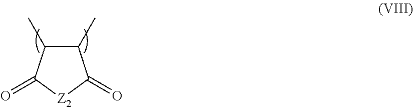

In the general formula (VIII), Z₂ represents —O— or —N(R₄₁)—. R₄₁ represents a hydrogen atom, a hydroxyl group, an alkyl group or —OSO₂—R₄₂. R₄₂ represents an alkyl group, a cycloalkyl group or a camphor residue. The alkyl groups represented by R₄₁ and R₄₂ may be substituted with, for example, a halogen atom. As the halogen atom, a fluorine atom is preferable.

Specific examples of the repeating units of the general formula (VIII) will be shown below, which however in no way limit the scope of the present invention.

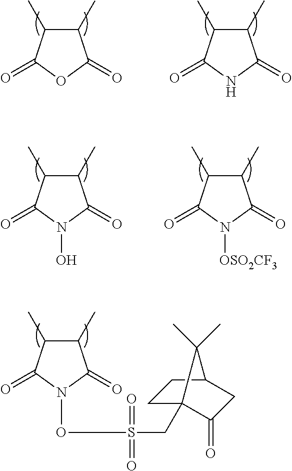

It is preferred for the alicyclic hydrocarbon based acid-decomposable resin to contain a repeating unit having an alkali-soluble group, especially a repeating unit having a carboxyl group. The introduction of the repeating unit having an alkali-soluble group would increase the resolving power in contact hole usage.

The repeating unit having a carboxyl group is preferably either a repeating unit wherein the carboxyl group is directly bonded to the principal chain of a resin or a repeating unit wherein the carboxyl group is bonded via a connecting group to the principal chain of a resin.

Examples of the former case include a repeating unit formed by acrylic acid or methacrylic acid. In the latter case, the connecting group may have a cycloalkyl structure of a single ring or multiple rings

As the repeating units having a carboxyl group, those formed by acrylic acid or methacrylic acid is most preferred.

The resin may further contain a repeating unit that has an alicyclic hydrocarbon structure having no polar group and does not exhibit any acid decomposability. As such, any of the repeating units represented by general formula (IV) below can be exemplified.

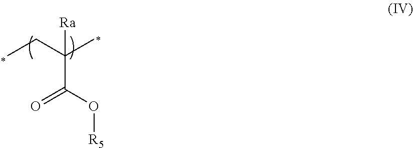

In the general formula (IV), R₅ represents a hydrocarbon group having at least one cyclic structure in which neither a hydroxyl group nor a cyano group is contained.

Ra represents a hydrogen atom, an alkyl group or a group of the formula —CH₂—O—Ra₂ in which Ra₂ represents a hydrogen atom, an alkyl group or an acyl group. Ra is preferably a hydrogen atom, a methyl group, a hydroxymethyl group or a trifluoromethyl group, further preferably a hydrogen atom or a methyl group.

The cyclic structures contained in R₅ include a monocyclic hydrocarbon group and a polycyclic hydrocarbon group.

As the monocyclic hydrocarbon group, a cycloalkyl group having 3 to 12 carbon atoms and a cycloalkenyl group having 3 to 12 carbon atoms can be exemplified. Preferably, the monocyclic hydrocarbon group is a monocyclic hydrocarbon group having 3 to 7 carbon atoms. As such, a cyclopentyl group and a cyclohexyl group can be exemplified.

The polycyclic hydrocarbon groups include ring-assembly hydrocarbon groups and crosslinked-ring hydrocarbon groups.

As the ring-assembly hydrocarbon groups, for example, a bicyclohexyl group and a perhydronaphthalenyl group can be exemplified.

As the crosslinked-ring hydrocarbon rings, there can be mentioned, for example, a norbornyl group, an adamantyl group, a bicyclooctanyl group, and a tricyclo[5,2,1,0^{2,6}]decanyl group. As more preferred crosslinked-ring hydrocarbon rings, for example, a norbornyl group and an adamantyl group can be exemplified.

The halogen atom is preferably a bromine, chlorine or fluorine atom.

The alkyl group is preferably a methyl, ethyl, butyl or t-butyl group. The alkyl group may further have one or more substituents. As the optional substituent, a halogen atom, an alkyl group, a hydroxyl group protected by a protective group, and an amino group protected by a protective group can be exemplified.

As the protective group, an alkyl group, a cycloalkyl group, an aralkyl group, a substituted methyl group, a substituted ethyl group, an alkoxycarbonyl group and an aralkyloxycarbonyl group can be exemplified. Preferred alkyl groups include alkyl groups having 1 to 4 carbon atoms. Preferred substituted methyl groups include methoxymethyl, methoxythiomethyl, benzyloxymethyl, t-butoxymethyl and 2-methoxyethoxymethyl groups. Preferred substituted ethyl groups include 1-ethoxyethyl and 1-methyl-1-methoxyethyl groups. Preferred acyl groups include aliphatic acyl groups having 1 to 6 carbon atoms, such as formyl, acetyl, propionyl, butyryl, isobutyryl, valeryl and pivaloyl groups. Preferred alkoxycarbonyl groups include alkoxycarbonyl groups having 1 to 4 carbon atoms and the like.

Specific examples of the repeating units that have an alicyclic hydrocarbon structure having no polar group and do not exhibit any acid decomposability will be shown below, which however in no way limit the scope of the present invention. In the formulae, Ra represents H, CH₃, CH₂OH or CF₃.

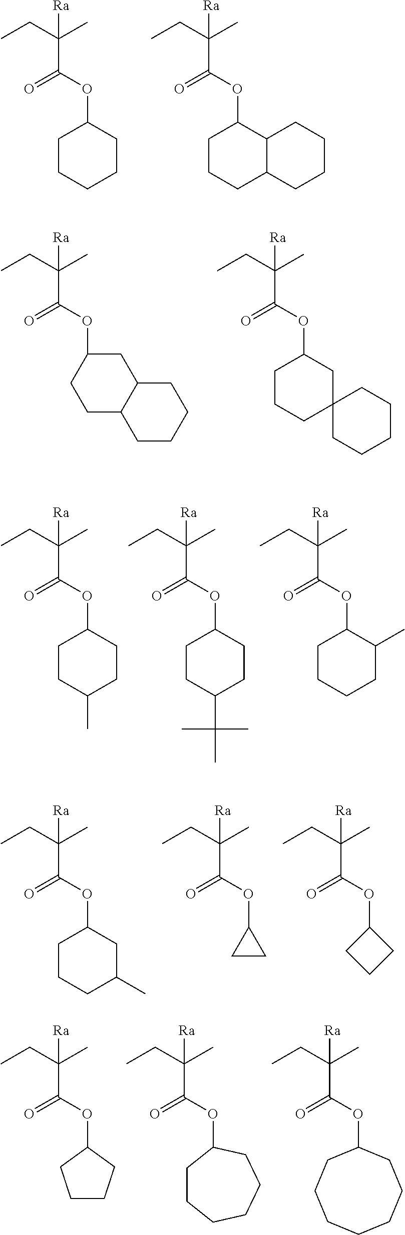

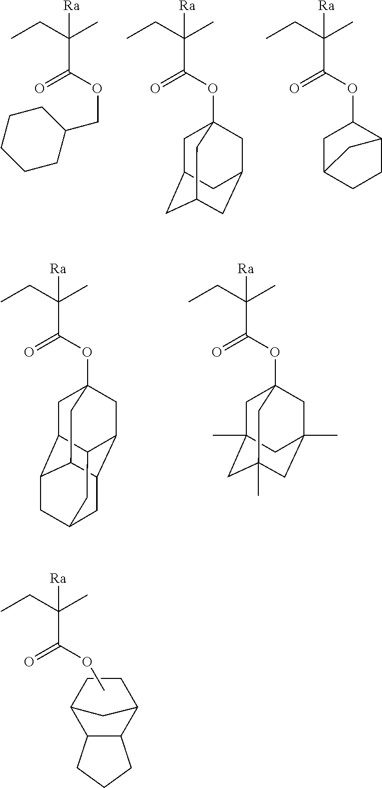

The content of the repeating unit that has an alicyclic hydrocarbon structure having no polar group and does not exhibit any acid decomposability based on all the repeating units of the resin is preferably in the range of 0 to 40 mol %, more preferably 5 to 20 mol %.

The resin may further contain repeating units other than those described above, especially when the composition is used for KrF, EB, or EUV exposure. As such, repeating units that is stable against the action of an acid can be exemplified.

As repeating units that is stable against the action of an acid, for example, repeating units in which the side chain of acrylic structure contains aryl or cycloalkyl structure which does not shown acid decomposability, such as those represented by the general formula (IV), can be exemplified. In the repeating units represented by the general formula (IV), R₅ preferably is a hydrocarbon group containing a cyclic structure therein. As the specific examples of the case, there can be mentioned, for example, a monocyclic or polycyclic cycloalkyl group (having preferably 3 to 12, more preferably 3 to 7 carbon atoms; a cyclohexyl group is especially preferred), a monocyclic or polycyclic cycloalkenyl group (having preferably 3 to 12 carbon atoms), an aryl group (having preferably 6 to 20, more preferably 6 to 12 carbon atoms; a phenyl group and a naphthyl group is especially preferred), and an aralkyl group (having preferably 7 to 20, more preferably 7 to 12 carbon atoms; a benzyl group is especially preferred). Incorporating such structures can lead to an adjustment of contrast and an enhancement of etching resistance.

The content of the repeating units that is stable against the action of an acid based on all the repeating units in the resin is preferably in the range of 0 to 40 mol %, and more preferably in the range of 1 to 20 mol %.

As the specific examples of the repeating units that is stable against the action of an acid, the followings can be exemplified in addition to the ones described for the repeating units represented by the general formula (IV). In the formulae, Ra represents H, CH₃, CH₂OH, or CF₃.

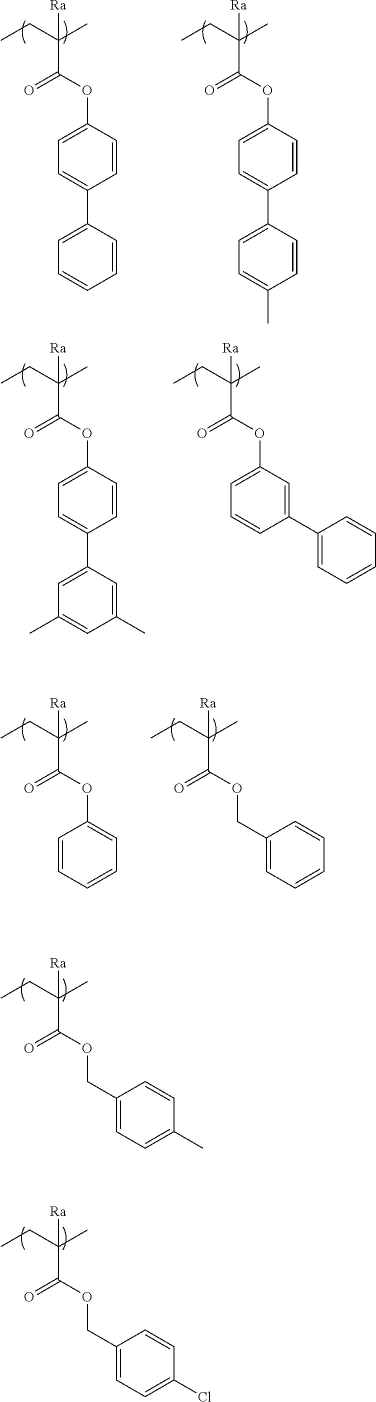

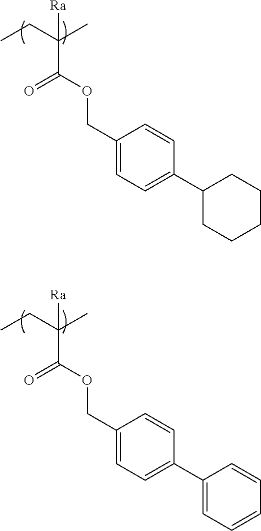

As specific examples of other repeating units which are stable for the action of the acid, for example, a compound containing at least one addition-polymerizable unsaturated bond, selected from among (meth)acrylamides, allyl compounds, vinyl ethers, vinyl esters, styrenes, crotonic esters and the like. Furthermore, the other repeating units include maleic anhydride, maleimide, acrylonitrile, methacrylonitrile and maleonitrile.

Preferred specific examples of the other repeating units will be shown below, which however in no way limit the scope of the present invention.

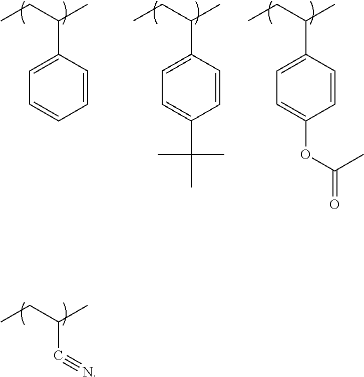

The molecular weight in terms of polystyrene molecular weight measured by GPC of the resin is not particularly limited. Preferably, the weight average molecular weight (Mw) thereof is in the range of 2000 to 200,000. By making Mw higher than 2,000, the heat resistance and dry etching resistance can be enhanced. By making Mw lower than 200,000, developability can be enhanced, and the viscosity of the composition can be decreased leading to better filth forming property.

More preferable Mw falls in between 2,500 to 50,000, and further more preferable Mw in between 3,000 to 25,000. In cases for pattern formation using an electron beam, X-ray, or high-energy beam whose wavelength is 50 nm or lower (for example, EUV), it is particularly preferable for Mw to fall within the range of 3,000 to 10,000. By adjusting the Mw, increase in the heat resistance, enhancement of the resolving power, and decrease of the development defect can simultaneously be realized.

The dispersity (Mw/Mn) of the resin is preferably in the range of 1.0 to 3.0, more preferably 1.2 to 2.5, and further preferably 1.2 to 1.6. By adjusting the dispersity, for example, line edge roughness characteristics can be enhanced.

The resin may be used alone, or two or more types thereof may be used in combination. The content of the resin is preferably in the range of 0 to 99.9 mass %, more preferably 50 to 95 mass %, and further preferably 60 to 93 mass % based on the total solids of the composition.

[3] Alkali-Soluble Resin

The alkali dissolution rate of the alkali-soluble resin as measured in a 0.261 N tetramethylammonium hydroxide (TMAH) (23° C.) is preferably 2 nm/sec or higher, especially preferably 20 nm/sec or higher.

As the alkali-soluble resin for use in the present invention, there can be mentioned, for example, a novolak resin, a hydrogenated novolak resin, an acetone-pyrogallol resin, an o-polyhydroxystyrene, a m-polyhydroxystyrene, a p-polyhydroxystyrene, a hydrogenated polyhydroxystyrene, a halogenated or alkylated polyhydroxystyrene, a hydroxystyrene-N-substituted maleimide copolymer, an o/p- and m/p-hydroxystyrene copolymer, a partial O-alkylation product of hydroxyl of polyhydroxystyrene (for example, a 5 to 30 mol % O-methylation product, O-(1-methoxy)ethylation product, O-(1-ethoxy)ethylation product, O-2-tetrahydropyranylation product, O-(t-butoxycarbonyl)methylation product, etc.), an O-acylation product thereof (for example, a 5 to 30 mol % O-acetylation product, O-(t-butoxy)carbonylation product, etc.), a styrene-maleic anhydride copolymer, a styrene-hydroxystyrene copolymer, an α-methylstyrene-hydroxystyrene copolymer, a carboxylated methacrylic resin or its derivative, or a polyvinyl alcohol derivative. However, the alkali-soluble resins are not limited to these.

Especially preferred alkali-soluble resins are a novolak resin, an o-polyhydroxystyrene, a m-polyhydroxystyrene, a p-polyhydroxystyrene, a copolymer of these polyhydroxystyrenes, an alkylated polyhydroxystyrene, a partial O-alkylation product or O-acylation product of polyhydroxystyrene, a styrene-hydroxystyrene copolymer and an α-methylstyrene-hydroxystyrene copolymer.

The resins containing one or more hydroxystyrene structures are particularly preferred. Of these, those containing one or more m-hydroxystyrene structures are especially preferred.

It is preferred for the alkali-soluble resin to contain any of the above-mentioned repeating units of general formula (VI). The inventors have found that the sensitivity of the composition of the present invention can be enhanced by using the alkali-soluble resin containing any of the repeating units of general formula (VI) in combination with any of the aforementioned compounds of general formula (I). The reason therefor is not necessarily apparent. However, the inventors presume that the reason would be that a chain reaction described in J. Org. Chem. 2005, 70, 6809-6819 is likely to occur, so that the amount of generated acid is increased.

The above novolak resin can be obtained by addition condensation of a given monomer as a main component with an aldehyde conducted in the presence of an acid catalyst.

The weight average molecular weight of the alkali-soluble resin is 2000 or greater, preferably from 5000 to 200,000 and more preferably 5000 to 100,000. Herein, the weight average molecular weight is in terms of polystyrene molecular weight measured by gel permeation chromatography (GPC).

The alkali-soluble resins can be used individually or in combination.

The amount of alkali-soluble resin added, based on the solid contents of the whole composition, is preferably in the range of 40 to 97 mass %, and more preferably in the range of 60 to 90 mass %.

In place of the foregoing resins, use may be made of any of compounds (A) described in JP-A-2009-222920 and/or any of fullerene derivatives described in JP-A-2010-024221.

[4] Dissolution-Inhibiting Compound

From the viewpoint or preventing any lowering of 220 nm or shorter transmission, the dissolution inhibiting compound is preferably an alicyclic or aliphatic compound containing an acid-decomposable group. As such, for example, cholic acid derivatives having an acid-decomposable group described in Proceeding of SPIE, 2724, 355 (1996) can be exemplified. The acid-decomposable group and alicyclic structure are the same as described above with respect to the alicyclic hydrocarbon based acid-decomposable resin.

When the composition according to the present invention is exposed to a KrF excimer laser or irradiated with electron beams, preferred use is made of a compound containing a structure resulting from substitution of the phenolic hydroxyl group of a phenol compound with an acid-decomposable group. The phenol compound preferably contains 1 to 9 phenol skeletons, more preferably 2 to 6 phenol skeletons.

Molecular weight of the dissolution-inhibiting compound is 3000 or less. The molecular weight is preferably in the range of 300 to 3000, and more preferably in the range of 500 to 2500.

The amount of dissolution inhibiting compound added is preferably in the range of 3 to 50 mass %, more preferably 5 to 40 mass % based on the solid contents of the composition.

Specific examples of the dissolution inhibiting compounds will be shown below, which however in no way limit the scope of the present invention.

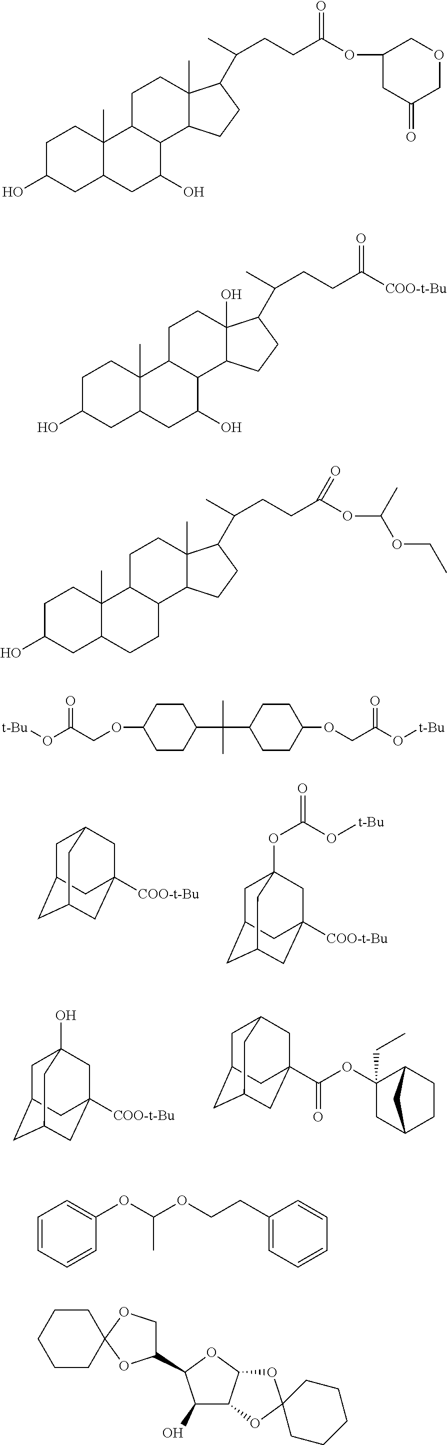

[5] Acid Crosslinking Agent

Any crosslinking agent can be used as long as it is a compound capable of crosslinking with the resin soluble in an alkali developer by the action of an acid. However, compounds (1) to (3) below are preferred.

(1) A hydroxymethylated form, alkoxymethylated or acyloxymethylated form of phenol derivative.

(2) A compound having an N-hydroxymethyl group, an N-alkoxymethyl group or an N-acyloxymethyl group.

(3) A compound having an epoxy group.

The alkoxymethyl group preferably has 6 or less carbon atoms, and the acyloxymethyl group preferably has 6 or less carbon atoms.

Those especially preferred among these crosslinking agents will be shown below.

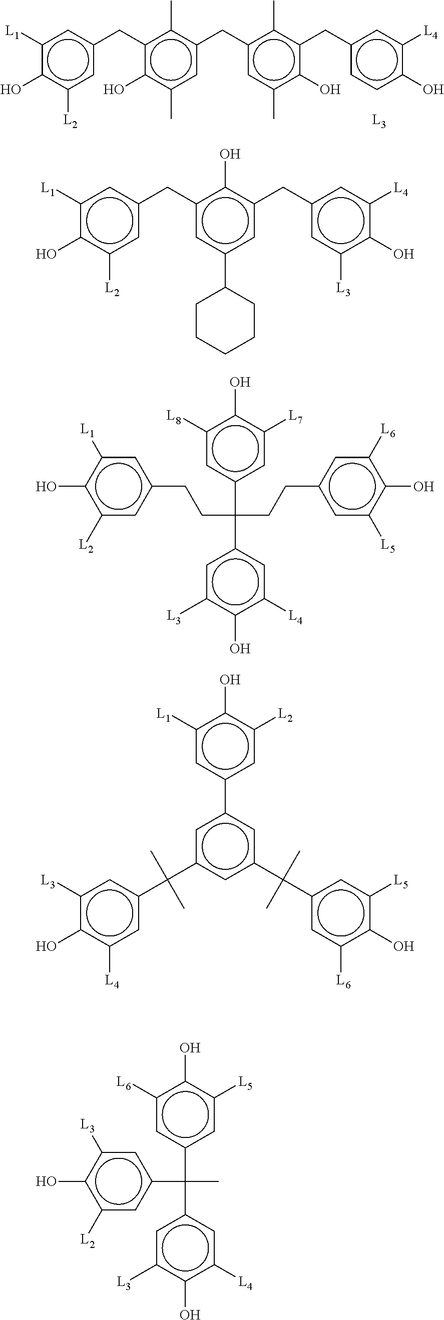

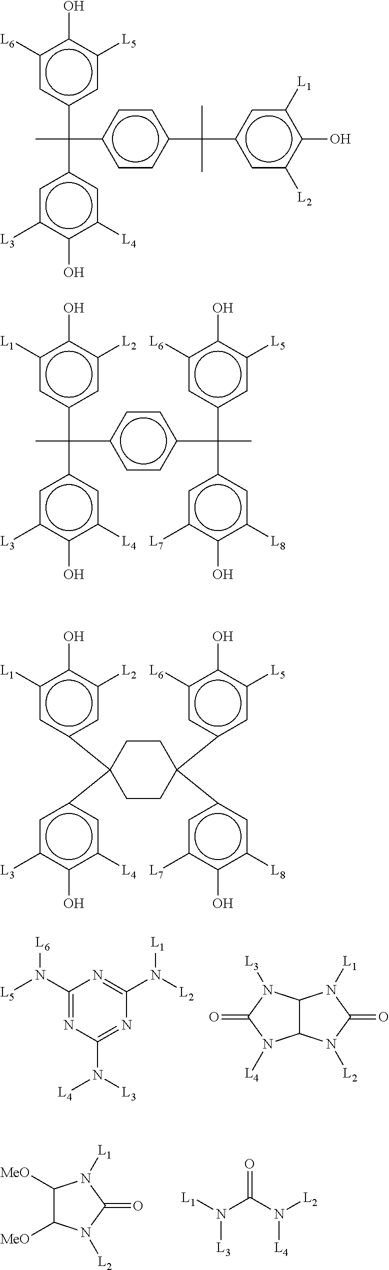

In the formulae, L₁ to L₈ may be identical to or different from each other, and each thereof represents a hydrogen atom, a hydroxymethyl group, a methoxymethyl group, an ethoxymethyl group or an alkyl group having 1 to 6 carbon atoms.

The crosslinking agent is generally added in an amount of 3 to 70 mass %, preferably 5 to 50 mass %, based on the solid content of the composition.

[6] Acid Amplifier

As mentioned above, the acid amplifier is a substance that when acted on by an acid, is decomposed to thereby generate an acid stronger than carboxylic acid.

It is preferred for the acid generated by the acid amplifier to exhibit a high acid strength. In particular, the dissociation constant (pKa) of the acid is preferably 3 or below, more preferably 2 or below. It is preferred for the acid generated by the acid amplifier to be a sulfonic acid.

The acid amplifiers described in International Publication Nos. 95/29968 and 98/24000, JP-A's H8-305262, H9-34106 and H8-248561, Jpn. PCT National Publication No. H8-503082, U.S. Pat. No. 5,445,917,

Jpn. PCT National Publication No. H8-503081, U.S. Pat. Nos. 5,534,393, 5,395,736, 5,741,630, 5,334,489, 5,582,956, 5,578,424, 5,453,345 and 5,445,917, European Patent Nos. 665,960, 757,628 and 665,961, U.S. Pat. No. 5,667,943 and JP-A's H10-1508, H10-282642, H9-512498, 2000-62337, 2005-17730 and 2008-209889, etc. can be used individually or in combination.

In particular, the compounds of general formulae (1) to (6) below are preferred.

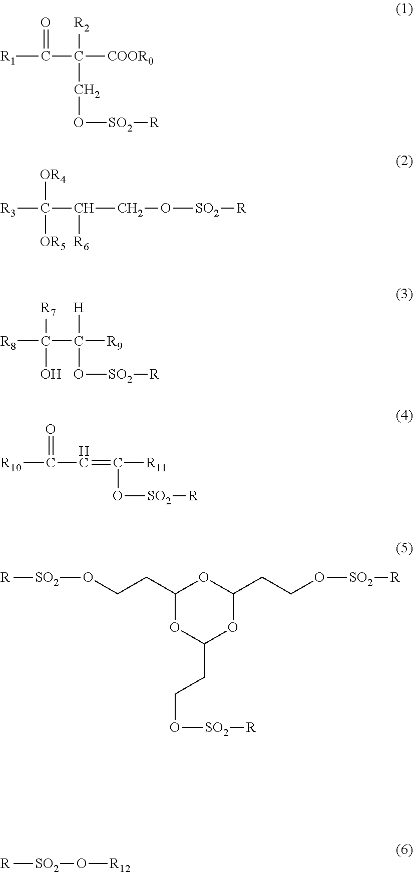

In general formulae (1) to (6),

R represents an alkyl group, a monovalent aliphatic hydrocarbon ring group, an aryl group or an aralkyl group.

R₀ represents a group that is cleaved under the action of an acid.

R₁ represents an alkyl group, a monovalent aliphatic hydrocarbon ring group, an aryl group, an aralkyl group, an alkoxy group or an aryloxy group.

R₂ represents an alkyl group or an aralkyl group.

R₃ represents an alkyl group, a monovalent aliphatic hydrocarbon ring group, an aryl group or an aralkyl group.

Each of R₄ and R₅ independently represents an alkyl group, provided that R₄ and R₅ may be bonded to each other to thereby form a ring.

R₆ represents a hydrogen atom or an alkyl group.

R₇ represents a hydrogen atom, an alkyl group, a monovalent aliphatic hydrocarbon ring group, an aryl group or an aralkyl group.

R₈ represents an alkyl group, a monovalent aliphatic hydrocarbon ring group, an aryl group or an aralkyl group.

R₉ represents a hydrogen atom, an alkyl group, a monovalent aliphatic hydrocarbon ring group, an aryl group or an aralkyl group.

R₉ may be bonded to R₇ to thereby form a ring.

R₁₀ represents an alkyl group, a monovalent aliphatic hydrocarbon ring group, an alkoxy group, an aryl group, an aralkyl group, an aryloxy group or an alkenyloxy group.

R₁₁ represents an alkyl group, a monovalent aliphatic hydrocarbon ring group, an alkoxy group, an aryl group, an aralkyl group, an aryloxy group or an alkenyl group.

R₁₀ and R₁₁ may be bonded to each other to thereby form a ring.

R₁₂ represents an alkyl group, a monovalent aliphatic hydrocarbon ring group, an aryl group, an alkenyl group, an alkynyl group or a cycloimido group.

Preferred examples of the groups represented by R, R₀ and R₁ to R₁₁ are as follows.

R: a methyl group, an ethyl group, a propyl group, a butyl group, an octyl group, a trifluoromethyl group, a nonafluorobutyl group, a heptadecafluorooctyl group, a 2,2,2-trifluoroethyl group, a phenyl group, a pentafluorophenyl group, a methoxyphenyl group, a toluoyl group, a mesityl group, a fluorophenyl group, a naphthyl group, a cyclohexyl group or a camphor group.

R₀: a t-butyl group, a methoxymethyl group, an ethoxymethyl group, a 1-ethoxyethyl group or a tetrahydropyranyl group.

R₁: a methyl group, an ethyl group, a propyl group, a cyclopropyl group, a cyclopentyl group, a cyclohexyl group, a phenyl group, a naphthyl group, a benzyl group, a phenethyl group, a methoxy group, an ethoxy group, a propoxy group, a phenoxy group or a naphthoxy group.

R₂: a methyl group, an ethyl group, a propyl group, a butyl group or a benzyl group.

R₃: a methyl group, an ethyl group, a propyl group, a cyclopropyl group, a cyclopentyl group, a cyclohexyl group, a phenyl group, a naphthyl group, a benzyl group, a phenethyl group or a naphthylmethyl group.

R₄, R₅: a methyl group, an ethyl group, a propyl group, or, formed by mutual bonding thereof, an ethylene group or propylene group.

R₆: a hydrogen atom, a methyl group or an ethyl group.

R₇, R₉: a hydrogen atom, a methyl group, an ethyl group, a propyl group, a butyl group, a pentyl group, a cyclopropyl group, a cyclopentyl group, a cyclohexyl group, a phenyl group, a naphthyl group, a benzyl group, a phenethyl group, or, formed by the mutual bonding thereof, a cyclopentyl ring or cyclohexyl ring.

R₈: a methyl group, an ethyl group, an isopropyl group, a t-butyl group, a neopentyl group, a cyclohexyl group, a phenyl group or a benzyl group.

R₁₀: a methyl group, an ethyl group, a propyl group, an isopropyl group, a butyl group, an isobutyl group, a cyclopropyl group, a cyclopentyl group, a cyclohexyl group, a methoxy group, an ethoxy group, a phenyl group, a naphthyl group, a benzyl group, a phenoxy group, a naphthoxy group, a vinyloxy group, a methylvinyloxy group, or, formed by the mutual bonding thereof and optionally containing an oxygen atom, a 3-oxocyclohexenyl ring or 3-oxoindenyl ring.

R₁₁: a methyl group, an ethyl group, a propyl group, an isopropyl group, a butyl group, an isobutyl group, a cyclopropyl group, a cyclopentyl group, a cyclohexyl group, a methoxy group, an ethoxy group, a phenyl group, a naphthyl group, a benzyl group, a phenoxy group, a naphthoxy group, a vinyl group, an allyl group, or, formed by the mutual bonding thereof and optionally containing an oxygen atom, a 3-oxocyclohexenyl ring or 3-oxoindenyl ring.

In general formula (6), when R₁₂ is an alkyl group, the alkyl group is preferably a linear one having 1 to 12 carbon atoms or a branched one having 3 to 12 carbon atoms.

When R₁₂ is a monovalent aliphatic hydrocarbon ring group, the monovalent aliphatic hydrocarbon ring group is preferably a monocyclic or polycyclic one having 5 to 10 carbon atoms.

When R₁₂ is an aryl group, as the aryl group, there can be mentioned a condensed ring formed by 1 to 3 benzene rings or a condensed ring formed by a benzene ring and a 5-membered unsaturated ring. Specific examples thereof include a phenyl group, a naphthyl group, an anthryl group, a phenanthryl group, an indenyl group, an acenaphthenyl group, a fluorenyl group and the like. Among these, a phenyl group and a naphthyl group are preferred. The aryl groups include not only these carbon-ring aryl groups but also heterocyclic aryl groups. As the heterocyclic aryl groups, there can be mentioned those each containing 3 to 20 carbon atoms and'1 to 5 heteroatoms, such as a pyridyl group and a furyl group as well as, resulting from condensation of a benzene ring, a quinolyl group, benzofuryl group, thioxanthone group and carbazole group.

When R₁₂ is an alkenyl group, a substituted alkenyl group [—C(R₁₄)═C(R₁₅)(R₁₆)], an alkynyl group or a substituted alkynyl group [—C≡C(R₁₇)], R₁₄ to R₁₇ can be monovalent nonmetallic atomic groups. As preferred examples of the atomic groups represented by R₁₄ to R₁₇, there can be mentioned a hydrogen atom, a halogen atom, an alkyl group, a substituted alkyl group, an aryl group and a substituted aryl group. As specific examples thereof, there can be mentioned those set forth above by way of example. As more preferred substituents represented by R₁₄ to R₁₇, there can be mentioned a hydrogen atom, a halogen atom and a linear, branched or cyclic alkyl group having 1 to 10 carbon atoms.

When R₁₂ is a cycloimido group, as the cycloimido, there can be mentioned one having 4 to 20 carbon atoms, such as succinimido, phthalimido, cyclohexanedicarboxyimido or norbornenedicarboxyimido.

As specific examples of the compounds of general formulae (1) to (6), there can be mentioned the compounds (1-1) to (1-11), (2-1) to (2-6), (3-1) to (3-6), (4-1) to (4-7), (5-1) to (5-4) and (6-1) to (6-20) set forth as examples in section [0215] et seq. of JP-A-2008-209889.

[7] Basic Compound

The composition of the present invention preferably contains a basic compound in order to reduce any performance change over time from exposure to bake. The role of the basic compound is to quench any deprotection reaction by the acid generated by exposure, and the diffusivity and basicity thereof would influence the substantial diffusivity of the acid.

The basic compound is preferably an organic compound containing a nitrogen atom. As such, an ammonium salt represented by formula (A) below and basic compounds with the structures of formulae (B) to (E) below.

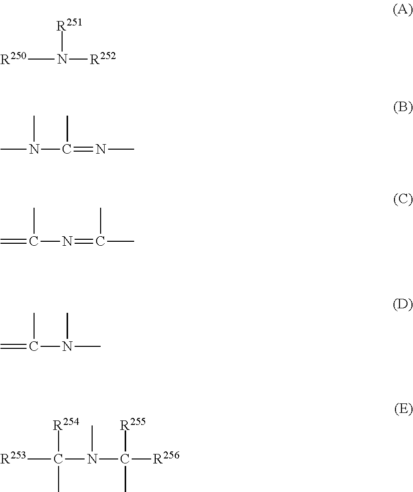

In the formula (A), each of R²⁵⁰, R²⁵¹ and R²⁵² independently represents a hydrogen atom, an alkyl group (preferably having 1 to 20 carbon atoms), a cycloalkyl group (preferably having 3 to 20 carbon atoms) or an aryl group (preferably having 6 to 20 carbon atoms). R²⁵⁰ and R²⁵¹ may be bonded to each other to thereby form a ring. These groups may contain one or more substituents.

The alkyl group and cycloalkyl group having substituents are preferably an aminoalkyl group having 1 to 20 carbon atoms, an aminocycloalkyl group having 3 to 20 carbon atoms, a hydroxyalkyl group having 1 to 20 carbon atoms and a hydroxycycloalkyl group having 3 to 20 carbon atoms.

These groups may contain an oxygen atom, a sulfur atom or a nitrogen atom in the alkyl chain thereof.

In the formula (E), each of R²⁵³, R²⁵⁴, R²⁵⁵ and R²⁵⁶ independently represents an alkyl group (preferably having 1 to 6 carbon atoms) or a cycloalkyl group (preferably having 3 to 6 carbon atoms). These may have substituents.

As preferred compounds, there can be mentioned guanidine, aminopyrrolidine, pyrazole, pyrazoline, piperazine, aminomorpholine, aminoalkylmorpholines, piperidine and the like. These may have substituents.

As further preferred compounds, there can be mentioned compounds with an imidazole structure, a diazabicyclo structure, an onium hydroxide structure, an onium carboxylate structure, a trialkylamine structure, an aniline structure or a pyridine structure, alkylamine derivatives having a hydroxyl group and/or an ether bond, aniline derivatives having a hydroxyl group and/or an ether bond and the like.

As the compounds with an imidazole structure, there can be mentioned imidazole, 2,4,5-triphenylimidazole, benzimidazole and the like.

As the compounds with a diazabicyclo structure, there can be mentioned 1,4-diazabicyclo[2,2,2]octane, 1,5-diazabicyclo[4,3,0]non-5-ene, 1,8-diazabicyclo[5,4,0]undec-7-ene and the like.

As the compounds with an onium hydroxide structure, there can be mentioned triarylsulfonium hydroxides, phenacylsulfonium hydroxide, and sulfonium hydroxides having a 2-oxoalkyl group such as triphenylsulfonium hydroxide, tris(t-butylphenyl)sulfonium hydroxide, bis(t-butylphenyl)iodonium hydroxide, phenacylthiophenium hydroxide, 2-oxopropylthiophenium hydroxide and the like.

As the compounds with an onium carboxylate structure, there can be mentioned those having a carboxylate at the anion moiety of the compounds with an onium hydroxide structure, for example, acetate, adamantane-1-carboxylate, perfluoroalkyl carboxylates and the like.

As the compounds with a trialkylamine structure, there can be mentioned tri(n-butyl)amine, tri(n-octyl)amine and the like.

As the aniline compounds, there can be mentioned 2,6-diisopropylaniline, N,N-dimethylaniline and the like.

As the alkylamine derivatives having a hydroxyl group and/or an ether bond, there can be mentioned ethanolamine, diethanolamine, triethanolamine, tris(methoxyethoxyethyl)amine and the like.

As the aniline derivatives having a hydroxyl group and/or an ether bond, there can be mentioned N,N-bis(hydroxyethyl)aniline and the like.

Further, as basic compounds, there can be mentioned at least one nitrogenous compound selected from among an amine compound having a phenoxy group, and an ammonium salt compound having a phenoxy group.

As the amine compound, use can be made of primary, secondary and tertiary amine compounds. An amine compound having at least one alkyl group bonded to the nitrogen atom thereof is preferred. Among the amine compounds, a tertiary amine compound is more preferred. In the amine compounds, as long as at least one alkyl group (preferably having 1 to 20 carbon atoms) is bonded to the nitrogen atom, a cycloalkyl group (preferably having 3 to 20 carbon atoms) or an aryl group (preferably having 6 to 12 carbon atoms) besides the alkyl group may be bonded to the nitrogen atom.

In the amine compounds, it is preferred for the alkyl chain thereof to contain an oxygen atom, thereby forming an oxyalkylene group. The number of oxyalkylene groups in each molecule is one or more, preferably 3 to 9, and more preferably 4 to 6. The oxyalkylene group is preferably an oxyethylene group (—CH₂CH₂O—) or an oxypropylene group (—CH(CH₃)CH₂O— or —CH₂CH₂CH₂O—), more preferably an oxyethylene group.

In the ammonium salt compounds, as long as at least one alkyl group (preferably having 1 to 20 carbon atoms) is bonded to the nitrogen atom, a cycloalkyl group (preferably having 3 to 20 carbon atoms) or an aryl group (preferably having 6 to 12 carbon atoms) besides the alkyl group may be bonded to the nitrogen atom.

In the ammonium salt compounds, it is preferred for the alkyl chain thereof to contain an oxygen atom, thereby forming an oxyalkylene group. The number of oxyalkylene groups in each molecule is one or more, preferably 3 to 9 and more preferably 4 to 6. The oxyalkylene group is preferably an oxyethylene group (—CH₂CH₂O—) or an oxypropylene group (—CH(CH₃)CH₂O— or —CH₂CH₂CH₂O—), more preferably an oxyethylene group.

As the anion of the ammonium salt compounds, there can be mentioned a halide, a sulfonate, a borate, a phosphate, a hydroxide or the like. Of these, a hydroxide is preferred.

Among halides, a chloride, a bromide and an iodide are especially preferred.

The amine compound having a phenoxy group can be obtained by first heating a primary or secondary amine having a phenoxy group and a haloalkyl ether so as to effect a reaction therebetween, subsequently adding an aqueous solution of a strong base, such as sodium hydroxide, potassium hydroxide or a tetraalkylammonium, and thereafter carrying out an extraction with an organic solvent, such as ethyl acetate or chloroform. Alternatively, the amine compound having a phenoxy group can be obtained by first heating a primary or secondary amine and a haloalkyl ether having a phenoxy group at its terminus so as to effect a reaction therebetween, subsequently adding an aqueous solution of a strong base, such as sodium hydroxide, potassium hydroxide or a tetraalkylammonium, and thereafter carrying out an extraction with an organic solvent, such as ethyl acetate or chloroform.

From the viewpoint of sensitivity, roughness and stability, an ammonium salt compound is preferred among the various basic compounds. A quaternary ammonium salt compound in its hydroxide form is most preferred.

(Compound Configured to Increase its Basicity Upon an Action of Acid)

Compounds configured to increase its basicity upon an action of acid can also be employed as the basic compound. As an example thereof, those having a structure represented by following formula (A) can be exemplified. In the compound below, electron withdrawing ester bond is adjacent to N atom. Therefore, the compound itself has relatively low basicity. However, an action of acid decomposes the moiety represented by —C(Rb)(Rb)(Rb), which is followed by decarboxylation of the ester bond moiety. Thus, the electron withdrawing ester group is removed, presumably making the compound below to show effective basicity.

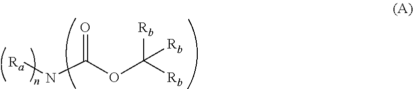

In general formula (A), Ra represents a hydrogen atom, an alkyl group, a monovalent alicyclic hydrocarbon group, an aryl group, or an aralkyl group. When n is 2, two Ra's may either be identical to or different from each other and two Ra's may be bonded to each other to form a bivalent heterocyclic hydrocarbon group (preferably having 20 or less carbon atoms) or its derivatives.

Rb or each of Rb's independently represents a hydrogen atom, an alkyl group, a monovalent alicyclic hydrocarbon group, an aryl group, or an aralkyl group, with the proviso that each of three Rb's in —C(Rb)(Rb)(Rb) does not simultaneously represent an hydrogen atom.

At least two of Rb's may be bonded to each other to form an alicyclic hydrocarbon group, an aromatic hydrocarbon group, a heterocyclic hydrocarbon group, or its derivatives.

In the above formula, n represents an integer of 0 to 2 and m represents an integer of 1 to 3, on condition that n+m=3.

The alkyl group, the monovalent alicyclic hydrocarbon group, the aryl group and the aralkyl group represented by Ra and Rb may further contain one or more substituents. As such, there can be mentioned, for example, functional groups such as a hydroxyl group, a cyano group, an amino group, a pyrrolidino group, a piperidino group, a morpholino group, or an oxo group; an alkoxy group; and a halogen atom.

As the alkyl group, the monovalent alicyclic hydrocarbon group, the aryl group and the aralkyl group, all of which may further have one or more substituents, there can be mentioned the followings:

a group derived from a linear or branched alkane such as methane, ethane, propane, butane, pentane, hexane, heptane, octane, nonane, decane, undecane, or dodecane; and the group derived from the alkane and substituted with, for example, one or more cycloalkyl groups;

a group derived from cycloalkane such as cyclobutane, cyclopentane, cyclohexane, cycloheptane, cyclooctane, norbornane, adamantane, or noradamantane; and the group derived from the cycloalkane and substituted with, for example, one or more linear or branched alkyl group;

a group derived from aromatic compound such as benzene, naphthalene, or anthracene; and the group derived from the atomatic compound and substituted with, for example, one or more linear or branched alkyl group;

a group derived from heterocyclic compound such as pyrrolidine, piperidine, morpholine, tetrahydrofuran, tetrahydropyrane, indole, indoline, quinoline, perhydroquinoline, indazole, or benzimidazole; the group derived from heterocyclic compound and substituted with one or more linear or branched alkyl group or a group derived from the aromatic compound;

a group derived from linear or branched alkane and substituted with, for example, a group derived from aromatic compound;

a group derived from cycloalkane and substituted with, for example, a group derived from aromatic compound; or

each of these groups substituted with a functional group such as a hydroxyl group, a cyano group, an amino group, a pyrrolidino group, a piperidino group, a morpholino group, or an oxo group.

Further, as the bivalent heterocyclic hydrocarbon group (preferably having 1 to 20 carbon atoms) or its derivative, formed by mutual binding of Ra's, for example, the followings can be exemplified:

a group derived from heterocyclic compound such as pyrrolidine, piperidine, morpholine, 1,4,5,6-tetrahydropyrimidine, 1,2,3,4-tetrahydroquinoline, 1,2,3,6-tetrahydroquinoline, homopiperadine, 4-azabenzimidazole, benztriazole, 5-azabenztriazole, 1H-1,2,3-triazole, 1,4,7-triazacyclononane, tetrazole, 7-azaindole, indazole, benzimidazole, imidazo[1,2-a]pyridine, (1S,4S)-(+)2,5-azabicyclo[2.2.1]heptane, 1,5,7-triazabicyclo[4.4.0]dec-5-en, indole, indoline, 1,2,3,4-tetrahydroquinoxaline, perhydroquinoline, or 1,5,9-triazacyclododecane; or

the group derived from heterocyclic compound and substituted with at least one of a group derived from linear or branched alkane, a group derived from cycloalkane, a group derived from aromatic compound, a group derived from heterocyclic compound, or a functional group such as a hydroxyl group, a cyano group, an amino group, a pyrrolidino group, a piperidino group, a morpholino group, or an oxo group.

Particularly preferred examples of the compound configured to increase its basicity upon an action of acid will be shown below, which however in no way limit the scope of the present invention.

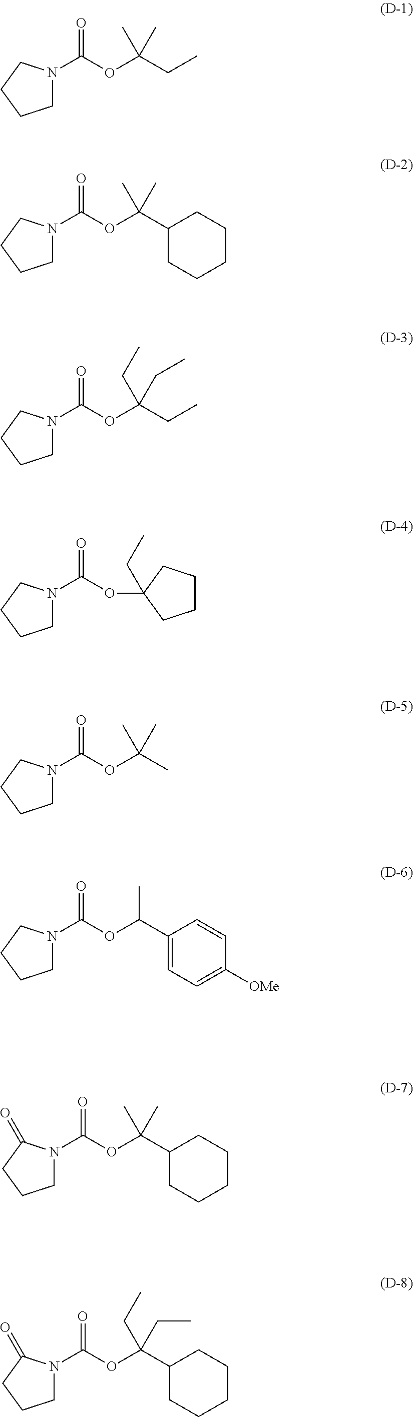

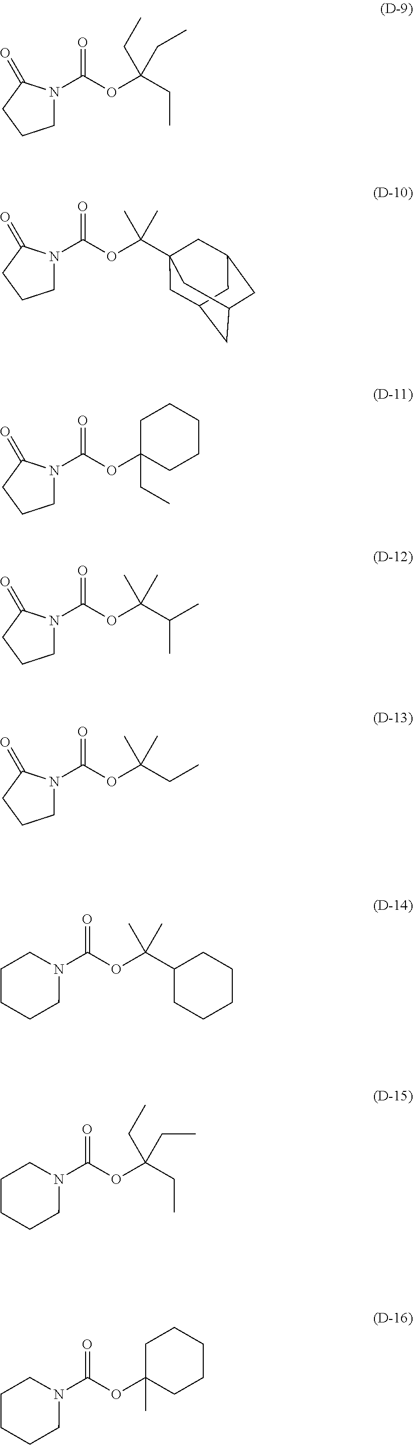

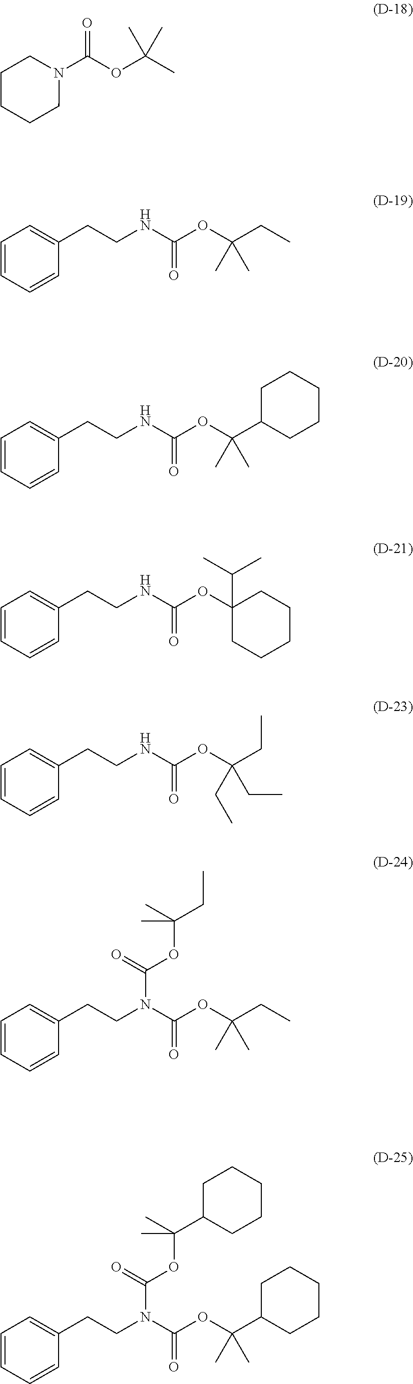

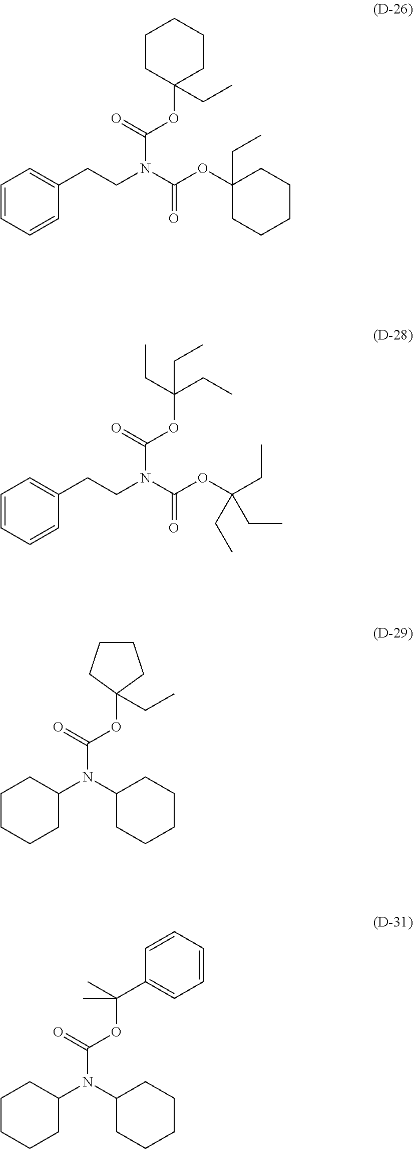

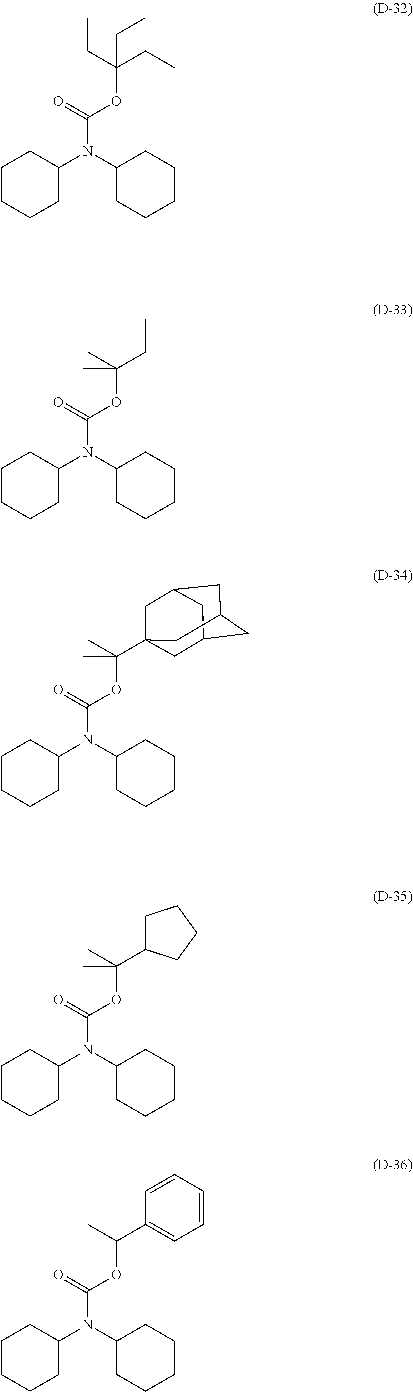

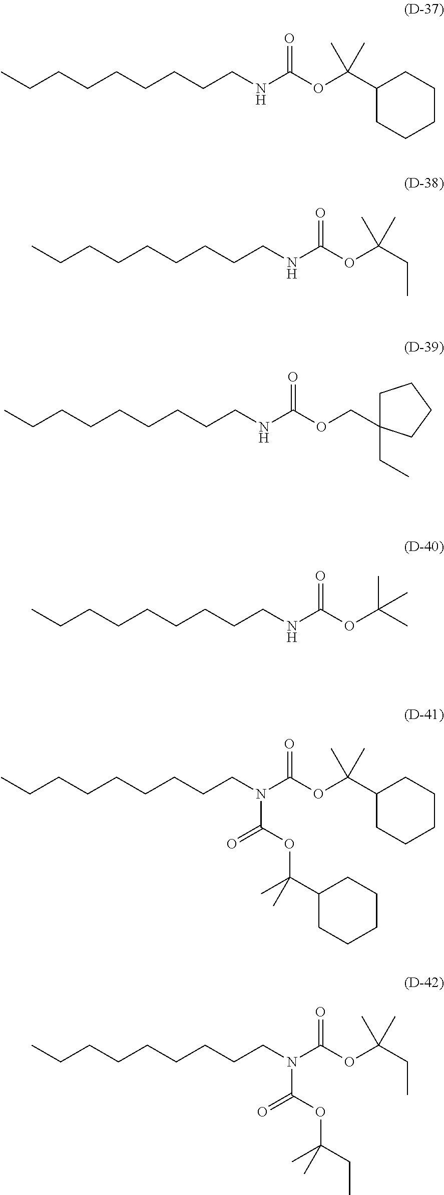

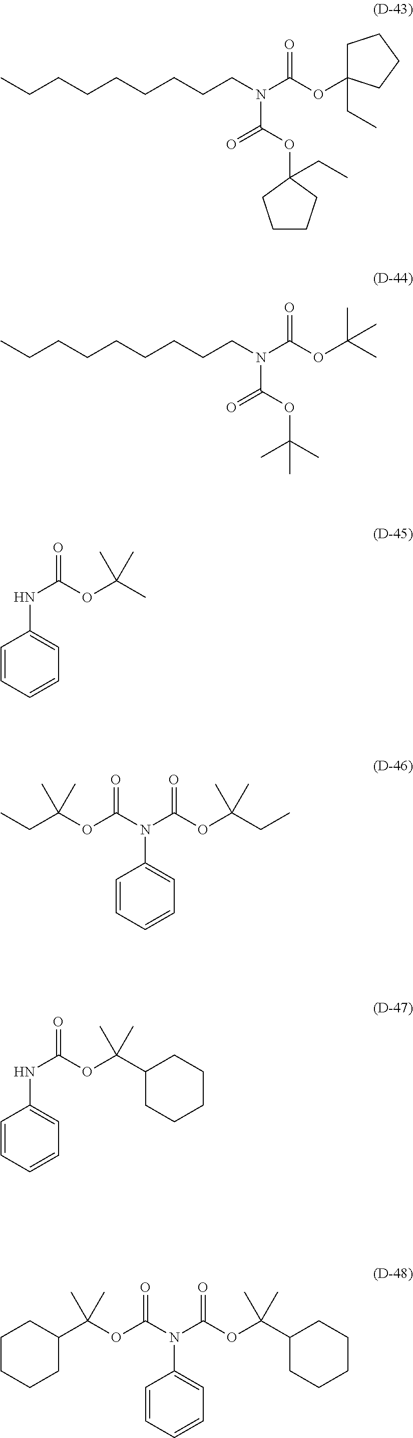

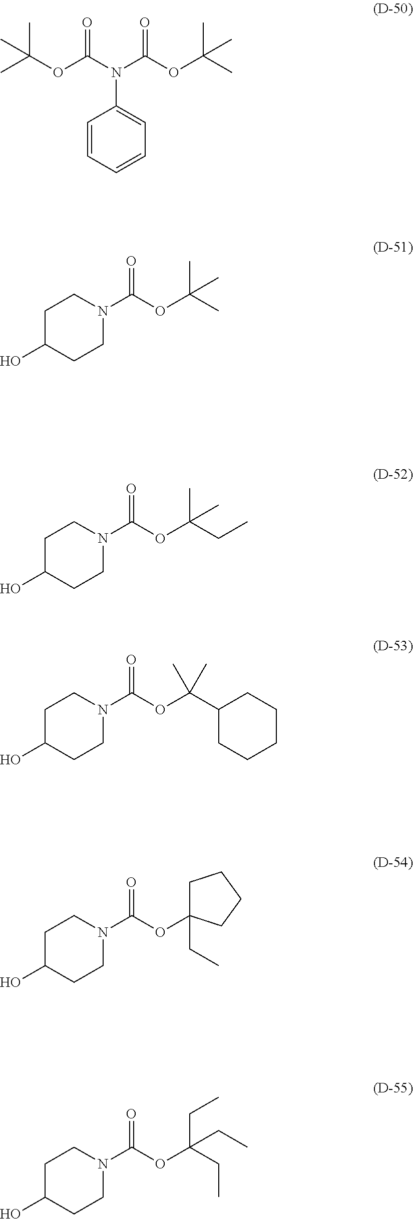

The basic compounds described above can be used either individually or in combination.

The molecular weight of the basic compound is preferably 250 to 1000, more preferably 250 to 800, and particularly preferably 400 to 800.

The total amount of basic compound used based on the solid contents of the composition is preferably in the range of 1.0 to 8.0 mass %, more preferably 1.5 to 5.0 mass %, and further more preferably 2.0 to 4.0 mass %.

[8] Fluorinated and/or Siliconized Surfactant

The composition according to the present invention may further contain one or more fluorinated and/or siliconized surfactants. As the fluorinated and/or siliconized surfactant, a fluorinated surfactant, a siliconized surfactant, a surfactant containing both fluorine and silicon atoms, and a mixture thereof can be exemplified.

The composition according to the present invention when containing the fluorinated and/or siliconized surfactant would, in the use of an exposure light source of 250 nm or below, especially 220 nm or below, realize favorable sensitivity and resolving power and produce a resist pattern of less adhesion and development defects.

As useful commercially available surfactants, there can be mentioned, for example, fluorinated or siliconized surfactants, such as Eftop EF301 and EF303 (produced by Shin-Akita Kasei Co., Ltd.), Florad FC 430 and 431 (produced by Sumitomo 3M Ltd.), Megafac F171, F173, F176, F189 and R08 (produced by Dainippon Ink & Chemicals, Inc.), Surflon S-382, SC101, 102, 103, 104, 105 and 106 (produced by Asahi Glass Co., Ltd.) and Troy Sol S-366 (produced by Troy Chemical Co., Ltd.). Further, polysiloxane polymer KP-341 (produced by Shin-Etsu Chemical Co., Ltd.) can be employed as the siliconized surfactant.

As the surfactants, besides the above publicly known surfactants, use can be made of a surfactant based on a polymer having a fluorinated aliphatic group derived from a fluorinated aliphatic compound produced by a telomerization technique (also called a telomer process) or an oligomerization technique (also called an oligomer process). The fluorinated aliphatic compound can be synthesized by the process described in JP-A-2002-90991.

The polymer having a fluorinated aliphatic group is preferably a copolymer from a monomer having a fluorinated aliphatic group and a poly(oxyalkylene) acrylate and/or poly(oxyalkylene) methacrylate, which copolymer may have an irregular distribution or may result from block copolymerization.

As the poly(oxyalkylene) group, there can be mentioned a poly(oxyethylene) group, a poly(oxypropylene) group, a poly(oxybutylene) group or the like. Further, use can be made of a unit having alkylene groups of different chain lengths in a single chain, such as poly(oxyethylene-oxypropylene-oxyethylene block concatenation) or poly(oxyethylene-oxypropylene block concatenation).

Moreover, the copolymer from a monomer having a fluorinated aliphatic group and a poly(oxyalkylene) acrylate (or methacrylate) is not limited to two-monomer copolymers and may be a three or more monomer copolymer obtained by simultaneous copolymerization of two or more different monomers having a fluorinated aliphatic group, two or more different poly(oxyalkylene) acrylates (or methacrylates), etc.

For example, as a commercially available surfactant, there can be mentioned Megafac F178, F-470, F-473, F-475, F-476 or F-472 (produced by Dainippon Ink & Chemicals, Inc.). Further, there can be mentioned a copolymer from an acrylate (or methacrylate) having a C₆F₁₃ group and a poly(oxyalkylene) acrylate (or methacrylate), a copolymer from an acrylate (or methacrylate) having a C₆F₁₃ group, poly(oxyethylene) acrylate (or methacrylate) and poly(oxypropylene) acrylate (or methacrylate), a copolymer from an acrylate (or methacrylate) having a C₈F₁₇ group and a poly(oxyalkylene) acrylate (or methacrylate), a copolymer from an acrylate (or methacrylate) having a C₈F₁₇ group, poly(oxyethylene) acrylate (or methacrylate) and poly(oxypropylene) acrylate (or methacrylate), or the like.

The amount of fluorinated and/or siliconized surfactant used is preferably in the range of 0.0001 to 2 mass %, more preferably 0.001 to 1 mass % based on the total solids of the composition.

[9] Hydrophobic Resin

As mentioned above, the composition according to the present invention may further contain a hydrophobic resin. When a hydrophobic resin is further contained, the hydrophobic resin is unevenly localized in the surface layer of the film formed from the composition. Thus, when water is used as a liquid for liquid immersion, the receding contact angle of the film with reference to the liquid for liquid immersion can be increased. Accordingly, the liquid-immersion liquid tracking property of the film can be enhanced.

The hydrophobic resin typically contains fluorine atom and/or silicone atom. The fluorine atom and/or silicon atom in the hydrophobic resin may be present in the principal chain of the resin or may be a substituent on the side chain thereof.

When the hydrophobic resin contains fluorine atom, the resin preferably has, as a partial structure containing one or more fluorine atoms, an alkyl group containing one or more fluorine atoms, a cycloalkyl group containing one or more fluorine atoms, or an aryl group containing one or more fluorine atoms.

The alkyl group containing one or more fluorine atoms is a linear or branched alkyl group having at least one hydrogen atom thereof substituted with one or more fluorine atoms. The group preferably has 1 to 10 carbon atoms, more preferably 1 to 4 carbon atoms.

The cycloalkyl group containing one or more fluorine atoms is a monocyclic or polycyclic alkyl group having at least one hydrogen atom thereof substituted with one or more fluorine atoms.

The aryl group containing one or more fluorine atoms is an aryl group having at least one hydrogen atom of an aryl group substituted with one or more fluorine atoms. As the aryl group, a phenyl or a naphthyl group can be exemplified.

As preferred alkyl groups containing one or more fluorine atoms, cycloalkyl groups containing one or more fluorine atoms and aryl groups containing one or more fluorine atoms, groups of the following general formulae (F2) to (F4) can be exemplified.

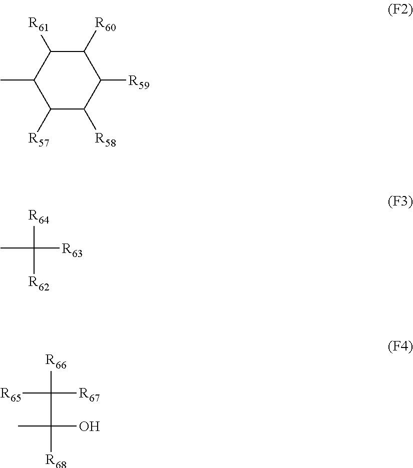

In the general formulae (F2) to (F4),

each of R₅₇ to R₆₈ independently represents a hydrogen atom, a fluorine atom or an alkyl group in condition that: at least one of R₅₇-R₆₁ represents a fluorine atom or an alkyl group having at least one hydrogen atom thereof substituted with one or more fluorine atoms; at least one of R₆₂-R₆₄ represents a fluorine atom or an alkyl group having at least one hydrogen atom thereof substituted with one or more fluorine atoms; and at least one of R₆₅-R₆₈ represents a fluorine atom or an alkyl group having at least one hydrogen atom thereof substituted with one or more fluorine atoms. These alkyl groups preferably are those having 1 to 4 carbon atoms.

It is preferred that all of R₅₇-R₆₁ and R₆₅-R₆₇ represent fluorine atoms.

Each of R₆₂, R₆₃ and R₆₈ preferably represents an alkyl group having at least one hydrogen atom thereof substituted with one or more fluorine atoms, more preferably a perfluoroalkyl group having 1 to 4 carbon atoms. R₆₂ and R₆₃ may be bonded to each other to form a ring.

Specific examples of the groups represented by the general formula (F2) include a p-fluorophenyl group, a pentafluorophenyl group, and a 3,5-di(trifluoromethyl)phenyl group.

Specific examples of the groups represented by the general formula (F3) include a hexafluoroisopropyl group, a heptafluoroisopropyl group, a hexafluoro(2-methyl)isopropyl group, an octafluoroisobutyl group, a nonafluoro-t-butyl group, a perfluoroisopentyl group. Of these, a hexafluoroisopropyl group and a heptafluoroisopropyl group are preferred.

Specific examples of the groups represented by the general formula (F4)

include —C(CF₃)₂OH, —C(C₂F₅)₂OH, —C(CF₃)(CH₃)OH, —CH(CF₃)OH and the like. Of these, —C(CF₃)₂OH is particularly preferred.

Specific examples of the repeating units having a fluorine atom will be shown below.

In the specific examples, X₁ represents a hydrogen atom, —CH₃, —F or —CF₃. X₂ represents —F or —CF₃.

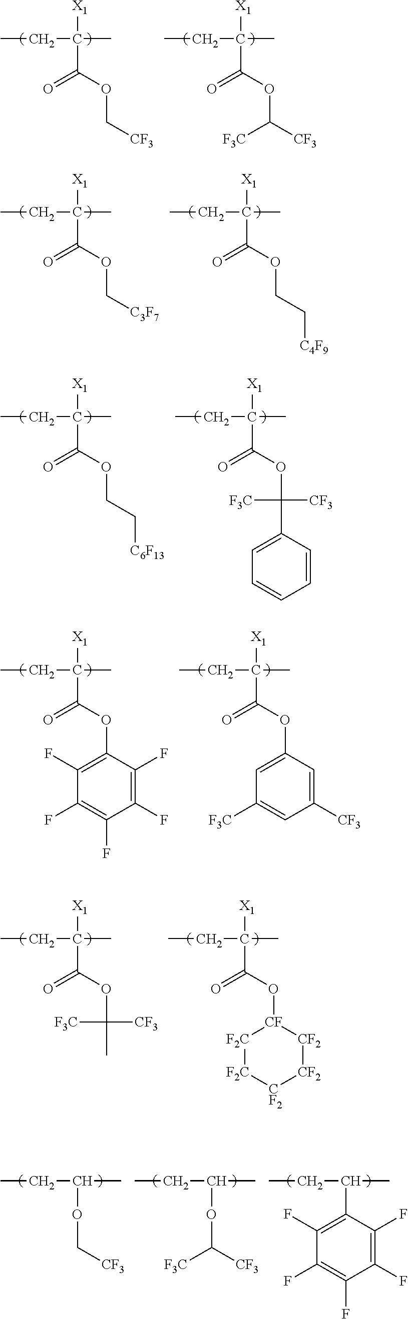

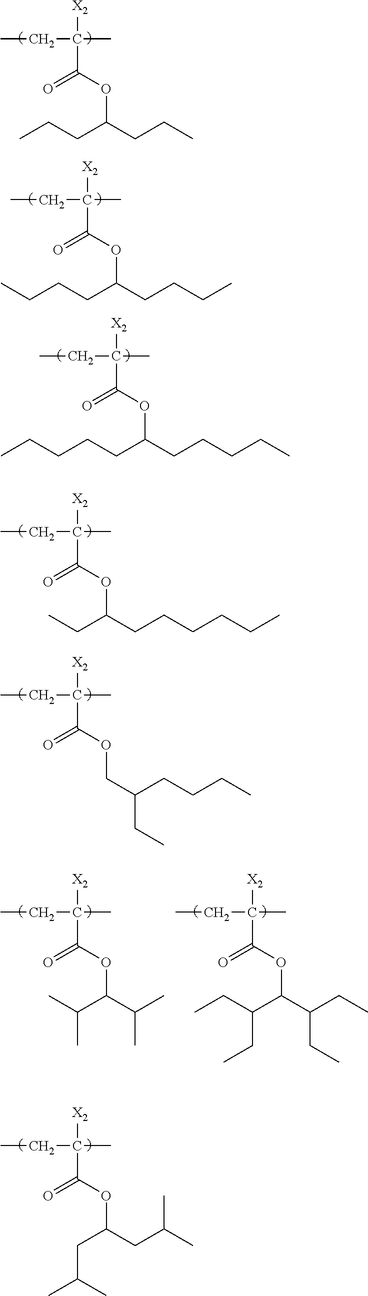

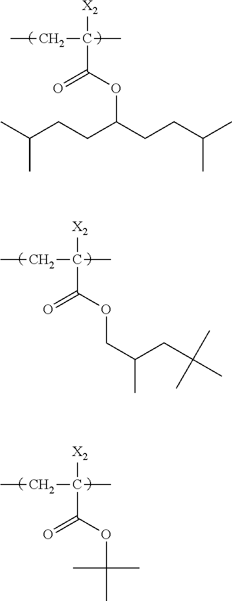

When the hydrophobic resin contains one or more silicon atoms, the resin preferably contains, as partial structure containing the silicon atom, an alkylsilyl structure or a cyclosiloxane structure. Preferred alkylsilyl structure is that containing one or more trialkylsilyl groups.

As the alkylsilyl structure and cyclosiloxane structure, any of the groups represented by the following general formulae (CS-1) to (CS-3) can be exemplified.

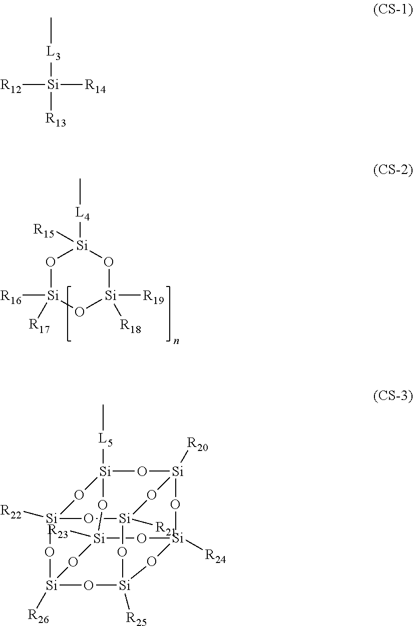

In the general formulae (CS-1) to (CS-3), each of R12 to R26 independently represents a linear or branched alkyl group or a cycloalkyl group. The alkyl group preferably has 1 to 20 carbon atoms. The cycloalkyl group preferably has 3 to 20 carbon atoms.

Each of L₃ to L₅ represents a single bond or a bivalent connecting group. As the bivalent connecting group, any one or a combination of two or more groups selected from the group consisting of an alkylene group, a phenylene group, an ether group, a thioether group, a carbonyl group, an ester group, an amido group, a urethane group and a urea group can be exemplified.

In the formulae, n is an integer of 1 to 5, and preferably an integer of 2 to 4.

Specific examples of the repeating units having the groups represented by the general formulae (CS-1) to (CS-3) will be shown below. In the specific examples, X₁ represents a hydrogen atom, —CH₃, —F or —CF₃.

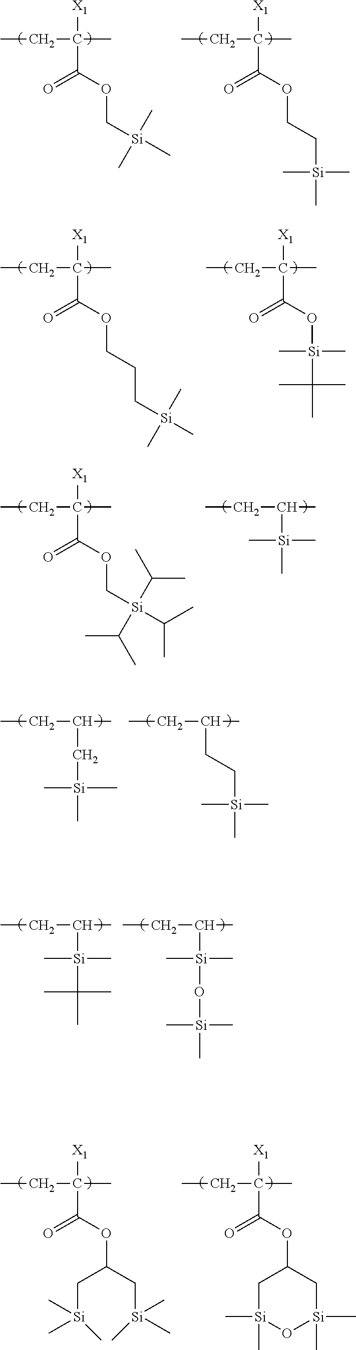

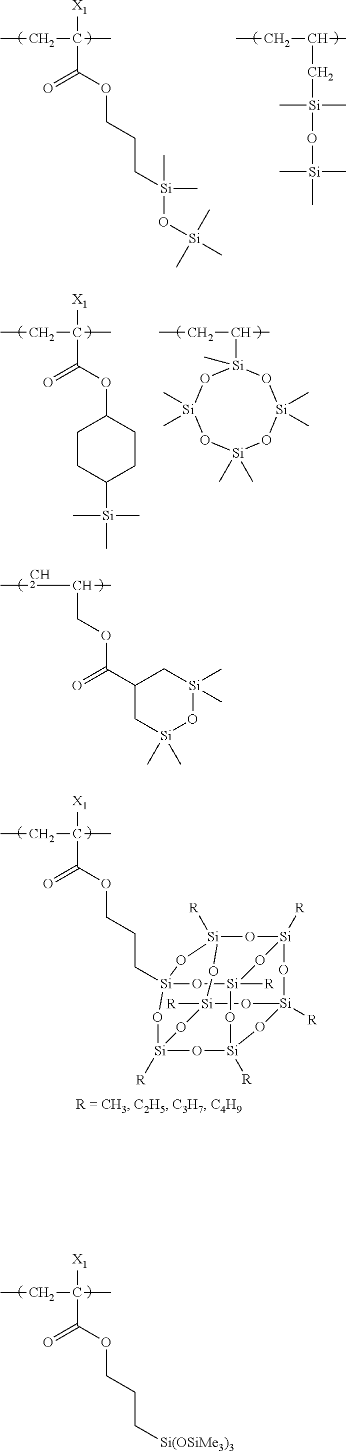

The hydrophobic resin may further contain at least one group selected from among the following groups (x) to (z):

(x) an alkali soluble group,

(y) a group that is decomposed by the action of an alkali developer, resulting in an increase of solubility in the alkali developer, and

(z) a group that is decomposed by the action of an acid.

As preferred (x) alkali soluble groups, a fluoroalcohol group, a sulfonimido group, and a bis(carbonyl)methylene group can be exemplified. As preferred fluoroalcohol group, a hexafluoroisopropanol group can be exemplified.

The content of repeating units having an alkali soluble group based on all the repeating units of the polymer is preferably in the range of 1 to 50 mol %, more preferably 3 to 35 mol %, and still more preferably 5 to 20 mol %.

Specific examples of the repeating units having an alkali soluble group will be shown below. In the formulae, Rx represents H, CH₃, CF₃, or CH₂OH.

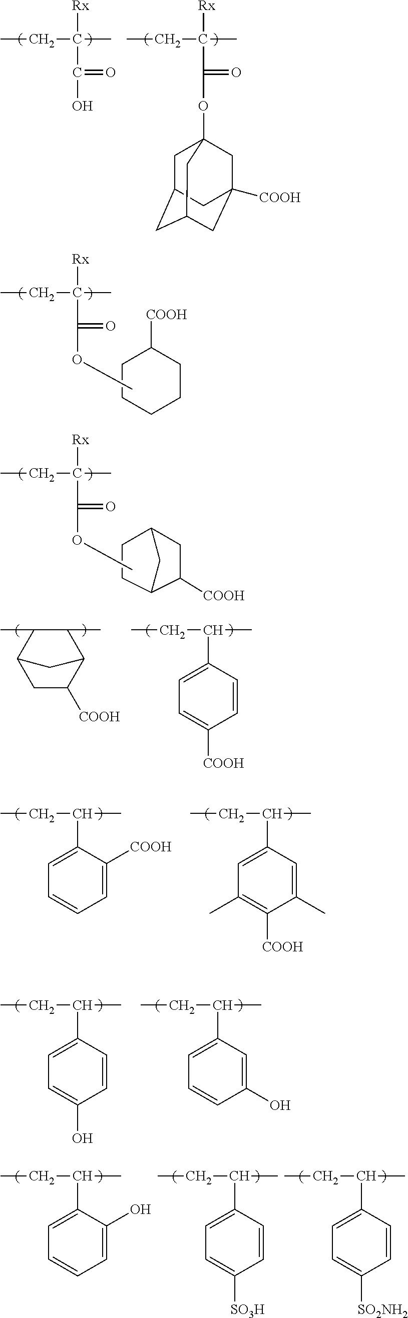

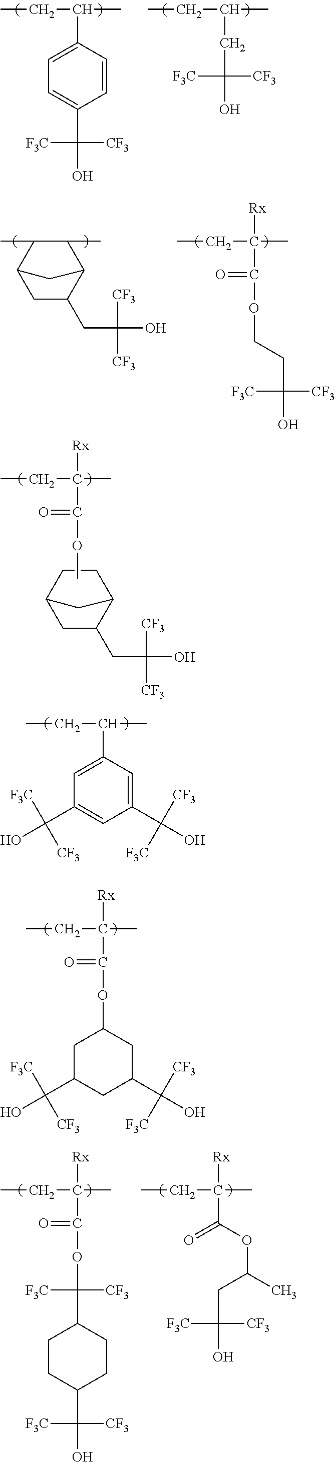

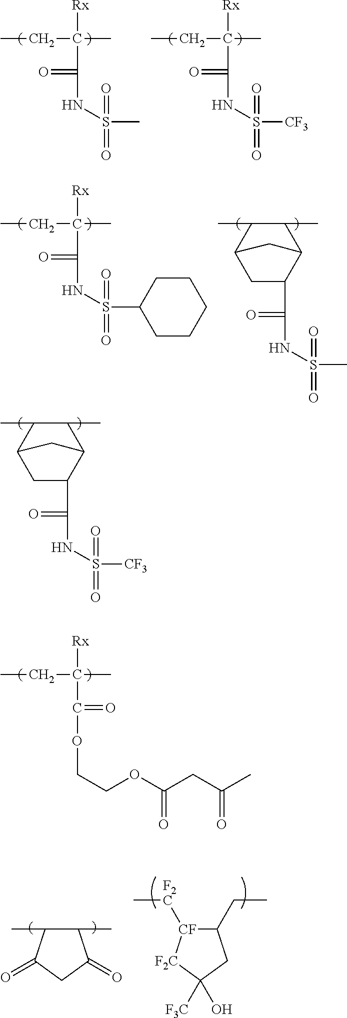

As the group (y) that is decomposed by the action of an alkali developer resulting in an increase of solubility in the alkali developer, a group having a lactone structure, an acid anhydride group, and an acid imide group can be exemplified. Of these, a group having a lactone structure is particularly preferred.

As the repeating unit having a group that is decomposed by the action of an alkali developer resulting in an increase of solubility in the alkali developer, for example, those explained in connection with section [3] can be exemplified.

The content of repeating units having a group resulting in an increase of solubility in an alkali developer based on all the repeating units of the polymer is preferably in the range of 1 to 40 mol %, more preferably 3 to 30 mol %, and still more preferably 5 to 15 mol %.

As the repeating unit (z) having a group that is decomposed by the action of an acid, those explained in connection with section [3] can be exemplified.

The content of repeating units having a group that is decomposed by the action of an acid in the hydrophobic resin based on all the repeating units of the polymer is preferably in the range of 1 to 80 mol %, more preferably 10 to 80 mol %, and still more preferably 20 to 60 mol %.

The hydrophobic resin may further have any of the repeating units represented by the following general formula (III).

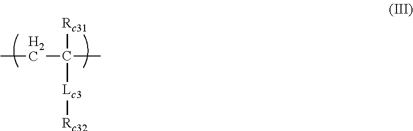

In the general formula (III), R_{c31} represents a hydrogen atom, an alkyl group, an alkyl group optionally substituted with one or more fluorine atoms, a cyano group or a group of the formula —CH₂—O—R_{ac2} in which R_{ac2} represents a hydrogen atom, an alkyl group or an acyl group.

R_{c31} is preferably a hydrogen atom, a methyl group, a hydroxymethyl group, or a trifluoromethyl group, more preferably a hydrogen atom or a methyl group.

R_{c32} represents a group containing an alkyl group, a cycloalkyl group, an alkenyl group, a cycloalkenyl group, or an aryl group. These groups may be substituted with fluorine atom and/or silicon atom.

The alkyl group represented by R_{c32} is preferably a linear or branched alkyl group having 3 to 20 carbon atoms.

The cycloalkyl group is preferably a cycloalkyl group having 3 to 20 carbon atoms.

The alkenyl group is preferably an alkenyl group having 3 to 20 carbon atoms.

The cycloalkenyl group is preferably a cycloalkenyl group having 3 to 20 carbon atoms.

The aryl group is preferably an aryl group having 6 to 20 carbon atoms, such as a phenyl group or a naphthyl group.

These groups may have one or more substituents.

Preferably, R_{c32} represents an unsubstituted alkyl group or an alkyl group substituted with one or more fluorine atoms.

L_{c3} represents a single bond or a bivalent connecting group. As the bivalent connecting group represented by L_{c3}, an ester group, an alkylene group (preferably having 1 to 5 carbon atoms), an oxy group, a phenylene group, and an ester bond (a group represented by —COO—) can be exemplified.

The hydrophobic resin may further have any of the repeating units represented by general formula (CII-AB) below.

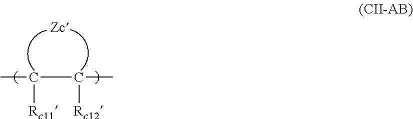

In the formula (CII-AB), each of R_{c11}′ and R_{c12}′ independently represents a hydrogen atom, a cyano group, a halogen atom or an alkyl group. Zc′ represents an atomic group required for forming an alicyclic structure in cooperation with two carbon atoms (C—C) to which R_{c11}′ and R_{c12}′ are respectively bonded.

Specific examples of the repeating units represented by the general formula (III) and general formula (CII-AB) will be shown below. In the specific examples, Ra represents H, CH₃, CH₂OH, CF₃ or CN.

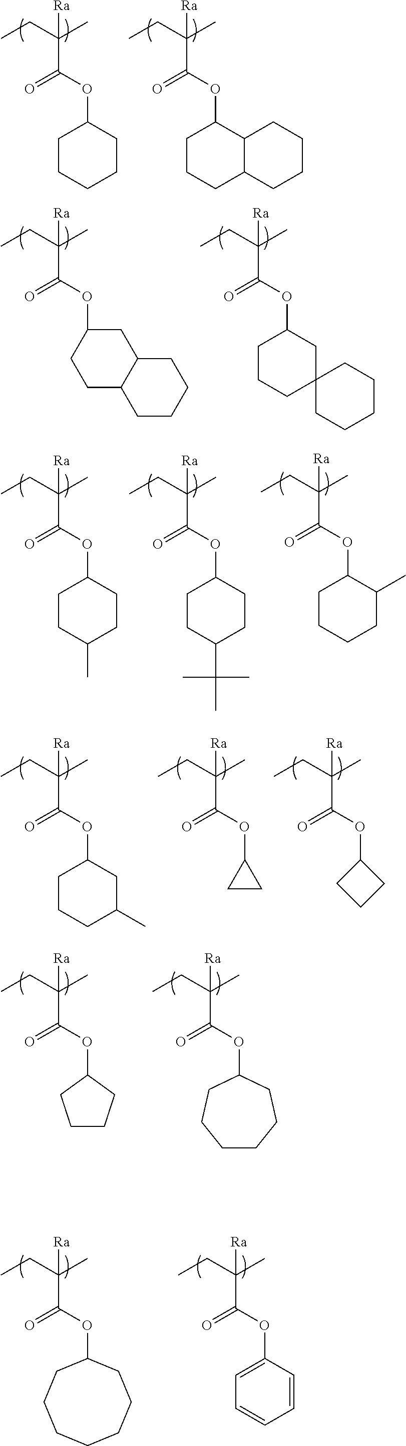

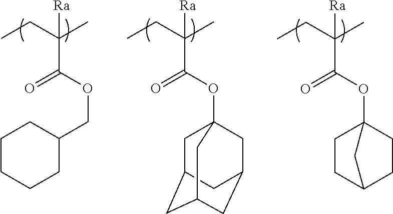

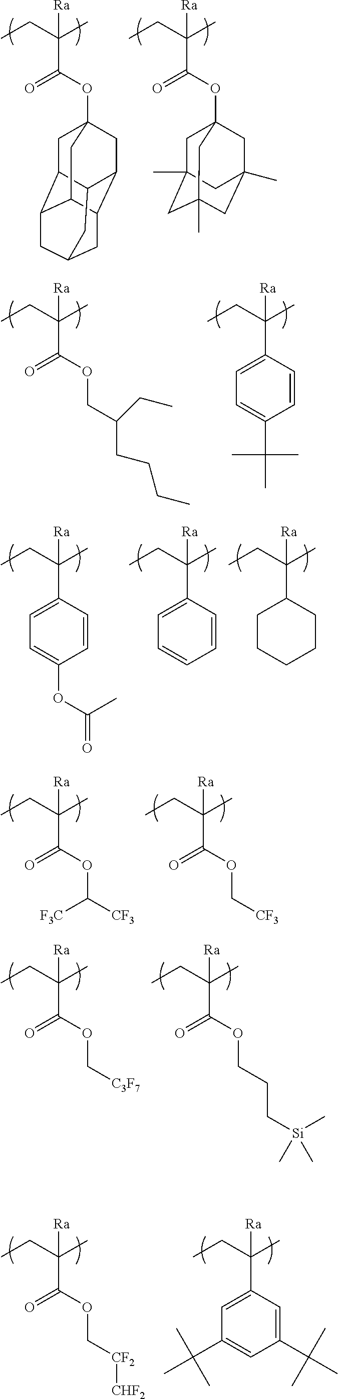

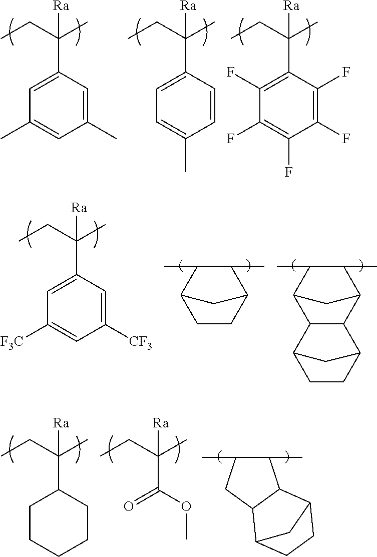

Specific examples of the hydrophobic resins will be shown below. The following Table 2 shows the molar ratio of individual repeating units (corresponding to individual repeating units in order from the left), weight average molecular weight, and degree of dispersal with respect to each of the resins.

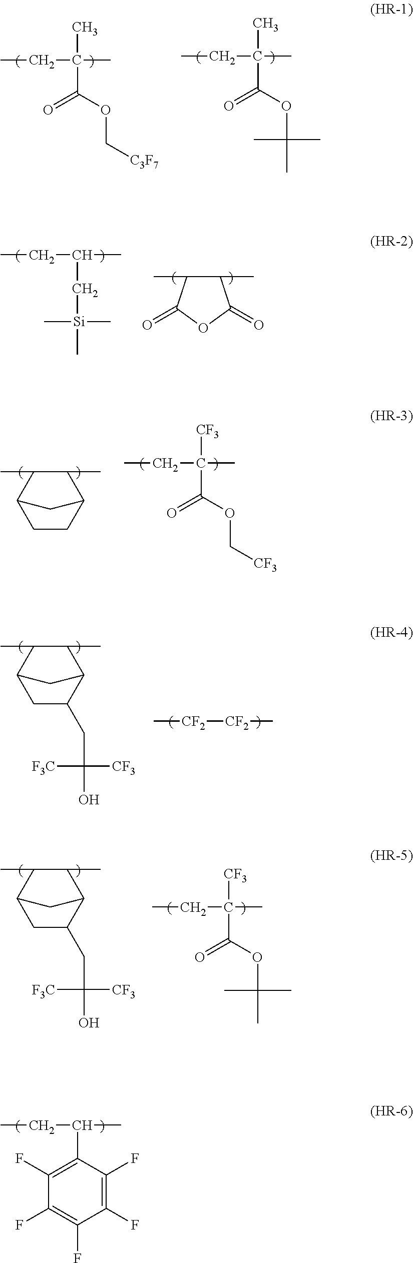

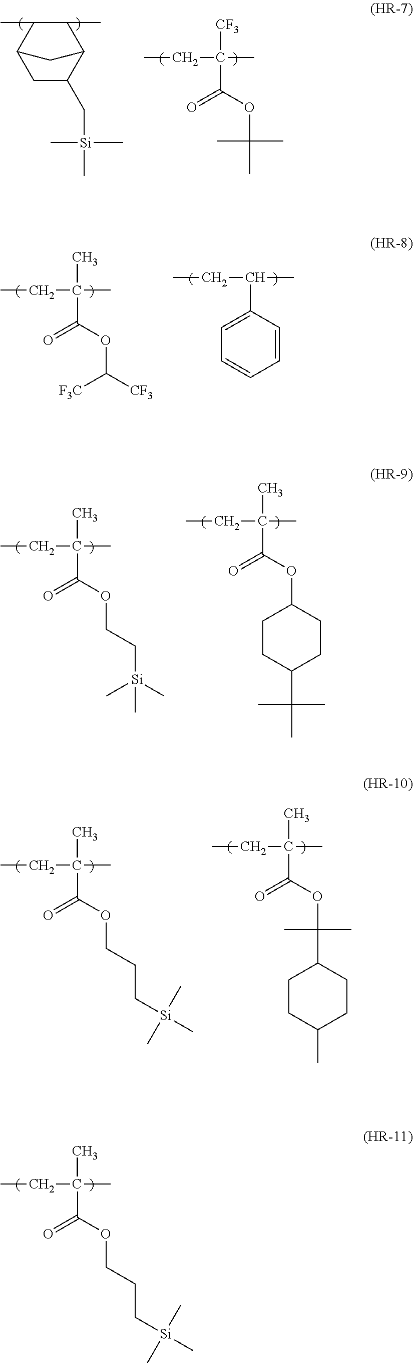

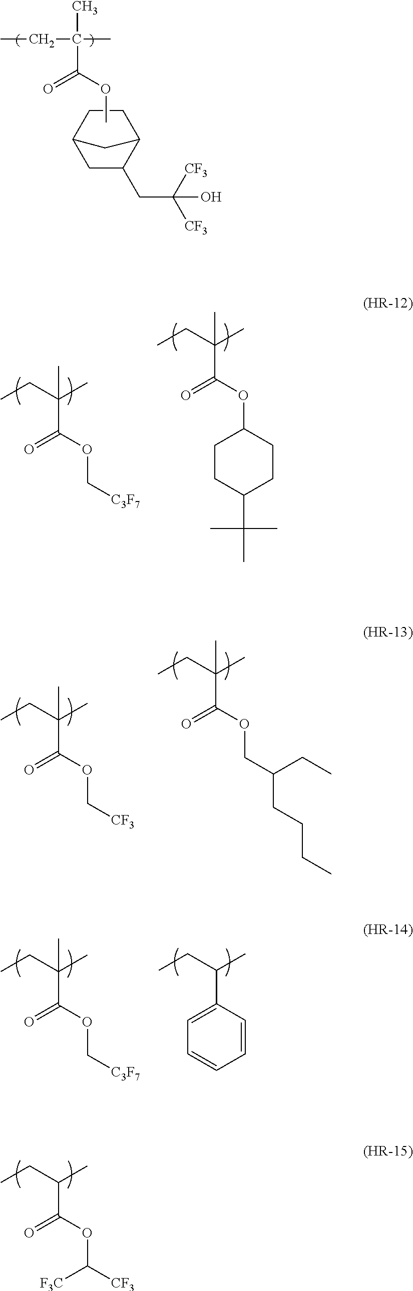

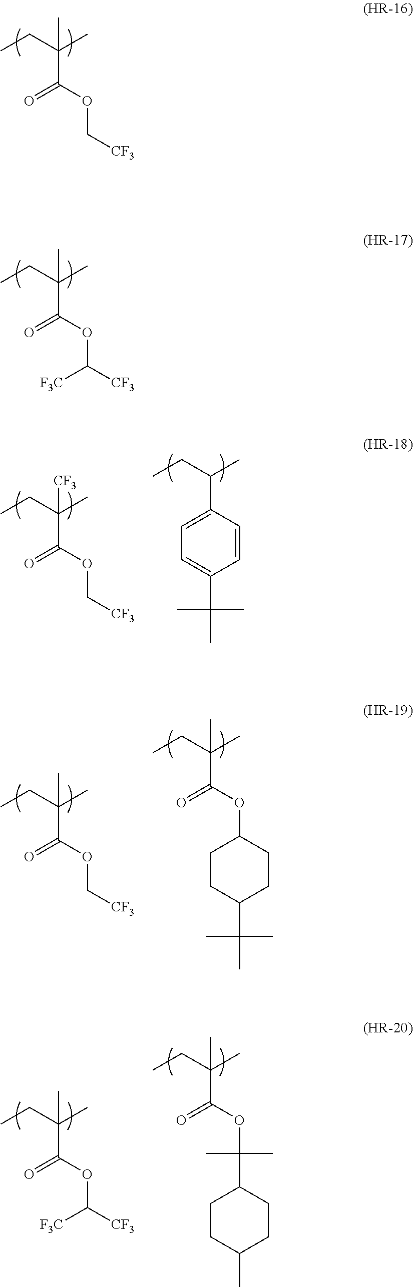

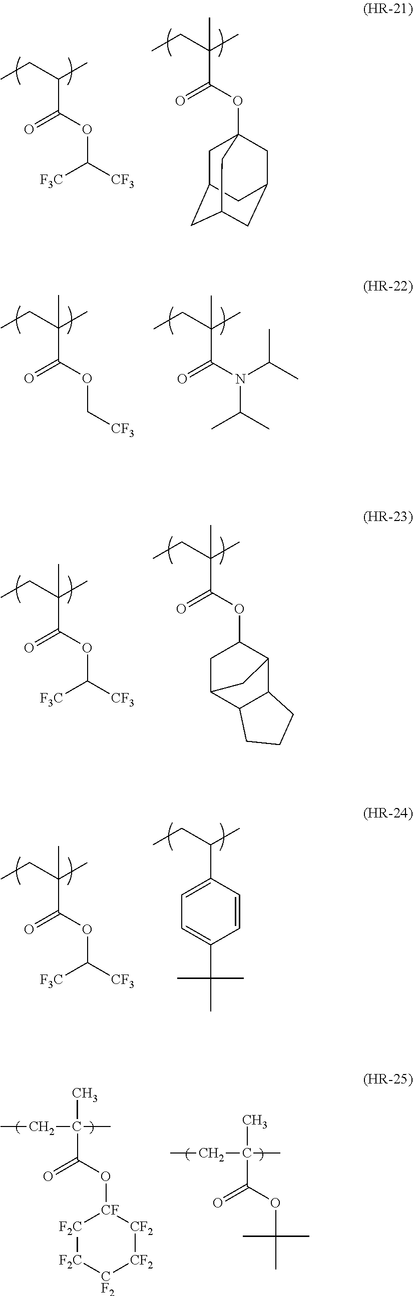

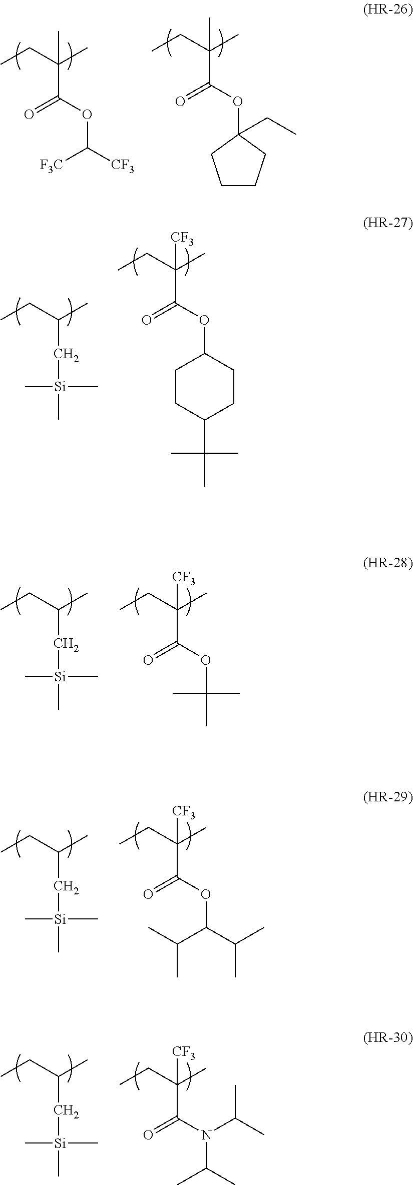

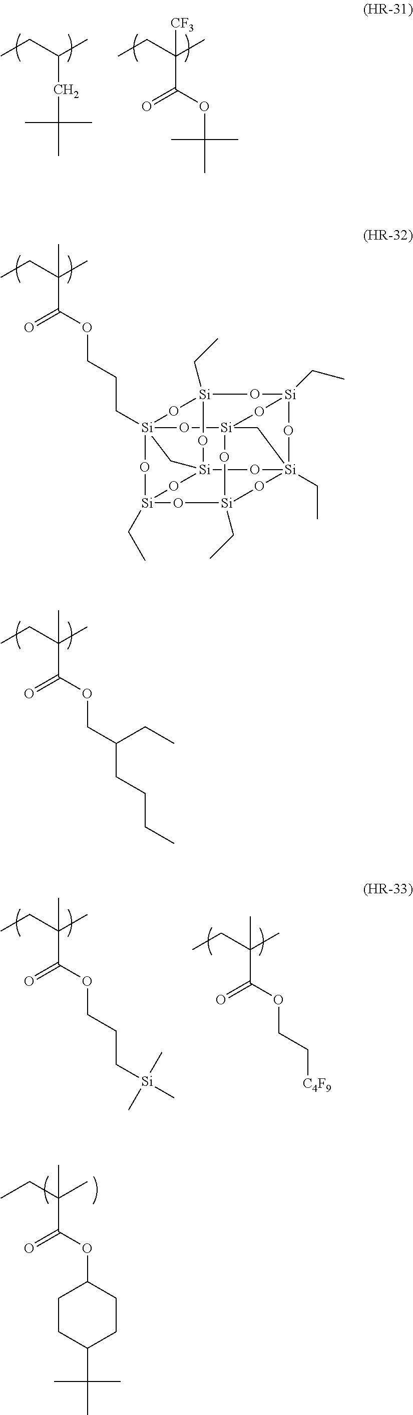

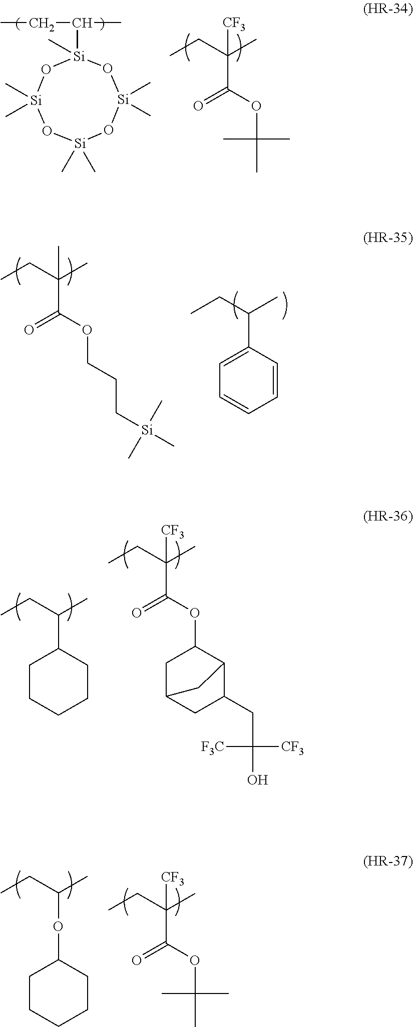

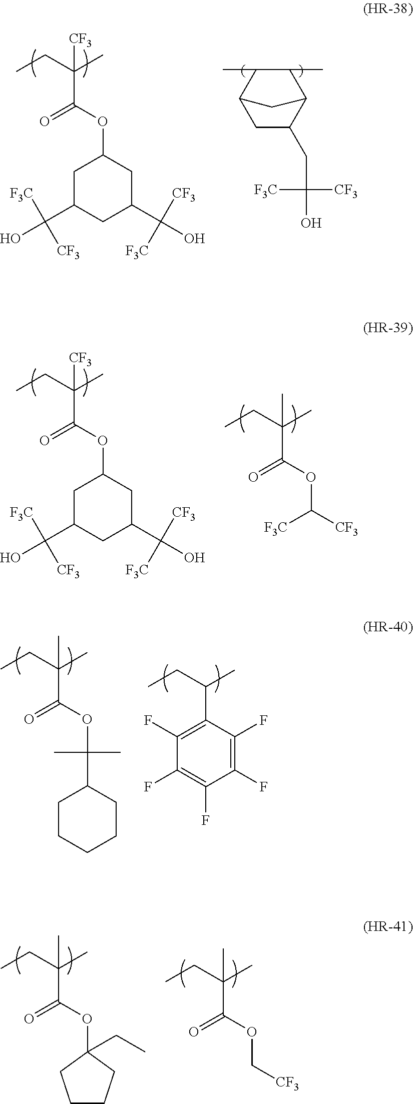

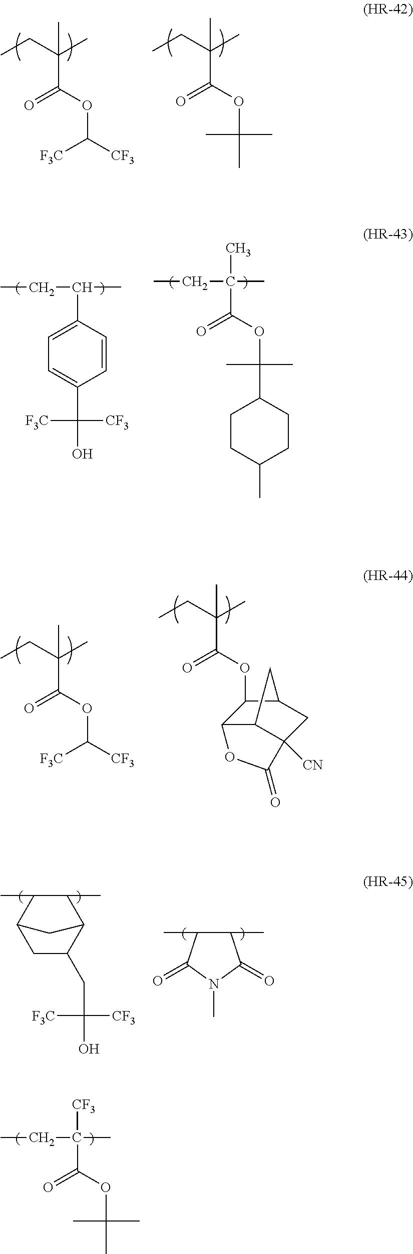

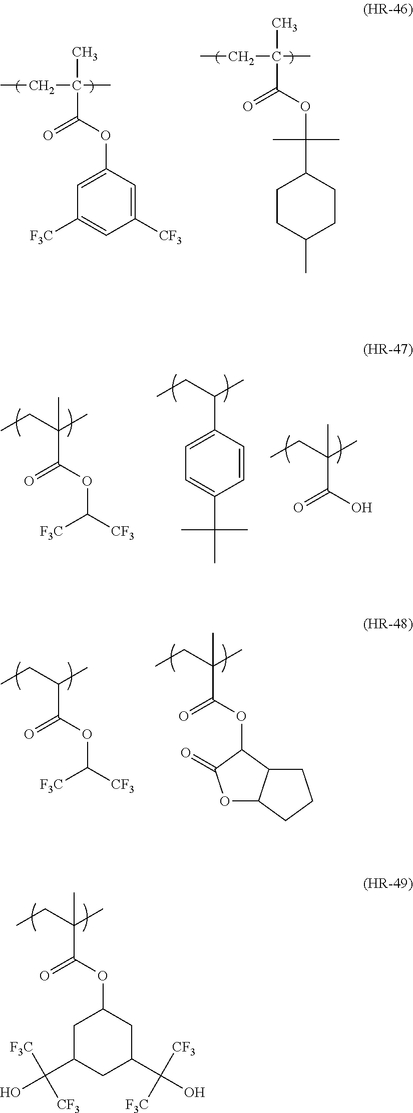

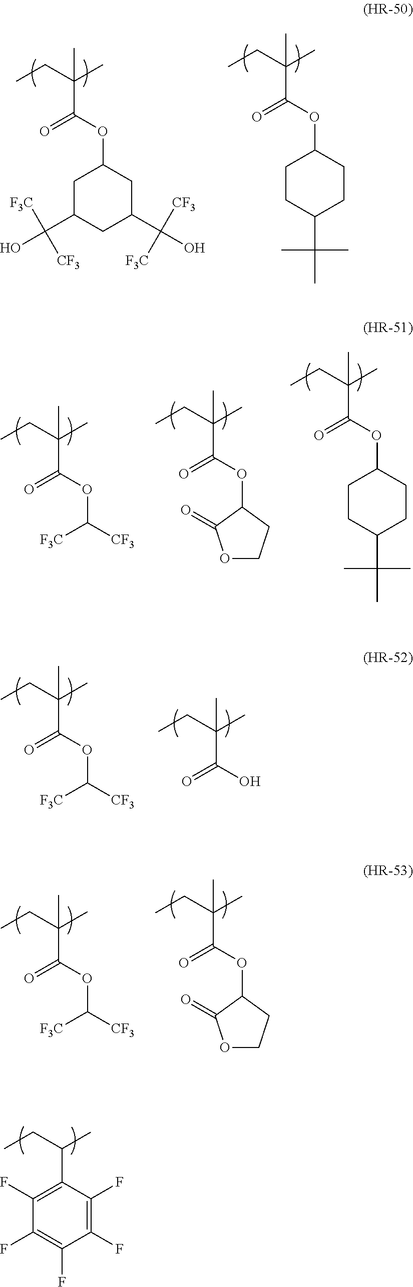

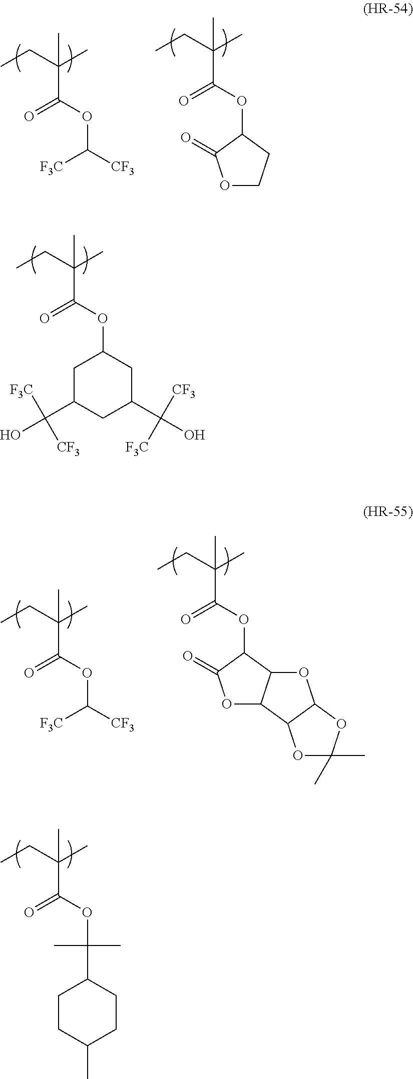

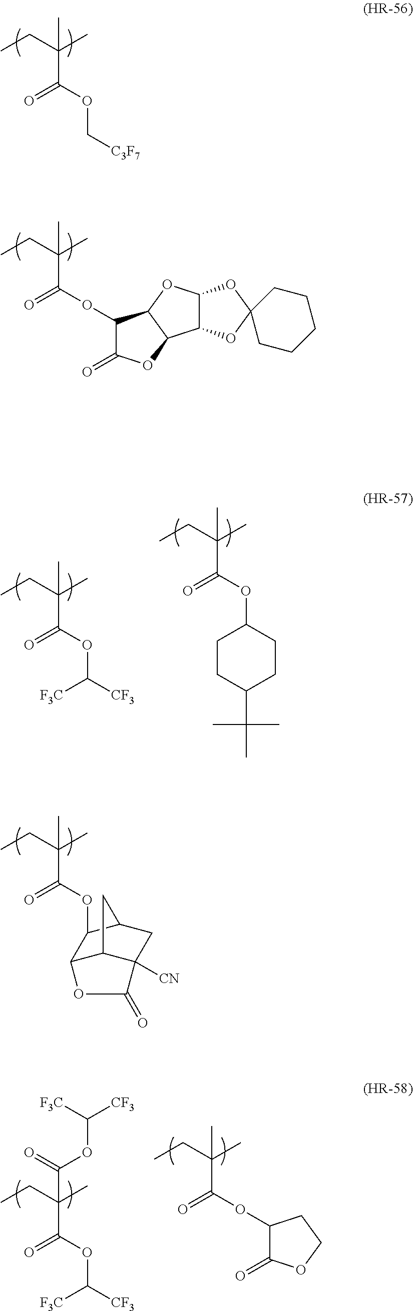

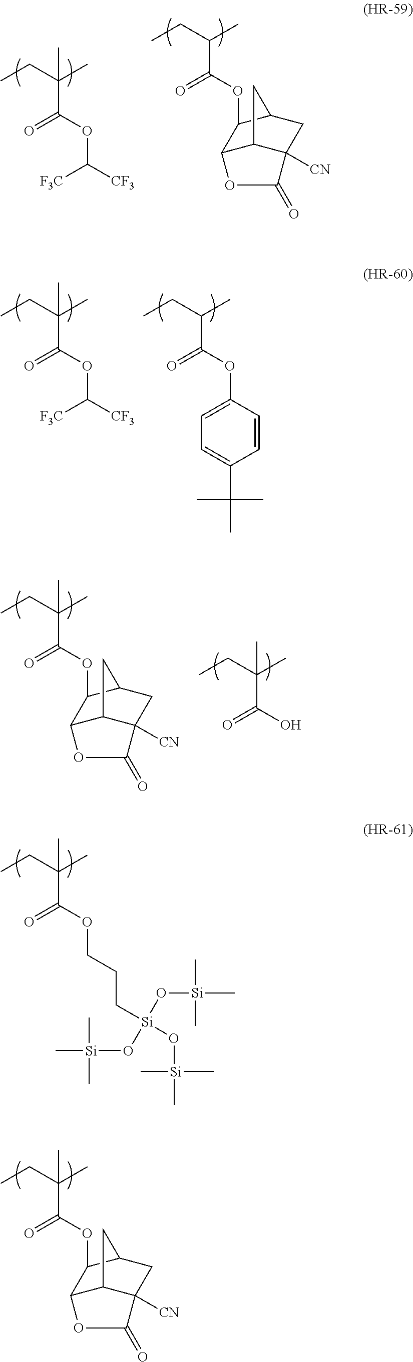

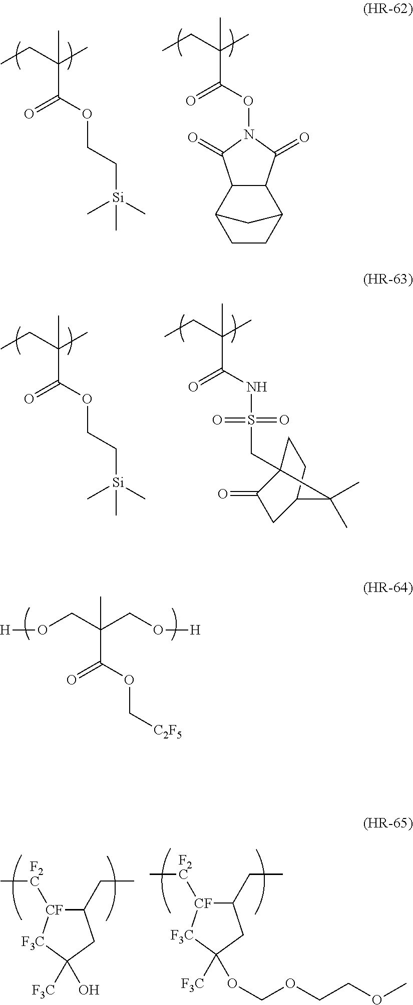

TABLE 2

| Resin | Composition | Mw | Mw/Mn |
|---|---|---|---|
| HR-1 | 50/50 | 4900 | 1.4 |
| HR-2 | 50/50 | 5100 | 1.6 |
| HR-3 | 50/50 | 4800 | 1.5 |
| HR-4 | 50/50 | 5300 | 1.6 |
| HR-5 | 50/50 | 4500 | 1.4 |
| HR-6 | 100 | 5500 | 1.6 |
| HR-7 | 50/50 | 6800 | 1.9 |
| HR-8 | 50/50 | 4200 | 1.3 |
| HR-9 | 50/50 | 5500 | 1.8 |
| HR-10 | 40/60 | 7500 | 1.6 |
| HR-11 | 70/30 | 6600 | 1.8 |
| HR-12 | 40/60 | 3900 | 1.3 |
| HR-13 | 50/50 | 9500 | 1.8 |
| HR-14 | 50/50 | 5300 | 1.6 |
| HR-15 | 100 | 6200 | 1.2 |
| HR-16 | 100 | 5600 | 1.6 |
| HR-17 | 100 | 4400 | 1.3 |
| HR-18 | 50/50 | 4300 | 1.3 |
| HR-19 | 50/50 | 6500 | 1.6 |
| HR-20 | 30/70 | 6500 | 1.5 |
| HR-21 | 50/50 | 6000 | 1.6 |
| HR-22 | 50/50 | 3000 | 1.2 |
| HR-23 | 50/50 | 5000 | 1.5 |
| HR-24 | 50/50 | 4500 | 1.4 |
| HR-25 | 30/70 | 5000 | 1.4 |
| HR-26 | 50/50 | 5500 | 1.6 |
| HR-27 | 50/50 | 3500 | 1.3 |
| HR-28 | 50/50 | 6200 | 1.4 |
| HR-29 | 50/50 | 6500 | 1.6 |
| HR-30 | 50/50 | 6500 | 1.6 |
| HR-31 | 50/50 | 4500 | 1.4 |
| HR-32 | 30/70 | 5000 | 1.6 |
| HR-33 | 30/30/40 | 6500 | 1.8 |
| HR-34 | 50/50 | 4000 | 1.3 |
| HR-35 | 50/50 | 6500 | 1.7 |
| HR-36 | 50/50 | 6000 | 1.5 |
| HR-37 | 50/50 | 5000 | 1.6 |
| HR-38 | 50/50 | 4000 | 1.4 |
| HR-39 | 20/80 | 6000 | 1.4 |
| HR-40 | 50/50 | 7000 | 1.4 |
| HR-41 | 50/50 | 6500 | 1.6 |
| HR-42 | 50/50 | 5200 | 1.6 |
| HR-43 | 50/50 | 6000 | 1.4 |
| HR-44 | 70/30 | 5500 | 1.6 |
| HR-45 | 50/20/30 | 4200 | 1.4 |
| HR-46 | 30/70 | 7500 | 1.6 |
| HR-47 | 40/58/2 | 4300 | 1.4 |
| HR-48 | 50/50 | 6800 | 1.6 |
| HR-49 | 100 | 6500 | 1.5 |
| HR-50 | 50/50 | 6600 | 1.6 |
| HR-51 | 30/20/50 | 6800 | 1.7 |
| HR-52 | 95/5 | 5900 | 1.6 |
| HR-53 | 40/30/30 | 4500 | 1.3 |
| HR-54 | 50/30/20 | 6500 | 1.8 |
| HR-55 | 30/40/30 | 7000 | 1.5 |
| HR-56 | 60/40 | 5500 | 1.7 |
| HR-57 | 40/40/20 | 4000 | 1.3 |
| HR-58 | 60/40 | 3800 | 1.4 |
| HR-59 | 80/20 | 7400 | 1.6 |
| HR-60 | 40/40/15/5 | 4800 | 1.5 |
| HR-61 | 60/40 | 5600 | 1.5 |
| HR-62 | 50/50 | 5900 | 2.1 |
| HR-63 | 80/20 | 7000 | 1.7 |
| HR-64 | 100 | 5500 | 1.8 |
| HR-65 | 50/50 | 9500 | 1.9 |

[10] Organic Solvent

The composition according to the present invention in its typical form further contains a specified organic solvent capable of dissolving the above components.

As useful organic solvents, there can be mentioned, for example, ethylene dichloride, cyclohexanone, cyclopentanone, 2-heptanone, γ-butyrolactone, methyl ethyl ketone, ethylene glycol monomethyl ether, ethylene glycol monoethyl ether, 2-methoxyethyl acetate, ethylene glycol monoethyl ether acetate, propylene glycol monomethyl ether, propylene glycol monomethyl ether acetate, toluene, ethyl acetate, methyl lactate, ethyl lactate, methyl methoxypropionate, ethyl ethoxypropionate, methyl pyruvate, ethyl pyruvate, propyl pyruvate, N,N-dimethylformamide, dimethyl sulfoxide, N-methylpyrrolidone and tetrahydrofuran.

The solvents having a ketone structure include a linear ketone solvent and a cycloketone solvent. Compounds having 5 to 8 carbon atoms in total are preferred from the viewpoint of high coatability.

As the linear ketone solvent, there can be mentioned, for example, 2-heptanone, methyl ethyl ketone or methyl isobutyl ketone. Of these, 2-heptanone is most preferred.

As the cycloketone solvent, there can be mentioned, for example, cyclopentanone, 3-methyl-2-cyclopentanone, cyclohexanone, 2-methylcyclohexanone, 2,6-dimethylcyclohexanone, cycloheptanone, cyclooctanone or isophorone. Of these, cyclohexanone and cycloheptanone are most preferred.

As the organic solvent, it is preferred to use either a single solvent having a ketone structure alone or a mixed solvent consisting of a solvent having a ketone structure and another solvent.

As another solvent to be mixed with the solvent having a ketone structure (joint solvent), there can be mentioned, for example, a propylene glycol monoalkyl ether carboxylate, an alkyl lactate, a propylene glycol monoalkyl ether, an alkyl alkoxypropionate or a lactone compound.

As the propylene glycol monoalkyl ether carboxylate, there can be mentioned, for example, propylene glycol monomethyl ether acetate, propylene glycol monomethyl ether propionate or propylene glycol monoethyl ether acetate.

As the alkyl lactate, there can be mentioned, for example, methyl lactate or ethyl lactate.

As the propylene glycol monoalkyl ether, there can be mentioned, for example, propylene glycol monomethyl ether or propylene glycol monoethyl ether.

As the alkyl alkoxypropionate, there can be mentioned, for example, methyl methoxypropionate, ethyl methoxypropionate, methyl ethoxypropionate or ethyl ethoxypropionate.

As the lactone compound, there can be mentioned, for example, γ-butyrolactone.

As preferred joint solvents, there can be mentioned a propylene glycol monoalkyl ether carboxylate, an alkyl lactate and a propylene glycol monoalkyl ether. A more preferred joint solvent is propylene glycol monomethyl ether acetate.

A solvent with a boiling point as high as 200° C. or above, such as ethylene carbonate or propylene carbonate, may be mixed into the solvent for use from the viewpoint of film thickness uniformity and development defect performance.

The amount of high-boiling-point solvent added, based on the total mass of solvents, is generally in the range of 0.1 to 15 mass %, preferably 0.5 to 10 mass % and more preferably 1 to 5 mass %.

In the present invention, typically, an actinic-ray- or radiation-sensitive resin composition is prepared using an organic solvent, preferably a mixed solvent consisting of two or more types of solvents.

The solid content of the composition is generally in the range of 1 to 25 mass %, preferably 2 to 20 mass % and more preferably 2.5 to 10 mass %. In particular, when the pattern formation is carried out using electron beams, EUV light or ArF light, it is preferred for the solid content to fall within the range of 2.5 to 20 mass %.

[11] Other Additives

The composition according to the present invention may further contain other additives, such as a dye, a plasticizer, a surfactant other than the above mentioned fluorized and/or siliconized surfactants, a photosensitizer, and a compound capable of increasing the solubility in a developer.

The compound capable of increasing the solubility in a developer (dissolution accelerating compound) is, for example, a low-molecular compound of 1000 or less molecular weight having two or more phenolic OH groups or one or more carboxyl groups. When a carboxyl group is contained, an alicyclic or aliphatic compound is preferred.

The amount of dissolution accelerating compound added, based on the mass of the resin, is preferably in the range of 2 to 50 mass %, more preferably 5 to 30 mass %. It is preferred for the amount to be up to 50 mass % from the viewpoint of suppression of any development residue and prevention of any pattern distortion at development.

The above phenolic compound of 1000 or less molecular weight can be easily synthesized by persons of ordinary skill in the art to which the present invention pertains while consulting the processes described in, for example, JP-A's 4-122938 and 2-28531, U.S. Pat. No. 4,916,210 and EP 219294.

As the carboxylated alicyclic or aliphatic compound, there can be mentioned, for example, a carboxylic acid derivative of steroid structure such as cholic acid, deoxycholic acid or lithocholic acid, an adamantanecarboxylic acid derivative, adamantanedicarboxylic acid, cyclohexanecarboxylic acid, cyclohexanedicarboxylic acid or the like. These are however nonlimiting.

Surfactants other than the above fluorinated and/or siliconized surfactants, there can be mentioned nonionic surfactants, such as a polyoxyethylene alkyl ether, a polyoxyethylene alkylallyl ether, a polyoxyethylene-polyoxypropylene block copolymer, a sorbitan aliphatic ester, a polyoxyethylene sorbitan aliphatic ester or the like. These surfactants may be added either individually or in combination.

[Method of Forming Pattern]

The method of forming a pattern using the composition according to the present invention will be described below.

The composition of the present invention is typically used in such a manner that the components are dissolved in a given organic solvent, preferably the above-mentioned mixed solvent, and applied onto a given support. For example, the composition is applied to a substrate (e.g., silicon, silicon/silicon dioxide coating, silicon nitride, a quartz with a Cr layer, TiN, WSi, BPSG, SOG, an organic antireflection film or the like) for use in the manufacturing of precision integrated circuit elements and imprint mold structures, etc., or a photomask blank [one provided with a light shielding layer (a material containing a transition metal silicide is used; as applicable transition metals, there can be mentioned molybdenum, tantalum, tungsten, titanium, chromium, hafnium, nickel, vanadium, zirconium, ruthenium, rhodium and the like) and provided with an antireflection film layer (there can be mentioned, for example, an oxide, nitride, oxynitride or carbide containing as a major component a transition metal selected from among molybdenum, chromium, tantalum, tungsten, titanium, hafnium, nickel, vanadium, zirconium, ruthenium, rhodium and the like; a transition metal silicide material containing any of these transition metals and silicon (Si); an oxide, nitride, oxynitride or carbide of the transition metal silicide material; or any of these transition metals)] by appropriate application means, such as a spinner or a coater. The thus applied composition is dried, thereby obtain an actinic-ray- or radiation-sensitive film (hereinafter also referred to as a photosensitive film). The drying temperature is preferably in the range of 60 to 150° C., more preferably 80 to 130° C. In advance, the support may be provided with an antireflection film known in the art.

Subsequently, the photosensitive film is exposed to actinic rays or radiation, preferably baked (heated), and developed. It is preferred for the baking temperature to range from 80 to 150° C., especially from 90 to 130° C. from the viewpoint of sensitivity and stability. Accordingly, a desirable pattern can be obtained.

As the actinic rays or radiation, there can be mentioned, for example, infrared radiation, visible light, ultraviolet radiation, far ultraviolet radiation, X-rays or electron beams. As the actinic rays or radiation, preferred use is made of one with, for example, 250 nm or less, especially 220 nm or less wavelength. As the actinic rays or radiation, there can be mentioned, for example, a KrF excimer laser (248 nm), an ArF excimer laser (193 nm), an F₂ excimer laser (157 nm), X-rays or electron beams. As especially preferred actinic rays or radiation, there can be mentioned an ArF excimer laser, an F₂ excimer laser, EUV (13 nm) or electron beams.

As especially preferred actinic rays or radiation, there can be mentioned electron beams, X-rays and EUV light. Especially excellent sensitivity and roughness characteristics can be attained when the composition of the present invention is exposed to such actinic rays or radiation. The inventors presume that the reason therefor is that the compounds of general formula (I) can generate an acid with an especially high efficiency when exposed to electron beams, X-rays or EUV light.

In the stage of irradiation with actinic rays or radiation, exposure (liquid immersion exposure) may be carried out after filling the interstice between the photosensitive film and a lens with a liquid of refractive index higher than that of air. This would realize an enhancement of resolving power. For the prevention of direct contact of the resist film with the liquid for liquid immersion, a film that is highly insoluble in the liquid for liquid immersion (hereinafter also referred to as a “top coat”) may be provided between the resist film formed by the composition of the present invention and the liquid for liquid immersion. As other means to prevent the contact between the resist film and the liquid for liquid immersion, a hydrophobic resin (HR) may be added to the composition. As the hydrophobic resin, in addition to those described above, the resins explained in paragraph 0172-0253 in US 2008/0305432 A1 can also be exemplified.

In the development step, an alkali developer is generally used.

As the alkali developer, use can be made of any of alkaline aqueous solutions containing, for example, an inorganic alkali compound such as sodium hydroxide, potassium hydroxide, sodium carbonate, sodium silicate, sodium metasilicate or aqueous ammonia; a primary amine such as ethylamine or n-propylamine; a secondary amine such as diethylamine or di-n-butylamine; a tertiary amine such as triethylamine or methyldiethylamine; an alcoholamine such as dimethylethanolamine or triethanolamine; a quaternary ammonium salt such as tetramethylammonium hydroxide or tetraethylammonium hydroxide; or a cycloamine such as pyrrole or piperidine.

Appropriate amounts of an alcohol and/or a surfactant may be added to the alkali developer.

The concentration of alkali developer is generally in the range of 0.1 to 20 mass %. The pH value of the alkali developer is generally in the range of 10.0 to 15.0.

With respect to the particulars of the process for fabricating an imprint mold using the composition according to the present invention, reference can be made to, for example, Japanese Patent No. 4109085, JP-A-2008-162101, “Fundamentals of nanoimprint and its technology development/application

deployment—technology of nanoimprint substrate and its latest technology deployment” edited by Yoshihiko Hirai, published by Frontier Publishing, etc.

### EXAMPLE

The present invention will be described in greater detail below by way of its examples. However, the gist of the present invention is in no way limited to these examples.

<Photoacid Generator>

Compounds 1 to 20 and comparative compounds 1 to 4 indicated in Table 3 below were provided as photoacid generators. Of these, compounds 1 to 20 are among the compounds of general formula (I) above. Table 3 also indicates the volumes, of corresponding generated acids. The volumes are those calculated by the method described above in section [1].

TABLE 3

| | | Volume of |
|---|---|---|
| | | generated |
| Compound | Structure | acid (Å³) |
| 1 | 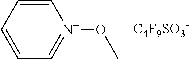 | 113 |
| 2 | 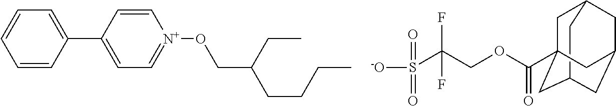 | 270 |
| 3 | 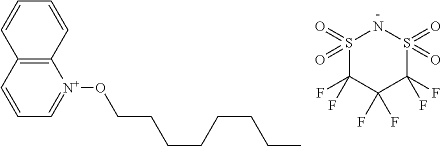 | 136 |
| 4 | 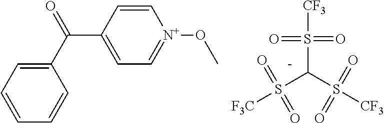 | 189 |
| 5 | 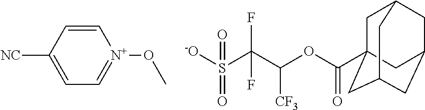 | 291 |
| 6 | 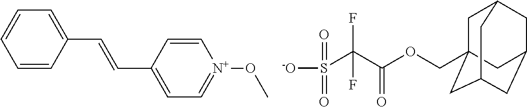 | 271 |
| 7 | 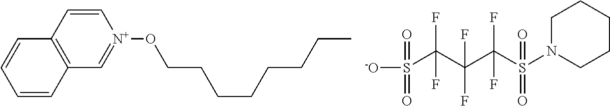 | 244 |
| 8 | 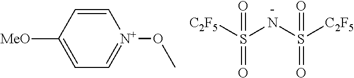 | 173 |
| 9 | 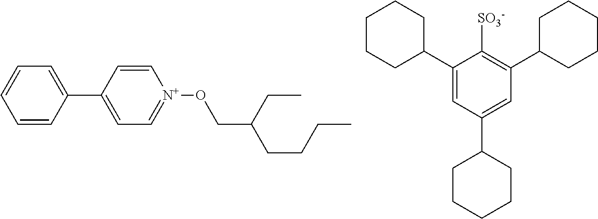 | 437 |
| 10 | 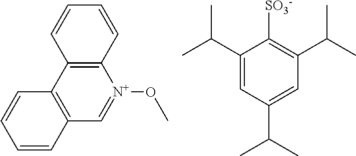 | 303 |
| 11 | 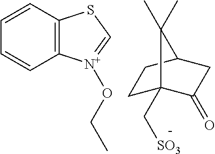 | 216 |
| 12 | 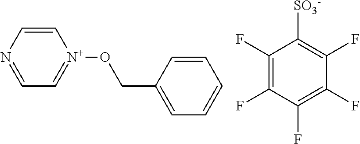 | 127 |
| 13 | 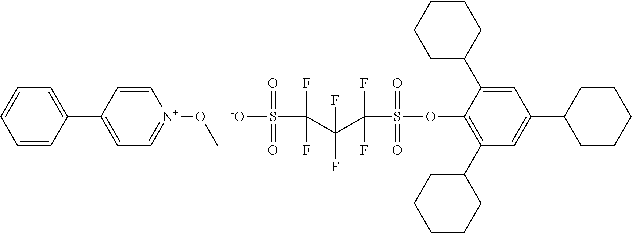 | 535 |
| 14 | 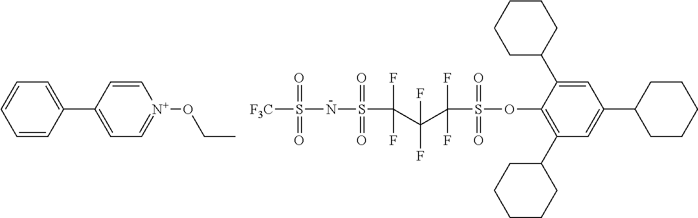 | 591 |
| 15 | 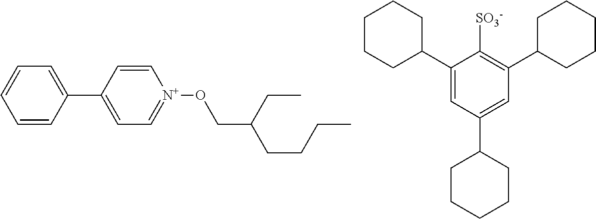 | 437 |
| 16 | 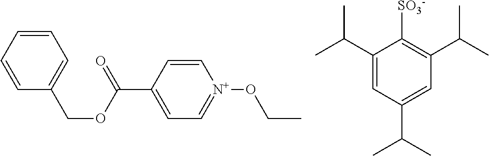 | 303 |
| 17 | 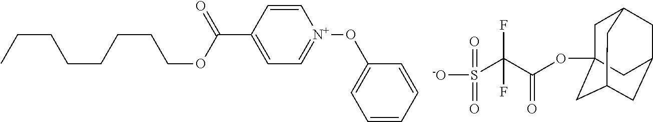 | 271 |
| 18 | 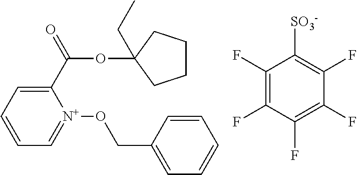 | 127 |
| 19 | 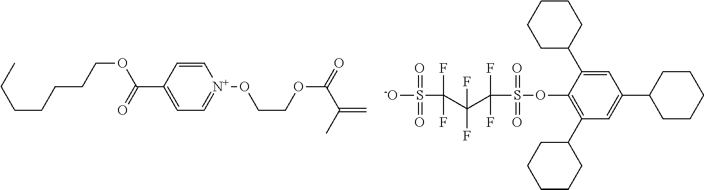 | 535 |
| 20 | 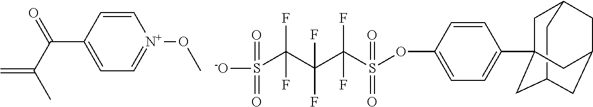 | 380 |
| Compar. 1 | 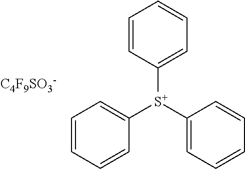 | 113 |
| Compar. 2 | 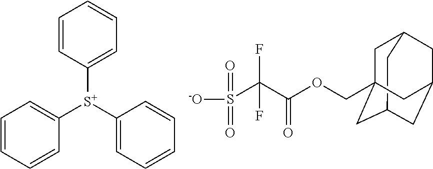 | 271 |
| Compar. 3 | 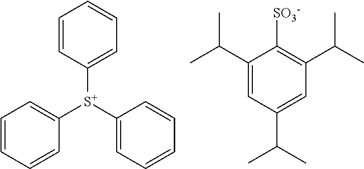 | 303 |
| Compar. 4 | 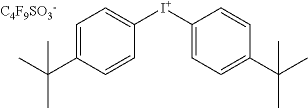 | 113 |
| Compar. 5 | 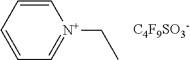 | 113 |

<Synthesis of Compound 1>

BF₄⁻ salt of the compound 1 was synthesized by the method described in J. AM. CHEM. SOC. 2004, 126, 14071-14078.

¹H NMR (300 MHz, CD₃CN): δ(ppm) 4.42 (s, 3H), 8.14 (t, 2H, J=7.2 Hz), 8.57 (t, 1H, J=7.5 Hz), 9.01 (dd, 2H, J=1.2, 7.5 Hz).

This was subject to a routine salt-exchange reaction to give the compound 1.

<Synthesis of Compound 2>

PF₆⁻ salt of the compound 2 was synthesized by the method described in J. AM. CHEM. SOC. 2002, 124, 15225-15238.

¹H NMR (400 MHz, CDCl₃): δ(ppm) 0.97-0.87 (m, 6H), 1.60-1.27 (m, 8H), 1.87-1.77 (m, 1H), 4.52 (t, 2H, J=5.8 Hz), 7.59-7.52 (m, 3H), 7.76 (d, 2H, J=8.5 Hz), 8.22 (d, 2H, J=7.2 Hz), 8.80 (d, 2H, J=7.2 Hz).

This was subject to a routine salt-exchange reaction to give the compound 2.

<Synthesis of Compound 3>

PF₆⁻ salt of the compound 3 was synthesized by the method described in J. AM. CHEM. SOC. 2002, 124, 15225-15238.

¹H NMR (400 MHz, CDCl₃): δ(ppm) 0.89 (t, 3H), 1.42-1.28 (m, 8H), 1.60-1.55 (m, 2H), 2.06-1.99 (m, 2H), 4.74 (t, 2H, J=6.4 Hz), 8.04-8.00 (m, 1H), 8.15 (dd, 1H, J=8.4, 6.3 Hz), 8.26-8.22 (m, 2H), 8.34 (dd, 1H, J=8.0, 3.2 Hz), 9.09 (d, 1H, J=8.4 Hz), 9.42 (d, 1H, J=7.2 Hz).

This was subject to a routine salt-exchange reaction to give the compound 3.

<Synthesis of Compound 4>

BF₄⁻ salt of the compound 4 was synthesized by the method described in J. AM. CHEM. SOC. 2002, 124, 15225-15238.

¹H NMR (300 MHz, CD₃CN): δ(ppm) 4.49 (s, 3H), 7.57-7.62 (m, 2H), 7.73-7.85 (m, 3H), 8.26 (dd, 2H, J=1.8, 5.1 Hz), 9.14 (dd, 2H, J=1.5, 4.8 Hz).

This was subject to a routine salt-exchange reaction to give the compound 4.

<Synthesis of Compound 5>

BF₄⁻ salt of the compound 5 was synthesized by the method described in J. AM. CHEM. SOC. 2002, 124, 15225-15238.

¹H NMR (300 MHz, DMSO-d₆): δ(ppm) 4.48 (s, 3H), 8.83 (d, 2H, J=6.6 Hz), 9.75 (d, 2H, J=6.6 Hz).

This was subject to a routine salt-exchange reaction to give the compound 5.

<Synthesis of Compound 6>

BF₄⁻ salt of the compound 6 was synthesized by the method described in J. AM. CHEM. SOC. 2002, 124, 15225-15238.

¹H NMR (300 MHz, CD₃CN): δ(ppm) 4.35 (s, 3H), 7.36 (d, 1H, J=16.5 Hz), 7.45-7.47 (m, 3H), 7.69-7.72 (m, 2H), 7.81 (d, 1H, J=16.5 Hz), 8.08 (d, 2H, J=7.2 Hz), 8.76 (d, 2H, J=7.2 Hz).

This was subject to a routine salt-exchange reaction to give the compound 6.

<Synthesis of Compound 7>

PF₆⁻ salt of the compound 7 was synthesized by the method described in J. AM. CHEM. SOC. 2002, 124, 15225-15238.

¹H NMR (300 MHz, CDCl₃): δ(ppm) 0.88 (t, 3H, J=16.5 Hz), 1.40-1.24 (m, 8H), 1.53-1.45 (m, 2H), 1.93-1.85 (m, 2H), 4.73 (t, 2H, J=6.8 Hz), 8.03-7.98 (m, 1H), 8.19-8.14 (m, 2H), 8.39 (d, 1H, J=6.8 Hz), 8.49 (dd, 2H, J=7.2, 2.4 Hz), 8.52 (d, 1H, J=8.8 Hz).

This was subject to a routine salt-exchange reaction to give the compound 7.

<Synthesis of Compound 8>

BF₄⁻ salt of the compound 8 was synthesized by the method described in J. AM. CHEM. SOC. 2002, 124, 15225-15238.

¹H NMR (300 MHz, CD₃CN): δ(ppm) 4.07 (s, 3H), 4.27 (s, 3H), 7.45 (d, 2H, J=7.8 Hz), 8.74 (d, 2H, J=7.8 Hz).

This was subject to a routine salt-exchange reaction to give the compound 8.

<Synthesis of Compound 9>

BF₄⁻ salt of the compound 9 was synthesized by the method described in J. AM. CHEM. SOC. 2004, 126, 14071-14078. This was subject to a routine salt-exchange reaction to give the compound 9.

¹H NMR (400 MHz, CDCl₃): δ(ppm) 2.10-1.80 (m, 45H), 2.50-2.20 (m, 2H), 4.40-4.20 (m, 2H), 4.62 (d, 2H, J=5.6 Hz), 7.00 (s, 2H), 7.58-7.50 (m, 3H), 7.84 (d, 2H, J=9.6 Hz, 8.51 (d, 2H, J=7.2 Hz), 9.39 (d, 2H, J=7.2 Hz).

<Synthesis of Compound 10>

BF₄⁻ salt of the compound 10 was synthesized by the method described in J. AM. CHEM. SOC. 2004, 126, 14071-14078.

¹H NMR (500 MHz, CD₃OD at 55° C.): δ(ppm) 4.67 (s, 3H), 8.08-8.20 (m, 3H), 8.38 (dt, 1H, J=1.5, 7.8 Hz), 8.58 (dt, 2H, J=1.0, 8.0 Hz), 9.02 (d, 1H, J=8.5 Hz), 9.07 (d, 1H, J=8.0 Hz), 10.46 (s, 1H).

This was subject to a routine salt-exchange reaction to give the compound 10.

<Synthesis of Compound 11>

BF₄⁻ salt of the compound 11 was synthesized by the method described in Chemical and Pharmaceutical Bulletin, 1970, vol. 18, p. 1176-1184. This was subject to a routine salt-exchange reaction to give the compound 11.

<Synthesis of Compound 12>

BF₄⁻ salt of the compound 12 was synthesized by the method described in J. AM. CHEM. SOC. 2002, 124, 15225-15238. This was subject to a routine salt-exchange reaction to give the compound 12.

### Example A

(Preparation of Resist)

Referring to Table 4 below, with respect to each of the resists, the individual components were dissolved in the solvent, thereby obtaining a solution of 4.0 mass % solid content. This solution was passed through a polytetrafluoroethylene filter of 0.1 μm pore size, thereby obtaining a positive resist solution.

(Evaluation of Resist)

An antireflection film DUV-42 produced by Brewer Science Inc. was uniformly applied at a thickness of 60 nm onto a silicon substrate having undergone a hexamethyldisilazane treatment by use of a spin coater, and dried on a hot plate at 100° C. for 90 seconds. Further drying was carried out by heating at 190° C. for 240 seconds. Thereafter, each of the positive resist solutions was applied thereonto by use of a spin coater and dried at 120° C. for 90 seconds, thereby obtaining a 0.12 μm-thick resist film.

The obtained resist film was exposed through a mask by means of an ArF excimer laser stepper (manufactured by ISI, NA=0.6). Immediately after the exposure, the resist film was baked on a hot plate at 120° C. for 90 seconds. Thereafter, the resist film was developed with a 2.38 mass % aqueous tetramethylammonium hydroxide solution at 23° C. for 60 seconds, rinsed with pure water for 30 seconds and dried. Thus, an intended line pattern was obtained.

[Sensitivity]

Surface exposure was carried out while changing the exposure amount by 0.5 mJ at a time within the range of 10 to 40 mJ/cm², and the exposed film was baked at 110° C. for 90 seconds. Thereafter, using a 2.38 mass % aqueous tetramethylammonium hydroxide (TMAH) solution, the dissolution rate in each of the exposure amounts was measured, thereby obtaining a dissolution rate curve.

The sensitivity was defined as the exposure amount in which the dissolution rate of the resist was saturated on the dissolution rate curve.

[Line Edge Roughness (LER)]

A 150 nm line pattern (L/S=1/1) was formed at the exposure amount realizing the above sensitivity. At arbitrary 30 points in a 50 μm region in the longitudinal direction thereof, the distance of actual edges from a reference line on which edges were to be present was measured by means of a scanning electron microscope (model S-9220, manufactured by Hitachi, Ltd.). The standard deviation of measured distances was determined, and 3σ was computed.

### [Configuration of Pattern]

The optimum exposure amount was defined as the exposure amount that reproduced a line-and-space (L/S=1/1) mask pattern of 150 nm line width. The profile at the optimum exposure amount was observed by means of a scanning electron microscope (SEM).

[Scum Defect]

A line pattern was formed in the above-mentioned manner. With respect to the line pattern, a cross-section micrograph was taken using SEM S4800 (manufactured by Hitachi High-Technologies Corporation). The presence of residue in inter-line spaces was checked, and the following evaluation was made.

x (poor): scum was observed, and there was partial pattern bridging.

Δ (relatively poor): scum was observed, but there was no pattern bridging.

o (good): scum was observed only partly, but there was no pattern bridging.

o* (excellent): no scum was observed at all.

The obtained measurement results are given in Table 4 below.

TABLE 4

| | | | | Surfactant | Solvent |
|---|---|---|---|---|---|
| | Acid | Resin | Basic | (0.1 | (mass |
| Ex. | generator | (9.6 g) | Compound | mass %) | ratio) |
| 1A | 1 (0.8 g) | RA-20 | B1 | W-1 | S1/S2 |
| | | | (0.02 g) | | (6/4) |
| 2A | 2 (0.8 g) | RA-20 | B2 | W-2 | S2/S1 |
| | | | (0.02 g) | | (6/4) |
| 3A | 3 (0.8 g) | RA-20 | B3 | W-3 | S2/S3 |
| | | | (0.02 g) | | (6/4) |
| 4A | 4 (0.8 g) | RA-20 | B1 | W-1 | S2/S1 |
| | | | (0.02 g) | | (6/4) |
| 5A | 5 (0.8 g) | RA-20 | B2 | W-4 | S2/S1 |
| | | | (0.02 g) | | (6/4) |
| 6A | 6 (0.8 g) | RA-20 | B3 | W-1 | S2/S4 |
| | | | (0.02 g) | | (6/4) |
| 7A | 7 (0.8 g) | RA-20 | B1 | W-1 | S2/S5 |
| | | | (0.02 g) | | (6/4) |
| 8A | 8 (0.8 g) | RA-20 | B2 | W-2 | S2/S6 |
| | | | (0.02 g) | | (6/4) |
| 9A | 1 (0.5 g) | RA-20 | B4 | W-3 | S2/S7 |
| | 2 (0.2 g) | | (0.02 g) | | (6/4) |
| 10A | 7 (0.5 g) | RA-20 | B1 | W-3 | S2/S1 |
| | 3 (0.2 g) | | (0.02 g) | | (6/4) |
| 11A | 6 (0.8 g) | RA-1 | B1 | W-3 | S2/S1 |
| | | | (0.02 g) | | (6/4) |
| 12A | 7 (0.8 g) | RA-23 | B1 | W-3 | S2/S1 |
| | | | (0.02 g) | | (6/4) |

| | Sensitivity | LER | Pattern | |
|---|---|---|---|---|
| Ex. | (mJ/cm²) | (nm) | configuration | Scum |
| 1A | 26.0 | 5.0 | Tapered | Δ |
| 2A | 24.1 | 4.1 | Rectangular | ◯* |
| 3A | 25.0 | 4.8 | Rectangular | ◯ |
| 4A | 24.0 | 4.8 | Rectangular | ◯ |
| 5A | 24.0 | 4.1 | Rectangular | ◯* |
| 6A | 24.2 | 4.1 | Rectangular | ◯* |
| 7A | 24.2 | 4.0 | Rectangular | ◯* |
| 8A | 24.0 | 4.8 | Rectangular | ◯ |
| 9A | 25.0 | 5.0 | Tapered | ◯ |
| 10A | 24.0 | 4.1 | Rectangular | ◯* |
| 11A | 24.1 | 4.1 | Rectangular | ◯* |
| 12A | 24.0 | 4.2 | Rectangular | ◯* |

| | | | | Surfactant | Solvent |
|---|---|---|---|---|---|
| | Acid | Resin | Basic | (0.1 | (mass |
| Ex. | generator | (9.6 g) | Compound | mass %) | ratio) |
| 13A | 3 (0.8 g) | RA-25 | B1 | W-3 | S2/S1 |
| | | | (0.02 g) | | (6/4) |
| 14A | 4 (0.8 g) | RA-26 | B4 | W-3 | S2/S1 |
| | | | (0.02 g) | | (6/4) |
| 15A | 5 (0.8 g) | RA-27 | B1 | W-3 | S2/S1 |
| | | | (0.02 g) | | (6/4) |
| 16A | 7 (0.8 g) | RA-28 | B2 | W-3 | S2/S1 |
| | | | (0.02 g) | | (6/4) |
| 17A | 7 (0.8 g) | RA-28 | B1 | W-3 | S2/S1 |
| | | | (0.02 g) | | (6/4) |
| 18A | 7 (0.8 g) | RA-28 | B1 | W-3 | S2/S1 |
| | | (4.6 g) | (0.02 g) | | (6/4) |
| | | RA-26 | | | |
| | | (5.0 g) | | | |
| 19A | 7 (0.8 g) | RA-28 | B1 | None | S2/S1 |
| | | | (0.02 g) | | (6/4) |
| 20A | 13 (0.8 g) | RA-20 | B1 | W-3 | S2/S1 |
| | | | (0.02 g) | | (6/4) |
| 21A | 16 (0.8 g) | RA-20 | B1 | W-3 | S2/S1 |
| | | | (0.02 g) | | (6/4) |
| Compar. 1A | Compar. | RA-20 | B1 | W-1 | S2/S1 |
| | 1 (0.8 g) | | (0.02 g) | | (6/4) |
| Compar. 2A | Compar. | RA-20 | B1 | W-1 | S2/S1 |
| | 2 (0.8 g) | | (0.02 g) | | (6/4) |
| Compar. 3A | Compar. | RA-20 | B1 | W-1 | S2/S1 |
| | 5 (0.8 g) | | (0.02 g) | | (6/4) |

| | Sensitivity | LER | Pattern | |
|---|---|---|---|---|
| Ex. | (mJ/cm²) | (nm) | configuration | Scum |
| 13A | 24.8 | 4.8 | Rectangular | ◯ |
| 14A | 24.9 | 4.8 | Rectangular | ◯ |
| 15A | 24.0 | 4.2 | Rectangular | ◯* |
| 16A | 24.2 | 4.1 | Rectangular | ◯* |
| 17A | 24.0 | 4.2 | Rectangular | ◯* |
| 18A | 24.1 | 4.3 | Rectangular | ◯* |
| 19A | 24.2 | 4.2 | Rectangular | ◯* |
| 20A | 24.0 | 4.3 | Rectangular | ◯* |
| 21A | 24.2 | 4.1 | Rectangular | ◯* |
| Compar. 1A | 30.0 | 5.7 | Tapered | X |
| Compar. 2A | 31.0 | 5.5 | Tapered | Δ |
| Compar. 3A | 51.0 | 5.6 | Tapered | X |

The acid-decomposable resin was selected from among the following resins (RA-1), (RA-20), (RA-23) and (RA-25) and used. In the following formulae, the numeral appearing on the right side of each repeating unit is a molar ratio. Mw represents a weight average molecular weight, and Mw/Mn represents a molecular weight dispersity.

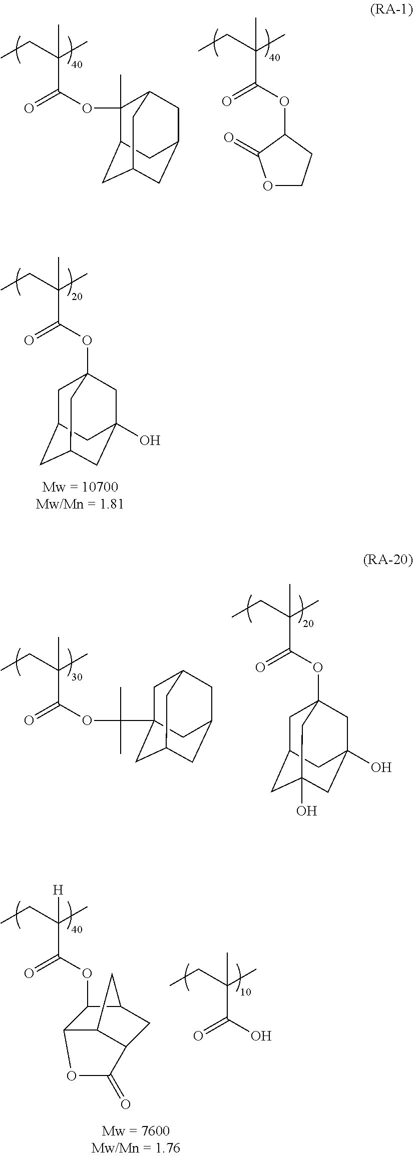

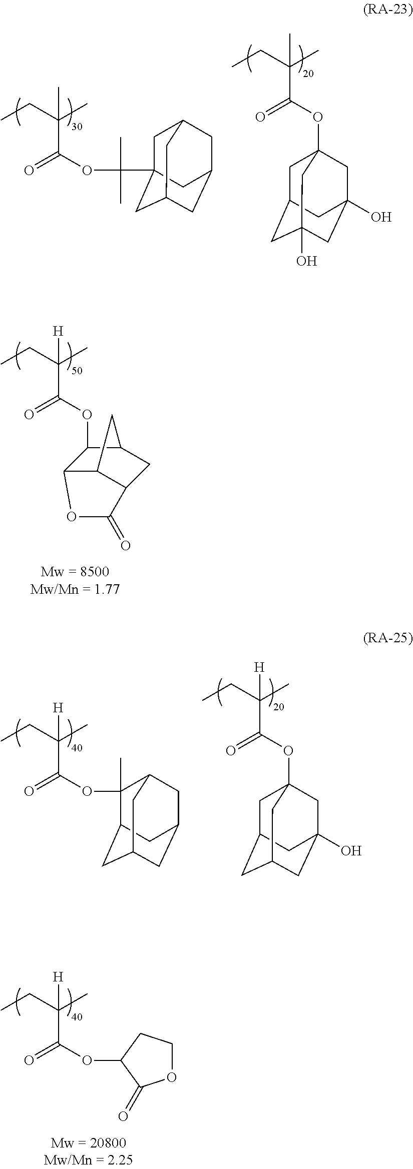

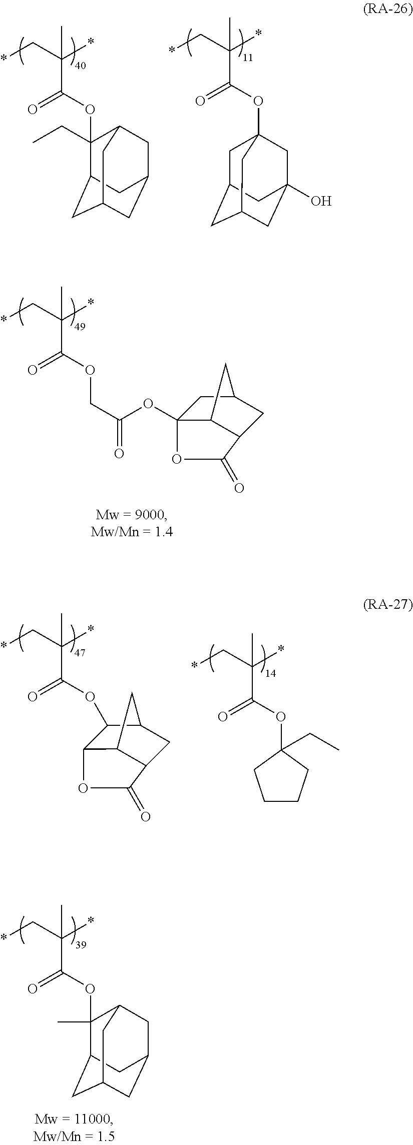

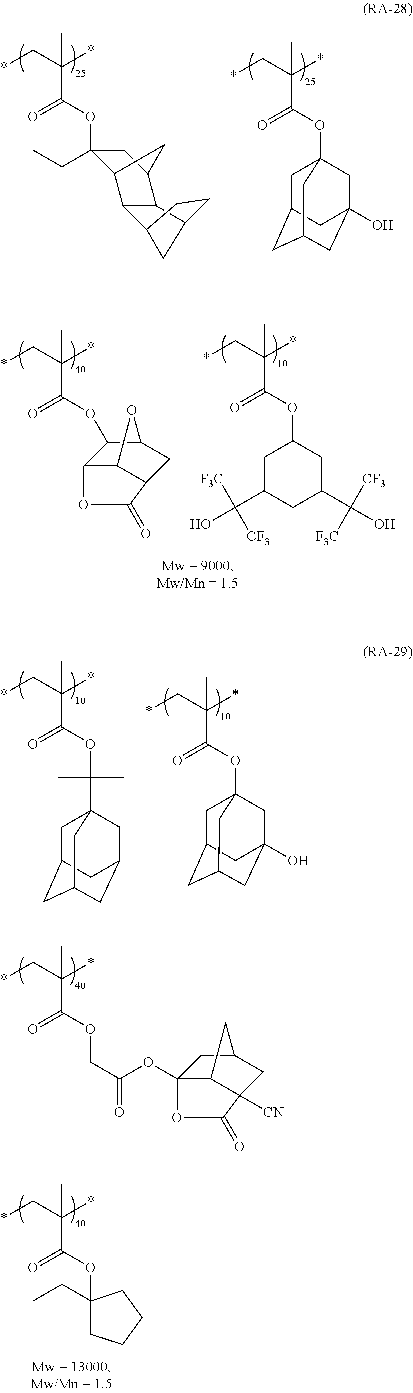

The basic compound, the surfactant and the solvent were appropriately selected from among those set forth hereinbelow.

[Basic Compound]

B1: tetrabutylammonium hydroxide

B2: tri(n-octyl)amine

B3: triphenylimidazole

B4:

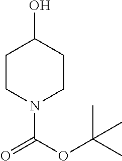

[Surfactant]

W-1: Megafac F176 (produced by Dainippon Ink & Chemicals, Inc.; fluorinated),

W-2: Megafac R08 (produced by Dainippon Ink & Chemicals, Inc.; fluorinated and siliconized),

W-3: polysiloxane polymer KP-341 (produced by Shin-Etsu Chemical Co., Ltd.; siliconized), and

W-4: PF6320 (produced by OMNOVA, Inc.; fluorinated).

[Solvent]

S1: propylene glycol monomethyl ether acetate (PGMEA; 1-methoxy-2-acetoxypropane),

S2: propylene glycol monomethyl ether (PGME; 1-methoxy-2-propanol),

S3: ethyl lactate,

S4: 2-heptanone,
S5: cyclohexanone,
S6: γ-butylolactone, and

S7: propylene carbonate.

As apparent from Table 4, the compositions of the present invention under ArF exposure exhibit excellent sensitivity, LER and pattern shape, being less in scum defects. Namely, it is apparent that the compositions of the present invention ensure excellent performance when used as positive resist compositions exposed to an ArF excimer laser.

### Example B1

A resist solution was prepared according to the same procedure as in Example A except that 0.06 g of polymer (1) shown below was added to the composition of Example 1A. The resist solution was applied in the same manner, thereby obtaining a resist film. The obtained resist film was patternwise exposed through an immersion liquid (pure water) by means of an ArF excimer laser liquid-immersion scanner (manufactured by ASML, XT1250i, NA 0.85), thereby accomplishing the same pattern formation as in Example A. With respect to the obtained pattern, it was ascertained that in all of the sensitivity, LER, pattern shape and scum defect performance, the same evaluation results as in Example A were obtained.

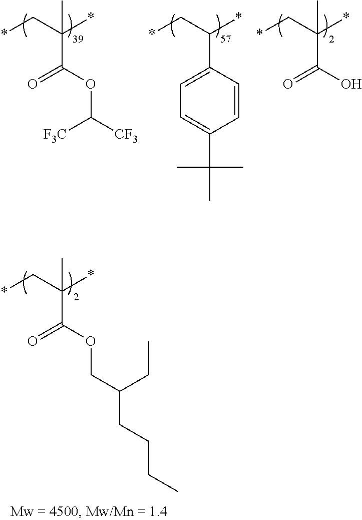

### Example B2

A resist solution was prepared according to the same procedure as in Example A except that 0.06 g of polymer (2) shown below was added to the composition of Example 1A. The resist solution was applied in the same manner, thereby obtaining a resist film. The obtained resist film was patternwise exposed through an immersion liquid (pure water) by means of an ArF excimer laser liquid-immersion scanner (manufactured by ASML, XT1250i, NA 0.85), thereby accomplishing the same pattern formation as in Example A. With respect to the obtained pattern, it was ascertained that in all of the sensitivity, LER, pattern shape and scum defect performance, the same evaluation results as in Example A were obtained.

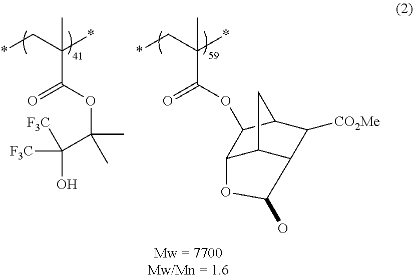

### Example B3

A resist solution was prepared according to the same procedure as in Example A except that 0.06 g of polymer (3) shown below was added to the composition of Example 1A. The resist solution was applied in the same manner, thereby obtaining a resist film. The obtained resist film was patternwise exposed through an immersion liquid (pure water) by means of an ArF excimer laser liquid-immersion scanner (manufactured by ASML, XT1250i, NA 0.85), thereby accomplishing the same pattern formation as in Example A. With respect to the obtained pattern, it was ascertained that in all of the sensitivity, LER, pattern shape and scum defect performance, the same evaluation results as in Example A were obtained.

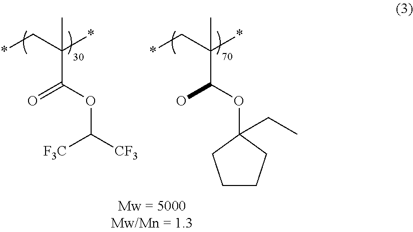

### Example C

(Preparation of Resist)

Referring to Table 5 below, with respect to each of the resists, the individual components were dissolved in the solvent and passed through a polytetrafluoroethylene filter of 0.1 μm pore size, thereby obtaining a positive resist solution of 4.5 mass % solid content.

(Evaluation of Resist)

Each of the obtained positive resist solutions was uniformly applied onto a silicon substrate having undergone a hexamethyldisilazane treatment by means of a spin coater, and heated and dried on a hot plate at 100° C. for 90 seconds, thereby obtaining a 0.4 μm resist film.

The obtained resist film was patternwise exposed through a line-and-space mask by means of a KrF excimer laser stepper (NA=0.63). Immediately after the exposure, the resist film was baked on a hot plate at 110° C. for 90 seconds. Thereafter, the resist film was developed with a 2.38 mass % aqueous tetramethylammonium hydroxide solution at 23° C. for 60 seconds, rinsed with pure water for 30 seconds and dried. Thus, an intended line pattern was obtained.

[Sensitivity]

The sensitivity was determined in the same manner as in Example A.

[Line Edge Roughness (LER)]

A 180 nm line pattern (L/S=1/1) was formed at the exposure amount realizing the above sensitivity. At arbitrary 30 points in a 50 μm region in the longitudinal direction thereof, the distance of actual edges from a reference line on which edges were to be present was measured by means of a scanning electron microscope (model S-9220, manufactured by Hitachi, Ltd.). The standard deviation of measured distances was determined, and 3σ was computed.

[Configuration of Pattern]

The optimum exposure amount was defined as the exposure amount that reproduced a line-and-space (L/S=1/1) mask pattern of 180 nm line width. The profile at the optimum exposure amount was observed by means of a scanning electron microscope (SEM).

[Scum Defect]

A line pattern was formed in the above-mentioned manner. With respect to the line pattern, a cross-section micrograph was taken using SEM S4800 (manufactured by Hitachi High-Technologies Corporation). The presence of residue in inter-line spaces was checked, and the following evaluation was made.

x (poor): scum was observed, and there was partial pattern bridging.

Δ (relatively poor): scum was observed, but there was no pattern bridging.

o (good): scum was observed only partly, but there was no pattern bridging.

o* (excellent): no scum was observed at all.

The obtained measurement results are given in Table 5 below.

TABLE 5

| | | | | Surfactant | Solvent |
|---|---|---|---|---|---|
| | Acid | Resin | Basic | (0.1 | (mass |
| Ex. | generator | (9.6 g) | Compound | mass %) | ratio) |
| 1C | 1 (0.6 g) | R-3 | B1 | W-1 | S1/S2 |
| | | | (0.02 g) | | (6/4) |
| 2C | 2 (0.6 g) | R-4 | B2 | W-2 | S2/S1 |
| | | | (0.02 g) | | (6/4) |
| 3C | 3 (0.6 g) | R-7 | B3 | W-3 | S2/S3 |
| | | | (0.02 g) | | (6/4) |
| 4C | 4 (0.6 g) | R-11 | B1 | W-1 | S2/S1 |
| | | | (0.02 g) | | (6/4) |
| 5C | 5 (0.6 g) | R-13 | B2 | W-4 | S2/S1 |
| | | | (0.02 g) | | (6/4) |
| 6C | 6 (0.6 g) | R-14 | B1 | W-1 | S1/S2 |
| | | | (0.02 g) | | (6/4) |
| 7C | 7 (0.6 g) | R-17 | B2 | W-2 | S2/S4 |
| | | | (0.02 g) | | (6/4) |
| 8C | 8 (0.6 g) | R-32 | B3 | W-3 | S2/S5 |
| | | | (0.02 g) | | (6/4) |
| 9C | 9 (0.6 g) | R-1 | B1 | W-1 | S2/S6 |
| | | | (0.02 g) | | (6/4) |
| 10C | 10 (0.6 g) | R-6 | B2 | W-4 | S2/S7 |
| | | | (0.02 g) | | (6/4) |
| 11C | 11 (0.6 g) | R-10 | B1 | W-4 | S2/S7 |
| | | | (0.02 g) | | (6/4) |
| 12C | 12 (0.6 g) | R-12 | B1 | W-4 | S2/S7 |
| | | | (0.02 g) | | (6/4) |

| | Sensitivity | LER | Pattern | |
|---|---|---|---|---|
| Ex. | (mJ/cm²) | (nm) | configuration | Scum |
| 1C | 18.0 | 5.0 | Tapered | Δ |
| 2C | 18.0 | 4.1 | Rectangular | ◯* |
| 3C | 20.0 | 4.5 | Rectangular | ◯ |
| 4C | 21.0 | 4.5 | Rectangular | ◯ |
| 5C | 21.0 | 4.2 | Rectangular | ◯* |
| 6C | 20.0 | 4.3 | Rectangular | ◯* |
| 7C | 20.0 | 4.0 | Rectangular | ◯* |
| 8C | 21.3 | 5.0 | Rectangular | ◯ |
| 9C | 20.0 | 4.1 | Rectangular | ◯* |
| 10C | 20.0 | 4.2 | Rectangular | ◯* |
| 11C | 21.0 | 4.8 | Rectangular | ◯* |
| 12C | 22.0 | 5.0 | Rectangular | ◯ |

| | | | | Surfactant | Solvent |
|---|---|---|---|---|---|
| | Acid | Resin | Basic | (0.1 | (mass |
| Ex. | generator | (9.6 g) | Compound | mass %) | ratio) |
| 13C | 1 (0.6 g) | R-18 | B1 | W-4 | S1/S2 |
| | | | (0.02 g) | | (6/4) |
| 14C | 7 (0.6 g) | R-29 | B1 | W-4 | S2/S1 |
| | | | (0.02 g) | | (6/4) |
| 15C | 5 (0.6 g) | R-22 | B1 | W-4 | S2/S3 |
| | | | (0.02 g) | | (6/4) |
| 16C | 11 (0.6 g) | R-27 | B1 | W-4 | S2/S1 |
| | | | (0.02 g) | | (6/4) |
| 17C | 9 (0.6 g) | R-30 | B1 | W-4 | S2/S1 |
| | | | (0.02 g) | | (6/4) |
| 18C | 9 (0.6 g) | R-28 | B1 | W-4 | S1/S2 |
| | | | (0.02 g) | | (6/4) |
| 19C | 6 (0.6 g) | R-31 | B2 | W-4 | S2/S4 |
| | | | (0.02 g) | | (6/4) |
| 20C | 7 (0.6 g) | RA-1 | B1 | W-4 | S2/S5 |
| | | | (0.02 g) | | (6/4) |
| 21C | 6 (0.3 g) | R-31 | B2 | W-4 | S2/S4 |
| | 7 (0.3 g) | | (0.02 g) | | (6/4) |
| 22C | 6 (0.6 g) | R-31 | B2 | W-4 | S2/S4 |
| | | (4.7 g) | (0.02 g) | | (6/4) |
| | | R-30 | | | |
| | | (5.0 g) | | | |
| 23C | 6 (0.6 g) | R-31 | B2 | None | S2/S4 |
| | | | (0.02 g) | | (6/4) |
| 24C | 13 (0.6 g) | R-27 | B1 | W-4 | S2/S1 |
| | | | (0.02 g) | | (6/4) |
| 25C | 14 (0.6 g) | R-35 | B2 | W-1 | S2/S1 |
| | | | (0.02 g) | | (6/4) |
| 26C | 18 (0.6 g) | R-40 | B1 | W-2 | S2/S1 |
| | | | (0.02 g) | | (6/4) |
| Compar. 1C | Compar. | R-1 | B1 | W-1 | S1/S2 |
| | 1 (0.6 g) | | (0.02 g) | | (6/4) |
| Compar. 2C | Compar. | R-1 | B1 | W-1 | S1/S2 |
| | 2 (0.6 g) | | (0.02 g) | | (6/4) |
| Compar. 3C | Compar. | R-1 | B1 | W-1 | S1/S2 |
| | 3 (0.6 g) | | (0.02 g) | | (6/4) |
| Compar. 4C | Compar. | R-1 | B1 | W-1 | S1/S2 |
| | 4 (0.6 g) | | (0.02 g) | | (6/4) |
| Compar. 5C | Compar. | R-1 | B1 | W-1 | S1/S2 |
| | 5 (0.6 g) | | (0.02 g) | | (6/4) |

| | Sensitivity | LER | Pattern | |
|---|---|---|---|---|
| Ex. | (mJ/cm²) | (nm) | configuration | Scum |
| 13C | 21.0 | 5.0 | Tapered | Δ |
| 14C | 22.0 | 4.2 | Rectangular | ◯* |
| 15C | 21.0 | 4.2 | Rectangular | ◯* |
| 16C | 21.0 | 4.8 | Rectangular | ◯* |
| 17C | 21.0 | 4.2 | Rectangular | ◯* |
| 18C | 22.0 | 4.2 | Rectangular | ◯* |
| 19C | 23.0 | 4.2 | Rectangular | ◯* |
| 20C | 24.0 | 5.2 | Rectangular | Δ |
| 21C | 23.2 | 4.2 | Rectangular | ◯* |
| 22C | 23.0 | 4.2 | Rectangular | ◯* |
| 23C | 23.0 | 4.2 | Rectangular | ◯* |
| 24C | 19.0 | 4.2 | Rectangular | ◯* |
| 25C | 21.0 | 4.1 | Rectangular | ◯* |
| 26C | 19.0 | 4.8 | Rectangular | ◯* |
| Compar. 1C | 25.5 | 6.5 | Tapered | X |
| Compar. 2C | 25.5 | 7.5 | Tapered | Δ |
| Compar. 3C | 26.0 | 7.2 | Tapered | Δ |
| Compar. 4C | 25.0 | 7.2 | Tapered | X |
| Compar. 5C | 50.0 | 7.3 | Tapered | X |

The basic compound, the surfactant and the solvent were appropriately selected from among those set forth above.

The acid-decomposable resin was appropriately selected from among the resins (R-1) to (R-43) and (RA-1) as set forth hereinbefore. The molar ratio, the weight average molecular weight (Mw), and the diversity (Mw/Mn) of the resin shown in Table 5 and the following tables are as shown in Table 6 below.

TABLE 6

| | Molar ratio | | |
|---|---|---|---|
| | (corresponding to | | |
| | individual repeating units | | |
| Resin | in order from the left) | Mw | Mw/Mn |
| R-2 | 60/20/20 | 12000 | 1.7 |
| R-10 | 70/30 | 11000 | 1.6 |
| R-14 | 15/60/25 | 12000 | 1.5 |
| R-17 | 80/20 | 15000 | 1.8 |
| R-18 | 65/35 | 9000 | 1.7 |
| R-18(H) | 60/40 | 10000 | 1.9 |
| R-18(L) | 60/40 | 4000 | 1.2 |
| R-22 | 70/30 | 10000 | 1.9 |
| R-23 | 65/35 | 11000 | 1.6 |
| R-27 | 50/40/10 | 12000 | 1.8 |
| R-32 | 25/25/40/10 | 10000 | 1.2 |
| R-33 | 40/11/49 | 9000 | 1.3 |
| R-34 | 47/14/39 | 13000 | 1.3 |
| R-35 | 35/45/20 | 12000 | 1.2 |
| R-36 | 40/60 | 11000 | 1.3 |
| R-37 | 30/35/20/15 | 14000 | 1.4 |
| R-38 | 30/30/20/20 | 12000 | 1.3 |
| R-39 | 65/30/5 | 13000 | 1.3 |
| R-40 | 50/45/5 | 12000 | 1.2 |
| R-41 | 50/45/5 | 13000 | 1.3 |
| R-42 | 50/45/5 | 14000 | 1.2 |
| R-43 | 10/40/50 | 4500 | 1.2 |

As apparent from Table 5, the compositions of the present invention under KrF exposure exhibit excellent sensitivity, LER and pattern shape, being less in scum defects. Namely, it is apparent that the compositions of the present invention ensure excellent performance when used as positive resist compositions exposed to an KrF excimer laser.

### Example D

(Preparation of Resist)

Referring to Table 7 below, with respect to each of the resists, the individual components were dissolved in the solvent and passed through a polytetrafluoroethylene filter of 0.1 μm pore size, thereby obtaining a positive resist solution of 4.0 mass % solid content.

(Evaluation of Resist)

Each of the prepared positive resist solutions was uniformly applied onto a silicon substrate laminated on its surface with Cr as a photomask blank model by means of a spin coater, and dried by heating on a hot plate at 100° C. for 60 seconds, thereby obtaining a 0.12 μm-thick resist film.

Each of the resist films was exposed to electron beams by means of an electron beam projection lithography system (acceleration voltage 100 KeV) manufactured by Nikon Corporation. Immediately after the exposure, the film was baked on a hot plate at 110° C. for 90 seconds. Thereafter, the baked film was developed with a 2.38 mass % aqueous tetramethylammonium hydroxide solution at 23° C. for 60 seconds. After the development, the film was rinsed with pure water for 30 seconds and dried. Thus, a line and space pattern was formed.

[Sensitivity]

Each of the obtained patterns was observed by means of a scanning electron microscope (model S-9220 manufactured by Hitachi, Ltd.). The sensitivity (Eo) was defined as the electron beam irradiation amount in which a line and space (L/S=1/1) of 100 nm line width was resolved.

[Line Edge Roughness (LER)]

A 100 nm line pattern (L/S=1/1) was formed at the exposure amount realizing the above sensitivity. At arbitrary 30 points in a 50 μm region in the longitudinal direction thereof, the distance of actual edges from a reference line on which edges were to be present was measured by means of a scanning electron microscope (model S-9220, manufactured by Hitachi, Ltd.). The standard deviation of measured distances was determined, and 3σ was computed.

[Configuration of Pattern]

The optimum exposure amount was defined as the exposure amount that reproduced a line-and-space (L/S=1/1) mask pattern of 50 nm line width. The profile at the optimum exposure amount was observed by means of a scanning electron microscope (SEM).

[Outgassing (Evaluation of Post-Exposure Film Thickness Change Ratio)]

Each of the resist films was exposed with the exposure amount equal to twice the exposure amount exhibiting the above sensitivity, and the film thickness after exposure (prior to post-baking) was measured. The ratio of change from the film thickness before exposure was calculated according to the following formula.

Ratio of film thickness change=100×[(film thickness before exposure−film thickness after exposure)/film thickness before exposure].

The less the ratio of film thickness change, the more favorable the outgassing reduction.

[Scum Defect]

A line pattern was formed in the above-mentioned manner. With respect to the line pattern, a cross-section micrograph was taken using an SEM S4800 (manufactured by Hitachi High-Technologies Corporation). The presence of residue in inter-line spaces was checked, and the following evaluation was made.

x (poor): scum was observed, and there was partial pattern bridging.

Δ (relatively poor): scum was observed, but there was no pattern bridging.

o (good): scum was observed only partly, but there was no pattern bridging.

o* (excellent): no scum was observed at all.

The obtained measurement results are given in Table 7 below.

TABLE 7

| | | | | Surfactant | Solvent |
|---|---|---|---|---|---|
| | Acid | Resin | Basic | (0.1 | (mass |
| Ex. | generator | (9.7 g) | Compound | mass %) | ratio) |
| 1D | 1 (0.6 g) | R-18(H) | B1 | W-1 | S1/S2 |
| | | | (0.02 g) | | (6/4) |
| 2D | 2 (0.6 g) | R-18(L) | B2 | W-2 | S2/S1 |
| | | | (0.02 g) | | (6/4) |
| 3D | 3 (0.6 g) | R-22 | B3 | W-3 | S2/S3 |
| | | | (0.02 g) | | (6/4) |
| 4D | 4 (0.6 g) | R-10 | B1 | W-1 | S2/S1 |
| | | | (0.02 g) | | (6/4) |
| 5D | 5 (0.6 g) | R-2 | B2 | W-4 | S2/S1 |
| | | | (0.02 g) | | (6/4) |
| 6D | 6 (0.6 g) | R-23 | B3 | W-1 | S2/S4 |
| | | | (0.02 g) | | (6/4) |
| 7D | 7 (0.6 g) | R-27 | B1 | W-1 | S2/S5 |
| | | | (0.02 g) | | (6/4) |
| 8D | 8 (0.6 g) | R-14 | B2 | W-2 | S2/S6 |
| | | | (0.02 g) | | (6/4) |
| 9D | 9 (0.6 g) | R-17 | B4 | W-3 | S2/S7 |
| | | | (0.02 g) | | (6/4) |
| 10D | 10 (0.5 g) | R-23 | B1 | W-3 | S2/S1 |
| | | | (0.02 g) | | (6/4) |
| 11D | 11 (0.6 g) | R-18(L) | B1 | W-1 | S2/S1 |
| | | | (0.02 g) | | (6/4) |
| 12D | 12 (0.6 g) | R-18(L) | B1 | W-1 | S2/S1 |
| | | | (0.02 g) | | (6/4) |
| 13D | 3 (0.3 g) | R-18(L) | B1 | W-1 | S2/S1 |
| | 7 (0.3 g) | | (0.02 g) | | (6/4) |
| 14D | 2 (0.6 g) | R-32 | B1 | W-1 | S2/S1 |
| | | | (0.02 g) | | (6/4) |
| 15D | 5 (0.6 g) | R-33 | B1 | W-1 | S2/S1 |
| | | | (0.02 g) | | (6/4) |
| 16D | 6 (0.6 g) | R-34 | B1 | W-1 | S2/S1 |
| | | | (0.02 g) | | (6/4) |

| | Sensitivity | Pattern | LER | Out- | |
|---|---|---|---|---|---|
| Ex. | (μC/cm²) | configuration | (nm) | gas (%) | Scum |
| 1D | 12.3 | Tapered | 5.0 | 1.5 | Δ |
| 2D | 12.5 | Rectangular | 4.2 | 2.0 | ◯* |
| 3D | 12.5 | Rectangular | 4.7 | 1.5 | ◯ |
| 4D | 12.0 | Rectangular | 4.7 | 1.5 | ◯ |
| 5D | 12.1 | Rectangular | 4.3 | 1.5 | ◯* |
| 6D | 12.2 | Rectangular | 4.2 | 2.0 | ◯* |
| 7D | 12.1 | Rectangular | 4.7 | 2.5 | ◯ |
| 8D | 12.3 | Rectangular | 4.0 | 2.5 | ◯* |
| 9D | 12.2 | Rectangular | 4.3 | 2.5 | ◯* |
| 10D | 12.3 | Rectangular | 4.2 | 1.5 | ◯* |
| 11D | 12.5 | Rectangular | 4.3 | 1.5 | ◯* |
| 12D | 12.2 | Rectangular | 4.7 | 1.5 | ◯ |
| 13D | 12.5 | Rectangular | 4.7 | 2.0 | ◯ |
| 14D | 12.5 | Rectangular | 4.2 | 2.5 | ◯* |
| 15D | 12.5 | Rectangular | 4.0 | 2.5 | ◯* |
| 16D | 12.5 | Rectangular | 4.2 | 1.5 | ◯* |

| | | | | Surfactant | Solvent |
|---|---|---|---|---|---|
| | Acid | Resin | Basic | (0.1 | (mass |
| Ex. | generator | (9.7 g) | Compound | mass %) | ratio) |
| 17D | 5 (0.6 g) | R-35 | B1 | W-1 | S2/S1 |
| | | | (0.02 g) | | (6/4) |
| 18D | 2 (0.6 g) | R-36 | B1 | W-1 | S2/S1 |
| | | | (0.02 g) | | (6/4) |
| 19D | 2 (0.6 g) | R-37 | B2 | W-2 | S2/S1 |
| | | | (0.02 g) | | (6/4) |
| 20D | 2 (0.6 g) | R-38 | B3 | W-1 | S2/S1 |
| | | | (0.02 g) | | (6/4) |
| 21D | 7 (0.6 g) | R-39 | B1 | W-3 | S2/S1 |
| | | | (0.02 g) | | (6/4) |
| 22D | 5 (0.6 g) | R-40 | B4 | W-1 | S2/S1 |
| | | | (0.02 g) | | (6/4) |
| 23D | 6 (0.6 g) | R-41 | B2 | W-1 | S2/S1 |
| | | | (0.02 g) | | (6/4) |
| 24D | 7 (0.6 g) | R-42 | B1 | W-4 | S2/S1 |
| | | | (0.02 g) | | (6/4) |
| 25D | 2 (0.6 g) | R-43 | B4 | W-1 | S2/S1 |
| | | | (0.02 g) | | (6/4) |
| 26D | 2 (0.6 g) | RA-1 | B1 | W-1 | S2/S1 |
| | | | (0.02 g) | | (6/4) |
| 27D | 2 (0.6 g) | R-43 | B4 | W-1 | S2/S1 |
| | | (4.7 g) | (0.02 g) | | (6/4) |
| | | R-42 | | | |
| | | (5.0 g) | | | |
| 28D | 2 (0.6 g) | R-43 | B4 | None | S2/S1 |
| | | | (0.02 g) | | (6/4) |
| 29D | 13 (0.6 g) | R-10 | B1 | W-1 | S2/S1 |
| | | | (0.02 g) | | (6/4) |
| 30D | 14 (0.6 g) | R-33 | B1 | W-3 | S2/S3 |
| | | | (0.02 g) | | (6/4) |
| 31D | 16 (0.6 g) | R-40 | B1 | W-1 | S2/S1 |
| | | | (0.02 g) | | (6/4) |
| 32D | 17 (0.6 g) | R-35 | B1 | W-1 | S2/S1 |
| | | | (0.02 g) | | (6/4) |
| 33D | 19 (0.6 g) | R-23 | B3 | W-1 | S2/S1 |
| | | | (0.02 g) | | (6/4) |

| | Sensitivity | Pattern | LER | Out- | |
|---|---|---|---|---|---|
| Ex. | (μC/cm²) | configuration | (nm) | gas (%) | Scum |
| 17D | 12.2 | Rectangular | 4.0 | 1.5 | ◯* |
| 18D | 12.5 | Rectangular | 4.3 | 1.5 | ◯* |
| 19D | 12.5 | Rectangular | 4.2 | 2.0 | ◯* |
| 20D | 12.5 | Rectangular | 4.3 | 2.5 | ◯* |
| 21D | 12.2 | Rectangular | 4.0 | 2.5 | ◯* |
| 22D | 12.5 | Rectangular | 4.3 | 2.3 | ◯* |
| 23D | 15.3 | Rectangular | 4.2 | 2.5 | ◯* |
| 24D | 12.5 | Rectangular | 4.3 | 2.5 | ◯* |
| 25D | 12.5 | Rectangular | 4.2 | 2.5 | ◯* |
| 26D | 15.5 | Rectangular | 5.0 | 1.5 | Δ |
| 27D | 12.3 | Rectangular | 4.3 | 2.5 | ◯* |
| 28D | 12.6 | Rectangular | 4.2 | 2.5 | ◯* |
| 29D | 12.3 | Rectangular | 4.1 | 1.5 | ◯ |
| 30D | 12.2 | Rectangular | 4.2 | 2.5 | ◯ |
| 31D | 12.2 | Rectangular | 4.5 | 2.5 | ◯ |
| 32D | 12.5 | Rectangular | 4.7 | 1.0 | ◯ |
| 33D | 12.6 | Rectangular | 4.8 | 1.0 | ◯ |

| | | | | Surfactant | Solvent |
|---|---|---|---|---|---|
| | Acid | Resin | Basic | (0.1 | (mass |
| Ex. | generator | (9.7 g) | Compound | mass %) | ratio) |
| Compar. | Compar. 1 | R-2 | B1 | W-1 | S2/S1 |
| 1D | (0.6 g) | | (0.02 g) | | (6/4) |
| Compar. | Compar. 2 | R-2 | B1 | W-1 | S2/S1 |
| 2D | (0.6 g) | | (0.02 g) | | (6/4) |
| Compar. | Compar. 3 | R-2 | B1 | W-1 | S2/S1 |
| 3D | (0.6 g) | | (0.02 g) | | (6/4) |
| Compar. | Compar. 4 | R-2 | B1 | W-1 | S2/S1 |
| 4D | (0.6 g) | | (0.02 g) | | (6/4) |
| Compar. | Compar. 5 | R-2 | B1 | W-1 | S2/S1 |
| 5D | (0.6 g) | | (0.02 g) | | (6/4) |

| | Sensitivity | Pattern | LER | Out- | |
|---|---|---|---|---|---|
| Ex. | (μC/cm²) | configuration | (nm) | gas (%) | Scum |
| Compar. | 19.8 | Tapered | 6.0 | 4.3 | X |
| 1D | | | | | |
| Compar. | 19.9 | Tapered | 6.0 | 4.2 | Δ |
| 2D | | | | | |
| Compar. | 19.1 | Tapered | 6.2 | 4.5 | Δ |
| 3D | | | | | |
| Compar. | 19.1 | Tapered | 6.2 | 4.3 | X |
| 4D | | | | | |
| Compar. | 30.1 | Tapered | 6.5 | 4.3 | X |
| 5D | | | | | |

It is apparent from the results of Table 7 that in the exposure to electron beams, the composition according to the present invention excels in the sensitivity, LER, pattern shape and outgas performance, being less in scum defects. That is, it is apparent that the composition according to the present invention can also exhibit excellent performance as a positive resist composition exposed to electron beams.

### Example E

(Preparation of Resist)

Referring to Table 8 below, with respect to each of the resists, the individual components were dissolved in the solvent and passed through a polytetrafluoroethylene filter of 0.1 μm pore size, thereby obtaining a negative resist solution of 12 mass % solid content.

(Evaluation of Resist)

Each of the prepared negative resist solutions was uniformly applied onto a silicon substrate laminated on its surface with Cr as a photomask blank model by means of a spin coater, and dried by heating on a hot plate at 120° C. for 60 seconds, thereby obtaining a 0.3 μm-thick resist film.

Each of the resist films was exposed to electron beams by means of an electron beam projection lithography system (acceleration voltage 100 KeV) manufactured by Nikon Corporation. Immediately after the exposure, the film was baked on a hot plate at 110° C. for 90 seconds. Thereafter, the baked film was developed with a 2.38 mass % aqueous tetramethylammonium hydroxide solution at 23° C. for 60 seconds. After the development, the film was rinsed with pure water for 30 seconds and dried. Thus, a line and space pattern was formed.

Evaluation was performed in the same way as set forth regarding to the Example D. The results are summarized in Table 8 below.

TABLE 8

| | | Alkali- | | Cross- | | |
|---|---|---|---|---|---|---|
| | | soluble | | linking | Surfactant | Solvent |
| | Acid | resin | Basic | agent | (0.1 | (mass |
| Ex. | generator | (9.7 g) | Compound | (3.0 g) | mass %) | ratio) |
| 1E | 1 | P-3 | B1 | CL-1 | W-1 | S1/S2 |
| | (0.6 g) | | (0.02 g) | | | (6/4) |
| 2E | 2 | P-2 | B1 | CL-1 | W-2 | S2/S1 |
| | (0.6 g) | | (0.02 g) | | | (6/4) |
| 3E | 3 | P-3 | B3 | CL-2 | W-3 | S2/S3 |
| | (0.6 g) | | (0.02 g) | | | (6/4) |
| 4E | 4 | P-2 | B1 | CL-1 | W-1 | S2/S1 |
| | (0.6 g) | | (0.02 g) | | | (6/4) |
| 5E | 10 | P-1 | B2 | CL-1 | W-3 | S2/S1 |
| | (0.6 g) | | (0.02 g) | | | (6/4) |
| 6E | 6 | P-3 | B4 | CL-2 | W-2 | S2/S4 |
| | (0.6 g) | | (0.02 g) | | | (6/4) |
| 7E | 12 | P-3 | B1 | CL-3 | W-4 | S2/S5 |
| | (0.6 g) | | (0.02 g) | | | (6/4) |
| Compar. | Compar. 1 | P-1 | B1 | CL-1 | W-1 | S2/S6 |
| 1E | (0.6 g) | | (0.02 g) | | | (6/4) |
| Compar. | Compar. 2 | P-1 | B1 | CL-1 | W-1 | S2/S7 |
| 2E | (0.6 g) | | (0.02 g) | | | (6/4) |
| Compar. | Compar. 3 | P-1 | B1 | CL-1 | W-1 | S2/S1 |
| 3E | (0.6 g) | | (0.02 g) | | | (6/4) |
| Compar. | Compar. 4 | P-1 | B1 | CL-1 | W-1 | S2/S1 |
| 4E | (0.6 g) | | (0.02 g) | | | (6/4) |
| Compar. | Compar. 5 | P-1 | B1 | CL-1 | W-1 | S2/S1 |
| 5E | (0.6 g) | | (0.02 g) | | | (6/4) |

| | Sensitivity | Pattern | LER | Outgas | |
|---|---|---|---|---|---|
| Ex. | (μC/cm²) | configuration | (nm) | (%) | Scum |
| 1E | 13.8 | Inverse | 5.0 | 1.8 | Δ |
| | | tapered | | | |
| 2E | 13.8 | Rectangular | 4.1 | 1.8 | ∘* |
| 3E | 13.7 | Rectangular | 4.9 | 1.5 | ∘* |
| 4E | 13.8 | Rectangular | 4.9 | 1.5 | ∘* |
| 5E | 13.9 | Rectangular | 4.2 | 1.7 | ∘* |
| 6E | 13.8 | Rectangular | 4.1 | 2.5 | ∘* |
| 7E | 13.8 | Rectangular | 5.0 | 2.5 | ∘* |
| Compar. | 20.8 | Inverse | 7.8 | 4.5 | x |
| 1E | | tapered | | | |
| Compar. | 22.8 | Inverse | 7.9 | 4.4 | x |
| 2E | | tapered | | | |
| Compar. | 19.8 | Inverse | 7.1 | 4.6 | x |
| 3E | | tapered | | | |
| Compar. | 19.8 | Inverse | 7.1 | 4.6 | x |
| 4E | | tapered | | | |
| Compar. | 26.8 | Inverse | 7.2 | 4.7 | x |
| 5E | | tapered | | | |

The structures, molecular weights and molecular weight distributions of employed alkali-soluble resins are shown below. Also, the structures of employed acid crosslinking agents are shown below.

| | | Mw | Mw/Mn |
|---|---|---|---|
| P-1 | 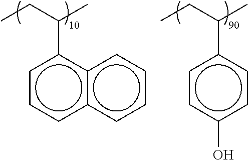 | 16000 | 2.30 |
| P-2 | 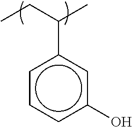 | 12000 | 1.2 |
| P-3 | 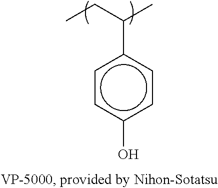 | 6000 | 1.2 |

| | |
|---|---|
| | CL-1 |
| 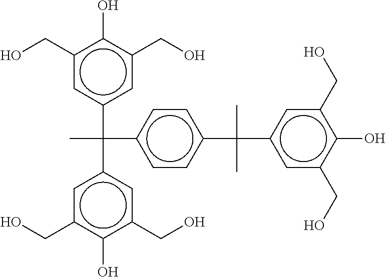 | |
| | CL-2 |
| 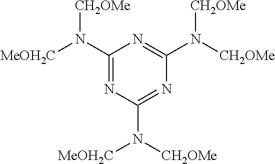 | |
| | CL-3 |
| 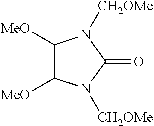 | |

It is apparent from the results of Table 8 that in the exposure to electron beams, the composition according to the present invention excels in the sensitivity, LER, pattern shape and outgas performance, being less in scum defects. That is, it is apparent that the composition according to the present invention can also exhibit excellent performance as a negative resist composition exposed to electron beams.

### Example F

(Preparation of Resist)

Referring to Table 9 below, with respect to each of the resists, the individual components were dissolved in the solvent and passed through a polytetrafluoroethylene filter of 0.1 μm pore size, thereby obtaining a positive resist solution of 8 mass % solid content.

(Evaluation of Resist)

Each of the prepared positive resist solutions was uniformly applied onto a silicon substrate having undergone a hexamethyldisilazane treatment by means of a spin coater, and heated and dried on a hot plate at 100° C. for 60 seconds, thereby obtaining a 0.12 μm-thick resist film.

[Sensitivity]

The surface exposure of each of the obtained resist films was carried out using EUV light (wavelength 13 nm) while changing the exposure amount by 0.5 mJ/cm² at a time within the range of 0 to 10.0 mJ/cm². The exposed film was baked at 110° C. for 90 seconds. Thereafter, using a 2.38 mass % aqueous tetramethylammonium hydroxide (TMAH) solution, the dissolution rate at each exposure amount was measured, thereby obtaining a dissolution rate curve.

The sensitivity was defined as the exposure amount in which the dissolution rate of the resist was saturated on the dissolution rate curve.

[Configuration of Pattern]

The optimum exposure amount was defined as the exposure amount that reproduced a line-and-space (L/S=1/1) mask pattern of 50 nm line width. The profile at the optimum exposure amount was observed by means of a scanning electron microscope (SEM).

[Line Edge Roughness (LER)]

A 50 nm line pattern (L/S=1/1) was formed at the exposure amount realizing the above sensitivity. At arbitrary 30 points in a 50 μm region in the longitudinal direction thereof, the distance of actual edges from a reference line on which edges were to be present was measured by means of a scanning electron microscope (model S-9220, manufactured by Hitachi, Ltd.). The standard deviation of measured distances was determined, and 3σ was computed.

[Outgassing (Evaluation of Post-Exposure Film Thickness Change Ratio)]

Each of the resist films was exposed with the exposure amount equal to twice the exposure amount exhibiting the above sensitivity, and the film thickness after exposure (prior to post-baking) was measured. The ratio of change from the film thickness before exposure was calculated according to the following formula.

Ratio of film thickness change=100×[(film thickness before exposure−film thickness after exposure)/film thickness before exposure].

The less the ratio of film thickness change, the more favorable the outgassing reduction.

[Scum Defect]

A line pattern was formed in the above-mentioned manner. With respect to the line pattern, a cross-section micrograph was taken using an SEM S4800 (manufactured by Hitachi High-Technologies Corporation). The presence of residue in inter-line spaces was checked, and the following evaluation was made.

x (poor): scum was observed, and there was partial pattern bridging.

Δ (relatively poor): scum was observed, but there was no pattern bridging.

o (good): scum was observed only partly, but there was no pattern bridging.

o* (excellent): no scum was observed at all.

The obtained measurement results are given in Table 9 below.

TABLE 9

| | | | | Surfactant | Solvent |
|---|---|---|---|---|---|
| | Acid | Resin | Basic | (0.1 | (mass |
| Ex. | generator | (9.7 g) | Compound | mass %) | ratio) |
| 1F | 1 (0.6 g) | R-18 (H) | B1 | W-1 | S1/S2 |
| | | | (0.02 g) | | (6/4) |
| 2F | 2 (0.6 g) | R-18 (L) | B2 | W-1 | S2/S1 |
| | | | (0.02 g) | | (6/4) |
| 3F | 3 (0.6 g) | R-22 | B3 | W-2 | S2/S3 |
| | | | (0.02 g) | | (6/4) |
| 4F | 4 (0.6 g) | R-10 | B1 | W-3 | S2/S1 |
| | | | (0.02 g) | | (6/4) |
| 5F | 5 (0.6 g) | R-2 | B2 | W-1 | S2/S1 |
| | | | (0.02 g) | | (6/4) |
| 6F | 6 (0.6 g) | R-23 | B3 | W-1 | S2/S4 |
| | | | (0.02 g) | | (6/4) |
| 7F | 7 (0.6 g) | R-27 | B1 | W-2 | S2/S5 |
| | | | (0.02 g) | | (6/4) |
| 8F | 8 (0.6 g) | R-14 | B2 | W-1 | S2/S6 |
| | | | (0.02 g) | | (6/4) |
| 9F | 9 (0.6 g) | R-17 | B1 | W-1 | S2/S7 |
| | | | (0.02 g) | | (6/4) |
| 10F | 10 (0.6 g) | R-23 | B1 | W-3 | S2/S1 |
| | | | (0.02 g) | | (6/4) |
| 11F | 11 (0.6 g) | R-32 | B4 | W-1 | S2/S1 |
| | | | (0.02 g) | | (6/4) |
| 12F | 12 (0.6 g) | R-33 | B2 | W-4 | S2/S4 |
| | | | (0.02 g) | | (6/4) |
| 13F | 3 (0.3 g) | R-34 | B4 | W-1 | S2/S5 |
| | 7 (0.3 g) | | (0.02 g) | | (6/4) |
| 14F | 2 (0.6 g) | R-35 | B1 | W-1 | S2/S5 |
| | | | (0.02 g) | | (6/4) |
| 15F | 7 (0.6 g) | R-37 | B1 | W-1 | S2/S5 |
| | | | (0.02 g) | | (6/4) |
| 16F | 7 (0.6 g) | RA-1 | B1 | W-1 | S2/S5 |
| | | | (0.02 g) | | (6/4) |
| 17F | 13 (0.6 g) | R-32 | B4 | W-1 | S2/S1 |
| | | | (0.02 g) | | (6/4) |
| 18F | 14 (0.6 g) | R-35 | B1 | W-1 | S2/S1 |
| | | | (0.02 g) | | (6/4) |
| 19F | 17 (0.6 g) | R-34 | B4 | W-1 | S2/S1 |
| | | | (0.02 g) | | (6/4) |
| 20F | 20 (0.6 g) | R-38 | B1 | W-1 | S2/S1 |
| | | | (0.02 g) | | (6/4) |

| | Sensitivity | Pattern | LER | Outgas | |
|---|---|---|---|---|---|
| Ex. | (mJ/cm²) | configuration | (nm) | (%) | Scum |
| 1F | 12.3 | Tapered | 5.9 | 1.2 | Δ |
| 2F | 12.5 | Rectangular | 5.0 | 2.0 | ◯* |
| 3F | 12.5 | Rectangular | 5.7 | 1.5 | ◯ |
| 4F | 12.0 | Rectangular | 5.7 | 1.3 | ◯ |
| 5F | 13.1 | Rectangular | 5.1 | 1.5 | ◯* |
| 6F | 12.2 | Rectangular | 5.1 | 2.0 | ◯* |
| 7F | 12.1 | Rectangular | 5.1 | 2.5 | ◯* |
| 8F | 12.3 | Rectangular | 5.7 | 2.5 | ◯ |
| 9F | 12.2 | Rectangular | 5.1 | 2.0 | ◯* |
| 10F | 12.3 | Rectangular | 5.1 | 1.4 | ◯* |
| 11F | 12.3 | Rectangular | 4.2 | 1.4 | ◯* |
| 12F | 13.3 | Rectangular | 4.9 | 1.4 | ◯ |
| 13F | 12.3 | Rectangular | 4.9 | 1.4 | ◯ |
| 14F | 12.3 | Rectangular | 4.5 | 1.4 | ◯* |
| 15F | 12.3 | Rectangular | 4.5 | 1.4 | ◯* |
| 16F | 15.3 | Rectangular | 4.5 | 1.4 | ◯* |
| 17F | 12.2 | Rectangular | 4.5 | 1.8 | ◯* |
| 18F | 12.3 | Rectangular | 4.7 | 1.9 | ◯* |
| 19F | 12.1 | Rectangular | 4.2 | 1.7 | ◯* |
| 20F | 12.2 | Rectangular | 4.8 | 1.5 | ◯* |

| | | | | Surfactant | Solvent |
|---|---|---|---|---|---|
| | Acid | Resin | Basic | (0.1 | (mass |
| Ex. | generator | (9.7 g) | Compound | mass %) | ratio) |
| Compar. | Compar. 1 | R-2 | B1 | W-1 | S2/S1 |
| 1F | (0.6 g) | | (0.02 g) | | (6/4) |
| Compar. | Compar. 2 | R-2 | B1 | W-1 | S2/S1 |
| 2F | (0.6 g) | | (0.02 g) | | (6/4) |
| Compar. | Compar. 3 | R-2 | B1 | W-1 | S2/S1 |
| 3F | (0.6 g) | | (0.02 g) | | (6/4) |
| Compar. | Compar. 4 | R-2 | B1 | W-1 | S2/S1 |
| 4F | (0.6 g) | | (0.02 g) | | (6/4) |
| Compar. | Compar. 5 | R-2 | B1 | W-1 | S2/S1 |
| 5F | (0.6 g) | | (0.02 g) | | (6/4) |

| | Sensitivity | Pattern | LER | Outgas | |
|---|---|---|---|---|---|
| Ex. | (mJ/cm²) | configuration | (nm) | (%) | Scum |
| Compar. | 19.0 | Tapered | 7.0 | 4.5 | X |
| 1F | | | | | |
| Compar. | 18.0 | Tapered | 7.0 | 4.4 | Δ |
| 2F | | | | | |
| Compar. | 19.0 | Tapered | 6.5 | 4.5 | Δ |
| 3F | | | | | |
| Compar. | 19.0 | Tapered | 6.5 | 4.5 | X |
| 4F | | | | | |
| Compar. | 30.0 | Tapered | 6.8 | 4.5 | X |
| 5F | | | | | |

It is apparent from the results of Table 9 that in the exposure to EUV light, the composition according to the present invention excels in the sensitivity, LER, pattern shape and outgas performance, being less in scum defects. That is, it is apparent that the composition according to the present invention can also exhibit excellent performance as a positive resist composition exposed to EUV light.

### Example G

(Preparation of Resist)

Referring to Table 10 below, with respect to each of the resists, the individual components were dissolved in the solvent and passed through a polytetrafluoroethylene filter of 0.1 μm pore size, thereby obtaining a negative resist solution of 8 mass % solid content. The negative resist solution was evaluated in the following manner.

(Evaluation of Resist)

Each of the prepared negative resist solutions was uniformly applied onto a silicon substrate having undergone a hexamethyldisilazane treatment by means of a spin coater, and heated and dried on a hot plate at 120° C. for 60 seconds, thereby obtaining a 0.15 μm-thick resist film.

The resist films were evaluated in the same manner as in Example F. The results are given in Table 10 below.

TABLE 10

| | | Alkali- | Cross- | | | |
|---|---|---|---|---|---|---|
| | | soluble | linking | | Surfactant | Solvent |
| | Acid | resin | agent | Basic | (0.1 | (mass |
| Ex. | generator | (9.7 g) | (3.0 g) | Compound | mass %) | ratio) |
| 1G | 1 | P-3 | CL-1 | B1 | W-1 | S1/S2 |
| | (0.6 g) | | | (0.02 g) | | (6/4) |
| 2G | 2 | P-3 | CL-1 | B2 | W-2 | S2/S1 |
| | (0.6 g) | | | (0.02 g) | | (6/4) |
| 3G | 3 | P-3 | CL-1 | B3 | W-3 | S2/S3 |
| | (0.6 g) | | | (0.02 g) | | (6/4) |
| 4G | 4 | P-3 | CL-1 | B1 | W-2 | S2/S1 |
| | (0.6 g) | | | (0.02 g) | | (6/4) |
| 5G | 8 | P-3 | CL-1 | B4 | W-4 | S2/S1 |
| | (0.6 g) | | | (0.02 g) | | (6/4) |
| 6G | 6 | P-3 | CL-1 | B3 | W-1 | S2/S4 |
| | (0.6 g) | | | (0.02 g) | | (6/4) |
| 7G | 7 | P-3 | CL-1 | B1 | W-2 | S2/S5 |
| | (0.6 g) | | | (0.02 g) | | (6/4) |
| Compar. | Compar. 1 | P-1 | None | B2 | W-1 | S2/S6 |
| 1G | (0.6 g) | | | (0.02 g) | | (6/4) |
| Compar. | Compar. 2 | P-1 | CL-1 | B1 | W-1 | S2/S7 |
| 2G | (0.6 g) | | | (0.02 g) | | (6/4) |
| Compar. | Compar. 3 | P-1 | CL-1 | B1 | W-1 | S2/S1 |
| 3G | (0.6 g) | | | (0.02 g) | | (6/4) |
| Compar. | Compar. 4 | P-1 | CL-1 | B1 | W-1 | S2/S1 |
| 4G | (0.6 g) | | | (0.02 g) | | (6/4) |
| Compar. | Compar. 5 | P-1 | CL-1 | B1 | W-1 | S2/S1 |
| 5G | (0.6 g) | | | (0.02 g) | | (6/4) |

| | Sensitivity | Pattern | LER | Outgas | |
|---|---|---|---|---|---|
| Ex. | (mJ/cm²) | configuration | (nm) | (%) | Scum |
| 1G | 10.3 | Inverse | 7.0 | 1.4 | Δ |
| | | tapered | | | |
| 2G | 10.8 | Rectangular | 7.0 | 2.0 | ∘* |
| 3G | 10.0 | Rectangular | 7.0 | 2.0 | ∘ |
| 4G | 12.3 | Rectangular | 7.5 | 2.0 | ∘ |
| 5G | 10.3 | Rectangular | 7.2 | 2.0 | ∘ |
| 6G | 10.3 | Rectangular | 6.5 | 2.0 | ∘* |
| 7G | 12.5 | Rectangular | 6.5 | 2.5 | ∘* |
| Compar. | 18.0 | Inverse | 8.1 | 4.0 | x |
| 1G | | tapered | | | |
| Compar. | 19.5 | Inverse | 8.3 | 4.5 | Δ |
| 2G | | tapered | | | |
| Compar. | 19.0 | Inverse | 8.1 | 4.3 | Δ |
| 3G | | tapered | | | |
| Compar. | 19.0 | Inverse | 8.2 | 4.5 | x |
| 4G | | tapered | | | |
| Compar. | 30.0 | Inverse | 8.4 | 4.5 | x |
| 5G | | tapered | | | |

It is apparent from the results of Table 10 that in the exposure to EUV light, the composition according to the present invention excels in the sensitivity, LER, pattern shape and outgas performance, being less in scum defects. That is, it is apparent that the composition according to the present invention can also exhibit excellent performance as a negative resist composition exposed to EUV light.

What is claimed is:

1. A resist composition for semiconductor comprising any of compounds of general formula (I) below: 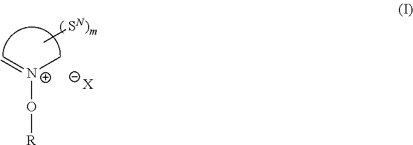
wherein
R represents a monovalent substituent,
the moiety: 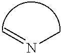
represents a monocyclic or polycyclic heterocycle containing a nitrogen atom,
S^{N} represents a substituent,
m is an integer of 0 or greater, and
X⁻ represents an anion.

2. The composition according to claim 1, wherein the anion, represented by X⁻ is selected from the group consisting of a sulfonate anion, an imidate anion and a methide anion.

3. The composition according to claim 1, wherein each of the compounds of general formula (I) above is a compound that is configured to decompose when exposed to actinic rays or radiation to thereby generate an acid expressed by general formula HX, the acid having a volume of 130 Å³ or greater.

4. The composition according to claim 1, wherein the anion represented by X⁻ is expressed by general formula (SA1) or (SA2) below: 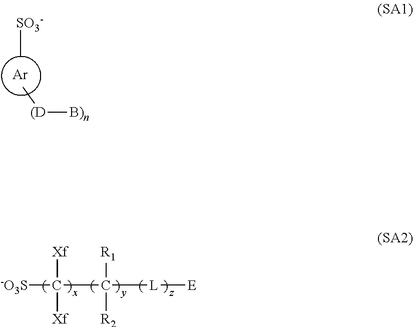
in formula (SA1),
Ar represents an aryl group, in which a substituent other than -(D-B) groups may further be introduced,
n is an integer of 1 or greater,
D represents a single bond or a bivalent connecting group, and
B represents a monovalent substituent, and
in formula (SA2),
each of Xf's independently represents a fluorine atom or an alkyl group having at least one hydrogen atom thereof replaced by a fluorine atom,
each of R₁ and R₂ independently represents a group selected from the group consisting of a hydrogen atom, a fluorine atom, an alkyl group and an alkyl group having at least one hydrogen atom thereof replaced by a fluorine atom,
L, or each of L's independently, represents a single bond or a bivalent connecting group,
E represents a group with a cyclic structure, and
x is an integer of 1 to 20, y an integer of 0 to 10, and z an integer of 0 to 10.

5. The composition according to claim 1, wherein the heterocycle is an aromatic ring.

6. The composition according to claim 1, further comprising a resin that is configured to decompose when acted on by an acid to thereby increase its solubility in an alkali developer.

7. The composition according to claim 6, wherein the resin contains any of repeating units of general formula (VI) below: 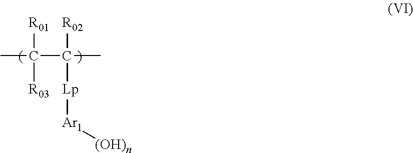
wherein
each of R₀₁, R₀₂ and R₀₃ independently represents a hydrogen atom, an alkyl group, a cycloalkyl group, a halogen atom, a cyano group or an alkoxycarbonyl group, provided that R₀₃ may represent an alkylene group and may be bonded to Lp or Ar₁ to thereby form a ring,
Ar₁ represents an aromatic ring group,
n is an integer of 1 or greater, and
Lp represents a single bond or a bivalent connecting group.

8. The composition according to claim 1, further comprising a resin that is soluble in an alkali developer and an acid crosslinking agent that is configured to crosslink with the resin when acted on by an acid.

9. The composition according to claim 8, wherein the resin contains any of repeating units of general formula (VI) below: 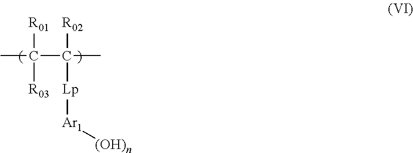
wherein
each of R₀₁, R₀₂ and R₀₃ independently represents a hydrogen atom, an alkyl group, a cycloalkyl group, a halogen atom, a cyano group or an alkoxycarbonyl group, provided that R₀₃ may represent an alkylene group and may be bonded to Lp or Ar₁ to thereby form a ring,
Ar₁ represents an aromatic ring group,
n is an integer of 1 or greater, and
Lp represents a single bond or a bivalent connecting group.

10. The composition according to claim 1, further comprising a basic compound.

11. The composition according to claim 1, which is to be exposed to electron beams, X-rays or EUV light.

12. A resist film formed from the composition according to claim 1.

13. A method of forming a pattern, comprising:
forming the composition according to claim 1 into a film,
exposing the film to light, and
developing the exposed film.

14. The method according to claim 13, wherein the exposure is performed using electron beams, X-rays or EUV light.
